(19) United States
(12) Patent Application Publication
(10) Pub. No.: US 2024/0052312 A1
(43) Pub. Date: Feb. 15, 2024

(54) COMPOSITION FOR REPROGRAMMING CELLS INTO PLASMACYTOID DENDRITIC CELLS OR INTERFERON TYPE I-PRODUCING CELLS, METHODS AND USES THEREOF

(71) Applicant: Asgard Therapeutics AB, Lund (SE)

(72) Inventors: Carlos Filipe Ribeiro Lemos Pereira, Caminha (PT); Cristiana Ferreira Pires, Palhaça (PT); Fábio Fiúza Rosa, Leiria (PT); Abigail Altman, Malmö (SE)

(21) Appl. No.: 17/766,369

(22) PCT Filed: Oct. 9, 2020
(86) PCT No.: PCT/EP2020/078429

§ 371 Date: Apr. 4, 2022

(30) Foreign Application Priority Data
Oct. 10, 2019 (PT) 115833
Jan. 10, 2020 (EP) 20151310.8

(51) Int. Cl. C12N 5/0784; C12N 15/86

(52) U.S. Cl. CPC C12N 5/0639; C12N 15/86; C12N 2501/60; C12N 2506/00

(57) ABSTRACT

The present disclosure relates to compositions, constructs and vectors for reprogramming cells into plasmacytoid dendritic cells or interferon type I-producing cells, methods and uses thereof. The present disclosure relates to the development of methods for making plasmacytoid dendritic cells or interferon type I-producing cells that promote antiviral and anti-tumoral immune responses from differentiated, multipotent or pluripotent stem cells by introducing and expressing isolated/synthetic transcription factors. More particularly, the disclosure provides methods for obtain plasmacytoid dendritic cells or interferon type I-producing cells by direct cellular reprogramming with the surprisingly use of combinations of specific transcription factors.

### TECHNICAL FIELD

The present disclosure relates to compositions for reprogramming cells into plasmacytoid dendritic cells or interferon type I-producing cells, methods and uses thereof.

The present disclosure relates to the development of methods for making plasmacytoid dendritic cells or interferon type I-producing cells that promote immune antiviral and anti-tumoral responses from differentiated, multipotent or pluripotent stem cells by introducing and expressing isolated/synthetic transcription factors. More particularly, the disclosure provides methods for obtain plasmacytoid dendritic cells or interferon type I-producing cells by direct cellular reprogramming with the surprisingly beneficial use of combinations of specific transcription factors.

### BACKGROUND

Cellular reprogramming relies on rewiring the epigenetic and transcriptional network of one cell state to that of a different cell type. Transcription factor (TF)-transduction experiments have highlighted the plasticity of adult somatic or differentiated cells, providing new technologies to generate any desired cell type. Through forced expression of TFs, it is possible to reprogram somatic or differentiated cells into induced pluripotent stem cells (iPSCs) that are remarkably similar to embryonic stem cells (Takahashi et al., 2006; Takahashi et al., 2007). Alternatively, a somatic cell can also be converted into another specialized cell type (Pereira et al., 2012). Direct lineage conversion has proven successful to reprogram mouse and human fibroblasts into several cell types, such as neurons, cardiomyocytes and hepatocytes, using TFs specifying the target-cell identity (Xu et al., 2015). Lineage conversions were also demonstrated in the hematopoietic system, where forced expression of TFs induced a macrophage fate in B cells and fibroblasts (Xie et al., 2004) and the direct reprogramming of mouse fibroblasts into clonogenic hematopoietic progenitors was achieved with the transcription factors Gata2, Gfi1b, cFos and Etv6 (Pereira et al., 2013). These four TFs induce a dynamic, multi-stage hemogenic process that progresses through an endothelial-like intermediate, recapitulating developmental hematopoiesis in vitro (Pereira et al., 2016).

Reprogrammed cells are very promising therapeutic tools for regenerative medicine, and cells obtained by differentiation of iPSCs are already being tested in clinical studies.

Cellular reprogramming strategies have highlighted the flexibility of cell fates with the possibility to use cell type-specific TFs to convert somatic cells into pluripotency. Direct lineage conversion of one differentiated cell type into another has also been demonstrated and explored to elucidate cell biology mechanisms and for regenerative medicine purposes. Recently, we have demonstrated that antigen presenting dendritic cells (DCs) can be reprogrammed from unrelated cell types by a small combination of TFs (Rosa et al., 2018). Classically, it is thought that a myeloid DC-committed progenitor gives rise to functionally different DC subsets: conventional DCs (cDCs) and plasmacytoid DCs (pDCs). cDCs are professional antigen presenting cells (APC) driving antigen-specific immune responses, while pDCs are professional producers of type I interferon during viral infection. They exhibit both innate and adaptive immune functions and can act as APCs impacting T cell responses directly. The timing and exact mechanisms regulating the divergence of the different subsets during DC development is still to be established.

Document EP 3 385 373 relates to compositions, nucleic acid constructs, methods and kits thereof for cell induction or reprogramming cells to conventional dendritic cell state or antigen presenting cell state, based, in part, on the surprising effect described in this document in of novel use of combinations of transcription factors that permit induction or reprogramming of differentiated or undifferentiated cells into DCs or APCs.

The combination of transcription factors described in document EP 3 385 373 induces DCs with surface phenotype, transcriptional signature and functional properties characteristic of the cDC1 subset of DCs. The ability to secrete interferon type I, e.g. interferon alpha (IFN-α) and beta (IFN-β) was not previously described.

The induced pDCs or interferon type I-producing cells described herein recapitulate typical pDC functional property of detecting nucleic acids and responding by secreting type I interferons (Reizis 2019).

These facts are disclosed in order to illustrate the technical problem addressed by the present disclosure.

### SUMMARY

The present subject matter identifies several isolated transcription factors that surprisingly reprogram or induce differentiated cells, multipotent or pluripotent stem cells into pDCs or interferon type I-producing cells, in vitro, ex vivo or in vivo.

Surprisingly, the induced pDCs or interferon type I-producing cells generated by reprograming as described in the present disclosure, are intrinsically able to secrete type I interferon required to induce an antiviral or anti-tumoral responses.

Surprisingly, the induced pDCs or interferon type I-producing cells generated by reprograming as described in the present disclosure, are intrinsically able to respond to toll-like receptor 7 (TLR7) and TLR9 ligands by secreting increased amounts of type I interferon, e.g. interferon alpha (IFN-α) and beta (IFN-β) required to induce an antiviral response.

DCs are professional APCs located throughout the body functioning at the interface of the innate and adaptive immune system. DCs are able to provide a crucial link between the external environment and the adaptive immune system through their ability to capture, process and present antigens loaded on major histocompatibility complex (MHC) class I and MHC class II molecules to T cells, targeting them to different types of immune responses. Firstly, DCs have to capture antigens and process them through major histocompatibility complex (MHC) class I and MHC class II. Following their activation, DCs are able to migrate towards the local draining lymph nodes priming multiple B cell and T cell responses, a key feature of adaptive immunity. The early protective efficacy is primarily conferred by the induction of antigen-specific antibodies produced by B lymphocytes. The long-term protection against specific antigens requires the persistence of specific antibodies and the generation of immunological memory that could provide a rapid and efficient response after subsequent antigen exposure. DCs, as professional APCs, have the ability to cross-present antigens, meaning that, in addition to its classical ability to present exogenous antigens on MHC class II and endogenous antigens on MHC class I, they are also able to present exogenous antigens on MHC class I, a critical step for the generation of cytotoxic T lymphocyte responses (CTL).

The ontogeny and/or microenvironment in which DCs are positioned may result in the expression of distinct combinations of surface receptors by DCs. For example, phenotypic criteria allow the classification of mouse DCs into different subpopulations. Of these, cDCs in lymphoid tissues are traditionally sub-divided into cDC1 and cDC2 subpopulations. It has been argued that different DC subsets may be involved in specific recognition of certain pathogens and/or regulate different immune responses, e.g. Th1 or Th2 (immunity) or regulatory T cells (tolerance). However, the phenotype and functional behavior of DCs is also significantly conditioned by external activating stimuli, denoting significant plasticity. cDC1 and cDC2 subsets differentially prime Th1 and Th2 responses in vivo. Immune therapy for cancer relies on using DCs to prime Th1 or cytotoxic T lymphocyte responses to promote tumor clearance. Alongside cDCs, pDCs emerged as a unique subset of DCs specialized in the production of type I interferons (IFNs) following viral infection or recognition of self nucleic acids through TLR7 and TLR9. In addition to contributing to antiviral immunity, pDCs can participate in the priming of both immunogenic and tolerogenic adaptive immune responses, being implicated in the pathology of autoimmune diseases and cancer.

pDCs have the unique feature of producing more type I interferons than any other cell type in response to viruses and/or virus derived nucleic acids. TLR7 and 9 are highly expressed in pDCs, in intracellular endosomal compartments. TLR7 recognizes viral single stranded RNA and TLR9 detects viral double stranded DNA rich in unmethylated CpG oligonucleotides. Upon activation with viral nucleic acids, they induce stimulation and recruitment of the adaptor protein MyD88 and two major TLR7/9 intracellular signalling pathways. The first pathway leads to type I interferon production, which requires the translocation of IRF7 to the nucleus promoting IFN-α and IFN-β transcription and secretion. Type I interferons, secreted by pDCs upon activation with nucleic acids, are able to bridge innate and adaptive immune systems, promoting long-term T-cell survival and memory, Th1 polarization, CD8 T-cell cytolytic activity, NK cell-mediated cytotoxicity and B-cell growth and differentiation. Alternatively, activation of the second pathway leads to NF-kb-dependent induction of expression of the pro-inflammatory cytokines, TNF-α and IL-6, as well as chemokines, for example CXCL9 (MIG), CXCL10 (IP-10), CCL3 (MIP-1a), CCL4 (MIP-1b) and CCL5 (RANTES). These are in turn able to attract activated CD4 and CD8 T cells to the sites of inflammation.

Currently, DC-based immunotherapies rely on autologous DC precursors: either monocytes, which are associated with the production of less-efficient DCs, or hematopoietic progenitors, which are isolated in very low numbers. In addition, these precursor cells are commonly compromised in cancer-bearing patients, resulting in the generation of a mixture of dysfunctional and heterogeneous DCs. In contrast, non-hematopoietic cell-types such as fibroblasts are usually not affected. Human dermal fibroblasts (HDFs) also exhibit other competitive advantages; they are easily obtained from a small skin punch biopsy, easily expanded in vitro for several passages (15-20 million cells after 4 weeks) and can be conserved frozen and used on-demand. Given the fundamental role of DCs functioning at the interface of the innate and adaptive immune system, there remains a clinical need to find alternative strategies to generate homogeneous populations of functional pDCs to prime antigen-specific responses.

In recent years, pDCs have gained a special interest, due to their ability to produce large amounts of IFN and to prime T cells. pDC-deficient mice were shown to be unable to efficiently clear the lymphocytic choriomeningitis virus due to impaired CD4+ and CD8+ T cell responses (Cervantes-Barragan et al., 2012). Similarly, a null mutation in IRF7 in a patient was reported to negatively affect the production of type I and III IFN by pDCs which led to severe influenza infection (Ciancanelli et al., 2015). These findings indicate that pDCs are important to fight viral infections, especially in the early stages (Swiecki and Colonna, 2015), where IFN production can limit viral replication. Moreover, several clinical trials have explored the potential of pDCs to induce anti-tumor immune responses (Tel et al., 2013). However, the therapeutic use of pDCs seems to be limited by the low number of these cells circulating in the blood. Thus, alternative sources of DCs, particularly pDCs are needed to fulfill the therapeutic needs.

In some embodiments, polypeptide variants or family members having the same or a similar activity as the reference polypeptide having the polypeptide sequences (SEQ. ID. 49-SEQ. ID. 96) or a polypeptide being encoded by the sequences SEQ. ID. 1-SEQ. ID. 48, can be used in the compositions, methods, and kits described herein. Generally, variants of a particular polypeptide encoding a pDC-inducing factor for use in the compositions, vectors, constructs, methods, and kits described herein will have at least about 90%, at least about 91%, at least about 92%, at least about 93%, at least about 94%, at least about 95%, at least about 96%, at least about 97%, at least about 98%, at least about 99% or more sequence identity to that particular reference polynucleotide or polypeptide as determined by sequence alignment programs and parameters described herein and known to those skilled in the art.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimize alignment between conserved motifs, as would be apparent to a person skilled in the art. The sequence identity values, which are indicated in the present subject-matter as a percentage were determined over the entire amino acid or nucleotide sequence, using BLAST with default parameters.

In an embodiment for better results, the transcription factors may be encoded by a polynucleotide selected from the group consisting of: IRF8 (SEQ. ID. 1, SEQ. ID. 2), SPIB (SEQ. ID. 3, SEQ. ID. 4), ARID5A (SEQ. ID. 5, SEQ. ID. 6), BCL11A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), HDAC5 (SEQ. ID. 15, SEQ. ID. 16), HHEX (SEQ. ID. 17, SEQ. ID. 18), HOXA5 (SEQ. ID. 19, SEQ. ID. 20), ID3 (SEQ. ID. 21, SEQ. ID. 22), IKZF1 (SEQ. ID. 23, SEQ. ID. 24), IKZF2 (SEQ. ID. 25, SEQ. ID. 26), IKZF3 (SEQ. ID. 27, SEQ. ID. 28), IKZF5 (SEQ. ID. 29, SEQ. ID. 30), IRF7 (SEQ. ID. 31, SEQ. ID. 32), MEF2C (SEQ. ID. 33, SEQ. ID. 34), MYB (SEQ. ID. 35, SEQ. ID. 36), RUNX2 (SEQ. ID. 37, SEQ. ID. 38), STAT3 (SEQ. ID. 39, SEQ. ID. 40), TCF4 (SEQ. ID. 41, SEQ. ID. 42), TCF12 (SEQ. ID. 43, SEQ. ID. 44), STAT1 (SEQ. ID. 45, SEQ. ID. 46), and TSC22D1 (SEQ. ID. 47, SEQ. ID. 48).

In an embodiment of the present disclosure, the transcription factor may be selected from the group consisting of: IRF8 (SEQ. ID. 49, SEQ. ID. 50), SPIB (SEQ. ID. 51, SEQ. ID. 52), ARID5A (SEQ. ID. 53, SEQ. ID. 54), BCL11A (SEQ. ID. 55, SEQ. ID. 56), CBFA2T3 (SEQ. ID. 57, SEQ. ID. 58), CREB3L2 (SEQ. ID. 59, SEQ. ID. 60), ETS1 (SEQ. ID. 61, SEQ. ID. 62), HDAC5 (SEQ. ID. 63, SEQ. ID. 64), HHEX (SEQ. ID. 65, SEQ. ID. 66), HOXA5 (SEQ. ID. 67, SEQ. ID. 68), ID3 (SEQ. ID. 69, SEQ. ID. 70), IKZF1 (SEQ. ID. 71, SEQ. ID. 72), IKZF2 (SEQ. ID. 73, SEQ. ID. 74), IKZF3 (SEQ. ID. 75, SEQ. ID. 76), IKZF5 (SEQ. ID. 77, SEQ. ID. 78), IRF7 (SEQ. ID. 79, SEQ. ID. 80), MEF2C (SEQ. ID. 81, SEQ. ID. 82), MYB (SEQ. ID. 83, SEQ. ID. 84), RUNX2 (SEQ. ID. 85, SEQ. ID. 86), STAT3 (SEQ. ID. 87, SEQ. ID. 88), TCF4 (SEQ. ID. 89, SEQ. ID. 90), TCF12 (SEQ. ID. 91, SEQ. ID. 92), STAT1 (SEQ. ID. 93, SEQ. ID. 94), and TSC22D1 (SEQ. ID. 95, SEQ. ID. 96).

In an embodiment, the transcription factors of the present disclosure may be used in veterinary or human medicine, in particular in infectious disease, or viral disease, or viral induced disease, or in cancer, or autoimmune disease or neurodegenerative disease.

In an embodiment for better results the cell may be selected from the group consisting of: a pluripotent stem cell, a multipotent stem cell, a hematopoietic stem cell, a differentiated cell, a tumor cell, a cancer cell, a cell line, in particular a mesoderm-derived cell line, such as a monocyte cell line, and mixtures thereof. In particular a mammalian cell, more in particular a mouse or a human cell.

In an embodiment for better results, the transcription factor of the present disclosure may be used as a reprogramming or inducing factor of a cell selected from the group consisting of: pluripotent stem cells, or multipotent stem cells, or differentiated cells, and mixtures thereof into a dendritic cell or interferon type I producing cell or antigen presenting cell, preferably a dendritic cell or interferon type I-producing cell.

In an embodiment for better results, the transcription factors of the present disclosure may be used as a reprogramming or inducing factor of a cell selected from the group consisting of: pluripotent stem cells, multipotent stem cells or differentiated cells, and mixtures thereof, into a dendritic cell or interferon type I-producing cell.

In an embodiment for better results, the transcription factor of the present disclosure may be used as a reprogramming or inducing factor of a cell selected from the group consisting of: a tumor cell, a cancer cell, and mixtures thereof, into antigen presenting cell.

In an embodiment, the results of the present disclosure shows that in Clec9a reporter mouse, pDCs are labeled with tdTomato fluorescent protein making this model suitable for screening pDC-inducing factor. IRF8 has been described to play a key role on cDC1 and pDC specification and is highly expressed on both DC subsets. Therefore, the present invention combined IRF8 with each of the individual 24 candidates and performed a progressive additive screen in Clec9a reporter mouse embryonic fibroblasts (MEFs).

In an embodiment, surprisingly, IRF8 combined with SPIB is sufficient to induce reporter activation. Moreover, the efficiency of reporter activation is surprisingly increased when IRF8 and SPIB are combined with BCL11A, CBFA2T3, CREB3L2, ETS1, STAT1, TCF4, TCF12 or TSC22D1. Expression of major histocompatibility complex (MHC) class II molecules, important for DC functionality, is also induced by the IRF8− and IRF8+SPIB-based combinations of pDC-inducing TFs.

In an embodiment, expression of IRF8 and SPIB is sufficient to induce surface expression of hematopoietic marker CD45. Remarkably, the surface expression of CD45 is surprisingly increased when IRF8 and SPIB are combined with IKZF1 or IKZF2.

In an embodiment, IRF8 and SPIB are able to induce the ability to secrete Interferon type I, namely IFN-α and IFN-β, and not anti-inflammatory IL-10, after stimulation with TLR7 and TLR9 ligands, a phenotypic property characteristic of pDCs. Interferon secretion is further increased when IRF8 and SPIB are combined with BCL1A, CBFA2T3, CREB3L2, ETS1, STAT1, TCF4, TCF12 or TSC22D1. Induced DCs generated with PU.1, IRF8 and BATF3 TFs do not show ability to secrete type I interferons.

In an embodiment, IRF8 and SPIB are able to induce the ability to secrete pro-inflammatory cytokines, in particular IL-6 and TNF-α, after stimulation with TLR7 and TLR9 ligands, a phenotypic property characteristic of pDCs. Cytokine secretion is further increased when IRF8 and SPIB are combined with ETS1, STAT1, TCF12 or TSC22D1.

In an embodiment, IRF8 and SPIB are able to induce the ability to secrete chemokines, namely CCL5 and CXCL10, after stimulation with TLR7 and TLR9 ligands, a phenotypic property characteristic of pDCs. Chemokine secretion is further increased when IRF8 and SPIB are combined with HOXA5, STAT1, TCF12 or TSC22D1.

An aspect of the present subject matter relates to a composition comprising a combination of at least two transcription factors encoded by an isolated or synthetic sequence at least 90% identical to a sequence from a list consisting of transcription factors selected from a list consisting of: IRF8 (SEQ. ID. 1, SEQ. ID. 2), SPIB (SEQ. ID. 3, SEQ. ID. 4), ARID5A (SEQ. ID. 5, SEQ. ID. 6), BCL11A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), IKZF1 (SEQ. ID. 23, SEQ. ID. 24), STAT1 (SEQ. ID. 45, SEQ. ID. 46), TCF4 (SEQ. ID. 41, SEQ. ID. 42), TCF12 (SEQ. ID. 43, SEQ. ID. 44), TSC22D1 (SEQ. ID. 47, SEQ. ID. 48), and mixtures thereof;

for use in reprogramming a stem cell or a differentiated cell, or mixtures thereof, into a plasmacytoid dendritic cell or interferon producing cell or antigen-presenting cell, preferably into a plasmacytoid dendritic cell or an interferon type I-producing cell in a subject.

An aspect of the present subject-matter relates to a composition comprising at least the two transcription factors IRF8 and SPIB, for use in reprogramming a stem cell or a differentiated cell, or mixtures thereof, into a plasmacytoid dendritic cell or an interferon type I-producing cell in a subject.

In an embodiment, the composition of the present disclosure comprises at least two transcription factors as herein described with the proviso that the combination of at least two transcription factors which may be isolated or synthetic transcription factors, is not TCF4 (SEQ. ID. 41, SEQ. ID. 42) and IRF8 (SEQ. ID. 1, SEQ. ID. 2).

In an embodiment, the composition of the present disclosure further comprises one or more transcription factors selected from the group consisting of: ARID5A, BCL1A, CBFA2T3, CREB3L2, ETS1, IKZF1, STAT1, TCF4, TCF12 and TSC22D1, preferably BCL1A, CBFA2T3, CREB3L2, ETS1, IKZF1, TCF4, TCF12 and TSC22D1.

In an embodiment, the composition of the present disclosure may comprise a combination of at least two transcription factors

encoded by an isolated or synthetic sequence at least 95% identical to a sequence from the group consisting of selected TFs from the group consisting of: IRF8 (SEQ. ID. 1, SEQ. ID. 2), SPIB (SEQ. ID. 3, SEQ. ID. 4), ARID5A (SEQ. ID. 5, SEQ. ID. 6), BCL1A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), IKZF1 (SEQ. ID. 23, SEQ. ID. 24), STAT1 (SEQ. ID. 45, SEQ. ID. 46), TCF4 (SEQ. ID. 41, SEQ. ID. 42), TCF12 (SEQ. ID. 43, SEQ. ID. 44), TSC22D1 (SEQ. ID. 47, SEQ. ID. 48), and mixtures thereof, preferably 96%, 97%, 98%; 99% or identical.

In an embodiment of the present disclosure the transcription factors are at least 90% identical to the sequence selected from the group consisting of: IRF8 (SEQ. ID. 1, SEQ. ID. 2), SPIB (SEQ. ID. 3, SEQ. ID. 4), ARID5A (SEQ. ID. 5, SEQ. ID. 6), BCL1A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), IKZF1 (SEQ. ID. 23, SEQ. ID. 24), STAT1 (SEQ. ID. 45, SEQ. ID. 46), TCF4 (SEQ. ID. 41, SEQ. ID. 42), TCF12 (SEQ. ID. X, SEQ. ID. X), TSC22D1 (SEQ. ID. 47, SEQ. ID. 48), and mixtures thereof, preferably 91%, 92%, 93%, 94%, 95%, 96%, 97%, 98%, 99% or identical.

In an embodiment, the composition of the present disclosure may comprise a combination of transcription factors selected from the following combinations:
IRF8 (SEQ. ID. 1, SEQ. ID. 2) and SPIB (SEQ. ID. 3, SEQ. ID. 4);
IRF8 (SEQ. ID. 1, SEQ. ID. 2), SPIB (SEQ. ID. 3, SEQ. ID. 4) and ARID5A (SEQ. ID. 5, SEQ. ID. 6);
IRF8 (SEQ. ID. 1, SEQ. ID. 2), SPIB (SEQ. ID. 5, SEQ. ID. 6) and BCL11A (SEQ. ID. 7, SEQ. ID. 8);
IRF8 (SEQ. ID. 1, SEQ. ID. 2), SPIB (SEQ. ID. 3, SEQ. ID. 4) and CBFA2T3 (SEQ. ID. 9, SEQ. ID. 10);
IRF8 (SEQ. ID. 1, SEQ. ID. 2), SPIB (SEQ. ID. 3, SEQ. ID. 4) and CREB3L2 (SEQ. ID. 11, SEQ. ID. 12);
IRF8 (SEQ. ID. 1, SEQ. ID. 2), SPIB (SEQ. ID. 3, SEQ. ID. 4) and ETS1 (SEQ. ID. 13, SEQ. ID. 14);
IRF8 (SEQ. ID. 1, SEQ. ID. 2), SPIB (SEQ. ID. 3, SEQ. ID. 4) and IKZF1 (SEQ. ID. 23, SEQ. ID. 24);
IRF8 (SEQ. ID. 1, SEQ. ID. 2), SPIB (SEQ. ID. 3, SEQ. ID. 4) and STAT1 (SEQ. ID. 45, SEQ. ID. 46);
IRF8 (SEQ. ID. 1, SEQ. ID. 2), SPIB (SEQ. ID. 3, SEQ. ID. 4) and TCF4 (SEQ. ID. 41, SEQ. ID. 42);
IRF8 (SEQ. ID. 1, SEQ. ID. 2), SPIB (SEQ. ID. 3, SEQ. ID. 4) and TCF12 (SEQ. ID. 43, SEQ. ID. 44);
IRF8 (SEQ. ID. 1, SEQ. ID. 2), SPIB (SEQ. ID. 3, SEQ. ID. 4) and TSC22D1 (SEQ. ID. 47, SEQ. ID. 48), and mixtures thereof.

In an embodiment, the composition of the present disclosure may comprise a combination of transcription factors selected from the following combinations:
IRF8 and SPIB;
IRF8, SPIB and ARID5A;
IRF8, SPIB and BCL1A;
IRF8, SPIB and CBFA2T3;
IRF8, SPIB and CREB3L2;
IRF8, SPIB and ETS1;
IRF8, SPIB and IKZF1;
IRF8, SPIB and STAT1;
IRF8, SPIB and TCF4;
IRF8, SPIB and TCF12;
IRF8, SPIB and TSC22D1; and mixtures thereof.

In an embodiment, the composition of the present disclosure may comprise the combination of transcription factors where the combination is IRF8 (SEQ. ID. 1, SEQ. ID. 2) and SPIB (SEQ. ID. 3, SEQ. ID. 4) or IRF8 (SEQ. ID. 1, SEQ. ID. 2), SPIB (SEQ. ID. 3, SEQ. ID. 4) and TCF12 (SEQ. ID. 43, SEQ. ID. 44).

In an embodiment, the composition of the present disclosure may comprise the combination of transcription factors where the combination is IRF8, SPIB and TCF12.

In an embodiment, the composition of the present disclosure may comprise the combination of transcription factors where the combination is IRF8, SPIB and TSC22D1.

In an embodiment, the composition of the present disclosure may comprise a pluripotent stem cell, or a multipotent stem cell, a differentiated cell, and mixtures thereof.

In an embodiment, the composition of the present disclosure may be used as a reprogramming or inducing factor of a cell selected from the group consisting of: a tumor cell, a cancer cell, and mixtures thereof, into an interferon type I-producing cell.

In an embodiment, the composition of the present disclosure may be used as reprogramming or inducing factors of pDCs, wherein the antigen is: a cancer antigen, a self-antigen, an allergen, an antigen from a pathogenic and/or infectious organism.

In an embodiment, the cells obtained by the use of the combination of transcriptions factors of the present disclosure have the ability to respond to TLR7 and TLR9 stimulation and/or the cells have the ability to secrete interferon type I cytokines, namely IFN-α and IFN-β.

Another aspect of the present disclosure relates to the use of the composition of the present disclosure in veterinary or human medicine, in particular in immunotherapy, or in neurodegenerative or ageing diseases, or in cancer or in infectious diseases, or as a drug screening.

Another aspect of the present disclosure relates to the use of the composition of the present disclosure in the diagnostic, therapy or treatment of cancer, antiviral immune responses and immune pathology, particularly in autoimmune diseases and immunodeficiency conditions.

In an embodiment, the pluripotent stem cell, multipotent stem cell or differentiated cell is a mammalian pluripotent stem cell, multipotent stem cell or differentiated cell, in particular a mouse or a human cell.

In an embodiment, the composition of the present disclosure may be used in the treatment, therapy or diagnosis of an infectious disease, a viral disease, or a viral-induced disease.

In an embodiment, the composition of the present disclosure may be used as an antiviral agent or as an immunogenic composition.

Another aspect of the present disclosure relates to a construct or a vector encoding at least the combination of two isolated transcription factors described in the present disclosure, preferably the encoded combination of three transcription factors, more preferably four transcription factors.

In an embodiment, the combination of at least two isolated transcription factors is in the following sequential order from 5′ to 3′:
SPIB (SEQ. ID. 3, SEQ. ID. 4) and IRF8 (SEQ. ID. 1, SEQ. ID. 2);
IRF8 (SEQ. ID. 1, SEQ. ID. 2) and SPIB (SEQ. ID. 3, SEQ. ID. 4);
SPIB (SEQ. ID. 3, SEQ. ID. 4), IRF8 (SEQ. ID. 1, SEQ. ID. 2) and ARID5A (SEQ. ID. 5, SEQ. ID. 6);
SPIB (SEQ. ID. 5, SEQ. ID. 6), IRF8 (SEQ. ID. 1, SEQ. ID. 2) and BCL11A (SEQ. ID. 7, SEQ. ID. 8);
SPIB (SEQ. ID. 3, SEQ. ID. 4), IRF8 (SEQ. ID. 1, SEQ. ID. 2) and CBFA2T3 (SEQ. ID. 9, SEQ. ID. 10);
SPIB (SEQ. ID. 3, SEQ. ID. 4), IRF8 (SEQ. ID. 1, SEQ. ID. 2) and CREB3L2 (SEQ. ID. 11, SEQ. ID. 12);
SPIB (SEQ. ID. 3, SEQ. ID. 4), IRF8 (SEQ. ID. 1, SEQ. ID. 2) and ETS1 (SEQ. ID. 13, SEQ. ID. 14);
SPIB (SEQ. ID. 3, SEQ. ID. 4), IRF8 (SEQ. ID. 1, SEQ. ID. 2) and IKZF1 (SEQ. ID. 23, SEQ. ID. 24);
SPIB (SEQ. ID. 3, SEQ. ID. 4), IRF8 (SEQ. ID. 1, SEQ. ID. 2) and STAT1 (SEQ. ID. 45, SEQ. ID. 46);
SPIB (SEQ. ID. 3, SEQ. ID. 4), IRF8 (SEQ. ID. 1, SEQ. ID. 2) and TCF4 (SEQ. ID. 41, SEQ. ID. 42);
SPIB (SEQ. ID. 3, SEQ. ID. 4), IRF8 (SEQ. ID. 1, SEQ. ID. 2) and TCF12 (SEQ. ID. 43, SEQ. ID. 44);
SPIB (SEQ. ID. 3, SEQ. ID. 4), IRF8 (SEQ. ID. 1, SEQ. ID. 2) and TSC22D1 (SEQ. ID. 47, SEQ. ID. 48).

Another aspect of the present disclosure relates to a construct or vector encoding at least the combination of two isolated transcription factors described in the present disclosure, preferably the encoded combination of three transcription factors, wherein the combination of at least two isolated transcription factors is in the following sequential order from 5′ to 3′:
SPIB (SEQ. ID. 3, SEQ. ID. 4) and IRF8 (SEQ. ID. 1, SEQ. ID. 2);
IRF8 (SEQ. ID. 1, SEQ. ID. 2) and SPIB (SEQ. ID. 3, SEQ. ID. 4);
SPIB (SEQ. ID. 3, SEQ. ID. 4), IRF8 (SEQ. ID. 1, SEQ. ID. 2) and ARID5A (SEQ. ID. 5, SEQ. ID. 6);
SPIB (SEQ. ID. 5, SEQ. ID. 6), IRF8 (SEQ. ID. 1, SEQ. ID. 2) and BCL11A (SEQ. ID. 7, SEQ. ID. 8);
SPIB (SEQ. ID. 3, SEQ. ID. 4), IRF8 (SEQ. ID. 1, SEQ. ID. 2) and CBFA2T3 (SEQ. ID. 9, SEQ. ID. 10);
SPIB (SEQ. ID. 3, SEQ. ID. 4), IRF8 (SEQ. ID. 1, SEQ. ID. 2) and CREB3L2 (SEQ. ID. 11, SEQ. ID. 12);
SPIB (SEQ. ID. 3, SEQ. ID. 4), IRF8 (SEQ. ID. 1, SEQ. ID. 2) and ETS1 (SEQ. ID. 13, SEQ. ID. 14);
SPIB (SEQ. ID. 3, SEQ. ID. 4), IRF8 (SEQ. ID. 1, SEQ. ID. 2) and IKZF1 (SED. ID. 23, SEQ. ID. 24);
SPIB (SEQ. ID. 3, SEQ. ID. 4), IRF8 (SEQ. ID. 1, SEQ. ID. 2) and STAT1 (SEQ. ID. 45, SEQ. ID. 46);
SPIB (SEQ. ID. 3, SEQ. ID. 4), IRF8 (SEQ. ID. 1, SEQ. ID. 2) and TCF4 (SEQ. ID. 41, SEQ. ID. 42);
SPIB (SEQ. ID. 3, SEQ. ID. 4), IRF8 (SEQ. ID. 1, SEQ. ID. 2) and TCF12 (SEQ. ID. 43, SEQ. ID. 44);
SPIB (SEQ. ID. 3, SEQ. ID. 4), IRF8 (SEQ. ID. 1, SEQ. ID. 2) and TSC22D1 (SEQ. ID. 47, SEQ. ID. 48).

In an embodiment, the vector is a viral vector; in particular a retroviral, an adenoviral, a lentiviral, a herpes viral, a pox viral, paramyxoviridae, rabdoviral, alphaviral, flaviral or adeno-associated viral vector.

In an embodiment, the vector or construct is synthetic mRNA, naked alphavirus RNA replicons or naked flavivirus RNA replicons.

In an aspect of the present disclosure, one or more vectors comprises at least two polynucleotide sequences encoding at least the two transcription factors IRF and SPIB, for use in reprogramming a stem cell or a differentiated cell, or mixtures thereof, into a plasmacytoid dendritic cells or an interferon type I-producing cell in a subject.

In an embodiment of the present disclosure, the one or more vectors further code for one or more transcription factors selected from the group consisting of: ARID5A, BCL1A, CBFA2T3, CREB3L2, ETS1, IKZF1, STAT1, TCF4, TCF12 and TSC22D1, preferably BCL1A, CBFA2T3, CREB3L2, ETS1, IKZF1, TCF4, TCF12 and TSC22D1.

In an embodiment of the present disclosure, the transcription factors of the one or more vectors are individually at least 90% identical to the sequences selected from the group consisting of: IRF8 (SEQ. ID. 49, SEQ. ID. 50), SPIB (SEQ. ID. 51, SEQ. ID. 52), ARID5A (SEQ. ID. 53, SEQ. ID. 54), BCL11A (SEQ. ID. 55, SEQ. ID. 56), CBFA2T3 (SEQ. ID. 57, SEQ. ID. 58), CREB3L2 (SEQ. ID. 59, SEQ. ID. 60), ETS1 (SEQ. ID. 61, SEQ. ID. 62), IKZF1 (SEQ. ID. 71, SEQ. ID. 72), TCF4 (SEQ. ID. 89, SEQ. ID. 90), TCF12 (SEQ. ID. 91, SEQ. ID. 92), STAT1 (SEQ. ID. 93, SEQ. ID. 94), and TSC22D1 (SEQ. ID. 95, SEQ. ID. 96).

In an embodiment of the present disclosure, the combination of transcription factors of the one or more vectors is selected from the following combinations:
IRF8 and SPIB;
IRF8, SPIB and ARID5A;
IRF8, SPIB and BCL1A;
IRF8, SPIB and CBFA2T3;
IRF8, SPIB and CREB3L2;
IRF8, SPIB and ETS1;
IRF8, SPIB and IKZF1;
IRF8, SPIB and STAT1;
IRF8, SPIB and TCF4;
IRF8, SPIB and TCF12; and
IRF8, SPIB and TSC22D1.

In an embodiment of the present disclosure, the combination of transcription factors of the one or more vectors is the combination of IRF8, SPIB and TCF12.

In an embodiment of the present disclosure, the combination of transcription factors of the one or more vectors is the combination of IRF8, SPIB and TSC22D1.

In an embodiment of the present disclosure, the cell used for reprogramming using the one or more vectors is selected from the group consisting of: pluripotent stem cell, multipotent stem cell, differentiated cell, tumor cell, cancer cell and mixtures thereof.

In an embodiment of the present disclosure, the one or more vectors are for use in veterinary or human medicine, in particular in the diagnosis, therapy or treatment of antiviral immune responses and immune pathology, particularly in autoimmune diseases and immunodeficiency conditions, neoplasia in particular cancer, namely solid or hematological tumors, of benign tumor, malignant tumor, early cancer, basal cell carcinoma, cervical dysplasia, soft tissue sarcoma, germ cell tumor, retinoblastoma, age-related macular degeneration, Hodgkin's lymphoma, blood cancer, prostate cancer, ovarian cancer, cervix cancer, uterus cancer, vaginal cancer, breast cancer, naso-pharynx cancer, trachea cancer, larynx cancer, bronchi cancer, bronchioles cancer, lung cancer, hollow organs cancer, esophagus cancer, stomach cancer, bile duct cancer, intestine cancer, colon cancer, colorectum cancer, rectum cancer, bladder cancer, ureter cancer, kidney cancer, liver cancer, gall bladder cancer, spleen cancer, brain cancer, lymphatic system cancer, bone cancer, pancreatic cancer, leukemia, skin cancer, or myeloma, infectious disease, viral disease or viral induced disease, central and peripheral nervous system disorder, fungal, viral, chlamydial, bacterial, nanobacterial or parasitic infectious disease, or viral disease, or viral induced disease, as an antiviral agent or as an immunogenic composition, immunotherapy, or in the treatment or therapy of neurodegenerative diseases, in the treatment or therapy of cancer or in the treatment or therapy of an infectious diseases, intradermal and transdermal therapies, HIV, infection with SARS coronavirus, Asian flu virus, herpes simplex, influenza virus, herpes zoster, hepatitis, human papillomavirus, Epstein-Barr virus, human T-lymphotropic virus or viral hepatitis, an amyloid disease in particular Amyloid A amyloidosis, Alzheimer's disease, Light-Chain (AL) amyloidosis, Type-2 Diabetes, Medullary Carcinoma of the Thyroid, Parkinson's disease, Polyneuropathy, or Spongiform Encephalopathy—Creutzfeldt Jakob disease or for drug screening.

Another aspect of the present disclosure relates to a method for reprogramming or inducing a cell into a plasmacytoid dendritic cell or interferon type I-producing cell, comprising the following steps:
transducing a cell selected from the group consisting of: a stem cell or a differentiated cell, and mixtures thereof,
with one or more vectors comprising at least two nucleic acid sequences encoding a sequence at least 90% identical, preferably at least 95% identical, to a sequence selected from the group consisting of IRF8 (SEQ. ID. 1, SEQ. ID. 2), SPIB (SEQ. ID. 3, SEQ. ID. 4), ARID5A (SEQ. ID. 5, SEQ. ID. 6), BCL11A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), IKZF1 (SEQ. ID. 23, SEQ. ID. 24), TCF4 (SEQ. ID. 41, SEQ. ID. 42), TCF12 (SEQ. ID. 43, SEQ. ID. 44), STAT1 (SEQ. ID. 45, SEQ. ID. 46), TSC22D1 (SEQ. ID. 47, SEQ. ID. 48) and mixtures thereof;
culturing the transduced cell in a cell media that supports growth of plasmacytoid dendritic cells or interferon type I-producing cell.

Another aspect of the present disclosure relates to an in vitro method for reprogramming or inducing a cell into a plasmacytoid dendritic cell or interferon type I-producing cell, comprising the following steps:
transducing a cell selected from the group consisting of: a stem cell or a differentiated cell, and mixtures thereof,
with one or more vectors comprising at least the two transcription factors IRF8 and SPIB,
   culturing the transduced cell in a cell media that supports growth of plasmacytoid dendritic cells or interferon type I-producing cell.

In an embodiment, the sequence is selected from the group of combinations consisting of
IRF8 (SEQ. ID. 1, SEQ. ID. 2) and SPIB (SEQ. ID. 3, SEQ. ID. 4);
IRF8 (SEQ. ID. 1, SEQ. ID. 2), SPIB (SEQ. ID. 3, SEQ. ID. 4) and ARID5A (SEQ. ID. 5, SEQ. ID. 6);
IRF8 (SEQ. ID. 1, SEQ. ID. 2), SPIB (SEQ. ID. 5, SEQ. ID. 6) and BCL11A (SEQ. ID. 7, SEQ. ID. 8);
IRF8 (SEQ. ID. 1, SEQ. ID. 2), SPIB (SEQ. ID. 3, SEQ. ID. 4) and CBFA2T3 (SEQ. ID. 9, SEQ. ID. 10);
IRF8 (SEQ. ID. 1, SEQ. ID. 2), SPIB (SEQ. ID. 3, SEQ. ID. 4) and CREB3L2 (SEQ. ID. 11, SEQ. ID. 12);
IRF8 (SEQ. ID. 1, SEQ. ID. 2), SPIB (SEQ. ID. 3, SEQ. ID. 4) and ETS1 (SEQ. ID. 13, SEQ. ID. 14);
IRF8 (SEQ. ID. 1, SEQ. ID. 2, SPIB (SEQ. ID. 3, SEQ. ID. 4) and IKZF1 (SEQ. ID. 23, SEQ. ID. 24);
IRF8 (SEQ. ID. 1, SEQ. ID. 2, SPIB (SEQ. ID. 3, SEQ. ID. 4) and STAT1 (SEQ. ID. 45, SEQ. ID. 46);
IRF8 (SEQ. ID. 1, SEQ. ID. 2), SPIB (SEQ. ID. 3, SEQ. ID. 4) and TCF4 (SEQ. ID. 41, SEQ. ID. 42);
IRF8 (SEQ. ID. 1, SEQ. ID. 2), SPIB (SEQ. ID. 3, SEQ. ID. 4) and TCF12 (SEQ. ID. 43, SEQ. ID. 44);
IRF8 (SEQ. ID. 1, SEQ. ID. 2), SPIB (SEQ. ID. 3, SEQ. ID. 4) and TSC22D1 (SEQ. ID. 47, SEQ. ID. 48) and mixtures thereof.

In an embodiment, the sequence is selected from the group of combinations consisting of:
IRF8 and SPIB;
IRF8, SPIB and ARID5A;
IRF8, SPIB and BCL1A;
IRF8, SPIB and CBFA2T3;
IRF8, SPIB and CREB3L2;
IRF8, SPIB and ETS1;
IRF8, SPIB and IKZF1;
IRF8, SPIB and STAT1;
IRF8, SPIB and TCF4;
IRF8, SPIB and TCF12;
IRF8, SPIB and TSC22D1, and mixtures thereof.

In an embodiment, the method may comprise the step of culturing the transduced cell during at least 2 days, preferably at least 5 days, more preferably at least 8 days, even more preferably at least 9 days.

In an embodiment, the transducing step further comprises at least one vector selected from the group consisting of: a nucleic acid sequence encoding IL-12; nucleic acid sequence encoding IL-4; a nucleic acid sequence encoding IFN-α; a nucleic acid sequence encoding IFN-β; a nucleic acid sequence encoding IFN-γ; a nucleic acid sequence encoding TNF; nucleic acid sequence encoding GM-CSF; nucleic acid sequence encoding siRNAs targeting IL-10 RNA, and mixtures thereof.

In an embodiment, the transducing step further comprises at least one vector comprising nucleic acids encoding immunostimulatory cytokines.

In an embodiment, the cell is selected from the group consisting of: a pluripotent stem cell, a multipotent stem cell, or a differentiated cell, and mixtures thereof; in particular a mammalian cell.

In an embodiment, the pluripotent stem cell, multipotent stem cell, or differentiated cell, is selected from the group consisting of: an endoderm-derived cell, a mesoderm-derived cell, an ectoderm-derived cell, a multipotent stem cell including a mesenchymal stem cell, a hematopoietic stem cell, an intestinal stem cell, a pluripotent stem cell and a cell line.

In an embodiment, the cell is human or non-human. In another embodiment, the cell is a mouse cell.

Another aspect of the present disclosure relates to an induced plasmacytoid dendritic cell or interferon type I-producing cell or an antigen presenting cell obtained by the in vitro method described in the present disclosure, preferably an induced plasmacytoid dendritic cell or interferon type I-producing cell.

Another aspect of the present disclosure relates to a composition comprising a plasmacytoid dendritic cell, or an interferon type I-producing cell, or an antigen presenting cell described in the present disclosure, preferably an induced plasmacytoid dendritic cell or interferon type I-producing cell, in a therapeutically effective amount and a pharmaceutically acceptable excipient.

In an embodiment, the composition may be used in veterinary or human medicine.

In an embodiment, the composition may be used in immunotherapy, or in the treatment or therapy of neurodegenerative diseases, or in the treatment or therapy of cancer or in the treatment or therapy of infectious diseases.

In an embodiment, the composition may further comprise an analgesic, an anti-inflammatory agent, a chemotherapy agent, a radiotherapy agent, an antibiotic, a diuretic, or mixtures thereof.

In an embodiment, the composition may further comprise a filler, a binder, a disintegrant, or a lubricant, or mixtures thereof.

In an embodiment, the composition may be used in intradermal and transdermal therapies.

In an embodiment, the composition may be in an injectable formulation, preferably an in-situ injection.

In an embodiment, the composition may be used in veterinary or human medicine, or in a drug screening.

In an embodiment, the composition may be used in the treatment, therapy or diagnosis of a central and peripheral nervous system disorder.

In an embodiment, the composition may be used in the treatment, therapy or diagnosis of neoplasia, in particular cancer, such as solid or hematological tumours.

In an embodiment, the composition, vectors or constructs may be used in the treatment, diagnostic or therapy of benign tumors, malignant tumors, early cancer, basal cell carcinoma, cervical dysplasia, soft tissue sarcoma, germ cell tumors, retinoblastoma, age-related macular degeneration, Hodgkin's lymphoma, blood cancer, prostate cancer, ovarian cancer, cervix cancer, uterus cancer, vaginal cancer, breast cancer, naso-pharynx cancer, trachea cancer, larynx cancer, bronchi cancer, bronchioles cancer, lung cancer, hollow organs cancer, esophagus cancer, stomach cancer, bile duct cancer, intestine cancer, colon cancer, colorectum cancer, rectum cancer, bladder cancer, ureter cancer, kidney cancer, liver cancer, gall bladder cancer, spleen cancer, brain cancer, lymphatic system cancer, bone cancer, pancreatic cancer, leukemia, skin cancer, or myeloma.

In an embodiment, the composition, vectors, or constructs may be used in the treatment, therapy or diagnosis of a fungal, viral, chlamydial, bacterial, nanobacterial or parasitic infectious disease, or a viral disease, or a viral-induced disease.

In an embodiment, the composition, vectors, or constructs may be used in the treatment, therapy or diagnosis of HIV, infection with a SARS coronavirus, Asian flu virus, herpes simplex, influenza virus, herpes zoster, hepatitis, human papillomavirus, Epstein-Barr virus, human T-lymphotropic virus or viral hepatitis.

In an embodiment, the composition, vectors, or constructs may be used in the treatment, therapy or diagnosis of an amyloid disease in particular Amyloid A amyloidosis, Alzheimer's disease, Light-Chain (AL) amyloidosis, Type-2 Diabetes, Medullary Carcinoma of the Thyroid, Parkinson's disease, Polyneuropathy, or Spongiform Encephalopathy, such as Creutzfeldt Jakob disease.

Another aspect of the present disclosure relates to a vaccine or an injectable formulation comprising the composition, vectors, or constructs described in the present disclosure, and/or an induced plasmacytoid dendritic cell or interferon type I-producing cell as described or antigen presenting cell described in the present disclosure or mixtures thereof.

Another aspect of the present disclosure relates to a kit comprising at least one of the following components:
the induced plasmacytoid dendritic cell or interferon type I-producing cell or antigen presenting cell of the present disclosure;
the composition as described in the present disclosure;
the one or more vector or construct as described in the present disclosure; or mixtures thereof.

Another aspect of the present disclosure relates to a kit comprising at least one of the following components:
the induced plasmacytoid dendritic cell or interferon type I-producing cell of the present disclosure;
the composition as described in the present disclosure;
the one or more vectors or construct as described in the present disclosure; or mixtures thereof.

In an embodiment, the present disclosure provides evidence that IRF8, when combined with SPIB, kick-start a pDC program in fibroblasts. These findings provide valuable insights into pDC specification. In the future, the generation of pDCs by direct reprogramming opens avenues for inducing anti-viral immune responses with autologous-engineered cells.

### DESCRIPTION OF DRAWINGS

The following figures provide preferred embodiments for illustrating the disclosure and should not be seen as limiting the scope of invention.

FIG. 1. Ontogeny of the main subsets of dendritic cells. While conventional dendritic cells type 1 (cDC1) and type 2 (cDC2) emerge from a pre-DC population derived from common dendritic cell Progenitors (CDP), plasmacytoid dendritic cells (pDCs) can derive either from common lymphocyte progenitors (CLP) or CDPs. pDCs emerge as a unique subset of DCs specialized in the production of type I Interferons (IFNs) following viral infection. cDC1 s excel on the ability to perform cross-presentation, inducing cytotoxic T cell responses, and polarize preferentially T helper 1 responses. cDC2s excel on presenting antigens to T helper cells polarizing T helper 2 or 17 responses.

FIG. 2. Generating pDC or interferon (IFN) type I-producing cells by direct cellular reprogramming. Observation of the effect of the transcription factor (TF) combination disclosure in the present subject-matter for the induction of pDCs from pluripotent stem cells, multipotent stem cells and somatic cells, such as fibroblasts. pDCs are specialized in producing type I IFNs, and so will be applied in the context of personalized immunotherapy. Induced pDCs or pDC-inducing TF combinations will be used to induce immunity in distinct diseased context including viral infection and cancer.

FIG. 3. Expression of Clec9a, Irf8, Tlr7 and Tlr9 is restricted to pDC. Heat map showing expression levels of Clec9a, Irf8, Tlr7 and Tlr9 in pDCs and other immune cells close in ontogeny, in particular cDC1 and cDC2, macrophages and monocytes. Expression profiles were obtained from data available in Immunological Genome Project (www.immaen.com).

FIG. 4. TF candidates to induce pDCs. Twenty-two candidate TFs are highly enriched in pDCs, when compared with cDC1 and cDC2. Heat map showing gene expression profiles extracted from data available in Immunological Genome Project.

FIG. 5. Clec9a is expressed in pDCs. Expression of tdTomato on 92% of pDCs (gated as CD11c^{low} BST2⁺ B220⁺ Siglec-H⁺ cells) isolated from spleens of double transgenic Clec9a-Cre×Rosa26-STOP-tdTomato.

FIG. 6. IRF8 combined with SPIB induces Clec9a reporter activation. Mouse embryonic fibroblasts (MEFs) isolated from double transgenic Clec9a-tdTomato mice were transduced with pools of inducible lentiviral vectors encoding IRF8 alone or combined with additional pDC-inducing TFs and analyzed by flow cytometry 5 days after addition of Dox. Quantification of tdTomato+ cells after transduction with IRF8 alone or combined with individual pDC-inducing candidate TFs. M2rtTA-transduced cells were included as control.

FIG. 7. Induced pDCs express antigen presenting molecules at cell surface. Flow cytometry analysis of major histocompatibility complex class II (MHC-II) expression of tdTomato+ cells generated after overexpression of IRF8 and SPIB at day 8.

FIG. 8. Combination of additional pDC-inducing TFs with IRF8 and SPIB increases the efficiency of Clec9a reporter activation. Quantification of tdTomato+ cells after transduction of double transgenic Clec9a-tdTomato MEFs with IRF8 and SPIB alone or combined with individual pDC-inducing factors. Flow cytometry analysis was performed at day 8. M2rtTa-transduced cells were included as control.

FIG. 9. Combination of additional pDC-inducing TFs with IRF8 and SPIB increases expression of MHC-II at cell surface. Flow cytometry analysis of MHC-II expression of MEFs transduced with M2rtTA, IRF8+SPIB alone or combined with individual pDC-inducing TFs, at day 8. Quantification of MHC-II levels is shown for tdTomato negative (tdT−) and positive (tdT+) populations.

FIG. 10. Induced pDCs secrete type I Interferon upon TLR7 and TLR9 stimulation. MEFs were transduced with M2rtTA, PIB (PU.1, IRF8 and BATF3), IRF8 and SPIB alone or combined with ARID5A, BCL11A, CBFA2T3, ETS1, HOXA5, TCF4 and TCF12 and tdTomato+ cells were sorted at day 9 after addition of doxycycline. Purified tdTomato+ cells were incubated overnight with TLR7 (R848) and TLR9 (CpG ODN 1668) and IFN-α (A), IFN-β (B) and IL-10 (C) cytokine secretion was quantified on supernatants using a cytometry bead array.

FIG. 11. Additional TF candidates to induce pDCs. Stat1 (A) and Tsc22d1 (B) are enriched in pDCs, when compared with cDC1 and cDC2. Gene expression profiles extracted from data available in Immunological Genome Project.

FIG. 12. Combination of additional pDC-inducing TFs with IRF8 and SPIB increases the efficiency of Clec9a reporter activation and MHC-1l surface expression. Flow cytometry analysis of (A) Clec9a reporter activation and (B) MHC-1l expression of MEFs transduced with M2rtTA (M2), IRF8+SPIB alone or combined with STAT1 or with TSC22D1. Flow cytometry analysis was performed at day 5. M2rtTa-transduced cells were included as control.

FIG. 13. Induced pDCs secrete type I Interferons upon TLR7 and TLR9 stimulation. MEFs were transduced with M2rtTA, IRF8 and SPIB alone or combined with STAT1 and TSC22D1 and tdTomato+ cells were sorted at day 9 after addition of doxycycline. Purified tdTomato+ cells were incubated overnight with triggers of TLR7 (R848) and TLR9 (CpG ODN 1668) and IFN-α (A), IFN-β (B) and IL-10 (C) cytokine secretion was quantified on supernatants using a cytometry bead array.

FIG. 14. Induced pDCs express CD45 at cell surface. Flow cytometry analysis of CD45 expression of MEFs transduced with M2rtTA, PIB (PU.1, IRF8 and BATF3), IRF8+SPIB alone or combined with individual pDC-inducing TFs, at day 5. M2rtTa (M2)-transduced cells were included as control.

FIG. 15. Induced pDCs secrete pro-inflammatory cytokines and chemokines upon TLR7 and TLR9 stimulation. MEFs were transduced with M2rtTA, IRF8 and SPIB alone or combined with ARID4A, BCL11A, CBFA2T3, ETS1, HOXA5, STAT1, TCF4, TCF12 and TSC22D1 and tdTomato+ cells were sorted at day 9 after addition of doxycycline. Purified tdTomato+ cells were incubated overnight with TLR7 (R848) and TLR9 (CpG ODN 1668) and IL-6 (A), TNF-α (B), CCL5 (C) and CXCL10 (D) cytokine secretion was quantified on supernatants using a cytometry bead array.

### DETAILED DESCRIPTION

The present disclosure relates to compositions, nucleic acid constructs, vectors, methods and kits thereof for reprogramming cells into pDCs or interferon type I-producing cells. The aim is to produce pDCs and interferon type I-producing cells with the ability to induce antiviral and anti-tumoral responses from differentiated, multipotent or pluripotent stem cells by introducing and expressing isolated/synthetic transcription factors. More particularly, the disclosure provides methods for obtaining pDCs or interferon type I-producing cells by direct cellular reprogramming with the surprising use of combinations of specific transcription factors.

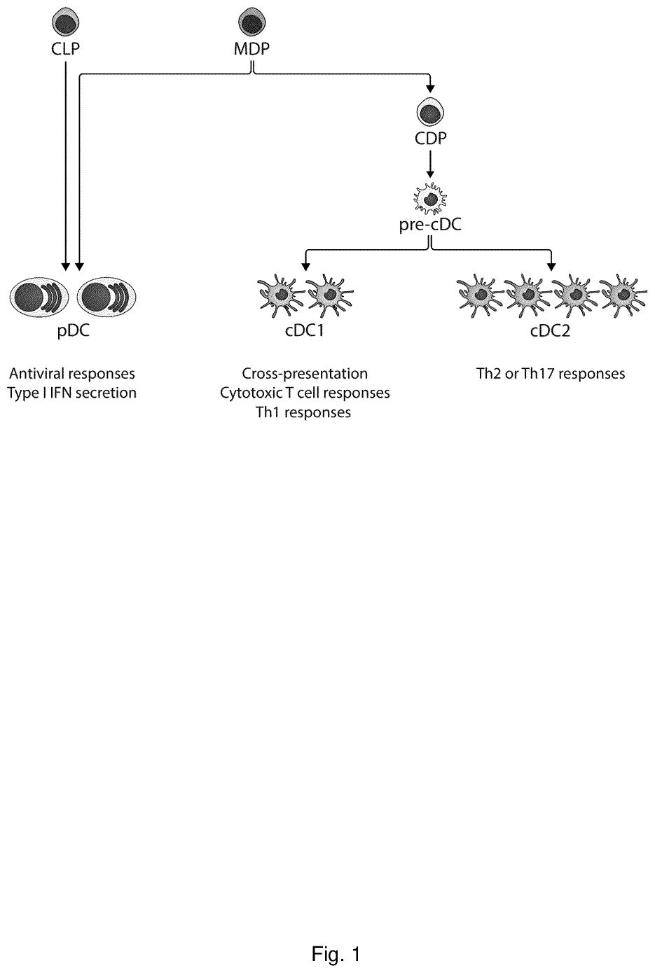

Natural DCs are bone marrow-derived cells that are seeded in all tissues. DCs are poised to sample the environment and to transmit the gathered information to cells of the adaptive immune system (T cells and B cells). Upon antigen engulfment, DCs initiate an immune response by presenting the processed antigen, which is in the form of peptide-major histocompatibility complex (MHC) molecule complexes, to naive (that is, antigen inexperienced) T cells in lymphoid tissues. DCs can be divided in 2 main subsets: plasmacytoid DCs (pDCs), and conventional or classical DCs (cDCs) that can be further sub-divided in cDCs type 1 (cDC1s) and type 2 (cDC2s) (FIG. 1). pDCs are typically characterized by a round morphology and by the expression of low levels of MHC-1l that are up-regulated after activation. After activation, pDCs typically overexpress co-stimulatory and MHC molecules in addition to secrete various cytokines and chemokines responsible for initiating and/or enhancing many T and B lymphocyte responses, i.e. tumor necrosis factor (TNF)-α, IFN-α, IFN-β, IL-12, IL-6 and chemokines such as CCL5 and CXCL10. pDCs express high levels of the nucleic-acid sensing toll-like receptors (TLRs) TLR7 and TLR9. TLR7 or TLR9 stimulation allows the detection of pathogen-derived nucleic acids by pDCs and the production of large amounts of type I interferon i.e. IFN-α and IFN-β. Although activated pDCs can actively produce cytokines and perform both antigen presentation and cross-presentation, it is still unclear whether these functional features are performed by the same cells or by distinct functionally specialized pDC subsets. cDCs are generally identified by their high expression of major histocompatibility complex class II molecules (MHC-II), co-stimulatory molecules, such as CD80/86 and CD40, and integrin CD11c, as well as their superior capacity to migrate from non-lymphoid to lymphoid organs and stimulate naïve T cells. DNGR-1, also known as CLEC9A, is a receptor for necrotic cells that favors cross-priming of cytotoxic T lymphocyte responses (CTL) to dead cell-associated antigens in mice. DNGR-1 is selectively expressed at high levels by mouse cDC1, cDC2 and pDCs, being responsible for recognizing an intracellular ligand exposed after cell death. Recently, expression of Clec9a was shown to allow the identification of DC precursors (CDPs) committed to the conventional or plasmacytoid DC lineages and their progeny in lymphoid tissues (Schraml et al., 2015).

The successful identification of pDC-inducing factors capable of reprogramming differentiated cells to induced pDCs, as described herein, can advance our basic understanding of pDC biology in a number of ways. This work will provide thorough insight into pDC minimal transcriptional networks. In addition, the identification of pDC-inducing factors offer unprecedented opportunities to understand how pDC state is established and how key regulatory machinery is put into place.

Transcription factors (TFs) play a critical role in the specification of all cell types during development. The success of direct reprogramming strategies using TF-mediated reprogramming indicates that it is equally plausible to direct the differentiation of pluripotent ES/iPS cells or multipotent stem cells to specific fates using such factors. Accordingly, using the pDC-inducing factors identified herein, directed differentiation of ES/iPS cells to a definitive DC fate by expression of the DC-enriched TFs can be achieved. Additionally, using the pDC-inducing factors identified herein, directed differentiation of multipotent hematopoietic stem and progenitor cells to a definitive DC fate by expression of the DC-enriched TFs can be achieved.

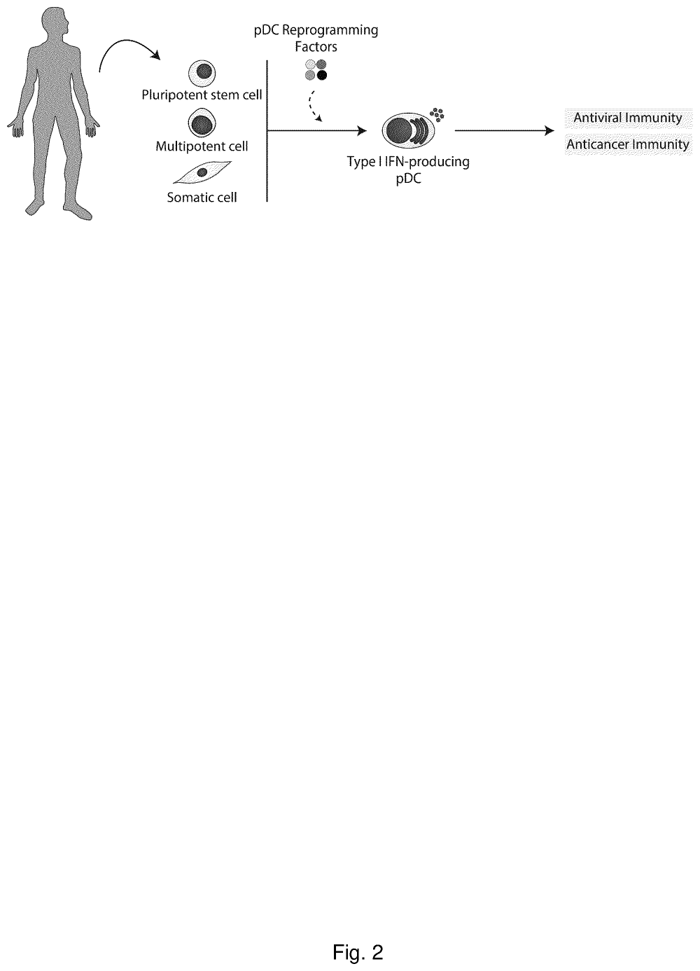

Typically, nucleic acids encoding the pDC-inducing factors, e.g., DNA or RNA, or constructs thereof, are introduced into a cell, using viral vectors or without viral vectors, via one or repeated transductions or transfections, and the expression of the gene products and/or translation of the RNA molecules result in cells that are morphologically, biochemically, and functionally similar to pDCs, as described herein. These induced pDCs after priming with the adequate antigens have the ability to capture, process and present them to effectors cells of the immune system (macrophages, T-cells, B-cells, NK cells) and secrete type I IFNs, eliciting antigen-specific immune responses against viral infections and cancer (FIG. 2).

An aspect of the present disclosure is the use of TFs or the use of a combination of TFs in cancer cells (in situ or ex vivo) to force them to secrete type I IFN, present their own antigens to immune cells and secrete other pro-inflammatory cytokines and chemoattractants within the tumor microenvironment. This method represents a feasible strategy to increase the clinical outcome of anticancer immunotherapies as it bypasses cancer evasion mechanisms and increases tumor immunogenicity.

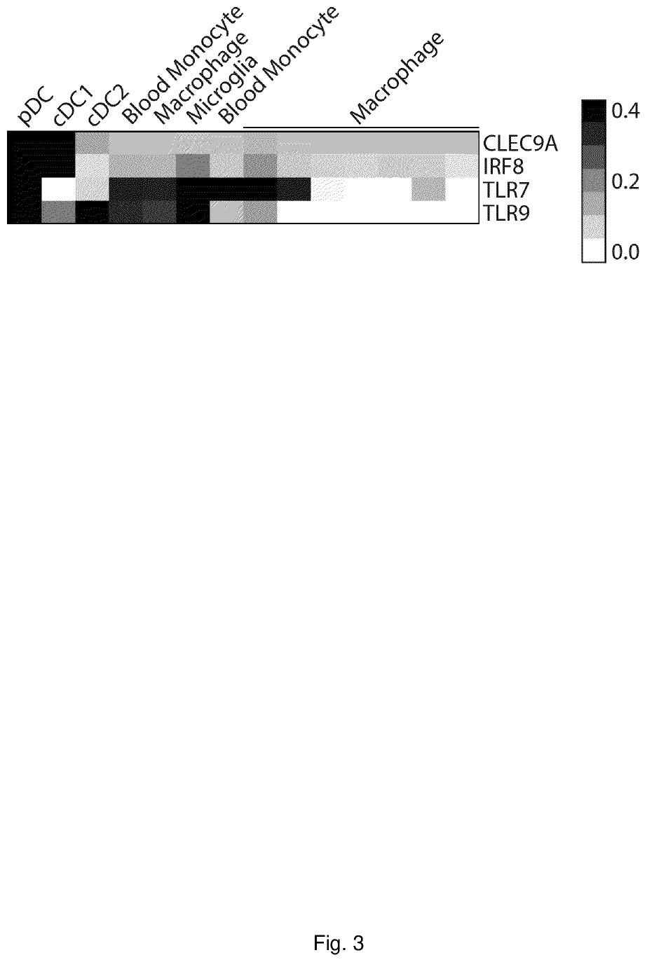

Plasmacytoid DCs specifically express Tlr7 and Tlr9 when compared to cDC1 s and cDC2 and other related mononuclear cells (FIG. 3). TLR7 and TRL9 receptors allow pDCs to sense viral DNA and respond by secreting type I IFNs. Plasmacytoid DCs, as well as cDC1 s, express Clec9a and Irf8.

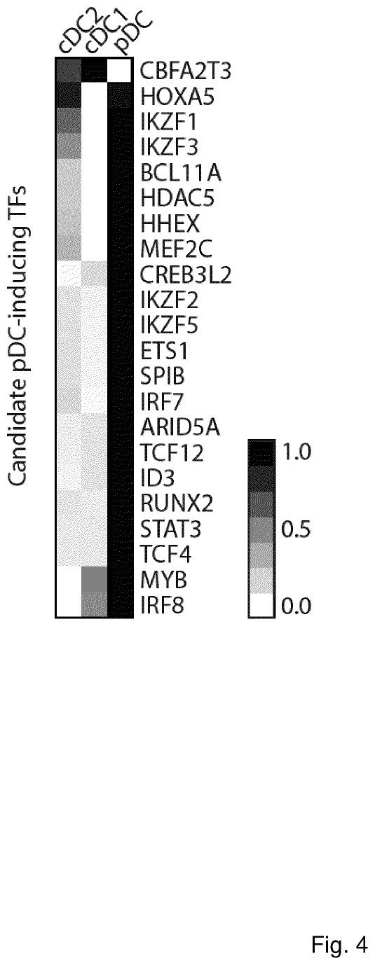

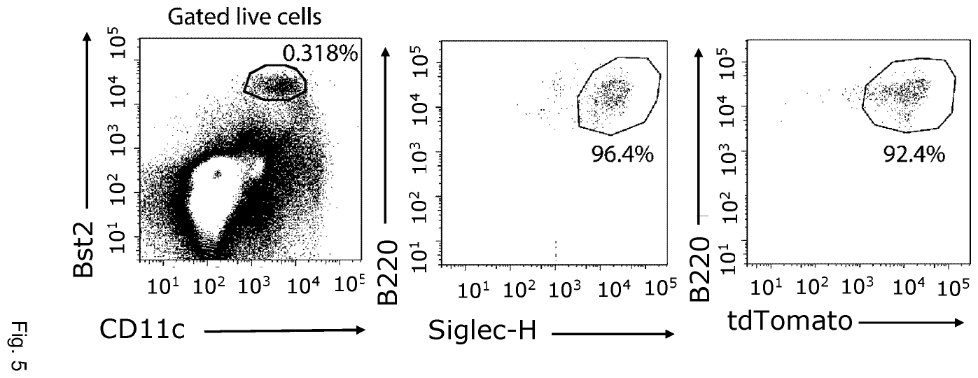

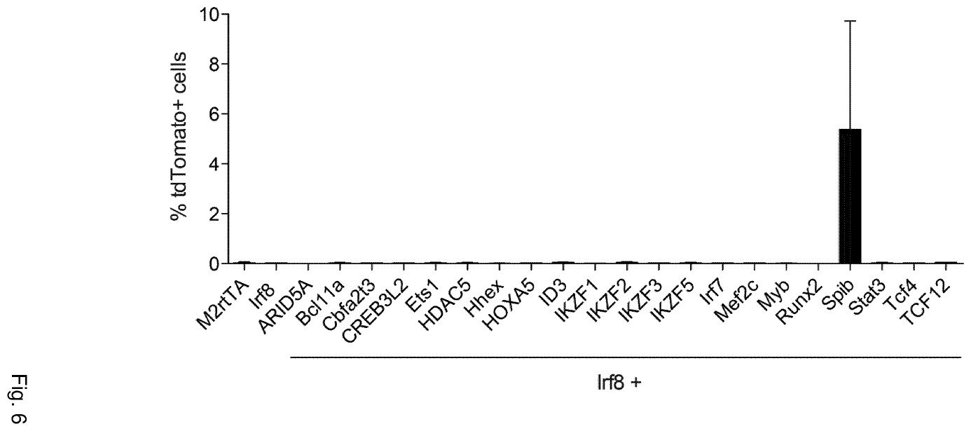

In an embodiment, 22 candidate TFs were selected due to their specific enriched gene expression in plasmacytoid DCs when compared to cDC1s and cDC2s (FIG. 4). 22 candidate TFs were cloned individually in a reprogramming proven Doxycycline (Dox)-inducible lentiviral vector.

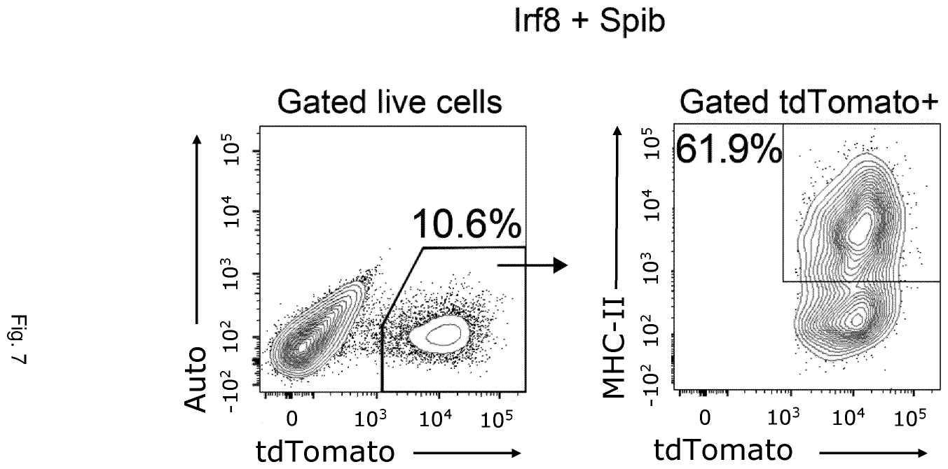

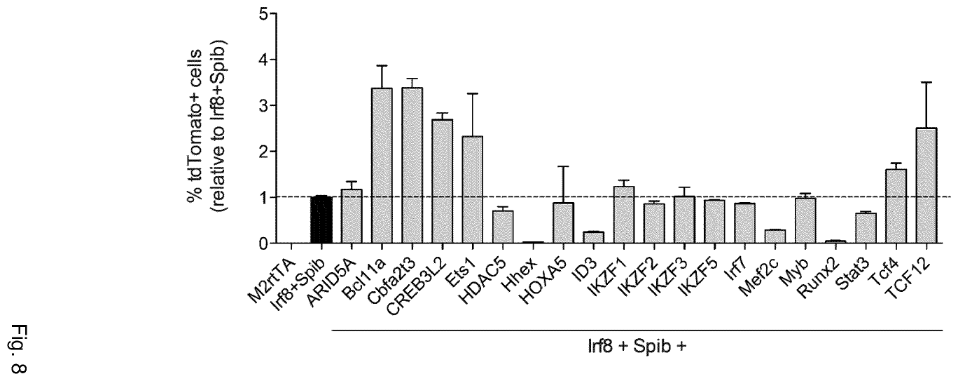

In an embodiment, in order to screen the effect of the pDC-inducing TFs and pDC-inducing TF combinations by cellular reprogramming, mouse embryonic fibroblasts (MEFs) harboring a DC-specific reporter (Clec9a-Cre×R26-stop-tdTomato) were used to screen pDC-inducing TFs ability to activate the DC-specific Clec9a-tomato reporter (FIG. 4). In Clec9a-tomato reporter mouse, the tdTomato fluorescent protein is expressed exclusively by CDPs, pre-DCs, cDCs and pDCs (Schraml et al., 2015). Macrophages, other immune lineages or monocyte-derived DCs in culture do not express Clec9a and therefore don't activate the reporter or expression of the tdTomato protein (FIG. 3). Spleen cells isolated from Clec9a reporter mice were analyzed, confirming that 92.4% of pDC cells (gated in CD11c^{low} BST2⁺ B220⁺ SiglecH⁺ cells) express the tdTomato fluorescent protein (FIG. 5).

Double transgenic Clec9a-tdTomato reporter MEFs were isolated from E13.5 embryos and excluded from any contaminating tdTomato+ or CD45+ cell that could be already committed to the hematopoietic lineage by fluorescent-activated cell sorting (FACS).

In an embodiment, SPIB and IRF8 are sufficient for Clec9a activation.

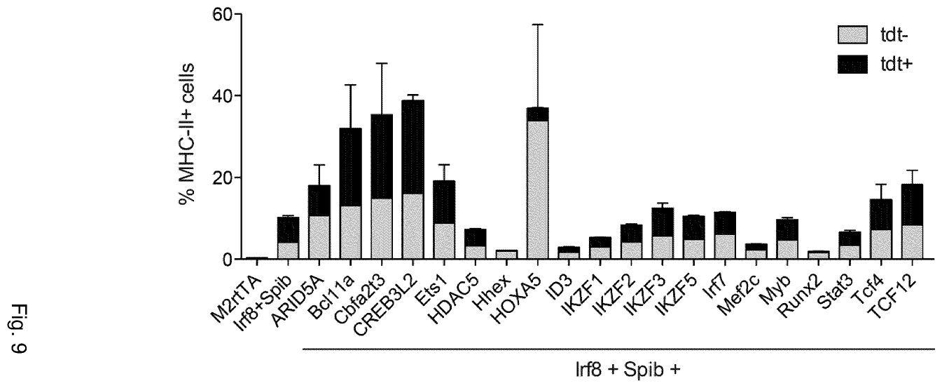

In an embodiment, Clec9a reporter MEFs were transduced with IRF8 alone or combinations of 2 with IRF8 plus one of the other 21 candidate TFs and evaluated for tdTomato expression. After transduction with the 21 combinations of 2 candidate TFs or IRF8 alone, it was observed the emergence of tdTomato+ cells 5 days after adding Dox (FIG. 6). The combination of IRF8 and SPIB generated 5% of tdTomato+ cells, suggesting that IRF8 and SPIB are a minimal combination of factors required to induce reporter activation. TdTomato+ cells were not detected after transduction with control M2rtTA vector neither with IRF8 alone.

In an embodiment, the antigen presenting cell marker MHC-II is expressed in approximately 60% of MEFs transduced with IRF8 and SPIB (FIG. 7).

In an embodiment the impact of expressing the remaining 20 candidates TFs together with IRF8 and SPIB was evaluated (FIG. 8). From the 20 TFs tested it was observed that HHEX, ID3, MEF2C and RUNX2 negatively impact the numbers of tdTomato+ cells generated. The addition of ARID5A, HDAC5, HOXA5, IKZF1, IKZF2, IKZF3, IKZF5, IRF7, MYB and STAT3 didn't impact the numbers of tdTomato+ cells generated. The addition of BCL11A, CBFA2T3, CREB3L2, ETS1, TCF12 and TCF4 positively impact the numbers of tdTomato+ cells generated, increasing the efficiency of reporter activation.

In an embodiment it was evaluated if the activation of the Clec9a-tdTomato reporter was reflected in the surface expression of MHC-II, a key component of the antigen presentation machinery. Remarkably, it was observed that tdTomato+ cells at day 8 expressed MHC-II at the surface (FIG. 9).

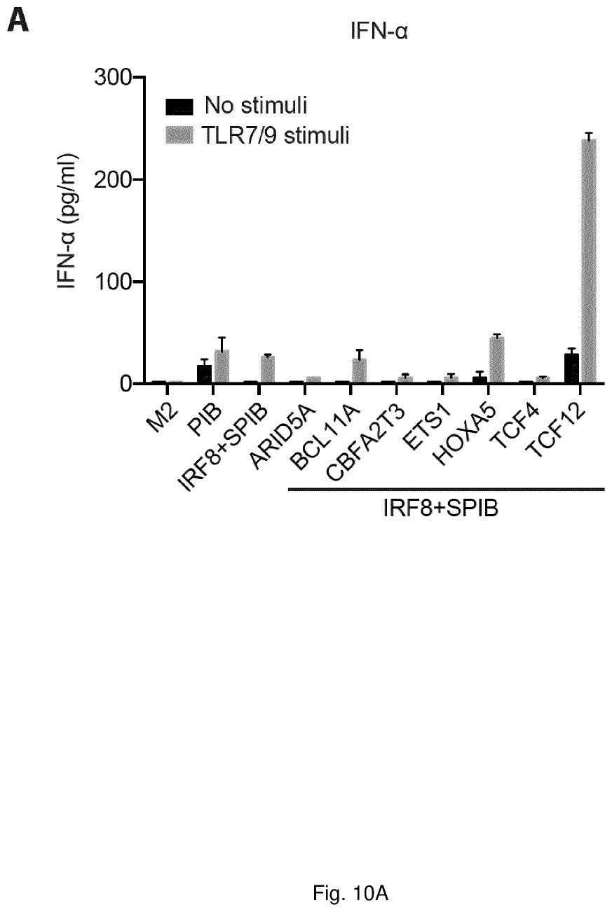

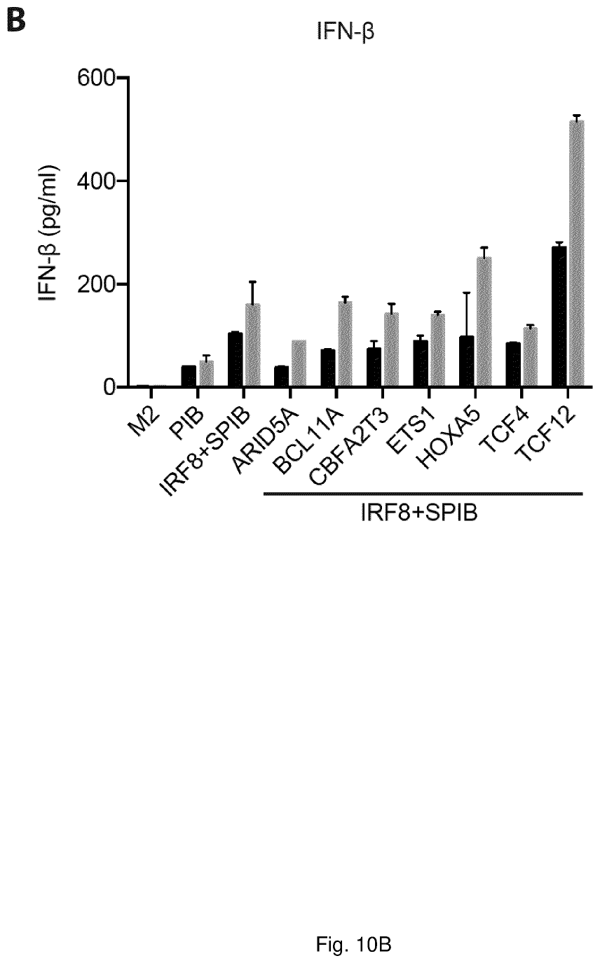

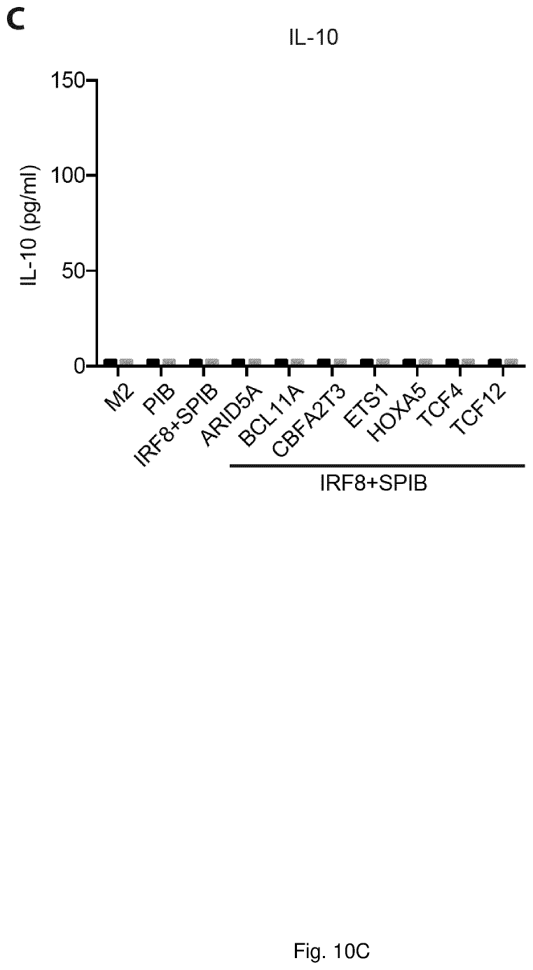

In an embodiment, in addition to MHC-II surface molecule, pDCs secrete cytokines with a pro-inflammatory function that are important for the development of T-cell responses. These responses can be initiated by the triggering of at least 11 different Toll-like receptors (TLRs), allowing the specific recognition of distinct conserved microbial or viral structures. Within the DC compartment, pDCs specifically respond to TLR7 and TLR9 triggering (FIG. 3). It was studied whether induced pDCs secrete cytokines to the media when challenged with TLR7 (using Resiquimod or R848) and TLR9 (using ODN 1668) stimulation (FIG. 10). Upon TLR challenge of induced pDCs it was observed an increase in the secretion of IFN-α by tdTomato+ cells reprogrammed with IRF8 and SPIB (16.6 fold); IRF8, SPIB and ARID5A (3.9 fold); IRF8, SPIB and BCL11A (14.8 fold); IRF8, SPIB and CBFA2T3 (3.5 fold); IRF8, SPIB and ETS1 (3.7 fold); IRF8, SPIB and TCF4 (3.6 fold); IRF8, SPIB and TCF12 (8.3 fold); and IRF8, SPIB and HOXA5 (7.7 fold), when comparing stimulated with non-stimulated cells generated by each TF combination. TLR challenge of induced pDCs also increased the secretion of IFN-β by tdTomato+ cells reprogrammed with IRF8 and SPIB (1.5 fold); IRF8, SPIB and ARID5A (2.3 fold); IRF8, SPIB and BCL11A (2.3 fold); IRF8, SPIB and CBFA2T3 (1.9 fold); IRF8, SPIB and ETS1 (1.6 fold); IRF8, SPIB and TCF4 (1.3 fold); IRF8, SPIB and TCF12 (1.9 fold); and IRF8, SPIB and HOXA5 (2.6 fold). Upon TLR stimulation of induced pDCs no increase in the secretion of the anti-inflammatory cytokine IL-10 was observed. Importantly, tdTomato+ cells obtained upon transduction with PU.1, IRF8 and BATF3 (PIB) combination of TFs did not respond to TLR7 and TLR9 and did not secrete IFN-α and IFN-β. Taken together, these data suggests that by combining Clec9a reporter activation with interferon secretion ability, we successfully identified combinations of TFs able to induce pDCs or interferon type I-secreting cells by direct cell reprogramming.

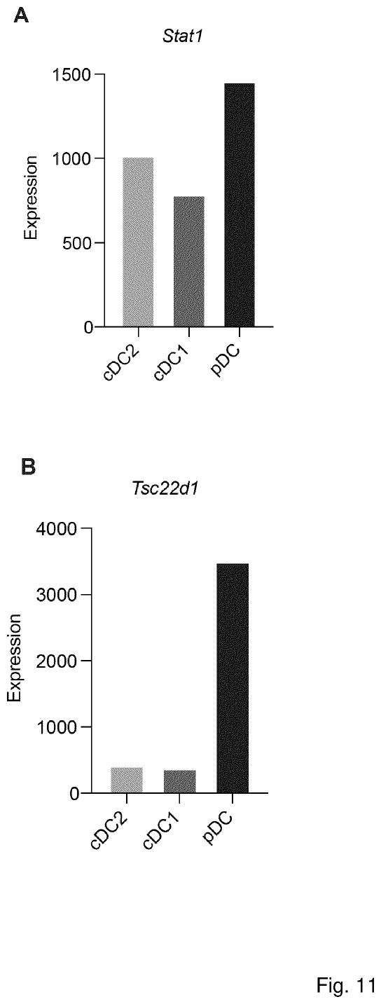

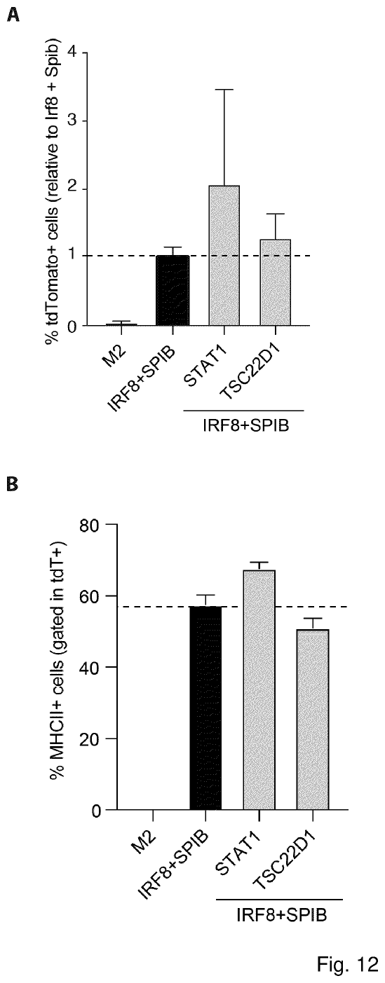

In an embodiment, two additional candidate TFs were selected due to their specific enriched gene expression in plasmacytoid DCs when compared to cDC1 s and cDC2s: STAT1 and TSC22D1 (FIG. 11). These two TFs were cloned individually in the same reprogramming proven Doxycycline (Dox)-inducible lentiviral vector and tested in combination with IRF8 and SPIB to access their ability to induce Clec9a-tdTomato reporter activation (FIG. 12). Interestingly, the addition of STAT1 or TSC22D1 to the IRF8 and SPIB combination increased Clec9a reporter activation when compared with IRF8 and SPIB alone (FIG. 12A). Induced pDCs generated by IRF8, SPIB and STAT1, and IRF8, SPIB and TSC22D1 also express MHC-II molecules at the cell surface (FIG. 12B), supporting the acquisition of pDC fate.

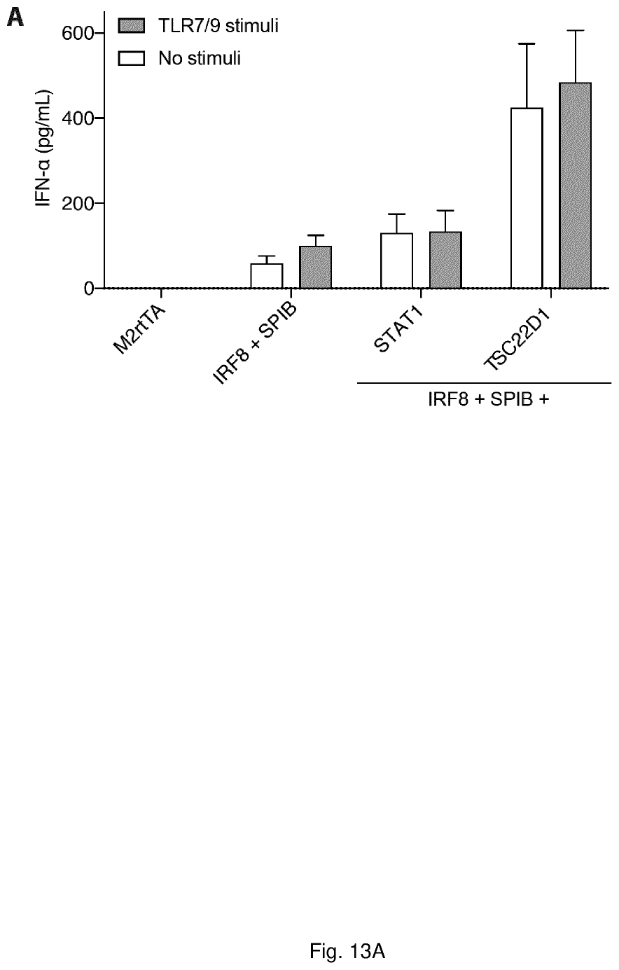

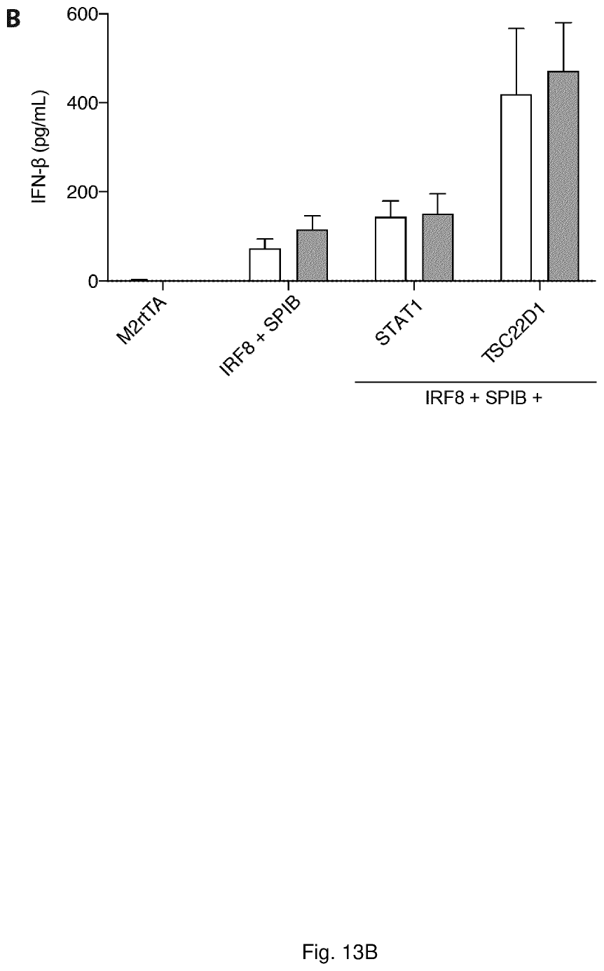

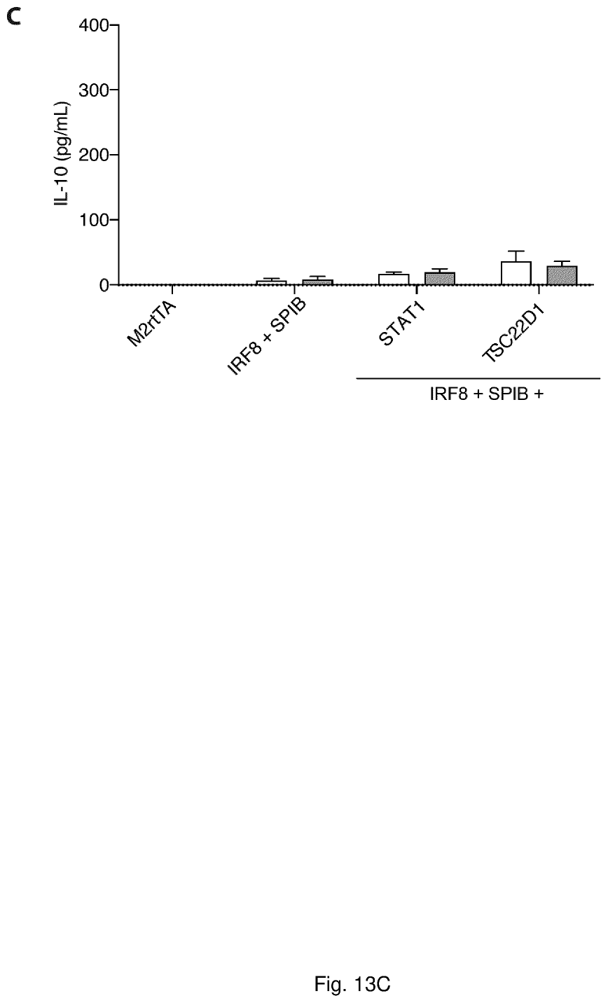

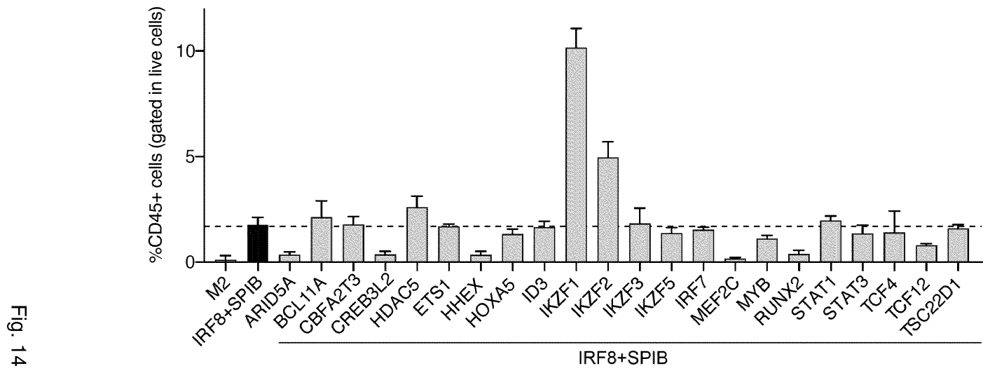

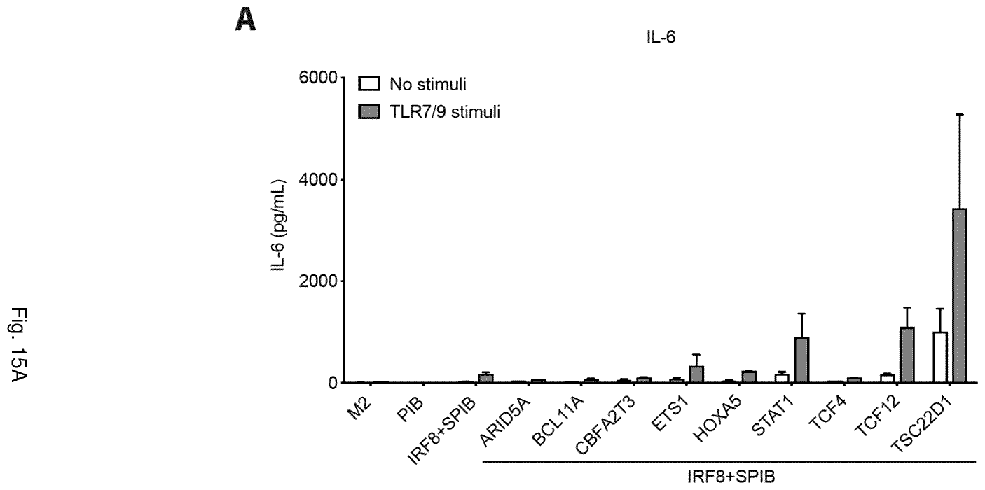

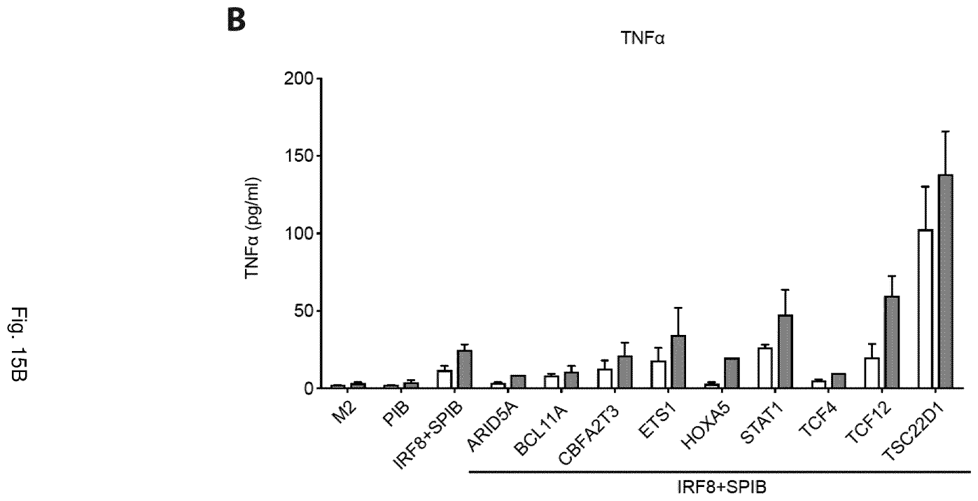

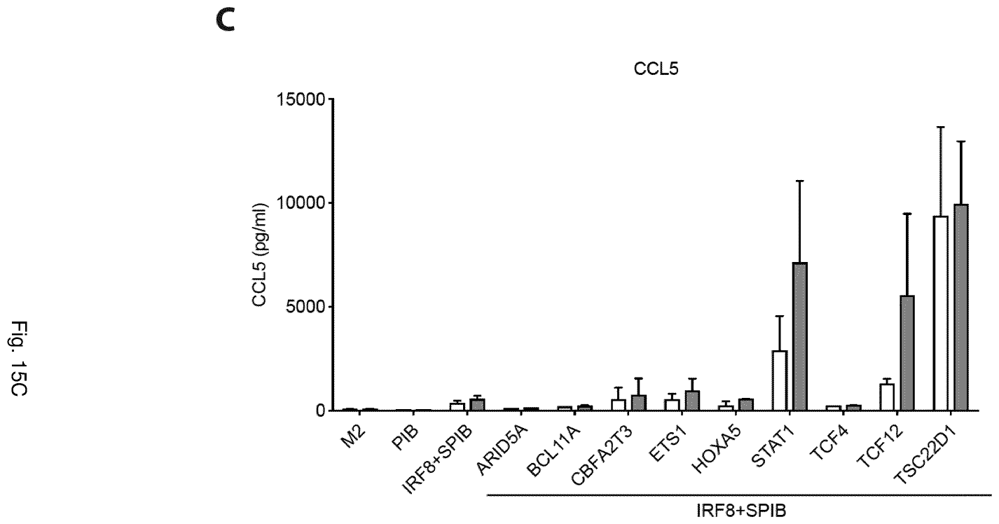

In an embodiment, induced pDCs were challenged with TLR7/9, at which an increased secretion of IFN-α by tdTomato+ cells reprogrammed with IRF8, SPIB and STAT1 (1.3 fold) and IRF8, SPIB and TSC22D1 (4.8 fold), when comparing with induced pDCs generated by IRF8 and SPIB (FIG. 13A), was observed. Addition of STAT1 or TSC22D1 also increased the secretion of IFN-β by tdTomato+ cells reprogrammed with IRF8, SPIB and STAT1 (1.3 fold) and IRF8, SPIB and TSC22D1 (4.1 fold) (FIG. 13B) in comparison with IRF8 and SPIB-induced cells. Induced pDCs generated by IRF8, SPIB and STAT1, and IRF8, SPIB and TSC22D1 were not able to secrete anti-inflammatory cytokine IL-10. These data support that IRF8, SPIB and STAT1, and IRF8, SPIB and TSC22D1 are able to induce reprogramming towards an induced pDC fate and ability to secrete type I-interferons.

In an embodiment, surface expression of hematopoietic marker CD45 is also a distinctive feature of pDCs. It was investigated whether combinations of pDC-inducing factors are able to induce surface expression of CD45. CD45 is expressed in 1.79±0.33% of induced pDCs generated with IRF8 and SPIB. Surprisingly, if IKZF1 is combined with IRF8 and SPIB, the population of CD45+ increases up to 10.16±0.91% (5.6 fold increase).

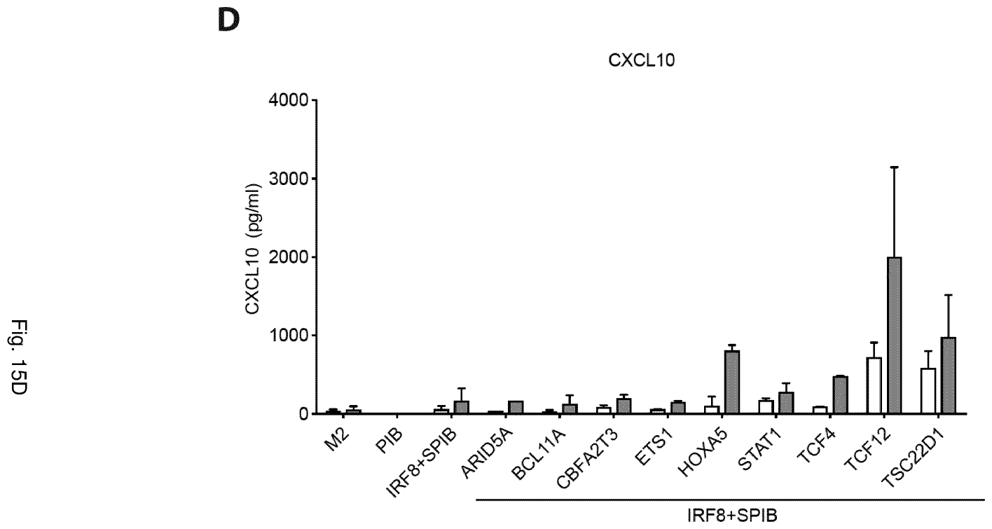

In an embodiment, the recognition of viruses or self nucleic acids by TLR7 and TLR9 in pDCs induces two alternative pathways dependent on the type of compartment in which TLR7 and TLR9 encounter their ligands. The MYD88-IRF7 pathway results in type I interferon secretion, whilst the MYD88-NF-κB pathway leads to production of pro-inflammatory cytokines and chemokines, It was then investigated if upon TLR7/9 stimulation, induced pDCs secrete additional cytokines (FIG. 15). Indeed, purified tdTomato+ cells generated by overexpression of IRF8 and SPIB secrete pro-inflammatory cytokines IL-6 and TNF-α and chemokines CCL5 and CXCL10. Importantly, the addition of STAT1, TCF12 or TSC22D1 to IRF8 and SPIB combination increases the secretion of IL-6, TNF-α, CCL5 and CXCL10, in a TLR-dependent manner. TLR challenge of induced pDCs increased the secretion of IL-6 by tdTomato+ cells reprogrammed with IRF8, SPIB and STAT1 (5.6 fold), IRF8, SPIB and TCF12 (7.5 fold) and IRF8, SPIB and TSC22D1 (3.5 fold), whilst similar increase was observed for TNF-α (1.8, 3.1, 1.3 fold, respectively). Secretion of the chemokine CCL5 by tdTomato+ cells reprogrammed with IRF8, SPIB and STAT1 (2.5 fold), IRF8, SPIB and TCF12 (3.1 fold) and IRF8, SPIB and TSC22D1 (2.4 fold) increased upon TLR challenge. As far as CXCL10, induced pDCs generated by overexpression of IRF8, SPIB and TCF12, and IRF8, SPIB and TSC22D1, also increased their secretion upon TLR stimulation, 2.8 and 1.7 fold, respectively. Taken together, this data support that we successfully identified combinations of TFs sufficient to induce pDCs in unrelated cell types with ability to respond to TLR7 and 9 stimulation through two intra-cellular pathways, leading to type-I interferon secretion and pro-inflammatory cytokines and chemokines.

In an embodiment, coding regions of each candidate TF were individually cloned into an inducible lentiviral pFUW-TetO vector in which the expression of the TFs is under the control of the tetracycline operator and a minimal CMV promoter. A previously described lentiviral vector containing the reverse tetracycline transactivator M2rtTA under the control of a constitutively active human ubiquitin C promoter (FUW-M2rtTA) was used in combination. Human embryonic kidney (HEK) 293T cells were transfected with a mixture of TF-encoding plasmids, packaging constructs and the VSV-G envelope protein. Viral supernatants were harvested after 36, 48 and 60 hours, filtered (0.45 μm, Corning) and used fresh or concentrated 40-fold with Amicon ultra centrifugal filters (Millipore).

In some embodiments, polypeptide variants or family members having the same or a similar activity as the reference polypeptide encoded by the sequences provided in the sequence list can be used in the compositions, methods, and kits described herein. Generally, variants of a particular polypeptide encoding a pDC-inducing factor for use in the compositions, methods, and kits described herein will have at least about 95%, at least about 96%, at least about 97%, at least about 98%, at least about 99% or more sequence identity to that particular reference polynucleotide or polypeptide as determined by sequence alignment programs and parameters described herein and known to those skilled in the art.

In an embodiment, Homo sapiens Interferon Regulatory Factor 8 (IRF8), mRNA (SEQ. ID. 1) and a codon optimized, or different codons encoding the same amino acids, are naturally also contemplated to be covered by the reference to the nucleic acid as set forth herein.

In an embodiment, Homo sapiens Spi-B (SPIB), mRNA (SEQ. ID. 3) and a codon optimized, or different codons encoding the same amino acids, are naturally also contemplated to be covered by the reference to the nucleic acid as set forth herein.

In an embodiment, Homo sapiens AT-Rich Interaction Domain 5A (ARID5A), mRNA (SEQ. ID. 5) and a codon optimized, or different codons encoding the same amino acids, are naturally also contemplated to be covered by the reference to the nucleic acid as set forth herein.

In an embodiment, Homo sapiens BAF Chromatin Remodeling Complex Subunit (BCL11A), mRNA (SEQ. ID. 7) and a codon optimized, or different codons encoding the same amino acids, are naturally also contemplated to be covered by the reference to the nucleic acid as set forth herein.

In an embodiment, Homo sapiens CBFA2/RUNX1 Partner Transcriptional Co-Repressor 3 (CBFA2T3), mRNA (SEQ. ID. 9) and a codon optimized, or different codons encoding the same amino acids, are naturally also contemplated to be covered by the reference to the nucleic acid as set forth herein.

In an embodiment, Homo sapiens CAMP Responsive Element Binding Protein 3 Like 2 (CREB3L2), mRNA (SEQ. ID. 11) and a codon optimized, or different codons encoding the same amino acids, are naturally also contemplated to be covered by the reference to the nucleic acid as set forth herein.

In an embodiment, Homo sapiens ETS Proto-Oncogene 1 (ETS1), mRNA (SEQ. ID. 13) and a codon optimized, or different codons encoding the same amino acids, are naturally also contemplated to be covered by the reference to the nucleic acid as set forth herein.

In an embodiment, Homo sapiens Homeobox A5 (HOXA5), mRNA (SEQ. ID. 19) and a codon optimized, or different codons encoding the same amino acids, are naturally also contemplated to be covered by the reference to the nucleic acid as set forth herein.

In an embodiment, Homo sapiens IKAROS Family Zinc Finger 1 (IKZF1) mRNA (SEQ. ID. 23) and a codon-optimized, or different codons encoding the same amino acids, are naturally contemplated to be covered by the reference to the nucleic acid as set forth herein.

In an embodiment, Homo sapiens Signal Transducer And Activator Of Transcription 1 (STAT1), mRNA (SEQ. ID. 45) and a codon-optimized, or different codons encoding the same amino acids, are naturally contemplated to be covered by the reference to the nucleic acid as set forth herein.

In an embodiment, Homo sapiens Transcription Factor 4 (TCF4), mRNA (SEQ. ID. 41) and a codon optimized, or different codons encoding the same amino acids, are naturally also contemplated to be covered by the reference to the nucleic acid as set forth herein.

In an embodiment, Homo sapiens Transcription Factor 12 (TCF12), mRNA (SEQ. ID. 43) and a codon optimized, or different codons encoding the same amino acids, are naturally also contemplated to be covered by the reference to the nucleic acid as set forth herein.

In an embodiment, Homo sapiens TSC22 domain family protein 1 (TSC22D1), mRNA (SEQ. ID. 47) and a codon-optimized, or different codons encoding the same amino acids, are naturally contemplated to be covered by the reference to the nucleic acid as set forth herein.

In some embodiments of the compositions, vectors, constructs, methods, and kids provided herein, the number of pDC-inducing factors used or selected to generate pDCs from a starting somatic cell, such as a fibroblast cell or hematopoietic lineage cell, a multipotent stem cell, an induced pluripotent stem cell, a cancer or tumor cell is at least two. In some embodiments, the number of pDC-inducing factors used or selected is at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least thirty, at least thirty three, at least thirty five, at least forty, or more.

In some embodiments of the compositions, vectors, constructs, methods, and kits described herein, the nucleic acid sequence or construct encoding the pDC-inducing factor(s), such as IRF8, SPIB, ARID5A, BCL1A, CBFA2T3, CREB3L2, ETS1, HOXA5, IZKF1, TCF4, TCF12 and TSC22D1 and, is inserted or operably linked into a suitable expression vector for transfection of cells using standard molecular biology techniques. As used herein, a “vector” refers to a nucleic acid molecule, such as a dsDNA molecule that provides a useful biological or biochemical property to an inserted nucleotide sequence, such as the nucleic acid constructs or replacement cassettes described herein. Examples include plasmids, phages, autonomously replicating sequences (ARS), centromeres, and other sequences that are able to replicate or be replicated in vitro or in a host cell, or to convey a desired nucleic acid segment to a desired location within a host cell. A vector can have one or more restriction endonuclease recognition sites (whether type I, II, IIs, III and IV) at which the sequences can be cut in a determinable fashion without loss of an essential biological function of the vector, and into which a nucleic acid fragment can be spliced or inserted in order to bring about its replication and cloning. Vectors can also comprise one or more recombination sites that permit exchange of nucleic acid sequences between two nucleic acid molecules. Vectors can further provide primer sites, e.g., for PCR, transcriptional and/or translational initiation and/or regulation sites, recombination signals, replicons, additional selectable markers, etc. A vector can further comprise one or more selectable markers suitable for use in the identification of cells transformed with the vector.

In some embodiments of the compositions, vectors, constructs, methods, and kits described herein, the expression vector is a viral vector. Some viral-mediated expression methods employ retrovirus, adenovirus, lentivirus, paramyxoviridae, rabdoviral, alphaviral, flaviral, herpes virus, pox virus, and adeno-associated virus (AAV) vectors, and such expression methods have been used in gene delivery and are well known in the art.

In some embodiments of the compositions, vectors, constructs, methods, and kits described herein, the viral vector is a retrovirus. Retroviruses provide a convenient platform for gene delivery. A selected gene can be inserted into a vector and packaged in retroviral particles using techniques known in the art. The recombinant virus can then be isolated and delivered to target cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09. In some embodiments of the compositions, vectors, methods, and kits described herein, the retrovirus is replication deficient. Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

In some embodiments of the compositions, vectors, constructs, methods, and kits described herein, the viral vector is an adenovirus-based expression vector. Unlike retroviruses, which integrate into the host genome, adenoviruses persist extra-chromosomally, thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76). Adenoviral vectors infect a wide variety of cells, have a broad host-range, exhibit high efficiencies of infectivity, direct expression of heterologous genes at high levels, and achieve long-term expression of those genes in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“E1”) of the viral genome. Adenovirus can also be produced in large quantities with relative ease. Adenoviral vectors for use in the compositions, vectors, methods, and kits described herein can be derived from any of the various adenoviral serotypes, including, without limitation, any of the over 40 serotype strains of adenovirus, such as serotypes 2, 5, 12, 40, and 41. The adenoviral vectors used herein are preferably replication-deficient and contain the pDC-inducing factor of interest operably linked to a suitable promoter.

In some embodiments of the compositions, vectors, constructs, methods, and kits described herein, the nucleic acid sequences encoding the pDC-inducing factor(s), such as IRF8, SPIB, ARID5A, BCL1A, CBFA2T3, ETS1, HOXA5, IKZF1, STAT1, TCF4, TCF12 and TSC22D1 are introduced or delivered using one or more inducible lentiviral vectors. Control of expression of pDC-inducing factors delivered using one or more inducible lentiviral vectors can be achieved, in some embodiments, by contacting a cell having at least one pDC-inducing factor in an expression vector under the control of or operably linked to an inducible promoter, with a regulatory agent (e.g., doxycycline) or other inducing agent. When using some types of inducible lentiviral vectors, contacting such a cell with an inducing agent induces expression of the pDC-inducing factors, while withdrawal of the regulatory agent inhibits expression. When using other types of inducible lentiviral vectors, the presence of the regulatory agent inhibits expression, while removal of the regulatory agent permits expression. As used herein, the term “induction of expression” refers to the expression of a gene, such as a pDC-inducing factor encoded by an inducible viral vector, in the presence of an inducing agent, for example, or in the presence of one or more agents or factors that cause endogenous expression of the gene in a cell.

In some embodiments of the aspects described herein, a doxycycline (Dox) inducible lentiviral system is used. Unlike retroviruses, lentiviruses are able to transduce quiescent cells making them amenable for transducing a wider variety of hematopoietic cell types. For example, the pFUW-tetO lentivirus system has been shown to transduce primary hematopoietic progenitor cells with high efficiency.

In some embodiments of the methods described herein, the nucleic acid sequences encoding the pDC-inducing factor(s), such as IRF8 (SEQ. ID. 1, SEQ. ID. 2), SPIB (SEQ. ID. 3, SEQ. ID. 4), ARID5A (SEQ. ID. 5, SEQ. ID. 6), BCL11A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), HDAC5 (SEQ. ID. 15, SEQ. ID. 16), HHEX (SEQ. ID. 17, SEQ. ID. 18), HOXA5 (SEQ. ID. 19, SEQ. ID. 20), ID3 (SEQ. ID. 21, SEQ. ID. 22), IKZF1 (SEQ. ID. 23, SEQ. ID. 24), IKZF2 (SEQ. ID. 25, SEQ. ID. 26), IKZF3 (SEQ. ID. 27, SEQ. ID. 28), IKZF5 (SEQ. ID. 29, SEQ. ID. 30), IRF7 (SEQ. ID. 31, SEQ. ID. 32), MEF2C (SEQ. ID. 33, SEQ. ID. 34), MYB (SEQ. ID. 35, SEQ. ID. 36), RUNX2 (SEQ. ID. 37, SEQ. ID. 38), STAT3 (SEQ. ID. 39, SEQ. ID. 40), TCF4 (SEQ. ID. 41, SEQ. ID. 42), TCF12 (SEQ. ID. 43, SEQ. ID. 44), STAT1 (SEQ. ID. 45, SEQ. ID. 46) and TSC22D1 (SEQ. ID. 47, SEQ. ID. 48) are introduced or delivered using a non-integrating vector (e.g., adenovirus). While integrating vectors, such as retroviral vectors, incorporate into the host cell genome and can potentially disrupt normal gene function, non-integrating vectors control expression of a gene product by extra-chromosomal transcription. Since non-integrating vectors do not become part of the host genome, non-integrating vectors tend to express a nucleic acid transiently in a cell population. This is due in part to the fact that the non-integrating vectors are often rendered replication deficient. Thus, non-integrating vectors have several advantages over retroviral vectors including, but not limited to: (1) no disruption of the host genome, and (2) transient expression, and (3) no remaining viral integration products. Some non-limiting examples of non-integrating vectors for use with the methods described herein include adenovirus, baculovirus, alphavirus, picornavirus, and vaccinia virus. In some embodiments of the methods described herein, the non-integrating viral vector is an adenovirus. Other advantages of non-integrating viral vectors include the ability to produce them in high titers, their stability in vivo, and their efficient infection of host cells.

Nucleic acid constructs and vectors for use in generating induced pDCs in the compositions, vectors, construts, methods, and kits described herein can further comprise, in some embodiments, one or more sequences encoding selection markers for positive and negative selection of cells. Such selection marker sequences can typically provide properties of resistance or sensitivity to antibiotics that are not normally found in the cells in the absence of introduction of the nucleic acid construct. A selectable marker can be used in conjunction with a selection agent, such as an antibiotic, to select in culture for cells expressing the inserted nucleic acid construct. Sequences encoding positive selection markers typically provide antibiotic resistance, i.e., when the positive selection marker sequence is present in the genome of a cell, the cell is sensitive to the antibiotic or agent. Sequences encoding negative selection markers typically provide sensitivity to an antibiotic or agent, i.e., when the negative selection marker is present in the genome of a cell, the cell is sensitive to the antibiotic or agent.

Nucleic acid constructs and vectors for use in making induced pDCs in the compositions, vectors, constructs, methods, and kits thereof described herein can further comprise, in some embodiments, other nucleic acid elements for the regulation, expression, stabilization of the construct or of other vector genetic elements, for example, promoters, enhancers, TATA-box, ribosome binding sites, IRES, 2A-like self-cleaving sequences as known to one of ordinary skill in the art.

Nucleic acid constructs and vectors for use in making induced pDCs in the compositions, vectors, constructs, methods, and kits described herein can further comprise a 2A-like self-cleaving sequences. Self-cleaving 18-22 amino acids long 2A peptides mediate ‘ribosomal skipping’ between the proline and glycine residues and inhibit peptide bond formation without affecting downstream translation. These peptides allow multiple proteins to be encoded as polyproteins, which dissociate into component proteins upon translation. Examples of 2A-like self cleaving sequences that can be used according to the invention include without limitation, those from Picomaviridae virus family, including aphthoviruses such as foot-and-mouth disease virus (FMDV), equine rhinitis A virus (ERAV), Thosea asigna virus (TaV) and porcine teschovirus-1 (PTV-1) and cardioviruses such as Theilovirus (e.g., Theiler's murine encephalomyelitis) and encephalomyocarditis viruses.

In some embodiments of the compositions, vectors, constructs, methods, and kits described herein, the pDC-inducing factor(s), such as IRF8 (SEQ. ID. 1, SEQ. ID. 2), SPIB (SEQ. ID. 3, SEQ. ID. 4), ARID5A (SEQ. ID. 5, SEQ. ID. 6), BCL11A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), HOXA5 (SEQ. ID. 19, SEQ. ID. 20), IKZF1 (SEQ. ID. 23, SEQ. ID. 24), TCF4 (SEQ. ID. 41, SEQ. ID. 42), TCF12 (SEQ. ID. 43, SEQ. ID. 44), STAT1 (SEQ. ID. 45, SEQ. ID. 46), and TSC22D1 (SEQ. ID. 47, SEQ. ID. 48) and, are provided as synthetic, modified RNAs, or introduced or delivered into a cell as a synthetic, modified RNA, as described in US Patent Publication 2012-0046346-A1, the contents of which are herein incorporated by reference in their entireties. In those embodiments where synthetic, modified RNAs are used to reprogram cells to induced pDCs according to the methods described herein, the methods can involve repeated contacting of the cells or involve repeated transfections of the synthetic, modified RNAs encoding pDC-inducing factors, such as for example, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, or more transfections.

In addition to one or more modified nucleosides, the modified mRNAs for use in the compositions, vectors, constructs, methods, and kits described herein can comprise any additional modifications known to one of skill in the art and as described in US Patent Publications 2012-0046346-A1 and 20120251618A1, and PCT Publication WO 2012/019168. Such other components include, for example, a 5′ cap (e.g., the Anti-Reverse Cap Analog (ARCA) cap, which contains a 5′-5′-triphosphate guanine-guanine linkage where one guanine contains an N7 methyl group as well as a 3′-O-methyl group; caps created using recombinant Vaccinia Virus Capping Enzyme and recombinant 2′-O-methyltransferase enzyme, which can create a canonical 5′-5′-triphosphate linkage between the 5′-most nucleotide of an mRNA and a guanine nucleotide where the guanine contains an N7 methylation and the ultimate 5′-nucleotide contains a 2′-O-methyl generating the Cap1 structure); a poly(A) tail (e.g., a poly-A tail greater than 30 nucleotides in length, greater than 35 nucleotides in length, at least 40 nucleotides, at least 45 nucleotides, at least 55 nucleotides, at least 60 nucleotide, at least 70 nucleotides, at least 80 nucleotides, at least 90 nucleotides, at least 100 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, or more); a Kozak sequence; a 3′ untranslated region (3′ UTR); a 5′ untranslated region (5′ UTR); one or more intronic nucleotide sequences capable of being excised from the nucleic acid, or any combination thereof.

The modified mRNAs for use in the compositions, vectors, constructs, methods, and kits described herein can further comprise an internal ribosome entry site (IRES). An IRES can act as the sole ribosome binding site, or can serve as one of multiple ribosome binding sites of an mRNA. An mRNA containing more than one functional ribosome binding site can encode several peptides or polypeptides, such as the pDC-inducing factors described herein, that are translated independently by the ribosomes (“multicistronic mRNA”). When nucleic acids are provided with an IRES, further optionally provided is a second translatable region. Examples of IRES sequences that can be used according to the invention include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SW) or cricket paralysis viruses (CrPV).

In some embodiments of the compositions, vectors, constructs, methods, and kits described herein, the synthetic, modified RNA molecule comprises at least one modified nucleoside. In some embodiments of the compositions, vectors, methods, and kits described herein, the synthetic, modified RNA molecule comprises at least two modified nucleosides.

In some embodiments of the compositions, vectors, constructs, methods, and kits described herein, the modified nucleosides are selected from the group consisting of 5-methylcytosine (5mC), N6-methyladenosine (m6A), 3,2′-O-dimethyluridine (m4U), 2-thiouridine (s2U), 2′ fluorouridine, pseudouridine, 2′-O-methyluridine (Um), 2′deoxy uridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m2,7G), N2,N2,7-trimethylguanosine (m2,2,7G), and inosine (I). In some embodiments, the modified nucleosides are 5-methylcytosine (5mC), pseudouracil, or a combination thereof.

Modified mRNAs need not be uniformly modified along the entire length of the molecule. Different nucleotide modifications and/or backbone structures can exist at various positions in the nucleic acid. One of ordinary skill in the art will appreciate that the nucleotide analogs or other modification(s) can be located at any position(s) of a nucleic acid such that the function of the nucleic acid is not substantially decreased. A modification can also be a 5′ or 3′ terminal modification. The nucleic acids can contain at a minimum one and at maximum 100% modified nucleotides, or any intervening percentage, such as at least 50% modified nucleotides, at least 80% modified nucleotides, or at least 90% modified nucleotides.

In some embodiments of the present disclosure, the vector or construct is a replicating viral vector or particle, such as a Paramyxoviridae vector, Rhabdoviral vector, Alphaviral and/or Flaviviral particles, or a non-viral vector or particle, such as a naked Alphaviral or Flaviviral RNA replicon.

In some embodiments, it is preferred, but not absolutely necessary, that each occurrence of a given nucleoside in a molecule is modified (e.g., each cytosine is a modified cytosine e.g., 5-methylcytosine, each uracil is a modified uracil, e.g., pseudouracil, etc.). For example, the modified mRNAs can comprise a modified pyrimidine such as uracil or cytosine. In some embodiments, at least 25%, at least 50%, at least 80%, at least 90% or 100% of the uracil in the nucleic acid are replaced with a modified uracil. It is also contemplated that different occurrences of the same nucleoside can be modified in a different way in a given synthetic, modified RNA molecule. The modified uracil can be replaced by a compound having a single unique structure, or can be replaced by a plurality of compounds having different structures (e.g., 2, 3, 4 or more unique structures). In some embodiments, at least 25%, at least 50%, at least 80%, at least 90% or 100% of the cytosine in the nucleic acid may be replaced with a modified cytosine. The modified cytosine can be replaced by a compound having a single unique structure, or can be replaced by a plurality of compounds having different structures (e.g., 2, 3, 4 or more unique structures) (e.g., some cytosines modified as 5mC, others modified as 2′-O-methylcytosine or other cytosine analog). Such multi-modified synthetic RNA molecules can be produced by using a ribonucleoside blend or mixture comprising all the desired modified nucleosides, such that when the RNA molecules are being synthesized, only the desired modified nucleosides are incorporated into the resulting RNA molecule encoding the pDC-inducing factor.

In certain embodiments it is desirable to intracellularly degrade a modified nucleic acid introduced into the cell, for example if precise timing of protein production is desired. Thus, in some embodiments of the compositions, vectors, methods, and kits described herein, provided herein are modified nucleic acids comprising a degradation domain, which is capable of being acted on in a directed manner within a cell.

While it is understood that induced pDCs can be generated by delivery of pDC-inducing factors in the form of nucleic acid (DNA or RNA) or amino acid sequences, in some embodiments of the compositions, vectors, methods, and kits described herein, induced pDCs can be induced using other methods, such as, for example, by treatment of cells with an agent, such as a small molecule or cocktail of small molecules, that induce expression of one or more of the pDC-inducing factors.

Detection of expression of pDC-inducing factors introduced into cells or induced in a cell population using the compositions, vectors, methods, and kits described herein, can be achieved by any of several techniques known to those of skill in the art including, for example, Western blot analysis, immunocytochemistry, and fluorescence-mediated detection.

In order to distinguish whether a given combination of pDC-inducing factors has generated induced pDCs, one or more DC activities or parameters can be measured, such as, in some embodiments, differential expression of surface antigens. The generation of induced DCs using the compositions, vectors, methods, and kits described herein preferably causes the appearance of the cell surface phenotype characteristic of endogenous pDCs, such as CD45 and MHC-II, for example.

DCs are most reliably distinguished from other immune cells by their functional behavior. Functional aspects of DC phenotypes, or DC activities, such as the ability of an induced pDC to secrete cytokines can be easily determined by one of skill in the art using routine methods known in the art. In some embodiments of the aspects described herein, functional assays to identify reprogramming factors can be used. For example, in some embodiments, cytokine secretion can be used to confirm immune-modulatory properties of induced pDCs generated using the compositions, vectors, methods, and kits described herein. In particular, ability to respond to intracellular DNA though TLR7 and TLR9 pathways and secrete type I interferons is particularly relevant to characterize pDC functionality.

As used herein, “cellular parameter,” “DC parameter,” or “cytokine secretion” refer to measureable components or qualities of endogenous or natural DCs, particularly components that can be accurately measured. A cellular parameter can be any measurable parameter related to a phenotype, function, or behavior of a cell. Such cellular parameters include, changes in characteristics and markers of a DC or DC population, including but not limited to changes in viability, cell growth, expression of one or more or a combination of markers, such as cell surface determinants, such as receptors, proteins, including conformational or posttranslational modification thereof, lipids, carbohydrates, organic or inorganic molecules, nucleic acids, e.g. mRNA, DNA, global gene expression patterns, etc. Such cellular parameters can be measured using any of a variety of assays known to one of skill in the art. For example, viability and cell growth can be measured by assays such as Trypan blue exclusion, CFSE dilution, and 3H-thymidine incorporation. Expression of protein or polypeptide markers can be measured, for example, using flow cytometric assays, Western blot techniques, or microscopy methods. Gene expression profiles can be assayed, for example, using RNA-sequencing methodologies and quantitative or semi-quantitative real-time PCR assays. A cellular parameter can also refer to a functional parameter or functional activity. While most cellular parameters will provide a quantitative readout, in some instances a semi-quantitative or qualitative result can be acceptable. Readouts can include a single determined value, or can include mean, median value or the variance, etc. Characteristically a range of parameter readout values can be obtained for each parameter from a multiplicity of the same assays. Variability is expected and a range of values for each of the set of test parameters will be obtained using standard statistical methods with a common statistical method used to provide single values.

In some embodiments of the compositions, vectors, methods, and kits described herein, additional factors and agents can be used to enhance induced pDC reprogramming. For example, factors and agents that modify epigenetic pathways can be used to facilitate reprogramming into induced pDCs.

Essentially any primary somatic cell type can be used for producing induced pDCs or reprogramming somatic cells to induced pDCs according to the presently described compositions, vectors, methods, and kits. Such primary somatic cell types also include other stem cell types, including pluripotent stem cells, such as induced pluripotent stem cells (iPS cells); other multipotent stem cells; oligopotent stem cells; and unipotent stem cells. Some non-limiting examples of primary somatic cells useful in the various aspects and embodiments of the methods described herein include, but are not limited to, fibroblasts, epithelial, endothelial, neuronal, adipose, cardiac, skeletal muscle, hematopoietic or immune cells, hepatic, splenic, lung, circulating blood cells, gastrointestinal, renal, bone marrow, and pancreatic cells, as well as stem cells from which those cells are derived. The cell can be a primary cell isolated from any somatic tissue including, but not limited to, spleen, bone marrow, blood, brain, liver, lung, gut, stomach, intestine, fat, muscle, uterus, skin, spleen, endocrine organ, bone, etc. The term “somatic cell” further encompasses, in some embodiments, primary cells grown in culture, provided that the somatic cells are not immortalized. Where the cell is maintained under in vitro conditions, conventional tissue culture conditions and methods can be used, and are known to those of skill in the art. Isolation and culture methods for various primary somatic cells are well within the abilities of one skilled in the art.

In some embodiments of these aspects and all such aspects described herein, the somatic cell is a fibroblast cell.

In some embodiments of these aspects and all such aspects described herein, the somatic cell can be a hematopoietic lineage cell.

In some embodiments of these aspects and all such aspects described herein, the somatic cell can be a cancer cell or a tumor cell.

In some embodiments of the compositions, vectors, constructs, methods, and kits described herein, a somatic cell to be reprogrammed or made into an induced pDC cell is a cell of hematopoietic origin. As used herein, the terms “hematopoietic-derived cell,” “hematopoietic-derived differentiated cell,” “hematopoietic lineage cell,” and “cell of hematopoietic origin” refer to cells derived or differentiated from a multipotent hematopoietic stem cell (HSC). Accordingly, hematopoietic lineage cells for use with the compositions, vectors, constructs, methods, and kits described herein include multipotent, oligopotent, and lineage-restricted hematopoietic progenitor cells, granulocytes (e.g., promyelocytes, neutrophils, eosinophils, basophils), erythrocytes (e.g., reticulocytes, erythrocytes), thrombocytes (e.g., megakaryoblasts, platelet-producing megakaryocytes, platelets), monocytes (e.g., monocytes, macrophages), dendritic cells, and lymphocytes (e.g., T-lymphocytes, which carry T-cell receptors (TCRs), B-lymphocytes or B cells, which express immunoglobulin and produce antibodies, NK cells, NKT cells, and innate lymphocytes). As used herein, the term “hematopoietic progenitor cells” refer to multipotent, oligopotent, and lineage-restricted hematopoietic cells capable of differentiating into two or more cell types of the hematopoietic system, including, but not limited to, granulocytes, monocytes, erythrocytes, megakaryocytes, and lymphocytes B-cells and T-cells. Hematopoietic progenitor cells encompass multi-potent progenitor cells (MPPs), common myeloid progenitor cells (CMPs), common lymphoid progenitor cells (CLPs), granulocyte-monocyte progenitor cells (GMPs), and pre-megakaryocyte-erythrocyte progenitor cell. Lineage-restricted hematopoietic progenitor cells include megakaryocyte-erythrocyte progenitor cells (MEP), ProB cells, PreB cells, PreProB cells, ProT cells, double-negative T cells, pro-NK cells, pre-granulocyte/macrophage cells, granulocyte/macrophage progenitor (GMP) cells, and pro-mast cells (ProMCs).

Cells of hematopoietic origin for use in the compositions, vectors, methods, and kits described herein can be obtained from any source known to comprise these cells, such as fetal tissues, umbilical cord blood, bone marrow, peripheral blood, mobilized peripheral blood, spleen, liver, thymus, lymph, etc. Cells obtained from these sources can be expanded ex vivo using any method acceptable to those skilled in the art prior to use in with the compositions, vectors, constructs, methods, and kits for making induced pDCs described herein. For example, cells can be sorted, fractionated, treated to remove specific cell types, or otherwise manipulated to obtain a population of cells for use in the methods described herein using any procedure acceptable to those skilled in the art. Mononuclear lymphocytes may be collected, for example, by repeated lymphocytophereses using a continuous flow cell separator as described in U.S. Pat. No. 4,690,915, or isolated using an affinity purification step of CLP method, such as flow-cytometry using a cytometer, magnetic separation, using antibody or protein coated beads, affinity chromatography, or solid-support affinity separation where cells are retained on a substrate according to their expression or lack of expression of a specific protein or type of protein, or batch purification using one or more antibodies against one or more surface antigens specifically expressed by the cell type of interest. Cells of hematopoietic origin can also be obtained from peripheral blood. Prior to harvest of the cells from peripheral blood, the subject can be treated with a cytokine, such as e.g., granulocyte-colony stimulating factor, to promote cell migration from the bone marrow to the blood compartment and/or promote activation and/or proliferation of the population of interest. Any method suitable for identifying surface proteins, for example, can be employed to isolate cells of hematopoietic origin from a heterogeneous population. In some embodiments, a clonal population of cells of hematopoietic origin, such as lymphocytes, is obtained. In some embodiments, the cells of hematopoietic origin are not a clonal population.

Further, in regard to the various aspects and embodiments of the compositions, vectors, constructs, methods, and kits described herein, a somatic cell can be obtained from any mammalian species, with non-limiting examples including a murine, bovine, simian, porcine, equine, ovine, or human cell. In some embodiments, the somatic cell is a human cell. In some embodiments, the cell is from a non-human organism, such as a non-human mammal.

In general, the methods for making induced pDCs described herein involve culturing or expanding somatic cells, such as cells of hematopoietic origin, in any culture medium that is available and well-known to one of ordinary skill in the art. Such media include, but is not limited to, Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®, and serum-free medium for culture and expansion of DCs. Many media are also available as low-glucose formulations, with or without sodium. The medium used with the methods described herein can, in some embodiments, be supplemented with one or more immunostimulatory cytokine. Commonly used growth factors include, but are not limited to, G-CSF, GM-CSF, TNF-α, IL-4, IL-3, the Flt-3 ligand and the kit ligand. In addition, in preferred embodiments, the immunostimulatory cytokine is selected from the group consisting of the interleukins (e.g., IL-1α, IL-1β, IL-2, IL-3, IL-4, IL-6, IL-8, IL-9, IL-10, IL-12, IL-18, IL-19, IL-20), the interferons (e.g., IFN-α, IFN-β, IFN-γ), tumor necrosis factor (TNF), transforming growth factor-3 (TGF-β), granulocyte colony stimulating factor (G-CSF), macrophage colony stimulating factor (M-CSF), granulocyte-macrophage colony stimulating factor (GM-CSF), the Flt-3 ligand and the kit ligand.

Cells in culture can be maintained either in suspension or attached to a solid support, such as extracellular matrix components or plating on feeder cells, for example. Cells being used in the methods described herein can require additional factors that encourage their attachment to a solid support, in some embodiments, such as type I and type II collagen, chondroitin sulfate, fibronectin, “superfibronectin” and fibronectin-like polymers, gelatin, poly-D and poly-L-lysine, thrombospondin and vitronectin. In some embodiments, the cells are suitable for growth in suspension cultures. Suspension-competent host cells are generally monodisperse or grow in loose aggregates without substantial aggregation. Suspension-competent host cells include cells that are suitable for suspension culture without adaptation or manipulation (e.g., cells of hematopoietic origin, such as lymphoid cells) and cells that have been made suspension-competent by modification or adaptation of attachment-dependent cells (e.g., epithelial cells, fibroblasts).

In some embodiments of these aspects and all such aspects described herein, the isolated induced pDC further comprise a pharmaceutically acceptable carrier for administration to a subject in need.

Also provided herein, in some aspects, are methods of treating a subject in need of treatment to induce antigen-specific immune responses to eliminate cancer cells or infectious agents using the pDC-inducing compositions and vectors, and methods of preparing induced pDCs described herein, or using the isolated induced pDCs and cell clones thereof produced using any of the combinations of pDC-inducing factors, pDC-inducing compositions and vectors, or methods of preparing induced pDCs described herein. In such methods of treatment, somatic cells, such as fibroblast cells or hematopoietic lineage cells, can first be isolated from the subject, and the isolated cells transduced or transfected, as described herein with a pDC-inducing composition comprising expression vectors or synthetic mRNAs, respectively. The isolated induced pDCs produced using any of the combinations of pDC-inducing factors, pDC-inducing compositions and vectors, or methods of preparing induced pDCs described herein, can then be administered to the subject, such as via systemic injection of the induced pDCs to the subject.

Also provided herein, in some aspects, are methods of treating a subject in need of treatment to induce antigen-specific immune responses to eliminate cancer cells or infectious agents using the pDC-inducing compositions and vectors and any of the combinations of pDC-inducing factors described herein. In such methods of treatment, cancer cells are transduced, as described herein with a pDC-inducing composition comprising expression vectors. Cancer cells can be first isolated from the subject, transduced with a pDC-inducing composition comprising expression vectors and then administered to the subject, such as via systemic injection. Alternatively, cancers cells can be transduced in situ or in vivo with pDC-inducing composition comprising viral expression vectors.

The reprogrammed induced pDCs generated using the compositions, vectors, constructs, methods, and kits described herein can, in some embodiments of the methods of treatment described herein, be used directly or administered to subjects in need of immunotherapies. Accordingly, various embodiments of the methods described herein involve administration of an effective amount of a induced pDC or a population of induced pDCs, generated using any of the compositions, vectors, methods, and kits described herein, to an individual or subject in need of a cellular therapy. The cell or population of cells being administered can be an autologous population, or be derived from one or more heterologous sources. Further, such induced pDCs can be administered in a manner that permits them to migrate to lymph node and activate effector T cells.

A variety of means for administering cells to subjects are known to those of skill in the art. Such methods can include systemic injection, for example, i.v. injection, or implantation of cells into a target site in a subject. Cells may be inserted into a delivery device which facilitates introduction by injection or implantation into the subject. Such delivery devices can include tubes, e.g., catheters, for injecting cells and fluids into the body of a recipient subject. In one preferred embodiment, the tubes additionally have a needle, e.g., through which the cells can be introduced into the subject at a desired location. The cells can be prepared for delivery in a variety of different forms. For example, the cells can be suspended in a solution or gel or embedded in a support matrix when contained in such a delivery device. Cells can be mixed with a pharmaceutically acceptable carrier or diluent in which the cells remain viable.

Accordingly, the cells produced by the methods described herein can be used to prepare cells to treat or alleviate several cancers and tumors including, but not limited to, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like.

In addition to the above, the methods of the invention can be used to prevent or eliminate infection by pathogens known to predispose to certain cancers. Pathogens of particular interest for use in the cancer vaccines provided herein include the hepatitis B virus (hepatocellular carcinoma), hepatitis C virus (heptomas), Epstein Barr virus (EBV) (Burkitt lymphoma, nasopharynx cancer, PTLD in immunosuppressed individuals), HTLVL (adult T cell leukemia), oncogenic human papilloma viruses types 16, 18, 33, 45 (adult cervical cancer), and the bacterium Helicobacter pylori (B cell gastric lymphoma). Other medically relevant microorganisms that may serve as antigens in mammals and more particularly humans are described extensively in the literature, e.g., C. G. A Thomas, Medical Microbiology, Bailliere Tindall, (1983).

In addition to the above, the methods of the invention can be used for viral infections. Exemplary viral pathogens include, but are not limited to, infectious virus that infect mammals, and more particularly humans. Examples of infectious virus include, but are not limited to: Retroviridae (e.g., human immunodeficiency viruses, such as HIV-1 (also referred to as HTLV-III, LAV or HTLV-III/LAV, or HIV-Ill; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses such as the SARS coronavirus); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bir-naviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, cytomegalovirus (CMV), herpes virus; P. oxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class I=interaally transmitted; class 2=parenterally transmitted (i.e. Hepatitis C); Norwalk and related viruses, and astro viruses).

In addition to the above, the methods of the invention can be used to target gram negative and gram positive bacteria in vertebrate animals. Such gram positive bacteria include, but are not limited to Pasteurella sp., Staphylococci sp., and Streptococcus sp. Gram negative bacteria include, but are not limited to, Escherichia coli, Pseudomonas sp., and Salmonella sp. Specific examples of infectious bacteria include but are not limited to: Helicobacter pyloris, Borella burgdorferi, Legionella pneumophilia, Mycobacteria sp. (e.g. M. tuberculosis, M. avium, M. intracellular e, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelii.

In addition to the above, the methods of the invention can be used to target pathogens that include, but are not limited to, infectious fungi and parasites that infect mammals, and more particularly humans. Examples of infectious fungi include, but are not limited to: Cryptococcus neoformans, Histoplasma capsulatum, Coccidioides immitis, Blastomyces dermatitidis, Chlamydia trachomatis, and Candida albicans.

In addition to the above, the methods of the invention can be used to target parasites such as intracellular parasites and obligate intracellular parasites. Examples of parasites include but are not limited to Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis, Leishmania tropica, Trypanosoma gambiense, Trypanosoma rhodesiense, Wuchereria bancrofti, Brugia malayi, Brugia timori, Ascaris lumbricoides, Onchocerca volvulus and Schistosoma mansoni.

If modified induced pDCs can be used to induce a tolerogenic response including the suppression of a future or existing immune response, to one or more target antigens. Thus, induced pDCs are useful for treating or preventing an undesirable immune response including, for example, transplant rejection, graft versus host disease, allergies, parasitic diseases, inflammatory diseases and autoimmune diseases. Examples of transplant rejection, which can be treated or prevented in accordance with the present invention, include rejections associated with transplantation of bone marrow and of organs such as heart, liver, pancreas, kidney, lung, eye, skin etc. Examples of allergies include seasonal respiratory allergies; allergy to aeroallergens such as hay fever; allergy treatable by reducing serum IgE and eosinophilia; asthma; eczema; animal allergies, food allergies; latex allergies; dermatitis; or allergies treatable by allergic desensitization. Autoimmune diseases that can be treated or prevented by the present invention include, for example, psoriasis, systemic lupus erythematosus, myasthenia gravis, stiff-man syndrome, thyroiditis, Sydenham chorea, rheumatoid arthritis, diabetes and multiple sclerosis. Examples of inflammatory disease include Crohn's disease, chronic inflammatory eye diseases, chronic inflammatory lung diseases and chronic inflammatory liver diseases, autoimmune hemolytic anemia, idiopathic leucopoenia, ulcerative colitis, dermatomyositis, scleroderma, mixed connective tissue disease, irritable bowel syndrome, systemic lupus erythromatosus (SLE), multiple sclerosis, myasthenia gravis, Guillan-Barre syndrome (antiphospholipid syndrome), primary myxoedema, thyrotoxicosis, pernicious anemia, autoimmune atrophic gastritis, Addison's disease, insulin-dependent diabetes mellitus (IDDM), Goodpasture's syndrome, Behcet's syndrome, Sjogren's syndrome, rheumatoid arthritis, sympathetic ophthalmia, Hashimoto's disease/hypothyroiditis, celiac disease/dermatitis herpetiformis, and demyelinating disease primary biliary cirrhosis, mixed connective tissue disease, chronic active hepatitis, Graves' disease/hyperthyroiditis, scleroderma, chronic idiopathic thrombocytopenic purpura, diabetic neuropathy and septic shock.

Pharmaceutically acceptable carriers and diluents include saline, aqueous buffer solutions, solvents and/or dispersion media. The use of such carriers and diluents is well known in the art. The solution is preferably sterile and fluid. Preferably, prior to the introduction of cells, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

It is preferred that the mode of cell administration is relatively non-invasive, for example by intravenous injection, pulmonary delivery through inhalation, topical, or intranasal administration. However, the route of cell administration will depend on the tissue to be treated and may include implantation. Methods for cell delivery are known to those of skill in the art and can be extrapolated by one skilled in the art of medicine for use with the methods and compositions described herein.

Also provided herein, in some aspects, are kits for making induced pDCs, the kits comprising any of the pDC-inducing compositions comprising one or more expression vector components described herein.

Also provided herein, in some aspects, are kits comprising one or more of the pDC-inducing factors described herein as components for the methods of making the induced pDCs described herein.

Accordingly, in some aspects, provided herein, are kits for preparing induced dendritic cells comprising the following components: (a) one or more expression vectors encoding at least one, two, three, four, five, six, seven, eight, or more pDC-inducing factors selected from: IRF8 (SEQ. ID. 1, SEQ. ID. 2), SPIB (SEQ. ID. 3, SEQ. ID. 4), ARID5A (SEQ. ID. 5, SEQ. ID. 6), BCL11A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), HDAC5 (SEQ. ID. 15, SEQ. ID. 16), HHEX (SEQ. ID. 17, SEQ. ID. 18), HOXA5 (SEQ. ID. 19, SEQ. ID. 20), ID3 (SEQ. ID. 21, SEQ. ID. 22), IKZF1 (SEQ. ID. 23, SEQ. ID. 24), IKZF2 (SEQ. ID. 25, SEQ. ID. 26), IKZF3 (SEQ. ID. 27, SEQ. ID. 28), IKZF5 (SEQ. ID. 29, SEQ. ID. 30), IRF7 (SEQ. ID. 31, SEQ. ID. 32), MEF2C (SEQ. ID. 33, SEQ. ID. 34), MYB (SEQ. ID. 35, SEQ. ID. 36), RUNX2 (SEQ. ID. 37, SEQ. ID. 38), STAT3 (SEQ. ID. 39, SEQ. ID. 40), TCF4 (SEQ. ID. 41, SEQ. ID. 42), TCF12 (SEQ. ID. 43, SEQ. ID. 44), STAT1 (SEQ. ID. 45, SEQ. ID. 46), TSC22D1 (SEQ. ID. 47, SEQ. ID. 48), and (b) packaging and instructions therefor.

The kits described herein, in some embodiments, can further provide the synthetic mRNAs or the one or more expression vectors encoding pDC-inducing factors in an admixture or as separate aliquots.

In some embodiments, the kits can further comprise an agent to enhance efficiency of reprogramming. In some embodiments, the kits can further comprise one or more antibodies or primer reagents to detect a cell-type specific marker to identify cells induced to the pDC state.

In some embodiments, the kits can further comprise a buffer. In some such embodiments, the buffer is RNase-free TE buffer at pH 7.0. In some embodiments, the kit further comprises a container with cell culture medium.

All kits described herein can further comprise a buffer, a cell culture medium, a transduction or transfection medium and/or a media supplement. In preferred embodiments, the buffers, cell culture mediums, transfection mediums, and/or media supplements are DNAse and RNase-free. In some embodiments, the synthetic, modified RNAs provided in the kits can be in a non-solution form of specific quantity or mass, e.g., 20 μg, such as a lyophilized powder form, such that the end-user adds a suitable amount of buffer or medium to bring the components to a desired concentration, e.g., 100 ng/μl.

All kits described herein can further comprise devices to facilitate single-administration or repeated or frequent infusions of the cells generated using the kits components described herein, such as a non-implantable delivery device, e.g., needle, syringe, pen device, or an implantatable delivery device, e.g., a pump, a semi-permanent stent (e.g., intravenous, intraperitoneal, intracisternal or intracapsular), or a reservoir. In some such embodiments, the delivery device can include a mechanism to dispense a unit dose of a pharmaceutical composition comprising the induced pDCs. In some embodiments, the device releases the composition continuously, e.g., by diffusion. In some embodiments, the device can include a sensor that monitors a parameter within a subject. For example, the device can include pump, e.g., and, optionally, associated electronics.

In an embodiment, induced pDCs are made by the hand of man by, e.g., modifying the gene expression of at least one of the factors disclosed herein of a somatic cell, a pluripotent cell, a progenitor cell or a stem cell, or by exposing any one of these cell types to at least one protein or RNA that produces at least one protein as disclosed herein. The cells can further be made by exposing them to small molecules that turn on at least one of the factors disclosed herein. In some aspects at least two, three, four, five, six, seven, or eight factors are used to make the induced pDCs.

In an embodiment, expression data for Clec9a, Tlr7, Tlr9 and candidate TFs in DC subsets and other myeloid cells (monocytes, macrophages and microglia) was obtained from Immgen database (www.immaen.ora). Expression data was analyzed by cluster 3.0 (log-transformed data; centered genes; centered, clustered and normalized cell types) and displayed by Treeview. Alternatively, expression values were displayed using GraphPad Prism.

In an embodiment, mouse Embryonic Fibroblasts (MEFs) were isolated and purified in the following way: Clec9aCre/Cre animals (10) were crossed with Rosa26-stopflox-tdTomato reporter mice (The Jackson Laboratory) to generate double homozygous Clec9aCre/Cre RosatdTomato/tdTomato (Clec9A-tdTomato) mice. All animals were housed under controlled temperature (23±2° C.), subject to a fixed 12-h light/dark cycle, with free access to food and water.

In an embodiment, primary cultures of MEFs were isolated from E13.5 embryos of Clec9A-tdTomato or C57BL/6 mice (6, 10). Head, fetal liver and all internal organs were removed and the remaining tissue was mechanically dissociated. Dissected tissue was enzymatic digested using 0.12% trypsin/0.1 mM Ethylenediaminetetraacetic acid (EDTA) solution (3 mL per embryo), and incubation at 37° C. for 15 min. Additional 3 mL of same solution per embryo were added, followed by another 15 min incubation period. A single cell suspension was obtained and plated in 0.1% gelatin-coated 10-cm tissue culture dishes in growth media. Cells were grown for 2-3 days until confluence, dissociated with Tryple Express and frozen in Fetal Bovine Serum (FBS) 10% dimethyl sulfoxide (DMSO). Before plating for lentiviral transduction, MEFs were sorted to remove residual CD45+ and tdTomato+ cells that could represent cells with hematopoietic potential.

In an embodiment, HEK293T cells and MEFs were maintained in growth medium [Dulbecco's modified eagle medium (DMEM) supplemented with 10% (v/v) FBS, 2 mM L-Glutamine and antibiotics (10 μg/ml Penicillin and Streptomycin)]. All cells were maintained at 37° C. and 5% (v/v) CO2. All tissue culture reagents were from Thermo Fisher Scientific unless stated otherwise.

In an embodiment, viral transduction and reprogramming experiments were performed in the following way: Clec9A-tdTomato MEFs were seeded at a density of 40,000 cells per well on 0.1% gelatin coated 6-well plates. Cells were incubated overnight with a ratio of 1:1 FUW-TetO-TFs and FUW-M2rtTA lentiviral particles in growth media supplemented with 8 μg/mL polybrene. When testing combinations of TFs, equal MOIs of each individual viral particle were applied. Cells were transduced twice in consecutive days and after overnight incubation, media was replaced with fresh growth media. After the second transduction, growth media was supplemented with Doxycycline (1 μg/mL)—day 0. Media was changed every 2-3 days for the duration of the cultures. Emerging tdTomato+ cells were analyzed 5-8 days post-transduction.

In an embodiment, flow cytometry analysis was performed in the following way: Transduced Clec9A-tdTomato MEFs were dissociated with TrypLE Express, resuspended in 200 μL PBS 5% FBS and kept at 4° C. prior analysis in BD FACS LSR II (BD Biosciences). For the analysis of MHC-1l cell surface marker expression, dissociated cells were incubated with APC-conjugated rat anti-mouse I-A/I-E antibody (Biolegend) or APC-Cy7-conjugated anti-CD45 antibody (Biolegend) diluted in PBS 5% FBS at 4° C. for 30 minutes in the presence of rat serum ( 1/100, GeneTex) to block unspecific binding. Cells were washed with PBS 5% FBS, resuspended in PBS 5% FBS and analyzed in a BD FACS LSR II. Flow cytometry data were analyzed using FlowJo software (FLOWJO, LLC, version 7.6).

In an embodiment, fluorescence-activated cell sorting (FACS) was performed in the following way: To purify Clec9A-tdTomato MEFs, cells were incubated at 4° C. for 30 minutes with APC-Cy7-conjugated anti-CD45 antibody (Biolegend) diluted in PBS 5% FBS. Subsequently, MEFs were washed with PBS 5% FBS, resuspended in PBS 5% FBS and tdTomato− CD45− MEFs were purified in BD FACSAria III. When described tdTomato+ cells were purified using BD FACSAria III. FACS data was processed in FlowJo software.

In an embodiment, freshly isolated spleens from Clec9a-tdTomato mice were homogenized using the frosted ends of 2 sterile slides. Cells were harvested in PBS supplemented with 5% FBS and filtered through a 70 μm cell strainer (BD Biosciences). Red blood cells were lysed with BD Pharm Lyse (BD Biosciences) for 8 min at room temperature. Lysis was stopped by addition of 5 volumes of PBS 5% FBS. Splenocytes were washed once to obtain single cell suspensions, which were incubated at 4° C. for 30 minutes with a mixture of FITC-conjugated anti-CD11c antibody (Biolegend), APC-conjugated anti-Bst2 antibody (Biolegend), APC-Cy7-conjugated anti-B220 antibody (Biolegend) and BV605-conjugated anti-Siglec-H antibody (Biolegend) diluted in PBS 5% FBS in the presence of rat serum. Single staining and fluorescent minus one controls were included for proper compensation and gating. After 30 minutes at 4° C., cells were washed and resuspended with PBS 3% FBS and analyzed in BD FACSAria III (BDiosciences). Data resulting from this analysis were treated using FlowJo software. Splenic pDCs are identified as live single cells CD11c^{low} Bst2⁺ B220⁺ Siglec-H⁺.

In an embodiment, an inflammatory cytokine assay was performed in the following way: tdTomato+ cells were FACS sorted at day 9 and cultured with 1 μg/mL of R848 (Invivogen) and 0.5 μM CpG ODN 1668 (Invivogen) for TLR 7 and TLR9 overnight stimulation, respectively. Levels of the cytokines interferon-α (IFN-α), interferon-3 (IFN-β), interleukin-10 (IL-10), interleukin-6 (IL-6), tumor necrosis factor-α (TNF-α), C-C Motif Chemokine Ligand 5 (CCL5) and C-X-C motif chemokine ligand 10 (CXCL10), were assessed at day 10. 50 μL of culture supernatants from a 96-well plate well were collected and analyzed by LEGENDplex™ Mouse Anti-Virus Response Panel (13-plex) with V Plate (Biolegend), according to manufacturer's instructions. Acquisition was performed with a BD FACS LSR II and data were analyzed using LEGENDplex™ Data Analysis, version 8.0 (Biolegend).

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

Where singular forms of elements or features are used in the specification of the claims, the plural form is also included, and vice versa, if not specifically excluded. For example, the term “a transcription factor” or “the transcription factor” also includes the plural forms “transcription factors” or “the transcription factors,” and vice versa. In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skilled in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The above described embodiments are combinable.

The following table provides an overview of the sequences included in the present disclosure.

TABLE 1
SEQ. ID.s included in the present disclosure.

| | Sequence description | | Sequence description |
|---|---|---|---|
| SEQ. ID. | (Polynucleotide) | SEQ. ID. | (Polypeptide) |
| 1 | Human IRF8 | 49 | Human IRF8 |
| 2 | Mouse IRF8 | 50 | Mouse IRF8 |
| 3 | Human SPIB | 51 | Human SPIB |
| 4 | Mouse SPIB | 52 | Mouse SPIB |
| 5 | Human ARID5A | 53 | Human ARID5A |
| 6 | Mouse ARID5A | 54 | Mouse ARID5A |
| 7 | Human BCL11A | 55 | Human BCL11A |
| 8 | Mouse BCL11A | 56 | Mouse BCL11A |
| 9 | Human CBFA2T3 | 57 | Human CBFA2T3 |
| 10 | Mouse CBFA2T3 | 58 | Mouse CBFA2T3 |
| 11 | Human CREB3L2 | 59 | Human CREB3L2 |
| 12 | Mouse CREB3L2 | 60 | Mouse CREB3L2 |
| 13 | Human ETS1 | 61 | Human ETS1 |
| 14 | Mouse ETS1 | 62 | Mouse ETS1 |
| 15 | Human HDAC5 | 63 | Human HDAC5 |
| 16 | Mouse HDAC5 | 64 | Mouse HDAC5 |
| 17 | Human HHEX | 65 | Human HHEX |
| 18 | Mouse HHEX | 66 | Mouse HHEX |
| 19 | Human HOXA5 | 67 | Human HOXA5 |
| 20 | Mouse HOXA5 | 68 | Mouse HOXA5 |
| 21 | Human ID3 | 69 | Human ID3 |
| 22 | Mouse ID3 | 70 | Mouse ID3 |
| 23 | Human IKZF1 | 71 | Human IKZF1 |
| 24 | Mouse IKZF1 | 72 | Mouse IKZF1 |
| 25 | Human IKZF2 | 73 | Human IKZF2 |
| 26 | Mouse IKZF2 | 74 | Mouse IKZF2 |
| 27 | Human IKZF3 | 75 | Human IKZF3 |
| 28 | Mouse IKZF3 | 76 | Mouse IKZF3 |
| 29 | Human IKZF5 | 77 | Human IKZF5 |
| 30 | Mouse IKZF5 | 78 | Mouse IKZF5 |
| 31 | Human IRF7 | 79 | Human IRF7 |
| 32 | Mouse IRF7 | 80 | Mouse IRF7 |
| 33 | Human MEF2C | 81 | Human MEF2C |
| 34 | Mouse MEF2C | 82 | Mouse MEF2C |
| 35 | Human MYB | 83 | Human MYB |
| 36 | Mouse MYB | 84 | Mouse MYB |
| 37 | Human RUNX2 | 85 | Human RUNX2 |
| 38 | Mouse RUNX2 | 86 | Mouse RUNX2 |
| 39 | Human STAT3 | 87 | Human STAT3 |
| 40 | Mouse STAT3 | 88 | Mouse STAT3 |
| 41 | Human TCF4 | 89 | Human TCF4 |
| 42 | Mouse TCF4 | 90 | Mouse TCF4 |
| 43 | Human TCF12 | 91 | Human TCF12 |
| 44 | Mouse TCF12 | 92 | Mouse TCF12 |
| 45 | Human STAT1 | 93 | Human STAT1 |
| 46 | Mouse STAT1 | 94 | Mouse STAT1 |
| 47 | Human TSC22D1 | 95 | Human TSC22D1 |
| 48 | Mouse TSC22D1 | 96 | Mouse TSC22D1 |

The following claims further set out particular embodiments of the disclosure.

1. A composition comprising at least the two transcription factors: IRF8 and SPIB; for use in reprogramming a stem cell or a differentiated cell, or mixtures thereof, into a plasmacytoid dendritic cell or an interferon type I-producing cell in a subject.

2. The composition according to claim 1, wherein the composition further comprises one or more other transcription factors selected from the group consisting of: ARID5A, BCL1A, CBFA2T3, CREB3L2, ETS1, IKZF1, STAT1, TCF4, TCF12 and TSC22D1.

3. The composition according to any one of the previous claims, wherein the transcription factors individually are at least 90% identical to the sequences selected from the group consisting of: IRF8 (SEQ. ID. 49, SEQ. ID. 50), SPIB (SEQ. ID. 51, SEQ. ID. 52), ARID5A (SEQ. ID. 53, SEQ. ID. 54), BCL11A (SEQ. ID. 55, SEQ. ID. 56), CBFA2T3 (SEQ. ID. 57, SEQ. ID. 58), CREB3L2 (SEQ. ID. 59, SEQ. ID. 60), ETS1 (SEQ. ID. 61, SEQ. ID. 62), IKZF1 (SEQ. ID. 71, SEQ. ID. 72), TCF4 (SEQ. ID. 89, SEQ. ID. 90), TCF12 (SEQ. ID. 91, SEQ. ID. 92), STAT1 (SEQ. ID. 93, SEQ. ID. 94) and TSC22D1 (SEQ. ID. 95, SEQ. ID. 96).

4. The composition according to any one of the previous claims wherein the combination of transcription factors is selected from the following combinations:
IRF8 and SPIB;
IRF8, SPIB and ARID5A;
IRF8, SPIB and BCL11A;
IRF8, SPIB and CBFA2T3;
IRF8, SPIB and CREB3L2;
IRF8, SPIB and ETS1;
IRF8, SPIB and IKZF1;
IRF8, SPIB and STAT1;
IRF8, SPIB and TCF4;
IRF8, SPIB and TCF12;
IRF8, SPIB and TSC22D1 or mixtures thereof.

5. The composition according to any one of the previous claims wherein the combination of transcription factors is the combination of IRF8, SPIB and TCF12.

6. The composition according to any one of the previous claims wherein the combination of transcription factors is the combination of IRF8, SPIB and TSC22D1.

7. The composition according to any one the previous claims, wherein the cell is selected from the group consisting of: pluripotent stem cell, multipotent stem cell, differentiated cell, tumor cell, cancer cell and mixtures thereof.

8. The composition according to any one of the previous claims, for use in veterinary or human medicine, in particular in the diagnosis, therapy or treatment of antiviral immune responses and immune pathology, particularly in autoimmune diseases and immunodeficiency conditions, neoplasia in particular cancer, namely solid or hematological tumors, of benign tumour, malignant tumour, early cancer, basal cell carcinoma, cervical dysplasia, soft tissue sarcoma, germ cell tumour, retinoblastoma, age-related macular degeneration, Hodgkin's lymphoma, blood cancer, prostate cancer, ovarian cancer, cervix cancer, uterus cancer, vaginal cancer, breast cancer, naso-pharynx cancer, trachea cancer, larynx cancer, bronchi cancer, bronchioles cancer, lung cancer, hollow organs cancer, esophagus cancer, stomach cancer, bile duct cancer, intestine cancer, colon cancer, colorectum cancer, rectum cancer, bladder cancer, ureter cancer, kidney cancer, liver cancer, gall bladder cancer, spleen cancer, brain cancer, lymphatic system cancer, bone cancer, pancreatic cancer, leukaemia, skin cancer, or myeloma, infectious disease, viral disease or viral induced disease, central and peripheral nervous system disorder, fungal, viral, chlamydial, bacterial, nanobacterial or parasitic infectious disease, or viral disease, or viral induced disease, as an antiviral agent or as an immunogenic composition, immunotherapy, or in the treatment or therapy of neurodegenerative diseases, in the treatment or therapy of cancer or in the treatment or therapy of an infectious diseases, intradermal and transdermal therapies, HIV, infection with SARS coronavirus, Asian flu virus, herpes simplex, influenza virus, herpes zoster, hepatitis, human papillomavirus, Epstein-Barr virus, human T-lymphotropic virus or viral hepatitis, an amyloid disease in particular Amyloid A amyloidosis, Alzheimer's disease, Light-Chain (AL) amyloidosis, Type-2 Diabetes, Medullary Carcinoma of the Thyroid, Parkinson's disease, Polyneuropathy, or Spongiform Encephalopathy-Creutzfeldt Jakob disease or for drug screening.

9. A construct or a vector encoding at least the two transcription factors SPIB and IRF8.

10. The construct or vector according to claim 9, wherein the vector is a viral vector; in particular a retroviral, adenoviral, lentiviral, herpes viral, pox viral, paramyxoviral, rabdoviral, alphaviral, flaviral or adeno-associated viral vector.

11. The construct or vector according to any one of the previous claims 9 to 10, wherein the vector or construct is synthetic mRNA, naked alphavirus RNA replicons or naked flavivirus RNA replicons.

12. One or more vectors comprising at least two polynucleotide sequences encoding at least the two transcription factors IRF8 and SPIB, for use in reprogramming a stem cell or a differentiated cell, or mixtures thereof, into a plasmacytoid dendritic cell or an interferon type I-producing cell in a subject.

13. The one or more vectors of claim 12, wherein the vectors further code for one or more other transcription factors selected from the group consisting of: ARID5A, BCL11A, CBFA2T3, CREB3L2, ETS1, IKZF1, STAT1, TCF4, TCF12 and TSC22D1.

14. The one or more vectors according to any one of the previous claims 12 to 13, wherein the transcription factors individually are at least 90% identical to the sequences selected from the group consisting of: IRF8 (SEQ. ID. 1, SEQ. ID. 2), SPIB (SEQ. ID. 3, SEQ. ID. 4), ARID5A (SEQ. ID. 5, SEQ. ID. 6), BCL11A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), IKZF1 (SEQ. ID. 23, SEQ. ID. 24), TCF4 (SEQ. ID. 41, SEQ. ID. 42), TCF12 (SEQ. ID. 43, SEQ. ID. 44), STAT1 (SEQ. ID. 45, SEQ. ID. 46) and TSC22D1 (SEQ. ID. 47, SEQ. ID. 48).

15. The one or more vectors according to any one of the previous claims 12 to 14, wherein the combination of transcription factors is selected from the following combinations:
IRF8 and SPIB;
IRF8, SPIB and ARID5A;
IRF8, SPIB and BCL11A;
IRF8, SPIB and CBFA2T3;
IRF8, SPIB and CREB3L2;
IRF8, SPIB and ETS1;
IRF8, SPIB and IKZF1;
IRF8, SPIB and STAT1;
IRF8, SPIB and TCF4;
IRF8, SPIB and TCF12;
IRF8, SPIB and TSC22D1, or mixtures thereof.

16. The one or more vectors according to any one of the previous claims 12 to 15, wherein the combination of transcription factors is the combination of IRF8, SPIB and TCF12.

17. The one or more vectors according to any one of the previous claims 12 to 16, wherein the combination of transcription factors is the combination of IRF8, SPIB and TSC22D1.

18. The one or more vectors according to any one of the previous claims 12 to 17, wherein the vector is a viral vector; in particular a retroviral, adenoviral, lentiviral, herpes viral, pox viral, paramyxoviral, rabdoviral, alphaviral, flaviviral or adeno-associated viral vector.

19. The one or more vectors according to any one of the previous claims 12 to 18, wherein the vectors is synthetic mRNA, naked alphaviral RNA replicons or naked flavivirus RNA replicons.

20. The one or more vectors according to any one the previous claims 12 to 19, wherein the cell is selected from the group consisting of: pluripotent stem cell, multipotent stem cell, differentiated cell, tumor cell, cancer cell and mixtures thereof.

21. The one or more vectors according to any one of the previous claims 12 to 20, for use in veterinary or human medicine, in particular in the diagnosis, therapy or treatment of antiviral immune responses and immune pathology, particularly in autoimmune diseases and immunodeficiency conditions, neoplasia in particular cancer, namely solid or hematological tumors, of benign tumor, malignant tumor, early cancer, basal cell carcinoma, cervical dysplasia, soft tissue sarcoma, germ cell tumor, retinoblastoma, age-related macular degeneration, Hodgkin's lymphoma, blood cancer, prostate cancer, ovarian cancer, cervix cancer, uterus cancer, vaginal cancer, breast cancer, naso-pharynx cancer, trachea cancer, larynx cancer, bronchi cancer, bronchioles cancer, lung cancer, hollow organs cancer, esophagus cancer, stomach cancer, bile duct cancer, intestine cancer, colon cancer, colorectum cancer, rectum cancer, bladder cancer, ureter cancer, kidney cancer, liver cancer, gall bladder cancer, spleen cancer, brain cancer, lymphatic system cancer, bone cancer, pancreatic cancer, leukemia, skin cancer, or myeloma, infectious disease, viral disease or viral induced disease, central and peripheral nervous system disorder, fungal, viral, chlamydial, bacterial, nanobacterial or parasitic infectious disease, or viral disease, or viral induced disease, as an antiviral agent or as an immunogenic composition, immunotherapy, or in the treatment or therapy of neurodegenerative diseases, in the treatment or therapy of cancer or in the treatment or therapy of an infectious diseases, intradermal and transdermal therapies, HIV, infection with SARS coronavirus, Asian flu virus, herpes simplex, influenza virus, herpes zoster, hepatitis, human papillomavirus, Epstein-Barr virus, human T-lymphotropic virus or viral hepatitis, an amyloid disease in particular Amyloid A amyloidosis, Alzheimer's disease, Light-Chain (AL) amyloidosis, Type-2 Diabetes, Medullary Carcinoma of the Thyroid, Parkinson's disease, Polyneuropathy, or Spongiform Encephalopathy-Creutzfeldt Jakob disease or for drug screening.

22. An in vitro method for reprogramming or inducing a cell into a plasmacytoid dendritic cell or interferon type I-producing cell, comprising the following steps:
transducing a cell selected from the group consisting of: a stem cell, a differentiated cell, and mixtures thereof, with one or more vectors comprising at least the two transcription factors selected from the group consisting of: IRF8 and SPIB.

23. The method according to claim 22, wherein the one or more vectors further comprise one or more transcription factors selected from the group consisting of: ARID5A, BCL11A, CBFA2T3, CREB3L2, ETS1, IKZF1, STAT1, TCF4, TCF12 and TSC22D1.

24. The method according to any one of the previous claims 22 to 23, wherein the transcription factors individually are at least 90% identical to the sequences selected from the group consisting of: IRF8 (SEQ. ID. 1, SEQ. ID. 2), SPIB (SEQ. ID. 3, SEQ. ID. 4), ARID5A (SEQ. ID. 5, SEQ. ID. 6), BCL11A (SEQ. ID. 7, SEQ. ID. 8), CBFA2T3 (SEQ. ID. 9, SEQ. ID. 10), CREB3L2 (SEQ. ID. 11, SEQ. ID. 12), ETS1 (SEQ. ID. 13, SEQ. ID. 14), IKZF1 (SEQ. ID. 23, SEQ. ID. 24), TCF4 (SEQ. ID. 41, SEQ. ID. 42), TCF12 (SEQ. ID. 43, SEQ. ID. 44), STAT1 (SEQ. ID. 45, SEQ. ID. 46) and TSC22D1 (SEQ. ID. 47, SEQ. ID. 48).

25. The method according to any one of the previous claims 22 to 24, wherein the combination of transcription factors is selected from the following combinations:
IRF8 and SPIB;
IRF8, SPIB and ARID5A;
IRF8, SPIB and BCL11A;
IRF8, SPIB and CBFA2T3;
IRF8, SPIB and CREB3L2;
IRF8, SPIB and ETS1;
IRF8, SPIB and IKZF1;
IRF8, SPIB and STAT1;
IRF8, SPIB and TCF4;
IRF8, SPIB and TCF12;
IRF8, SPIB and TSC22D1, or mixtures thereof.

26. The method according to any one of the previous claims 22 to 25, culturing the transduced cells during at least 2 days, preferably at least 5 days, more preferably at least 8 days, even more preferably at least 9 days.

27. The method according to any one of the previous claims 22 to 26, wherein the transducing step further comprises at least one vector selected from the group consisting of: a nucleic acid sequence encoding IL-12; nucleic acid sequence encoding IL-4; a nucleic acid sequence encoding IFN-α; a nucleic acid sequence encoding IFN-β; a nucleic acid sequence encoding IFN-γ; a nucleic acid sequence encoding TNF; nucleic acid sequence encoding GM-CSF; nucleic acid sequence encoding siRNAs targeting IL-10 RNA, and mixtures thereof, preferably comprising nucleic acids encoding immunostimulatory cytokines.

28. The method according to any one of the previous claims 22 to 27, wherein the cell is a mammalian cell, a human cell, a non-human cell or a mouse cell selected from the group consisting of: of pluripotent stem cell, or multipotent stem cell, differentiated cell, wherein the pluripotent stem cell, multipotent stem cell, or differentiated cell, is selected from the group consisting of: an endoderm derived cell, a mesoderm derived cell, or an ectoderm derived cell, a multipotent stem cell including mesenchymal stem cell, a hematopoietic stem cell, an intestinal stem cell, a pluripotent stem cell and a cell line; in particular a mesoderm-derived cell preferably a monocyte and mixtures thereof.

29. An induced plasmacytoid dendritic cell or interferon type I-producing cell obtained by the method described in any of the claims 22 to 28.

30. A composition comprising a plasmacytoid dendritic cell or an interferon type I-producing cell according to claim 29 in a therapeutically effective amount further comprising a pharmaceutically acceptable excipient, an analgesic, an anti-inflammatory agent, a chemotherapy agent, a radiotherapy agent, an antibiotic, a diuretic, a filler, a binder, a disintegrant, or a lubricant, or mixtures thereof.

31. A vaccine or an injectable formulation, in particular an in-situ injection comprising the composition according to any one of claims 1 to 8 and 30, and/or the induced plasmacytoid dendritic cell or interferon type I-producing cell according to claim 29, or mixtures thereof.

32. A kit comprising at least one of the following components:
the induced plasmacytoid dendritic cell or interferon type I-producing cell according to claim 29;
the composition according to any of claims 1 to 8 and 30;
the vector or a construct according to claims 9 to 21; or mixtures thereof.


SEQUENCE LISTING (96 sequences)

SEQ ID NO: 1 - DNA, 2669 bp, Homo sapiens
gtgaggtcatggaggccagcattgccttctcatggcaggtgtcccggagtccctgaatctgatgtgtgaccggaatggtggtcggcggcttcgacagtggctgatcgagcagattgacagtagcatgtatccaggactgatttgggagaatgaggagaagagcatgttccggatcccttggaaacacgctggcaagcaagattataatcaggaagtggatgcctccatttttaaggcctgggcagtttttaaagggaagtttaaagaaggggacaaagctgaaccagccacttggaagacgaggttacgctgtgctttgaataagagcccagattttgaggaagtgacggaccggtcccaactggacatttccgagccatacaaagtttaccgaattgttcctgaggaagagcaaaaatgcaaactaggcgtggcaactgctggctgcgtgaatgaagttacagagatggagtgcggtcgctctgaaatcgacgagctgatcaaggagccttctgtggacgattacatggggatgatcaaaaggagcccttccccgccggaggcctgtcggagtcagctccttccagactggtgggcgcagcagcccagcacaggcgtgccgctggtgacggggtacaccacctacgacgcgcaccattcagcattctcccagatggtgatcagcttctactatgggggcaagctggtgggccaggccaccaccacctgccccgagggctgccgcctgtccctgagccagcctgggctgcccggcaccaagctgtatgggcccgagggcctggagctggtgcgcttcccgccggccgacgccatccccagcgagcgacagaggcaggtgacgcggaagctgttcgggcacctggagcgcggggtgctgctgcacagcagccggcagggcgtgttcgtcaagcggctgtgccagggccgcgtgttctgcagcggcaacgccgtggtgtgcaaaggcaggcccaacaagctggagcgtgatgaggtggtccaggtcttcgacaccagccagttcttccgagagctgcagcagttctataacagccagggccggcttcctgacggcagggtggtgctgtgctttggggaagagtttccggatatggcccccttgcgctccaaactcattctcgtgcagattgagcagctgtatgtccggcaactggcagaagaggctgggaagagctgtggagccggctctgtgatgcaggcccccgaggagccgccgccagaccaggtcttccggatgtttccagatatttgtgcctcacaccagagatcatttttcagagaaaaccaacagatcaccgtctaagtgcgtcgcttgggcgccccaccccgtctgcgtcctgcatccatctccctgttacagtggcccgcatcatgattaaagaatgtggatccctctgtctggggtgggatgccttactttgcacttaatttaataagggcattctcggaggagtagacgtttaatacgaagtggcggcatagccctgccgagatgtcggtgatggcctggatgctgtaaccacaacctgtggctaaaaattttattttctatcctttacccgtcattatcattagttgctatgattctttctgcattttcggttaactatcatttccaaagacttgtcattcagtaatattagcagatagctgcttcgataaaggaatttggagtttaaaaatcaacttgtgaaaacaaggttgtttttgtctttatcgtttgttagagttatagatttatgatttcataggcttgattctatgtgaaatatctttttacttttatgcattttaataagatttaaaaatatttagattaaagccccctttaatgagtacaagaaaaactcttggcttgttagaagaaagtatattctttctagaatttggtgcaggaatatgtgttcatatccaggcaaacgggtgtgtttttatcttcagacaatgaaaccttctcctctggggctttgttgccaggaagattagaactaaatttatttttttcatttctgtcatgaaatcattccagatacctcttttcttctttccaaatggttttcacatgtgtttgaaatatttgtacttcgaattgtcggattttccatgtcctcctttctcctttgtgcccagcctgagtcagcaccaatcccgcattcagaacctcccagtgaaagggcagccttcattttgagaaggtggaaggtgttagggtttgggagacagctcatccaatctcccaagtctcatggtggatttgtgactgtgagagtttccggtttaaaatctgaaaagccagatatgcctgtttccttttcccagcaccatgcctgtggaggggacagtcagacccagaggtcctttacgtgtggatggagttcacaggcgaatagaggagaggaccaggggacgtggcttgtcccttttgtccaacaaagcattatatttttaagaatggcagacctgtttgctgaagtgttcataagataacaataggcttgaatctccaattcaaatgaatgtcaaagcacatatctttaatatgctgaatgaatatttatttttgtatccattaaaacagtatattgatctcttttattctttattaaaataaaatgctcttttttaaagct

SEQ ID NO: 2 - DNA, 3391 bp, Mus musculus
ttccaagcccactggtggcagttagggctgctgtgtggctaaaaccagccctggagagacctcatctccctcctctcaggctgctgccgtctttcctggccccactaccacctgcaagcggcctggagtttctgaccctcaggcctctttccagcaatcctgactttgtgggacatgctggctctggatgtgactatgttcttggggatatcttgacttctcactgatgataacttcttttttttttttttttccctaccccaagaggtcattttagttggaagactagaagtgctgtgttctggttttcagggtgggcgtctgtccaactgctttggggatttccaggctgttctagtaaatgctggctgcaagtccctgtgcacaaatgccctctctccccacgctgtaggaaaagcagacccgagagtgtccaagtgtgccattaggtgtctggggatggcgggcctggatatggagtccctgagctggctttgtactgtccctttgttctcactgagggctgattgtaagagggactccctgtgttgttggaggagtcctctaggatacaggctctgattgcaggatgtgtgaccggaacggcgggcggcggctgcggcagtggctgatcgaacagatcgacagcagcatgtacccggggctgatctgggaaaatgatgagaagaccatgttccgtatcccctggaagcatgccggcaagcaggattacaatcaggaggtggatgcttccatcttcaaggcctgggcagtttttaaagggaagtttaaagagggagacaaagctgaaccagccacgtggaagacgaggttacgctgtgctctgaacaagagcccagattttgaagaagtgactgaccggtcccagctggacatttctgagccatataaagtttaccgaattgtccccgaggaagaacaaaaatgcaagctgggcgtggcacctgcaggctgcatgagcgaagttcctgagatggagtgtggccgctcagagattgaggagctgatcaaggaaccttctgtggatgagtacatgggtatgaccaagaggagcccatccccaccagaggcctgcaggagccagatcctccctgactggtgggtccagcagcccagtgcaggcctgccactggtgaccggatatgccgcctatgacacacaccattcagctttctcccagatggtcatcagcttctactacgggggcaagctggtgggccaggccaccaccacctgccttgaaggctgccgtctctccctgagccagccggggctgcctaagttgtatgggccggatggcctggaacccgtgtgctttccgacggccgacaccatccccagtgagcggcagaggcaggtgacccggaagctgtttgggcacctggaacgtggcgtgctactgcacagcaaccgcaagggcgtgttcgtgaagcggctgtgccagggccgcgtgttctgcagcggcaacgcggtggtgtgcaagggcaggcccaacaagctggagcgggacgaggtggtgcaggtctttgacaccaaccagttcatccgagagctgcagcaattctacgccacccagagccgcctacctgacagcagggtggtcctgtgcttcggggaggagtttccggacactgtgcccttgcgctccaaactcattctggtgcaggtagagcagctgtatgccaggcagctggtggaggaagcgggcaagagctgcggtgctggctccctgatgccagccctggaggagccccagccggaccaggctttccgcatgtttccggatatctgtacctcacaccagagacccttttttagagaaaatcaacagatcaccgtctaagcctcagtccgggcaccccacctcgcctgagctcaagcttcaagagtctgtgactaagagaattccgaaaggatgtggagccctctgactggggtgggcgggtgtcctccaaggggcctccggaagcccacagagggatgcgctcctgctcaggcaggtgtcagaagcttgcaggggctgtggccgcaacctgtgattaaagcattcctttcctgcgtttcccccttcaccactaatggctggcctttctgtgtgctgaggtctttcgacagttcaaatcatctggtggcagcagactcgcctttgcccttctgcggccgagggcggagatttatgactttctctgcttggttggagaagaagaatctttactattcagcttcttttctttttggccagaactctgaaaaaaaaaaaaactctttttaagacaatatttgtattctcacaggctcagctgtcaatcacttgagaccttccctgtaaagtggggcagattttaaatatgggtgtagatactgcttgcagccttcgcaggaattttggttgtggttcattgattcacacagactctgtgtcagctgacagggctgtgtggggcatcaaaggaggaccaggcactgtggagaagacccattcactggcatctcacccttccttgtccagctccatacccagtcctaagacccagtgaaaagccacgtccaaactgtgctctgggctcatcagtgcccacccacgtaccagggaaaggcacacaccctacccagtgggcacagagcggaatgtccccctaccgcaccatttgcgcccccaatctggctgtccaacctagtttgtaagtaatctaaatcagtgactatagccccgcctaagggacacttcccggaggagggagccgctgaaaaggagttagtttgagggtcagtacacaacaggggcagaaagccaagcagatgtgggggcagggagagtcatcatctgcttttgtctgagagaaggagagcttctccgtttgttcaactttgtaacaagctgggttacatgctccacgcagctagagaagcctaggtgctctgcattccctggggaactgcaggaaagccttacctgctgactgttgctctggggaaaagcctgagggtccagagcagctacaagctacaggccataccttacaacctgaaaagctaaggaccacggtgaccttcccggctactgtgtgaaggtgctgggtggggcctgctcaacagacagggtcgacagagtgtgtgatacatgcaaacagaatccttggagtgtgtgatacatgcaaacagaatcctgggcccctgcttctccccctcagtcaaagcaggagtgtcccttccgaagccaggacaacctgttcacaaggccccttgtcacatgtcaccttccacctgcctcaaggagtgctagtgtccaaatatttatttttgtattctcttaagaagtattgatttcatcctttattaaaaaaagttgctctttcacaaaaaaaaaaaa

SEQ ID NO: 3 - DNA, 3792 bp, Homo sapiens
ggcaaacagcccgcccggcaccaccatgctcgccctggaggctgcacagctcgacgggccacacttcagctgtctgtacccagatggcgtcttctatgacctggacagctgcaagcattccagctaccctgattcagagggggctcctgactccctgtgggactggactgtggccccacctgtcccagccaccccctatgaagccttcgacccggcagcagccgcttttagccacccccaggctgcccagctctgctacgaaccccccacctacagccctgcagggaacctcgaactggcccccagcctggaggccccggggcctggcctccctgcataccccacggagaacttcgctagccagaccctggttcccccggcatatgccccgtaccccagccctgtgctatcagaggaggaagacttaccgttggacagccctgccctggaggtctcggacagcgagtcggatgaggccctcgtggctggccccgaggggaagggatccgaggcagggactcgcaagaagctgcgcctgtaccagttcctgctggggctactgacgcgcggggacatgcgtgagtgcgtgtggtgggtggagccaggcgccggcgtcttccagttctcctccaagcacaaggaactcctggcgcgccgctggggccagcagaaggggaaccgcaagcgcatgacctaccagaagctggcgcgcgccctccgaaactacgccaagaccggcgagatccgcaaggtcaagcgcaagctcacctaccagttcgacagcgcgctgctgcctgcagtccgccgggcctgagcacacccgaggctcccacctgcggagccgctgggggacctcacgtcccagccaggatccccctggaagaaaaagggcgtccccacactctaggtgataggacttacgcatccccaccttttggggtaaggggagtgctgccctgccataatccccaagcccagcccgggcctgtctgggattccccacttgtgcctggggtcctctgggatttctttgtcatgtacagactccctgggatcctcatgttttgggtgacaggacctatggaccactatactcggggaggcagggtagcagttcttccagaatcccaagagcttctctgggattttcttgtgatatctgattccccagtgaggcctgggacgtttttaagatcgctgtgtgtctgtaaaccctgaatctcatctggggtgggggccctgctggcaaccctgagccctgtccaaggttccctcttgtcagatctgagatttcctagttatgtctggggccctctgggagctgttatcatctcagatctcttcgcccatctatggctgtgttgtcacatctgtcccctcatttttgagatcccccaattctctggaactattctgctgcccctttttatgtgtctggagttccccaatcacatctagggctcctccaagatccttttgtcatgtctgaaatcactcttgagaggtctggggtggaggatggggagtcagtgaaatgtgtcatgtctgggccctgtcagggacacccttgttatatctgggatcctccaatcacatctgagacctcctaggctctccatctgatatgccctttcagggaccccacaaagactgagttctcatggggatcctacccttcctagtgccactccctatggccatgctgaagaccactctggccacgcgactgattttgggtgatcatggcagctccccacccatgtcatttctaaccagaagtctcaaggtcgtcacccccctgccccccaaccgaggccccggtcgctggtggtggtctctttagtgcactgtagcacttggtggtggaggtgtgagggatccacattaacagcaggccatcagctgggcaatggctcacacctgtaatcccagcactttgggaggcgaggcagggggaatggcttgaacccaggcattcaagaccagcctgggcaacataatgagacctcgtctctacaaaacataacaaaaacaattagccgagcgtgggggtgaacacctgtggtcccagctgctcaggaggctgaggtgggaggatctcttgagcccaggaagtaggaggctgtagtgagctgtaatcgtgccactgcactccagcctgggcgacagagtgagacaccgtcttaaaaacaaaaacaaggccgggcacggtggctcatgcctgttgtcccagcactttgggaggccgaggcaggcggatcacgaggtcgagagatcgagaccatcctggccaacatggtgaaaccctgtctctactaaaaatacagaaattagctgggcgtggtggcacgtgcctgtagtcccagctactcgggaggctgaggcaagagaatcgcttgaacgtgggaggcagaggttgcagtgagcctagattgtgccactgcactccagcctgggggacagagcgagactccgtctgaaaataaaaacaacaaaaacagcagaccattcaaaatagggagactttgcataatccagatttctgccttcacttaaaactttggacggtctggagagagtcggccagttttcggtggggggtggggagctggaacaggacagtagcctttcctaatgaggcatttgttctccaatctgccccagtcgctgccatccctggctatctcaccctagcagcttctcaagcctgttggctttagaccactgtataaacccagctggaactgaagcctgggtggactatggagccctggttgggacccccagggagtcaaaggctgcgggccaagaggccagaggtccttgagcctgggtgggcaggtggatctagggtgcatgacttgctgcttcccaaccttagtttgtcccttctgtgaaaaagggagagaaggaggaggaagatctcaaaaagactttccagcccagtgcggtggctcacgcctgtaatcccagcactttgggaggccgatgcaggtggatcacctgaggtaggagttcaagaccagcctgaccaacatagtgaagccccttctctactaaaaatacaaaattagctgggcgtggtggcatgtgcctgtactcccagctacttgggaggctgaggcaggagaatcgcttgaacctgggaggcggaggttgtagtgagctgagatcacaccactgcacaccagcctgggcgacaagagcgaaactccgtctcaaaaaaaaaaaactgttgcagccccgttgagcctttgacaccgcctgaaatccaccccactcccaggaggaggaggaggaaggaatgccaatgacctagagacacgagaagtccatgtggaggcacacagcagctgatggcagagcccaggctgggacctgcccttaagagaatgagtgggaagggggagggaggaagggcaggtaaaacgtcctccccagggccccctgcaacggggaaggtactttttacaaaagctatcattgtcaccctaaatgtggaataaaataagatgcatcgacgtagacaaacctcctgggaccttttgtcagggactgcaatcctgcccctccactgaggccgctggctctcagagacaccgtgacatcacgggtgatgatgagaggagttcaaagagagaattatatgctggcgcggtggctctgtaatcccaacactttggggggccaaggcaggaggatcgcttgagtacaggagtttgaaaccagcctgggcaagatagtgagatccccttcccacccgtctacaaaaaaaataaaaaattagcgggg

SEQ ID NO: 4 - DNA, 2786 bp, Mus musculus
agcctgctctgaaccaccatgcttgctctggaggctgcacagctggatggcccacacttaagctgtttgtacccagaaggagtcttctacgacctggacagctgcaagcccttcagttacccagattcagatgggggccttgactctacatggggctggacagaggccccgcctgctcctgccatcgctccctatgaagccttcgatcctgctactgctgcctttagccactcccaaactgttcagctctgttatagccatggtcctaacccctccacctatagccccatggggaccctcgacccagcccccagcttggaggccccagggcctggcctccaggtgtaccccccagaggacttcaccagccagaccctgggctccttggcttatgctccgtaccccagccctgtgctatcagaggaagaagacattatgctggacagccccgccctggaggtctcggacagtgagtcagacgaggccctcttggctggctccgaggggaggggatctgaggcaggtgcacgcaagaagctgcgcctgtaccagttcttgctggggttgctcctacgcggggacatgcgcgagtgcgtgtggtgggtggagccaggtgccggcgtcttccagttctcctccaagcacaaggagttgttggctcgccgctggggccagcagaagggcaaccgcaagcgcatgacgtatcagaagctggcccgagcgctgcgcaactatgccaagacaggcgaaatccgcaaggtcaaacgcaaactcacctaccagtttgacagcgcgctgctgccagcctcccggcatgtctgagcactccgctaaggacccctttctggcccctaagtcccatggagccccatatgagggcagtcagggttctcagctctccctagagcctccccagagtttcctgtgccgtgtataggattccaatctaggatggtcgtgtttgagggagcactggccattctacacggtttcagaatggcaggtttctcgggggggggggggatgggggagccctgatgtcgtctacggttccagaaaccgcagttcttgcgagtcctgtgagctcacatgacatctcaccagcaggtggcgctgtctacagccccccccaaacccttgttttgttggccagataggtcggtccctctgtactccccctgaagcccttgttagatctgaggtctagttatgtttggagctctctgagaaccctgtgccacctgtgtgtgacttttctctgcgtccgtttatgacttttgtttgtttgagacagggtctcattatgtagctcaggctggccccccaacttttaacaatcgtcctgcctcggcctcctgagtgctgggatgacaagggtgcaccatcacaccaggttttttccttttttgagagattttactatgtaacccgggctgggctattctcaagctagtggcagtcctcttgcctcaggctcctcttgcctcaggcaccccttgggaccctctgggacctatgtccgagatgaatggctgggtaaggtagggtgggaggttcagtgaaccttataggttgggcccttccttctgggatcccttgatcatatgggaagttctctaggctctcagcagccctgcattcacacactgactgaggcgcgacctgtatgttgtgtttgagggggatgtgtggcagaggtatggctgtggcaaggccggtgccttttattcttgagattgagtcttatgtagctcaggctggtcttgaactcactctcactgtgtagctagggaagaccttgagcccttgcctcagccagaatgctcagatgtcaggcagcgcaccacgtgactgtttctttccattgtcttgtctttttgttgttgttgttttcgagacagggtttctctgtgtagccctggccgttctggaactcactctgtagaccaggctggccttaaactcagaaatccacctgcctctgcctctcaaatgctgggattaaaggcgtgcaccaccacgccctttttcctttttcttaagtcaaggtctatttgtgtagcccaggctggcttcaagctcatgacactcttcctgcctctgcgtctggaactatgaacataccttactactctgtgcttacccacgccatgggtagacagacttctagacttggtcaacccccacccacaaggcaggcaaattaagtccctgcaggtgctttctttggaggaaagcccgctttcatagtgatctgtcaagctagaaagcactcccgccacccagatatctaagtgtgaatctttggacaactggtgacttctggccagcttttctggggccggcctctgtccaggtgtctgcccaggaccctgctcagtgcctgtctgtctcacaccagtgacttcctcacacccgcctgttcaggcccagtcttttccgttcaagtgctacaggccaagtaggctcaaactgtgggtttgtcttgggcatccacagcagaatcagaacccagagctttgaagcctgagtgaggggaggggggcactcaggcttccgtctctctgagaaacagacgatgaagaggcccttaaaactctttgcaaccccatgagcctccccaatgagcctgtgacacaccggaactcacctctgtgggtggccgggagggggaacagggtcatggaagatccagatgtccatgtggtcaaggctaaactgtactaaataaaattatttctcatcaccactatacatgcataataaataaagtgtacatcaaaatt

SEQ ID NO: 5 - DNA, 2343 bp, Homo sapiens
atctcagagagcgcggggtccggacagccgcgcgctgagggtctcggggcgggcgccgcgggacctctccgggccatggcagcccctgtcaaagggaacaggaagcagtccacggagggtgacgccctagacccacctgcatcccccaaacctgctggcaagcagaacggaatccagaaccccatctcgctggaggactcccccgaggcaggcggggagcgggaggaggagcaggagcgggaggaggagcaggccttcctggtcagcctctacaagttcatgaaggagcgacacacgcccatcgagagggtgccccatctcggcttcaagcagattaacctgtggaagatctacaaagcagtggagaagctgggggcctatgagctgcagtccatggccctaggagagagaatcggctggccgctgctggagccgcagagcagctgccaaactgcagtccttcgagtccctgcgagggcggccggagctgcaaggaccccgccgccagggggcgcccgccggccgcgccctcacgaggtgcccttgcaggtgaccgggcgccgcctctggaagaacgtgtacgacgagctggggggcagcccaggcagcaccagcgcggccacgtgcacgcgccgccactacgagaggctggtcctgccatacgtgcggcacctgaagggggaggatgacaagccgctgcccacctccaagcccaggaaacagtacaagatggctaaggagaacaggggggatgatggggccaccgagaggccgaagaaggccaaggaggagcggcgcatggaccagatgatgccaggaaagaccaaagcagatgctgctgacccagcaccacttcccagccaggagccccccaggaacagcacagaacagcagggcctggcctctgggtcttctgtgtcctttgtgggtgccagcggctgtcctgaggcctacaagcggctcctatccagcttctactgcaaggggacacacggcatcatgtcaccactggccaaaaagaagctcctggcccaggtgagcaaggtggaggccttgcagtgccaggaggagggctgccgccatggggcagagccccaggcgtccccagctgttcacctcccagagagtccccagagccccaaagggctgactgagaactccaggcaccggctgacccctcaggagggattgcaggccccaggtggcagcctcagagaggaggcgcaggcaggcccctgcccggcagcccccatcttcaagggctgcttctacacccaccccaccgaggtgctgaagcctgtcagccagcaccccagggacttcttctctagacttaaagatggggtgctattggggcctcctggcaaagaggggctgtcagtgaaagagccccagctggtgtggggcggagacgctaaccgcccttctgcgttccataaaggtggctccagaaagggcatcctctaccccaagcccaaagcctgctgggtgtcccccatggccaaggtcccagccgagagccccacgctcccgcccaccttccccagtagcccaggcctgggcagcaagcgcagcctggaggaagagggtgctgcccacagtgggaagagactgcgggccgtgtctccctttcttaaggaggcggatgccaagaagtgtggggccaaacctgcagggtccggcctggtctcctgccttctgggcccagccctggggcctgtgcccccagaggcctacaggggcaccatgctgcactgcccgctgaacttcactggcaccccgggccccttgaagggccaggctgcactccccttcagccccctggtcatcccggccttcccggcccacttcctggccaccgcaggcccctcgcccatggccgctggcctgatgcacttccccccaacgtccttcgacagtgccctccgccacagactttgcccggcctcatctgcctggcacgcaccaccagtcacaacctatgcagcgccccacttcttccacctcaacaccaagctgtaggccagcccatggtgttgtgtacactgtggagtcgacaggggcctacaacaggcaggtactgctgccagggggctctgaactagtgcctgctacccaggacacccgggccatgcccctggctgggcagcctggcacaagtgaagaagaaggcagtgggaaaactgggtttatctcaaggcagcagcctgagcccaggagcagaggacccagttgttataaggcgctgggagaggatgggcagctcccactgccccagagcggagctcgaagcacccaggttgcccacggaaaatccaataaaaagacaccagtgtgaatcca

SEQ ID NO: 6 - DNA, 5699 bp, Mus musculus
agtcgagcggcgccggtagtcagtcgcgccgagagcgctgggacaggacggctgtgcgtgtagagggtctctgcatagcgacgcgcgctctcgctgggccatggcagcacctccggccaaagggaacacagagcagtcagaagaaggtgacctcccgcagcttcctgtatcccccaagccagatgatgagcagagcaggagccagagccccacccagctccaggactcccctgaggcaggtggggagcaggaggaggaacaggccttcctggtcagcctctacaagttcatgaaggagcgacacacgcccatcgagagggtgccccatcttggcttcaagcagattaacctgtggaagatctacaaggcagtggagaagctgggggcctatgagctggtgacaggccgccgcctctggaagaacgtgtatgatgaacttggcggtagcccaggcagcaccagtgcggccacatgcacacgccgccactatgagaggctggtcctcccatatgtgcggcatctgaagggggaggacgacaagccactgcctcctaccaagcccaggaagcaatacaagatggccaaggagctgaggggagacgatgggaccactgagaagctgaagaaggccaaggactcagaggagaggcgggtggagcagaccacgccaggaaagaccaaatcagatgccactggccagacacagcttccctgccagggatcctcgagggacagcacagaacagctgggcccagtatctggaccctctccaccactcacgggtgctagtagctgccctgaggcctacaagcggctcttgtcaagcttttactgcaaaggggcgcatggcatcatgtcaccactggccaaaaagaaactcctggcccaggtcagcaaggcagaggccttgcagtgccaagaagagggctgtcgccatggagcaaggagccccaacaaggacattcaagacagtccccagaacctaagagggccggctgagaactctgaacaccagctaaccccccgggaaggattgcaggcccctggtgggagcaccaggatggaggcccaagtgggcccctgccctacagcccccatgttctcaggctgttttcatgcgtaccccaccgaggtgctgaaacctgtcagccagcaccctagggacttcttctccggccttaaagacagggtgctgttgggaccacctggtaaagaagaaggtccgacaaccaaagagtcccatctggtgtggggtggggatgccaaccacccctctgcattccataaaggcagcacaagaaaaagaagtttctaccccaaacccaaagcctgctgggtgtctcccatggccaaggtccctactgagaggcctggagccccatcccctcatcccagtagcccaggtcttggcagtaagcgcggcttggaagaagagggattcgctcatggtggcaagaaactgagggcagtgtctccctttctgaaggaggtggattccaaggagactgggggcaagcctgcagcccctggcttggctgtatcctgtctactgggcccaaccccggggcccactcctccagaggcctacaggggcaccatgctgcggtgtcctctaaacttcaccggtagcgcagaccctctgaagggccaggcctcactccccttcagccccctggtcatccctgctttcccagcccaccttctggctacaacaggctcctcacctatggctgccagcctgatgcatttccctcccacgccctatgacgctgtcctacgcaacagactgggtccagcttcgtctgcctggcacatgccacccgtcacaacctatgcggcacctcacttcttccacctcaacaccaaactgtaggccagagcctatcctgctatgctgtggaggatttgatgggcagctgccgccattatctcaggcctgagccgactacccagattcccaggccagtgaggctccccgagtctgtgtctctctggtacagacagcagggaggcagtggggcttgtctgaatgaagcagcccgggcccaaagccaggggaccaagttgtggtaagatcatgaaagtacctgagctggtattttctctccactgagaggccggggagctagttggcagctcaaggcatccaggttggccatgaaagacccaataaaaagacactggtgtgattgcactcagccctggggtgtacagcactggatgtttgcaaggggaggtgaggcaggagcaagtgtgtaggtgtggtgaccaatgtgcgagcaaagatgaaaggaggccactctggcctggctgccatcaccccactggaagggtggcagggccagagccaaactcagcctctggtgcacagatgaacagctgtgattcctttcgctccagctccagcctcggggcacctgactgttgaggttgccacggtgccaaagcccagagcacactggtgacctgtgagacaagatggaggggctgtgtcactgcaaccctctgagctgagccacacgcccccatcccagaaggcctcgggtctttcagtgaacggcatcacttgctctgagaggttcttgctgtgttgctcaggttagtctcggactcccgacctaaagaattcctgctgcggccttctcagtagctggaactactggtgtgaacctgtgaagaggggatttgtgggagctcacataaaacccaagtgaatctgtcctggtagtgggcagacctgtgagcccagctgcctagtgttcccagtgagcgacctgtcacattcacacctactttcctgttctacatactgggtgaagggtagaggggacccatctctacctgtgaatctgttgccaagtcacagtgatatttacctgtggttgctccaggtgcccacacagaaatcccatggcttggcacccattcctaccttggacatggcttgctctgccagtcccaaggagactgtcagtacagcttcacactgttcctggaaaggcagtgccactgtggaccagagatgccaggatgccagtgagccaagtcaacagatggttactgacctacatcatgggaactcaccctgttcctgacctcaggccatgggtaggagggcaagtttttatggcccccttagttcacaggcggaatggccaaggccagagacagcaggtgtcattgaggatgtctggctctgggaaacgggtggccagggaaagcagctgcctgtggaaaagagctgggacacaggtgtcctccaagaccaggtagctagagggtaagggtcccactgatgcatcttgcaactccccaccttccttctggagataccaggatgccagcctgagtattcagatgacaagccctgagctcctggctaccccactgcacccatgtgctacctgtctgggagcttttacagccccgtgaggagggattctaggctagcaaatgccctggctgttgggctgtacactgtggggtcaggtcccaggatagacagagggtaccttcgtgcgtgcgtggaaggatccccttcatctacaaagtaacactagtctcaacctgctcaaaggtggcgtatgacagacagcctgataatggtagcttgttccaagctgcaggaacggagccctctgcaggttcatctggagaatacacagctgggagggtagggtgtgtggctttctgggttgtactccactctgcttcagcaaaaactctagaaatgaatgcacatcaggcttcctgggaaatcttgggactttgcacccatgcgctagccccacatgattctccttccttctgtgaatgagagaactcagctcagaggtgactccaaacactgtggtacacacaccttcctatgttcacttgtgtccctaacaggttcccccagaggacccacaggccagcaaagctcttggcgtctgtgtccagggcataacttgtggtctgagcctacttccaaatcattcgtgacatttgagctaataatggtgggaggaggcagaggccactctggtatttgtgctgaccccacccctgtccgtggtcctggctcatttccctggaaacagcttaagtgtgatgagttgggcattcagtgggaaatacctttgtttgccttgggtgtcaaagcatgctgcccctgtaactgtcttaggtggcattggttagcattacatgtgcatctcccacacgaggcaagtatcaccaccgccatagttgtttggttggttggttttcttggcttctttttaagacaggatttctctgtgtagccctggctgacctggaacttactctgtagaccaggctggtctctaatagaggtccaccctctgcctctacttggattaaagatatgcaccaccatgcccagcaaccacctcgcccagcaaccatcagagttttaaaccgaatggctgggaattttcctttgaagaccttgcgaatggctacaggaggtgatggagaaacagcagcaagtccccgagggagtggcgagagagtggggccaggagccaggggggctgttgggcagggacggaggaagccagagatgacttaggctgcatttagaaccttctagtccaggaggcagttgggcttttatgtgaaataaagaaagatggaagagagtcctggctcccagatttggttgtttgctttatggtgctagggatggaaccctggacattttttatgctcagcaagctctgatcacttgctgcatcccagccctggactctggcaagtggtaggtgtggacactggagactagaacagagtagggaacgtatccttggagatggcagagtcaacatctggacacaggtctggagatgctgcggacacatctggccagcgtgtctgtttatactgtgagcagagtgagtgtgggggctgggcttggagaagggaagcgagctaggcagtgatcacagtagagcaaagccgtccaagggcacagccagggcacaagagctcgggtttccacttgaccttagcagcgtgagtatcattgcacctaggaggccggctggacacaatctcaaggcaagggacaactaagcaccactccagtgggagccgaagtggctgcatgaagggcatcaacagagaaccagggaatgaagtggggatggggggagggagttgggaacagtgggtaagatctctagactgcagatcttggtgtgctgtggggacttgatggggtgggtgcacatgagaggcttggctgaatcagcagctattggttgctggatgagctagagcaagaaggggtcaacagagctcaggcatgggggactttgaaacaagggcagcgggcatgctgggtgggcacagccgccagggtaaggtgtgtaggcacaggaaaggaggtgatggggcctcagcaaaaaacacagcacaggaggcaagaagcaaaggttaccttccacatgaagttgctgggctggccagagccttagactggcagaatagacattagagaaggccagaggcaatgaaaaacttcatggctttaaaggaattttaaatcaagagcagaggctgcctggcatgtttagatgtgtttgttttctagtgtgctggatggaacccagagccttgggtatgctgggcaagtatccaacactgagctatatcatttggtgttttttaaaaaaaagaattaacaatatttg

SEQ ID NO: 7 - DNA, 6102 bp, Homo sapiens
gtctctgtccatccagactcctgacgttcaagttcgcagggacgtcacgtccgcacttgaacttgcagctcaggggggcttttgccatttttttcatctctctctctctctctccctctatctctcttctctctctctccctctttttttttttttttttttttttttttttgcttaaaaaaaagccatgacggctctcccacaattcatcttccctgcgccatctttgtattatttctaatttattttggatgtcaaaaggcactgatgaagatattttctctggagtctccttctttctaacccggctctcccgatgtgaaccgagccgtcgtccgcccgccgccgccgccgccgccgccgccgcccgccccgcagcccaccatgtctcgccgcaagcaaggcaaaccccagcacttaagcaaacgggaattctcgcccgagcctcttgaagccattcttacagatgatgaaccagaccacggcccgttgggagctccagaaggggatcatgacctcctcacctgtgggcagtgccagatgaacttcccattgggggacattcttatttttatcgagcacaaacggaaacaatgcaatggcagcctctgcttagaaaaagctgtggataagccaccttccccttcaccaatcgagatgaaaaaagcatccaatcccgtggaggttggcatccaggtcacgccagaggatgacgattgtttatcaacgtcatctagaggaatttgccccaaacaggaacacatagcagataaacttctgcactggaggggcctctcctcccctcgttctgcacatggagctctaatccccacgcctgggatgagtgcagaatatgccccgcagggtatttgtaaagatgagcccagcagctacacatgtacaacttgcaaacagccattcaccagtgcatggtttctcttgcaacacgcacagaacactcatggattaagaatctacttagaaagcgaacacggaagtcccctgaccccgcgggttggtatcccttcaggactaggtgcagaatgtccttcccagccacctctccatgggattcatattgcagacaataacccctttaacctgctaagaataccaggatcagtatcgagagaggcttccggcctggcagaagggcgctttccacccactccccccctgtttagtccaccaccgagacatcacttggacccccaccgcatagagcgcctgggggcggaagagatggccctggccacccatcacccgagtgcctttgacagggtgctgcggttgaatccaatggctatggagcctcccgccatggatttctctaggagacttagagagctggcagggaacacgtctagcccaccgctgtccccaggccggcccagccctatgcaaaggttactgcaaccattccagccaggtagcaagccgcccttcctggcgacgccccccctccctcctctgcaatccgcccctcctccctcccagcccccggtcaagtccaagtcatgcgagttctgcggcaagacgttcaaatttcagagcaacctggtggtgcaccggcgcagccacacgggcgagaagccctacaagtgcaacctgtgcgaccacgcgtgcacccaggccagcaagctgaagcgccacatgaagacgcacatgcacaaatcgtcccccatgacggtcaagtccgacgacggtctctccaccgccagctccccggaacccggcaccagcgacttggtgggcagcgccagcagcgcgctcaagtccgtggtggccaagttcaagagcgagaacgaccccaacctgatcccggagaacggggacgaggaggaagaggaggacgacgaggaagaggaagaagaggaggaagaggaggaggaggagctgacggagagcgagagggtggactacggcttcgggctgagcctggaggcggcgcgccaccacgagaacagctcgcggggcgcggtcgtgggcgtgggcgacgagagccgcgccctgcccgacgtcatgcagggcatggtgctcagctccatgcagcacttcagcgaggccttccaccaggtcctgggcgagaagcataagcgcggccacctggccgaggccgagggccacagggacacttgcgacgaagactcggtggccggcgagtcggaccgcatagacgatggcactgttaatggccgcggctgctccccgggcgagtcggcctcggggggcctgtccaaaaagctgctgctgggcagccccagctcgctgagccccttctctaagcgcatcaagctcgagaaggagttcgacctgcccccggccgcgatgcccaacacggagaacgtgtactcgcagtggctcgccggctacgcggcctccaggcagctcaaagatcccttccttagcttcggagactccagacaatcgccttttgcctcctcgtcggagcactcctcggagaacgggagtttgcgcttctccacaccgcccggggagctggacggagggatctcggggcgcagcggcacgggaagtggagggagcacgccccatattagtggtccgggcccgggcaggcccagctcaaaagagggcagacgcagcgacacttgtgagtactgtgggaaagtcttcaagaactgtagcaatctcactgtccacaggagaagccacacgggcgaaaggccttataaatgcgagctgtgcaactatgcctgtgcccagagtagcaagctcaccaggcacatgaaaacgcatggccaggtggggaaggacgtttacaaatgtgaaatttgtaagatgccttttagcgtgtacagtaccctggagaaacacatgaaaaaatggcacagtgatcgagtgttgaataatgatataaaaactgaatagaggtatattaatacccctccctcactcccacctgacaccccctttttcaccactccccttccccatcgccctccagccccactccctgtaggatttttttctagtcccatgtgatttaaacaaacaaacaaacaaacagaagtaacgaagctaagaatatgagagtgcttgtcaccagcacacctgttttttttctttttctttttcttttttctttttccttttttttttttttcctttatgttctcaccgtttgaatgcatgatctgtatggggcaatactattgcattttacgcaaactttgagcctttctcttgtgcaataatttacatgttgtgtatgtttttttttaaacttagacagcatgtatggtatgttatggctattttaaattgtccctaattcgttgctgagcaaacatgttgctgtttccagttccgttctgagagaaaaagagagagagagagaaaaagaccatgctgcatacattctgtaatacatatcatgtacagttttattttataacgtgaggaggaaaaacagtctttggattaaccctctatagacagaatagatagcactgaaaaaaaatctctatgagctaaatgtctgtctctaaagggttaaatgtatcaattggaaaggaagaaaaaaggccttgaattgacaaattaacagaaaaacagaacaagtttattctatcatttggttttaaaatatgagtgccttggatctattaaaaccacatcgatggttctttctacttgttataaacttgtagcttaattcagcattgggtgaggtaataaaccttaggaactagcatataattctatattgtatttctcacaacaatggctacctaaaaagatgacccattatgtcctagttaatcatcatttttcctttagtttaattttataaacaaaactgattataccagtataaaagctactttgctcctggtgagagcttaaaagaaatgggctgttttgcccaaagttttattttttttaaacaatgattaaattgaatgtgtaatgtgcaaaagccctggaacgcaattaaatacactagtaaggagttcattttatgaagatatttgctttaataatgtctttttaaaaatactggcaccaaaagaaatagatccagatctacttggttgtcaagtggacaatcaaatgataaactttaagaccttgtataccatattgaaaggaagaggctgacaataaggtttgacagaggggaacagaagaaaataatatgatttattagcacaacgtggtactatttgccatttaaaactagaacaggtatataagctaatattgatacaatgatgattaactatgaattcttaagacttgcatttaaatgtgacattcttaaaaaaagaagagaaagaattttaagagtagcagtatatatgtctgtgctccctaaaagttgtacttcatttcttttccatacactgtgtgctatttgtgttaacatggaagaggattcattgtttttatttttatttttttaattttttcttttttattaagctagcatctgccccagttggtgttcaaatagcacttgactctgcctgtgatatctgtatcttttctctaatcagagatacagaggttgagtataaaataaacctgctcagataggacaattaagtgcactgtacaattttcccagtttacaggtctatacttaagggaaaagttgcaagaatgctgaaaaaaaattgaacacaatctcattgaggagcattttttaaaaactaaaaaaaaaaaaactttgccagccatttacttgactattgagcttacttacttggacgcaacattgcaagcgctgtgaatggaaacagaatacacttaacatagaaatgaatgattgctttcgcttctacagtgcaaggatttttttgtacaaaacttttttaaatataaatgttaagaaaaattttttttaaaaaacacttcattatgtttaggggggaactgcattttagggttccattgtcttggtggtgttacaagacttgttatccatttaaaaatggtagtggaaattctatgccttggatacacaccgctcttcaggttgtaaaaaaaaaaaacatacattggggaaaggtttaagattatatagtacttaaatataggaaaatgcacactcatgttgattcctatgctaaaatacatttatggtcttttttctgtatttctagaatggtatttgaattaaatgttcatctagtgttaggcactatagtatttatattgaagcttgtatttttaactgttgcttgttctcttaaaaggtatcaatgtaccttttttggtagtggaaaaaaaaaagacaggctgccacagtatatttttttaatttggcaggataatatagtgcaaattatttgtatgcttcaaaaaaaaaaaaaagagagaaacaaaaaagtgtgacattacagatgagaagccatataatggcggtttgggggagcctgctagaatgtcacatggatggctgtcataggggttgtacatatccttttttgttcctttttcctgctgccatactgtatgcagtactgcaagctaataacgttggtttgttatgtagtgtgctttttgtccctttccttctatcaccctacattccagcatcttaccttcatatgcagtaaaagaaagaaagaaaaaaaaaggaaaaaaaaaaaaaaaccaatgttttgcagtttttttcattgccaaaaactaaatggtgctttatatttagattggaaagaatttcatatgcaaagcatattaaagagaaagcccgctttagtcaatacttttttgtaaatggcaatgcagaatattttgttattggccttttctattcctgtaatgaaagctgtttgtcgtaacttgaaattttatcttttactatgggagtcactatttattattgcttatgtgccctgttcaaaacagaggcacttaatttgatcttttatttttctttgtttttatttttttttttatttagatgaccaaaggtcattacaacctggctttttattgtatttgtttctggtctttgttaagttctattggaaaaaccactgtctgtgtttttttggcagttgtctgcattaacctgttcatacacccattttgtccctttattgaaaaaataaaaaaaattaaagtaca

SEQ ID NO: 8 - DNA, 6122 bp, Mus musculus
gacgttcaagttcgcagggacgtcacgtccgcacttgaacttgcagctcaggggggcttttgccatttttttcatctctctctctccctctatccctcttctctcttcctctctctcttttttttccttaaaaaaaaaaaagccatgacggctctcccacaattcatcttccctgcgccatctttgtattatttctaatttattttggatgtcaaaaggcactgatgaagatattttctctggagtctccttctttctaacccggctctcccgatgtgaaccgagccgtcgtccgcacgccgccgccgccgccgccgcccgccccgcagcccaccatgtctcgccgcaagcaaggcaaaccccagcacttaagcaaacgggaattctcgcccgaacctcttgaagccattcttacagatgatgaaccagaccatggcccgttgggagctccagaaggggaccacgaccttctcacctgtgggcagtgccagatgaatttcccactgggggacattcttatttttatcgagcacaaacggaaacaatgcaatggcagcctctgcttagaaaaaggtgtggataagccgccttccccttctcccatcgagatgaaaaaggcatccaatcctgtggaggttggcatccaggtcacgccagaggatgacgattgtttatcaacgtcatctagaggaatttgccccaaacaggaacacatagcagataaacttctgcactggaggggcctgtcctctcctcggtctgcacacggagctctaatccccacgcccgggatgagtgcagaatatgccccgcagggtatttgtaaagatgagcccagcagctacacatgtacaacttgcaaacagccattcaccagtgcatggtttctcttgcaacacgcacagaacactcatggattaagaatctacttagaaagtgaacacggaagtcccctgaccccgcgggttggtatcccttcaggactaggtgcagaatgtccttcccagccacctctccatgggattcatattgcagacaataacccctttaacctgctaagaataccaggatcagtatcgagagaggcttccggcctggcagaagggcgctttccacccactccccccctgtttagtccaccaccgagacatcacttggacccccaccgcatagagcgcctgggggcggaagagatggccctggccacccatcacccgagtgcctttgacagggtgctgcggttgaatccaatggctatggagcctcccgccatggatttctctaggagacttagagagctggcagggaacacgtctagtccaccgctgtccccaggccggcccagtcctatgcaaaggttactgcaaccattccagccaggtagcaagccacccttcctggcgacgccccccctccctcctctgcaatccgcccctcctccctcccaacccccggtcaagtccaagtcatgcgagttctgcggcaagacgttcaaatttcagagcaacttggtggttcaccgacgcagccatactggtgagaagccctataagtgcaacctgtgcgaccacgcgtgcacacaggccagcaagctgaagcgtcacatgaagacacacatgcacaaatcgtcccccatgacagtcaagtccgacgatggcctctccacagccagctccccggaacctggtaccagcgacctggtgggcagcgccagcagtgcgctcaagtcagtggtggccaagttcaagagtgagaacgaccccaacttgatcccagagaacggggatgaggaggaagaggaggacgacgaggaagaagaagaagaggaggaagaggaggaggaggagctgacggagagcgagagggtggactacggcttcgggctgagcctggaggctgcacgccaccatgagaacagctctcggggcgcagtggtgggcgtgggcgacgagggccgcgccctgcccgatgtcatgcagggcatggtgctcagctccatgcagcacttcagcgaggccttccaccaggtcctgggcgaaaagcataagcgtagccacctggccgaggccgagggccatagggacacttgtgatgaagactcggtggccggtgagtcagaccgcatagacgatggcactgttaatggtcgtggctgctcccccggcgaatcggcttcggggggtctgtccaaaaagctgctgctgggtagccccagctcgctgagccccttctccaagcgcatcaagctggagaaggagtttgacctgcccccggccgcgatgcctaacacggagaacgtgtattcgcagtggctcgctggctatgcggcctccaggcagctcaaagatcccttccttactttcggagactccagacaatcgccttttgcctcctcatcagagcactcctcggagaacgggagcttgcgcttctccacaccgcccggggagctggacggagggatctcagggcgcagcggcacaggaagtggagggagcacgccccatattagtggtccgggcccgggcaggcccagctcaaaagagggcagacgcagcgacacttgtgagtactgtgggaaagtcttcaagaactgtagcaatctcactgtccacaggagaagccacacgggcgaaaggccttataaatgcgagctgtgcaactatgcctgtgcccagagtagcaagctcaccaggcacatgaaaacgcatggccaggtggggaaggacgtttacaaatgtgaaatttgtaagatgccttttagcgtgtacagtaccctggagaaacacatgaaaaaatggcacagtgatcgagtgttgaataatgatataaaaactgaatagaggtatattaataccctccctcactcccacttgatgccccccttccaccccttccccattgtccttccagccctactccctgtaggatttttctagtcccatgtgatcaaacaaacaaacaaacaaacaacagaggtaatggaagctaagaatatgaatgagtgcttgtcaccagcacacctggttttttgttttgtttttccttttttttttctttttcttcttcttttttttaaattttaaattctttatgttctcaccgtttgaatgcatgtttggggcaatactattgcattttacgcaaactttgagcctttctcttgtgcaataatttacatgttgtgtatgtttttccccccttaacttagacagcatgtatggtatgttacggctattttaaattgtccctaattcattatgagcaaacatgttgttgctgtttccagttccattctgagagtgtgagggagggagggtagaaaacaacgcatgctgcatacataattctgtaatacagatcatgtgcagctttattttataacacgaggagggaaacggtgtctgggttgactaaccctctgcagacagagcagatagcagtgaaaaaaaggtgctaaatgtctgtctctaaagggttacatgtattaattggagagggaaaaaaggccttgaattgacaaattaacagaagaacaagtttattctatcatttggcttttaaaatgagtgccttggatctattcaaaccatgttgatggttttttttctgcttgttataaacttgtagtttaatctagcattgggtgaggtaataaactttaggacctagcatgtaattctgtgttgtatttctcacaacaatggctacctaaaaatatgacccattatgtcctagttaatcattgatttttgcctttaactttgtgaacaaaactgattataccagtataaaagctactttgctccttgtgagagcataaaagaaatgggctgtttcgcctaaagttttattttattttatttttaaatggttatcaaattgaatgtgaaatgtgcaaaggccctggaatgtgatgaaatacattagcaagaagttcatcttgtgacaatacttgtttaaaatgatgcatttaatgaaattctggcgccaaaagaagtagatccggctctagttggttgttgagtggacaataaaatgataaagccctttagaggacatttgaaagaacaggctgatatgatgagagagagagagagagacagaaaacggtagtgattggttagcacgatgtagtactgtttgccatttgaaactagaacaggtgtataaggggccagcgatacaatgatgattaactctgaagctctaagacttgcatttaatgtgacagtcttcaaaaagaagaggaagacctttaagagcagcagtatctatgtctgtgctccctggaagttgtacttcatttcttttccatacactgtgtgctatttgtgttaacattgaagaggatttgtttttattttgttttattctttctttctttcttttttcttccttctttgttttaagctagcatctgccccagttggtgttcaaatagcacttgactctgcctatgatacctgtatcttttctctaatcagagatacagaggttgagtataaaataaacctgctcagataggacaattaagtgcactgtacagttttcccagtttacaggtctttattaagggaaacgttgcaagaatgctgaaaacaattgaacacaatctcaatgatgagcattaaaaaataataaaaaaaaacaagcaaacctaaaaaaaactaagacagacgttgccagccattgacttgactattgagcttcctcacttggatgcaacattgcaagcgctgtggatggaaacaacacacttaacatagaaacgaatgactcctttgcttctacagtgcaaggatttttgtacaaaacttttttaagtataaatgttaagaaaagaatttttaaaaagacacttcattatgtttaggggggaacagcattttagggttccattgtcttggtggtgttacaagacctgttatccatttaaaaatggtagtggaaattctatgccttggatcacacaccgctcttcaggttgtaaaaaaaaatgaaaacaaaacaaaacaaaaaaaaaacatacatggggaaaggtttaagattatatagtacttaaaaataggaaaacgcacactcatgttgattcctatgctaaaacacagttatggtcttctttctgtatttctagaatggtatttgaattaaatgttcatctagtgttaggcactatagtatttatattgaagcttgtatttttaactgttgcttgttcactcaaaaggtatcaatgtacctttttgttagtagaaaaaaaaagacaggctgccacagtatatttttttaatttggcaggataatatagtgcaaattatttgtatgctgagagagagagagagagagagagagagagagagagaggtgtgacattgtacagagaagccatataatggcggtttggggagcctgctagaatgtcacatggatggctgtcataggggttgtacatatccttttccccctttcctgctgccatgctgtacgcagcactgcaagctaatagcgttggtttgttatgtagtgtgctttggcccctcctccccgctcacccgacattccagcatcttaccttcatatgcagtaaaagaaagaaagaaaaaaaaaaaggaaaaaaaaaacaacaacaatgttttgcagtttttttcattgccaaaaactaaatggtgctttatatttagattggagagaatttcttatgcaaagcatattaaagagaaagcccgctttagtcaatacttttttgtaaatggcaatgcagaatattttgttattggccttttctattcctgtaatgaaagctgtttgtcgtaacttgaaattttatcttttactatgggagtcactatttattattgcttatatgccctgttcaaaacagaggcacttaatttgatcttttatttttctctgtttttattattattttttttaatttggatgaccaaaggtcattgcaacctggctttttactgtatttgtttctggtctttgttaagttctattggaaaaaccactgtctgtgtttttttggcagttgtctgcattaacctgttcatacacccattttgtccctttattgaaaaaaaataaaaaaattaaagtactaaaaaaaaaaa

SEQ ID NO: 9 - DNA, 4480 bp, Homo sapiens
aaaccggaatggcccgagggaaggccgcgcagggcagggccccagatggttcctgtcagggaagtggcgggcgcagctgcaggcctccggccccggcattatcacggggacacagctggctgcctcacccgcaggctgcagggagaccttccccagcctgcagccccaggcccgccccgcgtcacatgagccccagggctcccaccccctccccagggcagaggacacccagttggtggccgggagggcctcggctttccagggacagaggcccaactccaggacgccccagctggcccagcccctcctctttccctcaaggctgcaggaggtcgggaaaggcagtcctggtagaggcctgtcctgggctccaggttggcccctgagggtggccctcctcatgccggcttcaagactgagggacagggcagccagttcagcctcgggatccacctgtggctccatgtcccagacgcaccctgtgctggagagcggcctcctggcatctgccggctgctccgcaccccggggtcccaggaagggcggcccagccccagtggacaggaaagctaaggcctcagcgatgccggactccccagcggaggtgaagacgcagccccggtccacaccccccagcatgccgcccccaccgcctgccgcatcccagggggccacacgccccccctccttcacgccacacacacatcgagaggacgggcctgcgacgctgccccacggccgttttcatggctgcttaaaatggtctatggtctgtctcttgatgaacggcagcagccactcaccaacagccatcaatggtgcaccgtgcacacccaacggcttcagcaatggcccggccacctcgtccacagcctccttgtccacacagcacctgcccccagcctgcggggcccggcagctcagcaagctcaagcgcttcctcaccacactgcagcagtttggcagcgacatctccccagagattggggagcgcgtgcgcacactggtgctgggcctggtgaactcgacattgacgatcgaggagtttcattccaagcttcaggaggccaccaacttccctctgcggccgtttgtcattcccttcctgaaggcaaacctgcccttgctgcagcgggagctcctgcactgtgcacgcctggccaagcagacgcccgcccagtacttggcccagcatgagcagctcctgctggacgccagcgcctcctcccccatcgactcctcagagctgctactggaagtcaacgagaacggcaagaggaggacgcccgacaggaccaaagagaacgggtcagaccgcgacccgctgcaccccgagcacctcagcaaacggccatgcaccctgaaccctgcccagcgctacagccccagcaacgggccaccgcagcccacaccgccgccgcactaccgcctggaggacatagccatggcccaccacttccgagatgcctaccgccacccagacccccgggagctacgagagcgccatcggccgcttgtggtgcctgggtcccggcaggaagaagtgatcgaccacaagctcacagagcgtgagtgggcagaagagtggaagcacctcaacaacctcctgaactgcatcatggacatggtggagaagacgcggcgctcgctcacggtgctgcgcaggtgccaggaggccgaccgcgaggagctcaaccactgggcgcggcgctacagcgacgccgaggacacaaagaagggccccgctcccgccgcggcccggccccgcagcagctccgccggtcccgaagggcctcagctagacgtgcctcgcgagttcctgccgaggaccctcaccggctacgtgcctgaggacatctggaggaaggctgaagaggccgtgaatgaggtgaagcggcaggccatgtcggagctgcagaaagccgtgtcggacgcggagcgcaaagcgcacgagctcatcaccacggagcgtgccaagatggagcgggccctggccgaggcgaagcggcaggcctccgaggacgccctgacggtcatcaaccagcaggaggactccagcgagagctgctggaactgcgggcggaaagccagtgagacgtgcagcggctgcaacgcggcacgctactgcgggtccttctgccagcatcgggactgggagaagcatcaccacgtgtgtggccagagcctgcagggccccacagccgtggtggccgacccggtgcctggaccgcccgaagccgcccacagcctgggcccctccctgcctgtgggtgctgccagccccagcgaagccggctctgcggggccttctcgccccggctcccccagcccacctggcccactggacaccgtgccccgctgaccccactggcccctggcctgccggacacagcaccgtgccaaccccacccagctccaggcccaccggatgctgtgcctggcctccgatgcctggcctgccagacactgcgccccgcctgacctgggggagccgaccaattagtcactgctgctactgcccctctccgaaagaagacacagaaccaacaaaaccgcattcagtgcacctgcctcagctacctaatgattccgcgcggagacctcctgacaacgtctcttcaagcatcctcagaagcctcgactgagctttagacagcagagcagatgccgcaggcgcggcggctctgcccacctctcttttcctctctgtctgtctctccccctctgtcttctctatcctctctctctctatgactatcacacactttctcttcaatgaaaaaatcgaattggtggcttatattttcagcaaagaattttggggggttttgtgtgttggcaaaagagctactcagaaatggacaaagaaaacgggggggttctccccctcctgattaaaaagggagaaagaaaactgcgattttatagctggagatctgaacccagctgtgcccctcccccaggggcgtgaggctgatcagcgaagacgggaggaaagatttcgatttctgactcaagatgcatttttggtttcagatttttttttcctgtaatgttaaactctttggctttaagtaaaaatccaaaaagtttttttaaaaaagcaaaggaagcatacttgtgaactaccttgctagctagccagccaaggataccggacacacctctgctccaaaggaaatccaaaaaagcaaacacaagaaatcaaaatccaaaatttgtttgtcactgccaaagtatttttttcactgtttcacttgctcttgggtttgtttggatgtgggtctttttctcttctgttctgattttgtttgtgggtgtcgggatatttgggtgcagagggtttgtgcccagttagaagcgacttttgttctcttctgcgtaggcgttggtgcgtccgccgcgtgtgcgtggtccgtgtgccgttgctccggcctgcgtctccatatgtgtaggaaaggacacgccgtctgtcctcacgccccctgtgacttttcatatttccgttttccacttgtggaaaaaaagtgctaaagttttcttcccagagagagcataattccgaaacaaaactgtgacaatcttttgggttgattctcgactgcttttcgagcatgcggagccagcaggcctccctgaaacactgcttctcggccagcccgtcctcctctacctctctcctctccgcgccctccgacctctctcggccccctcaccccagctccgacctctctcagccccatcgccccaactccaacctctcggccccatcgccccaccgcagctactcccctttcttccaaacttttgcagaaaaaacaaaaaaactacaaacaaaagcagccctctgcctcctccccagggaagaccctgaccgtgtacatagccctggtgctcctgcccagccacccctcagatgcgttcgcctctggccctggggtgtgtctcggtgacgttttctatcagacgtgctccctcccatcctccagccctgcccaccctccctccactcctctcaactgcctcagcgatttcaagaaggaaataaagggataaagaaattcatgcttgcaccgagtacaaggacagacagcaggcacggcccgcagcctggcatctgtgcgtgtggcgtggcccgtggcttggcatctgtgtgcgtggtgtggcccgtggcctggcatctgtgtgcgtggcgtggcccgtggcctggcatctgtgtgtgtggcgtggcccgtggcctggcatctgtgcgcgtggcgtggcccgtggcctggcatctgtgtgcgtggctatcaggagttctaggaactcagtgcaatacgggagtgacccagctactgaaccagccacgaacagcccgccagaggcctgaagctgagcgtgtacgttaatgtgaatgtatatagtctttgcagaggtccaaatgatattcatgatggtaataaacgagatgtttgccaaataaaaaacagaaaccgca

SEQ ID NO: 10 - DNA, 7659 bp, Mus musculus
agggccacaacccagctctgccggctgtagtgactagaaaggcctggagcctccaaggaacagaggcacgggctccgagacgccaaagctcctccagccctcctgttctctccagaccccacgaaatcagaaaaggtgcttcagggtgggctctggaggccggccagctccctccagccccaggtaccatcaatctccggctccatctgcagctccatgtcccaggcatccaccaccactctggagagtggggcactcctctcgggaccccggggtcttcagaatggaagcccagctcacaggaaggagaaggctgcagcaatgccagactccccggctgaagtgaagacgcagccccggtccacaccccccagcatgccgcccccacctcccacctcatcccagggagccacacgaccaccctccttcacacctcacacacatggcgaggatggacctgcgacgtctcttccccatggccgtttccacggctgcttaaagtggtccatggtctgtctcttgatgaacggcagcagccactcacccacggccatccacggtgccccatctacacccaatggcttcagcaacggcccagccacctcatccacggcctcgctctccacacagcacctgcccccggcgtgcggggcacggcagctcagcaagcttaagcgtttcctcaccaccctacagcagtttggcagtgacatctcacctgagatcggggagcgcgtgcgcacactggtgctgggcctggtgaactcaactctgacgatcgaagagtttcatgccaagctccaggaagccaccaactttccactgaggccgtttgttatcccttttctgaaggctaatcttccactgctgcagcgtgagctcctgcactgtgcccgcctggccaaacagacacctgcccagtacctggcccagcacgaacagctgctgctggacgccagcgccacctcccctgtcgactcgtctgagctcctgctggaagtcaacgagaacggcaaaaggagaacacctgacaggaccaaagagaatggatcagaccgggaccctctgcaccccgaccacctcagtaagcggtcctgcaccctgagccccgcccagcgctgcagccccagcaatgggctgccccacccgacgccacccccacccccgcactatcgcctggaggacatggccatggcccaccatttccgggactcctaccgccatcctgatccccgagagctacgggaacgccaccggcccctggccatacctgggtctcgacaagaagaagtgattgatcacaggctcacagaacgcgagtgggcagaagaatggaagcacctcaacagtcttctgaactgcatcatggacatggtggagaagacccggcgatccctcaccgtcctgcgccggtgtcaggaggccgaccgtgaggaactcaaccactggatccggtgctacagtgactctgaggaggggaagaagggccctacccccatctctgcccggtccctcaacagctgcagtggccctgaggggtctcagctagatgttcaccgggacttcacgcccaggaccctgtctggctacatgcctgaagagatctggaggaaggctgaagaagctgtgaatgaggtgaagcgccaggccatgtcagaactacagaaagctgtgtctgatgcggagcgcaaagcccatgaactcatcaccacagagcgtgccaagatggaacgagccctggcggaggccaagcgacaggcctcggaggatgccctgactgtcatcaaccagcaagaggactccagcgagagctgctggaactgcgggcgcaaggccagcgagacgtgcagtggctgtaacgccgcccgctactgcgggtccttctgtcagcacaaagactgggagaaacaccatcacgtgtgcggccagagtctgcagggccccgcggctgcagtggctgacccactacctggacagcctgacgccactgccagccccagcgaagccggctcggcagggccctctcgtccctgctctccggggccgccaggcccgctggacgctgctgtgccccgctgacctccagatctgacacccagcccatggacgccatgccctgccaacctcctggccccaccgtggcccaccagttgcctggagccattgctgctactgcttctctccaaaagaaaacacagatccaacagaactgcatccgtgcagccccagctacctgacaaggtctgccgggacctctacagcctctcgtccatcgcaagcaccctcagaaagcatcgcagagcgtcagacagtggcaccagcaggagtgtggccagctccgccgctgtctgtcccctctgtgtccgtctctctgtgtccgtctgtctttctgcgtctgcctctctctgtgtgtctctccgtgtctttgtctcctgccgtttccctgactctccggcgtctctgtctttgtaaagtccacatgatctctctgtcatcagagaaacctagttggtagcttttcttttccatgaagaacttttggagattttgttttgttttgttggcaaacaagctacttagaaatggacaaagaagactgtggggttctccccattcctcatgagtaagggaagaaactgtgatttttctatccagagttgctgtatcgcccagccagccccaggggcatcagcgagcacacagcagagataatgagagcaactgcaatttccaacttaagaagcagcttttgtttcaggttttactcctttaatgtcaaactctttggctttaagtgaaaaaaaaaaaaaggaaaaaaaaatccaaaaagttttttttttttttaagcaaaagaaacacacctgtaaactaccttgccagctagccagccaaggatgccagacacacctcgctccaaaggaagcccaaaaaagcaaactcaagaaattgaaatccaaaatttatttttatcactgccaaagtattttcttcatggtctctccgccctgggttggtttgcatgtcagtctttgatttgggttttgattttgttcagggggtgttgggatgctgggatttggtgggtggggccaagaagaaaccttttgttcctttttgactagtaggcagcatccttgctgggcagagccatcggcagcaaggccacgtgaccctcagcaccgcatccagaagccccagtcagcgtgagcctgagcgttgtgtgccctagggctccgtccatgcgttccctacatgcaagggaggcacctccttccatcggtccccacaccttcctgggacttctcataagtcatttccacttgtgaaaaaaaaaaaagtgctaaagtgctcttcccagagagagcacacttcctaaacgaaactgtgacaatctttttggttgattcttgactgcttttgagagcataaggagacagcaaatcacgctttctcagttttccctcctctacctctctgctcttccatcccaccctacctccatctcggccccatcaccctgcccccagtgtcttgctgtggctaccctcatttctttcaaacttttacagaaaaaaaggaaaaagaaaaaaaaaaaacacgaaagccagcagccacccgcacctcacccggaagactccagcccggctgtacatatccctgtgttgtatccccaatgttggtctccagtcctgggggtgacggtgatgcttgcccatctacactctagcccctgcccaccctgcccttcgcccgctcgactccctcaaggattcaaggaggaaatgaaggaagaaggctcatgcctgtaccaaacaaaaagagacagcagactcagtccaggtctgcacctctgttgtggctgcagcaagccgcaggcctagcgtgcaatatgggggattgctcctgaaccctgggcccgggggggccctgcccagagcaaggtgtgcagcgtgtgttaatgtgaatgtacatagtctccacagaggtcccaaaccgtggcatcagtaccgaggagatgtttgccaaataaaaagaaattaagacaaaccacggaagtctgtgaggtttaggaaacgctttggaaggagtctgagtcctataggcttctttggtggctggtgtttcttcacagctgtatgaccattgtttaacagaacgttctatggattctgttgggtagcccggaatggagcctgacagcaacccttgctcggaaggctggggtagatctcattccctggaaggttcccgcggccgcagccctccaaggccgcagagatgtattcttgtgggtttcagagaagccaggacacaaggcggcctcctgcctgcccgcccttctctagtggaccagtcctcctgagcgtctgcctgcaaacactgagtctgtgtgctggtagagcgagacctgcctcccgtcgcaaggagcatcactggattccattcccaactgtaccccatgcggtacttcttgtctctgctactccactgcatgcagcaaccttggcccttccagaagggcaggccagtgaccactggtaggaaagagctctgggtgatggggtggtgagcaccatgttgggggggggggggaacaaagctaagcccctcaggttgtgcttaagagctgtagtctctagaagtccgggccaggagtcctgaggtggcattcctgatgacctacgtccctggtgtcaggacttcggagcacctgctcctatgtggtccagatattctgaggtaggtcccaaagggtcaccagtttgctttagttcaaatacctcctttcttacccttaccagccttacccaccttttaggaacagagggcccaccacagacaacaaaatgctgcccagccaaattcatctccatgagccaggttacatatttagggtttagcagagacaccacccccccatctatactctagccgttgggagcgacagcccctcaaggcggctgtcagatggcattggaccacagtgagtctcctgtgcatgggatttgactagcaattgtagacatcgagggtgagatgccatgtctgctaggcagagctcaggagtcaaggcaagtgcagaatcagggtggcagggagatggggctggaaagggttaggtgtcgcctgaggcaaggaggctggaggataaggtggggttgctaagccgggcaccgaggccctggctaagctgtgagttgtgctttgaccccagctacccagagacctcacagcacaaccagacaacctcacagctttgaagggacagggaccatgggtgttctttattgggaggtataccctgggtggccataaccgcagcctacccttggtttcaacctacagtctgcctggggctctccacagagctgggggcggggcatggtactccatccccatccctgctacctttctctggttcacacaccatggacccaggccctgcctcctgggaaccacagccaagcatgaaccaaaccccctaggcgatgtggctctcacagagccttgctcccccagagcctccaactcagaataccactttacttcctgttctgtggccaaagctaaaccaggaaggctgagtagggagggcaggtagcaggcagagctctccaacccccaaggcccagcccagatcccatctcatgaccttatggcttcttggcacaagtcctgtgtgtcccagccctgcctcagacccagactggcctttccgtgaggactacccaagagactactttccagctcagtcctaagctctgctcaggagcggaggccagtgtttctcctcccaggtgtggtcacagctggataggaactggctgaatgacaccacagctcaggtcccacccactgcctgacaagtgagaggagccagcccccaggggagcacccagaaggattcagttaacagggacatctgtcagaagacctgagagggttagctggggatggagaaggccaagaatgggaggcggggcctctgtgggcagtgtcaccttaaggccacagtgggcagggatcaggatgggacccaaagtttagtttctccaaataaatcattaggctgacaccaaagtatcatcccggcacccatctgtcctggcatccatctgtccactcttgtactcctcagcctaacggacagatcaacgctttcctgagctcctccttctgacacgtgcagccatcttcccatgaacccgtttgcctctgaccattcatcatcctctgcctcccttccagccaactacccccatctatctatcttgacccattggcaaatccatctttccacctgtccattgctccatccaaactgtctctccaccccaccatcccagcatactatacctacgcccttacaccttccagccccatgcacacaaccatgttccctatcatctctctattcactgtactcagagttctgtcagctccatcagtgtaccaaagacaccaggtcctcctacgccctctactctgcttggcctaaactgtccagggctgctctaggaatatctacccatgaggcaacaggcgtctcccacccctggcactctgccccaccatggtgttcagatacctccagaaatatgagttcttaccgtaggcatcgtgggcaggttggatacattcttgtggcagctgtgacacatctggagctgcttggggagagaagggaacaagaattgtgctgtggggtgggtctgtacgcctgctcacgagctctggaagcctgggttaggcagccacgttccctgaaattagggcaaagtccttgtttccaggagtctgtccagaagagagcaagccagggctggcctcccttgacaccggggtgctcttagtcccctgtgcccctggacttttgtgcaccattcccatcttctctgttaggatcccgaggagctcagagttggacctgtccggtctgcccctcctcaatccacggatggaaaggagtatttgctgggtgtttgggaccattcacaagtccccctgggaactccttgctcatcctgaagctcctactatgtagggaagcttctgaaatgaggctgggcagagacccctgggtacccatcatgctggcaagaggaagagaagatagggagagaagcctgaggcgagctgggctcagggggactgtctattcctggagacagctcacagataaagcccaggacagtcaaccaacaactcgcacaagtccccaaatcctagaaacctctgtcttgtgttctgttccttttccttccgttcagctggcccttagggtaccaagccagactgggagacaggaatgcagcatctgagtcaaagcatggcctgctcagcacatcctcctgctcccaacccagacaagaggcccaggagccctcagctcacaccatacctggggtgagcatagcatcacacagcctgcctggggagccactgcttctccccctacaggagctgggaggagagtactggatgttttgttcatgaaaagctgtgttccatgccctacacatctatctgggtgctgggtcccgccacccacctctatctactgctgtcctatatgtttgccctgtgggccacggatgtggcgatcaggttaattacatctgtgtaataaagtaagcatttgctacgacgaaaaaaaaa

SEQ ID NO: 11 - DNA, 7441 bp, Homo sapiens
agagccgaggagccctggggtccctcaaagtttgtgtctggagccgtagcggcaagtgggcttgcggctaagggattttcctgggatgagagcgggtcttctgccttcattttggatgcacatcccgctttagccccggcagcctttggtccggctcgtgtccctggggattctcggatctccgaggacaccggacgggagcgcttggccatcctctctccggcagaggagcagacgtttgctttccaagtgcaaaactacagacacgcgcgcgcacacacgcaagcacacgcggagagagaggaaccttgccggtccgaggcagctctgcgcgtcccctcctgcgcttagcatcctcggcccagcgcggcccgcaccgccatggaggtgctggagagcggggagcagggcgtgctgcagtgggaccgcaagctgagcgagctgtcagagcccggggacggcgaggccctcatgtaccacacgcacttctcagaacttctggatgagttttcccagaacgtcttgggtcagctcctgaatgatcctttcctctcagagaagagtgtgtcaatggaggtggaaccttccccgacgtccccggcgcctctcatccaggctgagcacagctactccctgtgcgaggagcctcgggcccagtcgcccttcacccacattaccaccagtgacagcttcaatgacgatgaggtggaaagtgagaaatggtacctgtctacagacttcccttcaacatccatcaagacagagccagttacagacgaaccacccccaggactcgttccgtctgtcactctgaccatcacagccatctccaccccgttggaaaaggaggaacctcctctggaaatgaacactggggttgattcctcgtgccagaccattattcctaaaattaagctggagcctcatgaagtggatcagtttctaaacttctctcctaaagaagccccagtggaccacctgcatttgccgcccacccctccgagcagtcacggcagtgactcagagggcagcctgagtcccaacccacgcctgcaccccttcagcctgcctcagacccacagcccctccagagctgcaccccgggccccctccgccctctccagctcccctctcctcacggctcctcataaactgcagggatcaggccctctggtcctgacagaggaggagaagaggaccctgatcgctgagggctatcccatccccaccaaattgcccctgtcaaaatcagaggagaaggccctgaagaaaattcggaggaagatcaagaataagatttctgctcaggaaagtaggagaaagaagaaagaatacatggacagcctggagaaaaaagtggagtcttgttcaactgagaacttggagcttcggaagaaggtagaggttctagagaacactaataggactctccttcagcaactccagaagcttcagactttggtgatgggcaaggtttctcgaacctgcaagttagctggcacgcagactggcacctgcctcatggttgtggtgctgtgctttgccgttgcattcggcagcttctttcaaggctacgggccctatccttctgccaccaagatggctctgcccagccagcattccctgcaggagccctacacagcctccgtggtgagatccagaaacctgctgatctacgaggaacattctcccccagaggagtcatccagcccgggctcggctggggagctggggggctgggatagaggttcctccctgctcagggtgtcagggctggagtccaggccggatgtggatcttccccatttcattatctcgaatgagaccagcctggagaagtcagtgcttttggagctgcagcagcacctggtcagcgccaaactggaggggaatgaaacactaaaagttgtagaactcgacagaagagtgaacaccactttctaaagaggctgcctgcaccccctccctttcccttaactctacttttacatccccaaaccacctttgtcatcagcttttcctctttgccactggatcttcatggagacatgggcaagcattagtggcttcagattggagaccagcctgggacttccctgcagtgagagagcatctccccctggtccatgcccctcctgtgcagaagggagcctgcatccctcccttcctttctcttactgccataggaaattattttaggggttggaggtgggacaagcaggcttgtttccaccaatagtgccaaaaagatattgcctaatgtgcacctgtgaggtgtaaccccccgctttggagacgagatggctcttgttcagtcaagaccccagactctggccacaaaaatgccataatgcctgttggtatttggcaaagcactgacccgtgtcctccgttgctcgcactggggtctctggtgtgaacacccccgacagcagccctccgcccactctgccccctgggagccctcgctggatcgtctcgtctcctgcagcagcactggcaggcgagggctctcgttcatattctcaggccgcaagtgcaatgcctgaggggatcaggcttttctactccaggcaaacctgccccatcttgtcgcttttaggacctcccacaacctggttccccacacatccatagttctgcctccccagcttctcctccccagttgtaaatagtatttattagcttgccgaggcttcctgctagcaaccacactgaagagatcgatgcctcctttcaagctagccaagttttctgcgagccttcagagctaggagggcaccctaggctctgggatcccgtgtctttccagacaatgttttgtttcctttcctttgttttttcttttaactggaataattaccattgaaaaagaagttcctttgagcatgtatgtgtctgcctctaggatgagctcagagcgagagatgacacaatgcctcactcaggccccgggctccctggccacaagctttttctatcctgttttcatgacagagaaggggaagccctgttctgacaacagacatttcagacaaccttgctggctttccacacctgcctggccccctcctccctccacacttccactttgtcctcctcgtcccctacctcaacaaagcagggtggggtaggtgacatttgtgtatccacattcttacctttggtagtcaggtttggctactttgcagctcgcccaaagagatacaacctaatccccaacctacttttagtttttttgttttttttttatggttaaaagtaacttttgtagtttaaaaaaatctttcctctttcatataaataagaagtggaaattgcctttttattgtgtaatgtagaaaaccctcaagtgttttttccgagcttgggaaagattttgtgtaggaaatgtgcatagagtttgtattttatttttattagcagctgaaatgcctttggttttggcttctctctctccctctctctctctgtctctccttctctctctccccccaccacccacccccacacacgtcatctgcattgttattggagcctgtacttagagggattaagcccacaccctggcttccattccatatcaggtacaggatttgatgttattaacatttgtcgtcatacctcataagtcggtccctgccttgtctgtctaggcccatttggggctccctgtgagtgattcccctctctctgctatgctggagacggttccagcctggaaagcggccaagttcatcttctcactgtgagtggaagctggatcgggcccccgtagtcctggcagccctgttgtctggagggttcttgttgtccctcccattagccagggcggagactgtctgagctgtgcaggaggagggttgctagtaggttctgcttctgcttctctctgctccactgtctgcagcccagatcctgttgggcctggctggtgtctggtaaccatgggcctccactgacccatccctctcttttaaactgtcaggtcattatcaggcataggcagcctatagggcccaaagaaggcaaaaagataagatttactcaagtagcatttgggcaatgaggaaggaaaggtttcaaatttaggggcagaagtgagagaatgagccaacccatgtacctgctgcaactgaaccagactgggttttcaaggctcccagacgtagagtaggaaacgtgctcttctaaatgaggagggagaagataaaggaaacttctagcccctgtccttagtgctttgaggattttattttctcccttactacgcttgcttgacgtcactctctctcgacctccaaacagcaggactctttctctgggaaaccatccttccaaaacggaatctatgtagacaatgggacgttaggcagagagctcagatggcccttttaagggggctccaagaaccaacatcactgctcttttagataaacctctgccctccactccttgcttgagtgggttaaaggaactaacagttgtccctttaggaggacaaaatggggtcaagaggacacagaagagttgtatagcaccagattggttccaaatagttaatggatgtgtgcacattttctgttcagggattaagaccagaatatcagtggatttgttttccccaccaagtggcctcttagactagtcattaacttatgattagctctaaagatttcaaatagtggcagacagtgtcttctgaatgtaagttttgagaaatacgagtctgtcagagcggccataagccataaagagtcaatctcttaattatatttttcatcatgtaaacaagtttcccatttccctttcttagattgcaccagtgaaggagatgttttgcaaagattcagagaactaatttttcactggataagacctgagtaacccagaccccccaccgtggttcttttcacagccctcgactttgcacttaaaaagggatattgtaaatgaaaggctgcagtgccagttttaagaaagaatttctgtgaagtgtgaggactctggagtctagctcacataaagagagtgttatataaaaatccgacagctgaactaggttgctcttttttggcagggagtggggatgagatttgacaccaatatgggcaaaattagataaccttttggttaatataaatgattttgatttggaggcctaatttgtagattgtgaaagcagcttttagtttaacttattcacagaccccttataattaccatgtttttttttttcttcctaaatctcttggttcagcttgtgaatcttacgtgcccgtaaagttgggatgttgaattggctcttctttgttctggcagtgagtcaagtgtccagcattttttcataagtgttttttaaaattgttctccagcattttatggctcctccctcccatgtcctcagacccagcaaaagcgtagaggcagaattagaggcctctccaggccagctcctctgcccacatgtcatacaaggtgtgaatttgagcacagtccagaaatggagacatcccacccccagttgaataatggcccattcatgccaaccttgccaacacggagagggcagagatgcactagaagaccttcatcctccccttcctctgccccaagtcactacagttggttctattgaagccagtctttaagaaacctgggttaaagacaccagcacttctgcttgctgggctggctggacctgtgaagccatgggcaggtagtgccctcttgagagtcattttatttggccaccttcaggtgagactatccatagacacatgctaggataggccccgctgggagggcagttacaggagagagtaggtggtggtgacgtgagggctgtgaaggatccagagacaagacttagatgtttcgttcattcactcactcattcagttactcctaagacttttcagtttcataaggaagagtgttgcctgaggccctagggaatattggggaatagaagggattgaggaaacattaataatagttattcaaaagacccaaatgcttatacttctctctcccttcttctctctctgacacacacacacacacacacacacacacacacacacacgtgcacattcctcccttacatgctcatttgtgccttaaatgtgccttataggtaaatccaggatgactgaggaatccctcgtcactgggagattttgtatatattcttttattattagattgagttgggtgtggggaaaaatttttttctgaaggctcaaaagtggtttcctaaaagtgagccactatcagatttgcacatcaggagaaaagaaatagggttacgtccattaggaaaatcccagtttgcaggagtgcaatcacatcaaaaaaacaaccagccaggattaaaggtattataaatcctcatagcggaacatttctcagggcaaaggaacctggctcatttgaagattaatgttccatgcctttgtggtcaaagggtcagcacttaacacaggaaaaaactaggtgttgttttgttttgttattttggacaacataaaattcaggaatgttttatttagccttggtttctagaaggaagggaaataatatttcttgagcatttactagggtgttgcgtgctgtgctaagtaaattttaagtctttcagttttatagatacggaaaacaagggtgactctttaccacaggatgaataaagaactaagtaatatgggaaatgcagcaatttctggactagctgagccgattccttcctgtgagcacactgtaagctttcaagttctctgggcaggaattacagcacctgtcccctgcaatggccctgctgtgtgatgctcatcgcttcccttcgtgctggagcagtcccccaggtgtccatctcctatctttttgttccaatcttctgtgagttccagctagcaggctttacatctggggaaaggaaaaccaggggttttagctctgttctctgctcccatccttcgctcaccagctgagtgagaacatgaactttttgcaccatgtacccatggcttacactacttagaaaatcaccttttcagataaaacagtttatgagttcatagagaacaccagcactctttgacaaaactgtgagtgaccctttttaaacaatgctgagcaggccctgagctataatcaacggtgagctttaatgtctatgctgacagttaggttttgctctcttttgtaacaggttacgtagaccagcagtgtttaaatctaaatacgttgtgagtctgttatctgtcctatcgcgttttttaaatgactttttattctttatcatagctaagtaaataccaaaaaaaaaaaaaagctttgtaggacacttgtacttagtttgggaaaaaaaaataaattgaaattgttatgcttttgtatttccatttcttgcaaataaatattttttcttaaatagtaa

SEQ ID NO: 12 - DNA, 3318 bp, Mus musculus
gggagtctcaaagtttgtctggagccctagtggggagtgggcttgttgcgcgaggactttcttgggatgagcgctgcctttttgccttccttttggatgcacagcccgatttaacccctgcaccttccgcccgatcccagcaggcttgtcctccccggggagtcacagatttccgaggacaagggtcgcgtagccttcggcagggctctcccgagttcctgctccagtgcataagttccacgcgcgcacacgccaagtacacggggagaagcgtctcaccggcccgcggcggctctgcgcggtcccctcctgcctcagcatcctcggccctgcgcggcgcccaccgccatggaggtgctggagagcggggagcagagcgtcctgcagtgggaccgcaagctgagcgagctgtcagagcccggagagactgaggccctcatgtaccacacgcacttctcggagctcctagacgagttttcccagaacgtcctgggtcagctcctgagtgaccctttcctctcagagaagagcgagtcaatggaggtggagccatctccaacatcaccagcgcctctcatccaggctgaacacagctactctctgagcgaggagccccggactcagtcaccatttacccatgcggctaccagcgacagcttcaatgacgaggaggtggagagtgaaaaatggtacctgtctacagagtttccttcagctaccatcaagacagagccaatcacagaggagcagcccccgggacttgtcccttctgtcactctgaccatcacagccatttccactccttttgaaaaagaagagtcccctctggatatgaatgctgggggggactcctcatgccagacgcttattcctaagattaagctggagccgcacgaagtggatcagttcttaaacttctccccgaaagaagcctccgtggatcaactgcacttaccaccaacaccacccagtagtcacagcagtgactctgagggcagcttgagccccaacccacgcctgcatcccttcagcctgtctcaggcccacagccctgccagagccatgccccggggcccctctgccttgtccacatctcctctcctcacagctccacataagctgcagggatcgggccccctggtcctgacagaagaggagaagaggaccctggttgccgagggctatcccattcccaccaagctgcctctgacaaaatctgaggagaaggccctgaagaaaatccggagaaagatcaagaataagatttctgcccaagaaagcaggagaaagaagaaagaatacatggacagcctggagaaaaaagtggagtcttgttcaactgagaacttggagcttcggaagaaggtggaggtgctggagaacaccaataggactctccttcagcaacttcagaagcttcagactttggtgatggggaaggtctctcgaacctgcaagttagctggcacacagactggcacctgcctcatggtcgttgtgctttgctttgctgttgcatttggaagcttctttcaaggctatgggccttatccttctgccaccaagatggctctgcccagccagcatcctctgtcagagccatacacagcctccgtggtgagatccaggaacctgctaatctatgaggaacacgctcccctggaagagtcgtcgagcccagcctcagccggggagctggggggctgggacagaggctcctctctgctcagggcatcgtcggggcttgaggccctgccagaggtggatcttccccatttccttatctccaatgagacgagcttggagaagtcagtactgttggagcttcagcagcacctggtcagcagcaaactggaagggaacgaaacactcaaggttgtagagctggagaggagagtgaacgccaccttctgaggagagctccaccctcctcttctcctaactccatctgatcgtcctttcagtttccccttcaccactggatctcgaggaggagatggcctagtgttacggctcgagacaggaggccagcccagggggttctgcttatgtgtccccgtggctctccacaaaagggagctagcacctctccatccctttctcttactgccattggaaattattttagggctgagataggggtggaacgagcaggcttgtttccaccaatagtgccaagaagacactgcctgattcttccccgggaggagtgactcctctgaagaagacatgactcatgttcagttgagaccccagactctagccacacacatgccacagacatgccagggagtggcaaagcactgactcctgagctcccttcctcactaggactccagtgtgaccctgcactgagaggaccaaagcgtcattgcagtcttctctccaccctgtaccccggagtcctgattggatgtctgcagaggcagatggggctcccaccatattttcaggccgcaagtgcaattcctgaaggcatcaggctcttctctcccaggctctcctgcccactgtgttgtttgtaggacacccccacacccactcatacacagcctgcatctccacaggacaatagctctgtctccctggcctcccctccccatttgtaaatagtatttattagcttgctcaagctcccagctggccatagtgaaaagatttcccctttcaaccagcaaagtcttctgttggcctttggaacaggagagtccccggaatctaggaccctagtctttgtacttgatgccttgtttccccccttttcttctttaaaattggggacctataacatcatcgctgttgcggaatccacttaggcatgtgtcccctgatggatgaatacatgggaatggtggatactgtcttctgactcaggctctaggctccatggcttcctctctctggtcctgccacacagaaggaaagccctgtccaggataatgagcgttgctgacacccttgctagcttgtcctgcctacctgcttaccccactccctcaccttcctccttcccttctgccctccatccacctgccttaactaattggggctggagttggtcatttttgtacacccacagtggtaccttttacagtcaggtttggatactttgcagctcatccaaagagacataactaaaccctaaactctttttttgttgttgttgttgttgttttttttttttatgattaaaaagtaaaaattgtagtttaaaaaatcctttcctctttcatacaaataagaaatggaaattgctcgtttattgtataagatagaagattcgtttaaagtgtttccccacccccacccccagcttgt

SEQ ID NO: 13 - DNA, 5139 bp, Homo sapiens
agttggaaagagaccacagactttgagggaagctcactcaggatctgctctccggcaaagtagtaagtgaggtgctgagagcagaatgagctactttgtggattctgctgggagcagccccgtcccttactcagcgcctcgtcctgcagtggtgaggcaaggacctagcaacacttatgaagatcctcgaatgaactgtggtttccagtccaattatcaccagcaaagaccttgctaccccttttgggatgagatggcaactcaggaagttcctactggtcttgaacactgtgtctcagatatggaatgtgcagatgtcccactattaactccaagcagcaaagaaatgatgtctcaagcattaaaagctactttcagtggtttcactaaagaacagcaacgactggggatcccaaaagacccccggcagtggacagaaacccatgttcgggactgggtgatgtgggctgtgaatgaattcagcctgaaaggtgtagacttccagaagttctgtatgaatggagcagccctctgcgccctgggtaaagactgctttctcgagctggccccagactttgttggggacatcttatgggaacatctagagatcctgcagaaagaggatgtgaaaccatatcaagttaatggagtcaacccagcctatccagaatcccgctatacctcggattacttcattagctatggtattgagcatgcccagtgtgttccaccatcggagttctcagagcccagcttcatcacagagtcctatcagacgctccatcccatcagctcggaagagctcctctccctcaagtatgagaatgactacccctcggtcattctccgagaccctctccagacagacaccttgcagaatgactactttgctatcaaacaagaagtcgtcaccccagacaacatgtgcatggggaggaccagtcgtggtaaactcgggggccaggactcttttgaaagcatagagagctacgatagttgtgatcgcctcacccagtcctggagcagccagtcatctttcaacagcctgcagcgtgttccctcctatgacagcttcgactcagaggactatccggctgccctgcccaaccacaagcccaagggcaccttcaaggactatgtgcgggaccgtgctgacctcaataaggacaagcctgtcattcctgctgctgccctagctggctacacaggcagtggaccaatccagctatggcagtttcttctggaattactcactgataaatcctgtcagtcttttatcagctggacaggagatggctgggaattcaaactttctgacccagatgaggtggccaggagatggggaaagaggaaaaacaaacctaagatgaattatgagaaactgagccgtggcctacgctactattacgacaaaaacatcatccacaagacagcggggaaacgctacgtgtaccgctttgtgtgtgacctgcagagcctgctggggtacacccctgaggagctgcacgccatgctggacgtcaagccagatgccgacgagtgatggcactgaaggggctggggaaaccctgctgagaccttccaaggacagccgtgttggttggactctgaattttgaattgttattctattttttattttccagaactcattttttaccttcaggggtgggagctaagtcagttgcagctgtaatcaattgtgcgcagttgggaaaggaaagccaggacttgtggggtgggtgggaccagaaattcttgagcaaattttcaggagagggagaagggccttctcagaagcttgaaggctctggcttaacagagaaagagactaatgtgtccaatcatttttaaaaatcatccatgaaaaagtgtcttgagttgtggacccattagcaagtgacattgtcacatcagaactcatgaaactgatgtaaggcaattaatttgcttctgtttttaggtctgggagggcaaaaaagaggtgggtgggatgaaacatgttttggggggggatgcactgaaaatctgagaactatttacctatcactctagttttgaagcaaagatggacttcagtggggaggggccaaaaccgttgttgtgttaaaatttattttattaaattttgtgccagtattttttttcttaaaaatcgtcttaagctctaaggtggtctcagtattgcaatatcatgtaagtttgtttttatttgccggctgaggattctgtcacaatgaaagaaaactgtttatatagaccccattggaaaagcaaaacgctctcactgagatcagggatcccaaattcatgggacttatataagaaggacaattaatgctgatttgggtacaggggaattatgtgtgtgaatgtcatctacaattaaaaaaaattagcacatccctttacttacttgttatcagtggattctcggggtttggacttaatgttgagctaagaagcattaagtctttgaactgaatgtattttgcatccctggttttggacgacagtaaacgtaggagcactgttgaagtcctggaagggagatcgaaggaggaagattgacttggttctttcttagtcctatatctgtagcatagatgacttggaataaaagctgtatgcatgggcattacccctcaggtcctaagaaataagtcctgaatgcatgtcgttccaaactaacactctgtaatttttcttttatgtcttattttccaagagtcctccattttttgcaccccctcaccgccaactctgttattcagtagagagaagtgtacggctttctgattggtgagtgaaaaagtaacttgagacacgacctaagttgaagagtttagacttgctgagttttagaagtgatggaaattaagagagcatttcaataaaatgtgacttggctgtctttggaagagaagtgcaaggctttcctttgaagaatttaaattagtccggtaggatgtcaggtgagactgtgtatgcaaaatgaatggcacaggtgatgccagggcctcttgcttgggtctgatgtcttggcacagggtaagtgaaggttaattccagaagagaggaatgacttgaaggcaaaggaaactaaggaaggaggttcagtgaggaaaataaggttgtccatgagatttgaatagatttttagttcccccaaggtttaaatacaaacatagtcaagcaaggtagtcatctttctgctggttgtgagggggaatctgaaaatggagttttagaggaaaagtcaacatctaactagtgaggaaaagtgcctaatacaattagaatctccctcactctatagttgcccagttgaaaggataaggaggaggggtggcttttatggacttccatgagagaaggaaagaaatatttcaggtaagcttctcagggctggccctttttgggatttggatgagaaattggaagtactaactactttctagcatatctttaagaaaattgattgttatttactcccagatcctcttgcagacccagaattatcaggaacatagctctgtgattcatgagtgtccccatactgatgaattggagcatccatatggaaagcaaaggcagaattatcccagctgtattattttgatcttttggatgcaggtgccttaatgaagctctcaaaatattttaggagctgctcagggagtgttgggtggaactgtttggactacattgttttctcttagattatgtgatttttgttgggcactggcaaaaggtgtgtgtgtgaatgtgtgcatgtgtgtgaatgttgtgtgtgtgtgtgtgtgtgtgtgtgtgtgtgtgtgtgtgtgtgtttgcagacatgcaaaactgcagctgaaataataccttagatttctaggtaagtctttccacatttcaataatgggtaagagtagaaccagggccgggtatcaattattgcttgctgtttgcaaccaggcataaaatcactttctcaaatcatccaccgttcctattaaatttatgccggaaactctccttctgtgagtataactcctgcagttcctatagcagataagatataagaaagtgcctcctagtgctcctccgcccgcttgtttgctaaaattccctttctctctaagtccaccattttcaagatttgtagatagtgtattagttaagacagctttgtcgatctggccagatgttttttctcctttgtccaaaggccagagaccatcccaggaagagtggtgggtggtttatacactggaaatgttgcgtttatgctttttaaaaacacacgttaacttcagaggaaggatgggcaaatctggtctagctgggtgaaacccttattttcccagagatgccttaacctttgttggttttggctttagggttcagagtcacttttgttcccttctccattctggagagggacttcccctacatagagccctgatttttgtggctgtggggattggaggtagcattcaaagatcagatgtgcttttcctcactttggagatgaacactctgggttttacagcattaacctgcctaaccttcatggtgagaaatacaccatctctcttctagtcatgctgtgcatgccgcttactctgttggggtctatataaatttgttgaactcttacctacattccaaagaagtttcaaggaaccataaatatatgtatacatatacatatataaaatatatatattaaaataaaattatcaggaatactgcctcagttattgaacttttttttttaagaatactttttttttaagctgagaagtatagggatgaaaaagatgttatattgtgtttgactattttccaacttgtattttcatataatttatattttttaaaagctgaaaatttagaagcaagatgaaaaaaaggaaaagcaggtgctttttaaaaatcagaactgaggtagcttagagatgtagcgatgtaagtgtcgatgtttttttaaaaaaaaatgcaaaaaaattcttatggcggagttttttgtttgtttattttagtagctgatgctggcacatcattttgctggagagttttttatatactgtagcctgatttcatattgtattttaaactgtgtgaaattaaaaacaaagaatttcattcataa

SEQ ID NO: 14 - DNA, 5065 bp, Mus musculus
taaagcggaggcgagctgcactgcgccagcgccggccagccgtgcgagcgagcaagggagcgagcgccccggacggaggagggagcgagcgcccgggacagaggagcgagcgggcgggcgaggcgcgggcgaggctgggacctaagcgcgctctctgctccgccaagtgccaacttcgcgcggactggctgggcgcgcaccttcccgcggggcggtcagcgggaatttgagattttagggttgcgctctgtccccttccactgggttcaaaaatccccattaaaaagcaaaacaacagttacagcaaacttactctcatcccgccggccccctcaactccgggcaccatgaaggcggccgtcgatctcaagccgactctcaccatcatcaagacagaaaaagtggatctcgagcttttcccttccccggacatggaatgtgcagatgtcccgctgttaactccgagcagcaaagaaatgatgtcccaagccttgaaagctactttcagtggtttcacaaaagaacagcagcgactgggaatccccaaagacccccggcagtggacagaaacccacgtccgggactgggtgatgtgggctgtgaatgagttcagcctgaaaggtgtggacttccagaagttctgtatgagtggagcagcactgtgtgccctgggtaaagaatgcttcctcgagctggctccagactttgttggggatatcctgtgggagcatctagagatcctgcagaaagaggatgtgaaaccatatcaggttaatggagccaaccctacctacccagaatcctgttacacctcggattacttcatcagctacggtatcgagcatgctcagtgtgttcctccctcagagttctcagagcccagcttcatcacagagtcctatcagacgctgcatcctatcagctcggaagaactcctgtccctcaagtatgagaacgactacccttctgtcattctccaggaccctctccagacagacaccttgcagacagactactttgccatcaagcaagaggtgttaactccagacaacatgtgcctggggagagccagtcgtggtaaactcgggggccaggactcttttgagagcgtagagagctacgatagttgtgaccgcctcacccagtcctggagcagccagtcatccttcaacagcctgcagcgggtcccctcctatgacagcttcgactacgaggattatcccgctgccctgcccaaccacaagcccaagggcaccttcaaggactatgtgcgtgaccgtgctgacctcaacaaggacaagcctgtcattcctgctgctgccctggctggctacacaggaagtgggccgatccagctgtggcagtttcttctggaattactcactgataagtcttgtcagtcctttatcagctggacaggagatggctgggaattcaagctttctgacccagatgaggtggccaggagatggggaaagaggaaaaacaaacctaagatgaattatgagaaactgagccgtggccttcgctactattatgacaaaaatatcatccacaagacggcgggcaagcgctacgtataccgctttgtgtgcgacctgcagagcctgctgggatacacccctgaagagctgcacgccatgctggatgtaaagccggatgctgactagtcatggacagacgcgcagaaggaaggggctgggggaaccctgctgagacctttcaaagagcaaccctgttggttggactcttcatttttaattgttattcaatgttttattttccagaactcatttttcacattcaggggtgggagctgagggagcgccgctgtattccattggccattggtgggccggaaagaggaggtcaggacctgcggggtgggcggggcaatagctcctgagcagactttcagaagggagaggtcttctcagaagcctgttggacctggcttgcagaggaaaaaaaaacttaagtgtccgttttttttttttttttttttaaatcaaagaaaaaaaaattcatcttgagttgtggatctactagtgggaggaatgatcacattgagatgaaagagattgatgaaagccagtcagttttgtgggtgggctgaaaacagtttcttcaggggattactaaactcaagaattattaacctttctactttttgaaacaatgatggactttgattgaaggggtccaaaactgtttttatgttgaagtttattttattaaattttgtgccagtatttttttttccttaaaaaaatatcgtcttaagctctaaggtggtctcagtattgcagtattgtgagtttgttgttatttgctggctgaggactcttgtcacagtgaaagacaactgtttatatagaccccattggaaaaatcccagttctgtactgagatcagagaccccaaactcttacagctaaaggaaacgaatgctgatctggggacagggggactgtgcgtgtgacttccacctgcggtttgtaaaaactgttttcctttgattcactgtcaggctttggacttagtgttagttacggggcgttaagtctctgcactgaatgtattctgcagctcttctatggaatcgtcggcatcatagcacagttcaagtcttggaaacgaaatccaaggagcaggattaacctgggttttggttaaaccgctacccgaaacatggaagactcagaataaaagccgtttgcatgggcattaccccgagggtcttaaggcctcaatgcatgccattccaaactaacattctcagttctcctctactttgtcttatgttccccccggcatccctcattgctgcccctccctcatcactgatcagtgaaagtggagtgtaatgcatcctgacacgtgactgaaagtgtagtttcagccatcacaacacaaatccaagagtccagacttgctagggctcggaggccacggcagtggagagggtatttcagtagcatgtgactcggcctcctttgaagacttggtgctcgtcaggtaggatttcaggtgagactgcttgccgggaagtggcagggagcactggcacttgacctgttaccacctcagcagagggcaagcccaggtcaactgcggtgagagggagacggagaaaatacaagggaaggcgcttaggtaaggaaaataaggtggcccgagacagttagatagatgtttagtttcctccaagtttaaactcagacttaattgaggcagccatcatcctgcctgatgggagaggagacctgaaaataagccattgtgggttaaaaaaaaaagtaaggagacatgcctgggatcattagaatcccttccactgtatagctgcctagttaagacagtgacagtgacatccctgagagaagagaggcaattggctgaggtgggcctctccaggctcacccttcggggtttgtagctccctggtatcatcttccaggagagttgatggctgactcccacattcccttgaagactctgaattactaagactataactcttgtccgtgggtgttctgtactgactgagtggagcatccccacaaggaagcaaaggccaaacattccagctatatattttgatcttacaaatgcaggtgccttaatgaagctctcaaaatatttaggagctgctcagggagtgttaggtgggatcatttggattatgctgttttctcttatgttatgtgatctttgttgggcactgacagtgtgagtgtgtgtgtgtgtgtgtgtgtacatttgtataaagttgcagctgaaatagtactgaattttctacttagagtctgttcacatttccataatggtgaatgaaagagtacagccggggctggtattgttgtttgctctttgcaacagggcttaaaaaaatcacttccccaagtcttcacccttctccatattgggttcctgctgggaatgcccctcctgagagcctaccacctgcagtcccctagcagagaagacataagaaagtgtctcccacactcctcagcgtgctgacttgcctgccttgatgccctttctccctaggtccaccacttttcaggatttgtagatagtatattagtcagacagctttgttgtccatctggccagatgctttcccccatctcctccgaaggccagagaccatcccaggaagagtggcgggtggtttatacactggaaatgtagcagcattgctgcattgatgctcttttaaaacacgttcacttcacaggaaggatgaacagatctgacctagctgggcgactccatcattttcccagagaaatgctttaacctgtgtggctggctttgggctcagaggcggggctaaggatgctccctgcggagagctctgatggtgtggccatggagaccttgtaagcattcaggatcagagatgctcttcctcactttggagaccagcactctgggttttaaagcattaaccttcatggtgaaatcacaccttctctctcctagccatgctgtgcatgcggccttctctgttggggtctatataaacctgttgaactcttacgtacattccaaagacgtttcaaggaaccacaagtatatgtatacaaatacatatatgaagtatatatgttaaagtgaatttatctctatcaggaatactgcctcagttattgaattttttttaaggatacttttttttttaaagctgagaattattgaggtgaaaaagatgttatattgtgtttgactttttccaacttgtattttcatataatttatattttttaaatgctgaaaatttaaaagcaagatttaaaaaggaaaagcaggtgctttttaaaaatcagaactgaggtagcttagagatgtagcgatgtaagtgtcttaaatgttttttgttttttttttaaaaacaaatgcaaaaaattcttatgggggagtttttggtttgtttcatttaagtagctgatgctggcacatcattttgctggaaaattttttatatactgtaggctgatttcatattgtattttaaactgtgtgaaattaaagaaacaaagaaattcattcataa

SEQ ID NO: 15 - DNA, 5322 bp, Homo sapiens
gttgttgttggtggcggcggcgagcggagccggaggagccgccgcaaagatggaggagccgtcgaggaggtgctgccgccgctgccgccgccgctgctgccgccgccgcccgcgaagccggagctcgagccgcagcggggatgccgttctgagtgcctgactgcctcgccccgaaggatggcctcggatgggcattagaggcacggcggccccgggctcccgtcccgtccgtctgtctgttatcgtctgtctctcttgacatcaccgcagctccaccccctcccgtcccagcccccaacgccagcttcctgcaggcccagagccggcatgaactctcccaacgagtcggcagatgggatgtcaggtcgggaaccatccttggaaatcctgccgcggacttctctgcacagcatccctgtgacagtggaggtgaagccggtgctgccaagagccatgcccagttccatggggggtgggggtggaggcagccccagccctgtggagctacggggggctctggtgggctctgtggaccccacactgcgggagcagcaactgcagcaggagctcctggcgctcaagcagcagcagcagctgcagaagcagctcctgttcgctgagttccagaaacagcatgaccacctgacaaggcagcatgaggtccagctgcagaagcacctcaagcagcagcaggagatgctggcagccaagcagcagcaggagatgctggcagccaagcggcagcaggagctggagcagcagcggcagcgggagcagcagcggcaggaagagctggagaagcagcggctggagcagcagctgctcatcctgcggaacaaggagaagagcaaagagagtgccattgccagcactgaggtaaagctgaggctccaggaattcctcttgtcgaagtcaaaggagcccacaccaggcggcctcaaccattccctcccacagcaccccaaatgctggggagcccaccatgcttctttggaccagagttcccctccccagagcggcccccctgggacgcctccctcctacaaactgcctttgcctgggccctacgacagtcgagacgacttccccctccgcaaaacagcctctgaacccaacttgaaagtgcgttcaaggctaaaacagaaggtggctgagcggagaagcagtcccctcctgcgtcgcaaggatgggactgttattagcacctttaagaagagagctgttgagatcacaggtgccgggcctggggcgtcgtccgtgtgtaacagcgcacccggctccggccccagctctcccaacagctcccacagcaccatcgctgagaatggctttactggctcagtccccaacatccccactgagatgctccctcagcaccgagccctccctctggacagctcccccaaccagttcagcctctacacgtctccttctctgcccaacatctccctagggctgcaggccacggtcactgtcaccaactcacacctcactgcctccccgaagctgtcgacacagcaggaggccgagaggcaggccctccagtccctgcggcagggtggcacgctgaccggcaagttcatgagcacatcctctattcctggctgcctgctgggcgtggcactggagggcgacgggagcccccacgggcatgcctccctgctgcagcatgtgctgttgctggagcaggcccggcagcagagcaccctcattgctgtgccactccacgggcagtccccactagtgacgggtgaacgtgtggccaccagcatgcggacggtaggcaagctcccgcggcatcggcccctgagccgcactcagtcctcaccgctgccgcagagtccccaggccctgcagcagctggtcatgcaacaacagcaccagcagttcctggagaagcagaagcagcagcagctacagctgggcaagatcctcaccaagacaggggagctgcccaggcagcccaccacccaccctgaggagacagaggaggagctgacggagcagcaggaggtcttgctgggggagggagccctgaccatgccccgggagggctccacagagagtgagagcacacaggaagacctggaggaggaggacgaggaagacgatggggaggaggaggaggattgcatccaggttaaggacgaggagggcgagagtggtgctgaggaggggcccgacttggaggagcctggtgctggatacaaaaaactgttctcagatgcccagccgctgcagcctttgcaggtgtaccaggcgcccctcagcctggccactgtgccccaccaggccctgggccgtacccagtcctcccctgctgcccctgggggcatgaagagccccccagaccagcccgtcaagcacctcttcaccacaggtgtggtctacgacacgttcatgctaaagcaccagtgcatgtgcgggaacacacacgtgcaccctgagcatgctggccggatccagagcatctggtcccggctgcaggagacaggcctgcttagcaagtgcgagcggatccgaggtcgcaaagccacgctagatgagatccagacagtgcactctgaataccacaccctgctctatgggaccagtcccctcaaccggcagaagctagacagcaagaagttgctcggccccatcagccagaagatgtatgctgtgctgccttgtgggggcatcggggtggacagtgacaccgtgtggaatgagatgcactcctccagtgctgtgcgcatggcagtgggctgcctgctggagctggccttcaaggtggctgcaggagagctcaagaatggatttgccatcatccggcccccaggacaccacgccgaggaatccacagccatgggattctgcttcttcaactctgtagccatcaccgcaaaactcctacagcagaagttgaacgtgggcaaggtcctcatcgtggactgggacattcaccatggcaatggcacccagcaggcgttctacaatgacccctctgtgctctacatctctctgcatcgctatgacaacgggaacttctttccaggctctggggctcctgaagaggttggtggaggaccaggcgtggggtacaatgtgaacgtggcatggacaggaggtgtggacccccccattggagacgtggagtaccttacagccttcaggacagtggtgatgcccattgcccacgagttctcacctgatgtggtcctagtctccgccgggtttgatgctgttgaaggacatctgtctcctctgggtggctactctgtcaccgccagatgttttggccacttgaccaggcagctgatgaccctggcagggggccgggtggtgctggccctggagggaggccatgacttgaccgccatctgtgatgcctctgaggcttgtgtctcggctctgctcagtgtagagctgcagcccttggatgaggcagtcttgcagcaaaagcccaacatcaacgcagtggccacgctagagaaagtcatcgagatccagagcaaacactggagctgtgtgcagaagttcgccgctggtctgggccggtccctgcgagaggcccaagcaggtgagaccgaggaggccgagactgtgagcgccatggccttgctgtcggtgggggccgagcaggcccaggctgcggcagcccgggaacacagccccaggccggcagaggagcccatggagcaggagcctgccctgtgacgccccggcccccatccctctgggcttcaccattgtgattttgtttattttttctattaaaaacaaaaagtcacacattcaacaaggtgtgccgtgtgggtctctcagccttgcccctcctgctcctctacgctgcctcaggcccccagccctgtggcttccacctcagctctagaagcctgctccctctgcagggggtggtggtgtcttcccagccctgtcccatgtgtccctccccccattttcctgcattctgtctgtccttttcctccttggagcctgggccagctcaaggtgggcacgggggcccagacagtactctccagttctggggccccccgagtgaggagggaacgggaagtcggtgccttggtttcagctgatttggggggaaatgccttaatttcactctcctcccttctccagcctcaggggaggatctggaggatccactactgtctttaagatgcagagtggaggggaggtgggcacccaccctgcgattctccaccctttccccttctttcgtcctcaccatctctgcagacccctctcctcctccttcctcttggtctcagcactgatgggaggctggtgcccaagctgtggcctgcagtctgtgaggagggctgtcttgcctcacactcctcacagcctacttccccttccccggggctgagagggtgaaagtgtgtggggaaggagaggactggtttcctgggttctcaggggccaggaggagtaacagaaccaggtctgctccccaccttactcggatggcctccctgcccctctgctggcacagcctgggcaaggggagaaggtggtccctgcagaggggctccaggctggtgagagcccccctgctgtcaggaccagattttcccagccatccagcatgctgcggggagaaggggcagaggctcacctccctcctggggccttttgttttggatcctggggatggtgagaatggaggttctagaaggggtaaggccagaacccagggatccaggagtcggctctcagctggagcttccataccttctgggctccctttgctgaccaccagcccaagggagctaagaccaggagggggctgggcgctgtcccttctctttcccaggagccctgccaggggctgtgggcctacaaggcttccaggggatgccatccagcctgtaggaaaccaaagatgggaagtggctcctagggggctgactcttccttcctcctcctccccagtaccacatatactttctctccttctatctccagggccccaccaatctgtttacatatttattatcctatgggggcctgagcaggattgagggagccaggggaggggcaggagtcccagcaccatcggttcatagtgtgcttgtgtgtttgttttagatcctcctgggggatggggatggggccaggctcagtgtactaggcctctctgtgctgagccccaggctcccggccccttacccactctctccctgtggctggtctggttctcatgtaaacccactccttgctttgtctccctggatatggatttcagttaagtattttgtaacccgttacactgtgtgtccttgtgtaaataaacttgtttctggcagtgcc

SEQ ID NO: 16 - DNA, 4332 bp, Mus musculus
ggtcacgtgacgagagtgacgtctccgaatgttgttgttggtggcggcggcgagcggagccggaggagccgccgcaaagatggaggagccgtcgaggaggcgctgccgctgctgttgccgccgctgctgccgccgccgcccgcgaagccggagctcgagccgcagcggggatgccgttctgagtgcctgactgcctcgccccgccccgaaggatggccttggatgggcattagaggcactgcggccctgggctcgggctcctgtcccgtccgtctgtctgttatcgtctgtctctcttgacatcacagcagctccgccccctcccgtcccagcccccaacgtcagcttcctgcaggcccagagccggcatgaactctcccaacgagtcggcttccttccagagggattggaggctcaggactggctggtgaggagaggcagtatcaacccagagaggaagtagaagatgacaaaaaagggggaagagagaagatccaggctggctgcttggtgtccagtccgttgtttgcccccagcagtggccaaagctgaccccagatgcatgggtcctctgcctgcacccttctctagtccagccactggccacccagactttggtccacgctcctggagcggagagcaggcccttcagcctcattagcctccctgcgtctgctgcctccggagcgcgcccaggccggatgaggtgcctccttgcctcgtgggtcccagcggcagtggcctgagggagtccttgagcagccagctggccccgctgaccaccttttccctcaccggcttccgcctcgggcggcccctccctggcatgctgctggtgcccaaggcacaggggcttgtggagatgctgcagaccatctatgagaccgagtcctgtttctcagcagatggcatgtcaggccgggaaccatccttggaaatcctgccacggactcctctgcacagcatccctgtggcagtggaggtgaagccggtgctgccaggagccatgcccagctccatggggggtggaggtggaggtagccccagccccgtggagcttcggggggctctggcgggccccatggaccctgcgctacgggagcagcaactgcagcaggagctcctggtcctcaagcagcagcagcagctccagaagcagctcctgttcgccgagttccagaagcagcacgaccacttgacgcggcagcacgaggtccagctgcagaagcacctcaagcagcagcaggagatgctggcggctaagaggcagcaggagctggagcagcagcggcagcgggagcagcagcggcaggaggagctggagaaacagcggctggagcagcagctgctcatcctgcgcaacaaggagaagagcaaagagagtgccatcgccagcaccgaggtaaagctgaggctccaggaattcctgttgtccaagtcaaaggagcccacgccaggcggcctcaaccattccctcccacagcaccccaaatgctggggagcccaccacgcttctttggaccagagttcccctccccagagcggccctcctgggacgcctccctcctacaaattgcctttgcttgggccctatgacagccgtgatgactttcccctccgtaaaacggcctcggaacccaacttaaaagtacgttcgaggctaaaacagaaggtagccgagaggagaagcagtcccctcctgcgtcgaaaggatggcactgttattagtacttttaagaagagagcagttgagatcaccggcacggggcctggggtgtcgtccgtgtgtaacagtgcgcccggctctggccccagctctcccaacagttcccacagcaccatcgctgagaacggctttactggctcagtccccaacatccccactgagatgatcccccagcaccgggccctccctctggacagttccccaaaccagttcagcctctatacgtctccttctctgcccaacatctccctagggctgcaggccactgtcactgtcaccaactcgcacctcaccgcctccccgaagctgtcaacacagcaggaggctgagaggcaggcccttcagtccctgcggcagggcggcacactgaccggcaagttcatgagcacatcctccatccctggctgcctgttgggagtggcactggagggtgacacaagcccccacgggcacgcttccctgctgcagcacgttttgctcctggagcaggcccggcaacagagcacgctcatagcagtgccgctccatgggcagtccccactggtgacgggtgaacgtgtggccaccagcatgaggacggtgggtaagctcccgaggcaccgacctctgagccgcactcagtcctccccgctgccgcagagtccccaggccctgcagcagctggtcatgcagcagcagcaccagcagttcctggagaagcagaagcagcagcagatgcagctgggcaagatccttaccaaaactggggagctgtcaaggcagcccaccactcacccggaggagacagaagaggagctgacggagcagcaggaggccttgctgggagagggggccctgaccattccccgggaaggctctacagaaagtgagagcacccaggaagacctagaagaggaggaggaggaggaggaggaggaagaggaggactgcattcaggtcaaggatgaggatggcgagagtggtcctgatgaaggccctgacttagaagagtccagtgctggttacaaaaagttgttcgcagatgcccagcagttacagcccctccaggtgtaccaggcacccctcagcctggccactgtgcctcatcaggccctgggccgcacccagtcctcacctgctgctcctgggagcatgaagagccccacagaccaacccactgtggtgaagcacctcttcaccacaggtgtggtctatgacacgttcatgctgaagcaccagtgtatgtgcggaaacacacacgtgcacccagagcacgccggccgcatccagagcatctggtcccggctgcaggaaactggtctgctcggcaagtgtgagcggatccggggtcgtaaagccacactggatgaaatccagaccgtgcactctgagtaccacaccctgctctatgggaccagcccccttaaccggcagaagctggacagcaagaagctgcttggccccatcagccagaagatgtacgccatgctgccctgtgggggcattggggtggacagtgacacggtgtggaatgagatgcactcctcaagtgccgtgcgaatggcagtgggctgcctggtggagctggccttcaaggtggctgcaggagagctcaagaatggatttgctatcatccggcccccaggacaccatgctgaggagtccacagccatgggattctgcttcttcaactccgtagccatcacagctaaactcctgcagcagaagctgagcgtgggcaaggtcctcatcgtggactgggatattcaccatggcaacggcacccagcaagcattctacaacgatccctctgtgctctacatctccctgcatcgctacgacaacgggaacttctttccaggctctggggctcctgaagaggttggtggagggccaggtgtggggtacaacgtaaatgtggcgtggacaggaggtgtggatccccccattggagatgtggaatacctgacagccttcaggacagtggtgatgcccattgcccaggagttctcacctgacgtcgtcctagtctccgctgggtttgatgctgttgaaggacatctgtctccactgggtggctattctgtcaccgccagatgttttggccacttgaccaggcagctcatgacactggctgggggccgggtggtgctggccctggagggaggccatgacttgaccgccatctgtgatgcctctgaggcctgtgtctcggctctgctcagcgtggagctgcagcccttggatgaagcagtcttgcagcaaaagcccagcgtcaatgcggttgccacactagagaaagtcatcgagatccagagcaaacactggagctgtgtacagaggtttgccgctggtctgggctgctcgctgcgggaggctcagacaggtgagaaagaggaggccgagactgtgagcgccatggccctgctttccgtgggggctgagcaggcccaggctgttgccactcaagagcacagccccaggtaagccagcagaggagcccatggagcaggagcctgccctgtgacaccctggcccccatccctctgggcttcatcattgtgattttgtttattttttctattaaaaacaaaaagtcacacattcaaaaaaaaaaaaaaaaaaaa

SEQ ID NO: 17 - DNA, 1724 bp, Homo sapiens
agctctgcgaggggccggagcgcggcggagccatgcagtacccgcaccccgggccggcggcgggcgccgtgggggtgccgctgtacgcgcccacgccgctgctgcaacccgcacacccgacgcccttttacatcgaggacatcctgggccgcgggcccgccgcgcccacgcccgcccccacgctgccgtcccccaactcctccttcaccagcctcgtgtccccctaccggaccccggtgtacgagcccacgccgatccatccagccttctcgcaccactccgccgccgcgctggccgctgcctacggacccggcggcttcgggggccctctgtaccccttcccgcggacggtgaacgactacacgcacgccctgctccgccacgaccccctgggcaaacctctactctggagccccttcttgcagaggcctctgcataaaaggaaaggcggccaggtgagattctccaacgaccagaccatcgagctggagaagaaattcgagacgcagaaatatctctctccgcccgagaggaagcgtctggccaagatgctgcagctcagcgagagacaggtcaaaacctggtttcagaatcgacgcgctaaatggaggagactaaaacaggagaaccctcaaagcaataaaaaagaagaactggaaagtttggacagttcctgtgatcagaggcaagatttgcccagtgaacagaataaaggtgcttctttggatagctctcaatgttcgccctcccctgcctcccaggaagaccttgaatcagagatttcagaggattctgatcaggaagtggacattgagggcgataaaagctattttaatgctggatgatgaccactggcattggcatgttcagaaaactggatttaggaataatgttttgctacagaaaatcttcatagaagaactggaaggctatataagaaagggaatcaattctctggtattctggaaacctaaaaatatttggtgcactgctcaattaacaaacctacatggagaccttaattttgacttaacaaatagtttatgtactgctcttaggttgttttgataaagtgacattatagtgattaaattcttccccctttaaaaaaacagttagtggttttcactatttataaaaaattaattttgaactttttgttaaatttttaagttatagctttaaaggttttaataggaccttcttgaacgacttttctgtaatctgtttatctcccacttaatggaaaggcaaaggggtaccccaaatccagaggtgcctacatttcaggcagccttggagtattttaaaaggaaaacattctttacttttatatgacattcttatactgctgtctcaaatccaaaaacatttcagagctcttgtctcagagatgtgtgttctttttgtcagagatatggttgatgagaatcttaaatgcttgttttgcactatcacttagtacctgtttgaccaaggtgttaaggggatagtacctcccaattcaagcagagaaactgacctgactaaagttaatcgcagatgaactagaagtcacaggttaattaaatgtaagtagattgtagatactgttttatatcaaacaatgtttataatgtgtatatagaattgttcactgtaaaaaaaatggccaaaatgtgttttttttttaataagtaacttgactataaaataaagccgtccgtgggacgactgacctcgtt

SEQ ID NO: 18 - DNA, 1771 bp, Mus musculus
agctctctgcgaggggctgcggagcggccatgcagttcccgcacccggggcccgcggctgcgcccgccgtgggagtcccgctgtatgcgcccacgccgctgctgcagcccgctcacccgacgcccttctacatcgacgacatcttgggtcgcgggcccgccgcccccacgcccactcccacgctgccgtcccccaactcctccttcaccagcctcgtgtcctcctaccggaccccggtgtacgagcccacgccggtccaccccgccttctcgcaccacccggccgccgcgctggccgccgcctacggccccagtggcttcggaggccctctgtacccgttcccgcggacggtgaacgactacacgcacgccctactccgccacgaccccctgggcaagcccttgctctggagccccttcctccagcgacctctgcacaaaaggaaaggcggtcaagtgaggttctccaacgaccagaccgtcgagctggagaagaagttcgagactcagaaatacctctccccacccgagagaaagcgtctggccaagatgttacagctcagtgagagacaggtcaaaacctggtttcagaatcgccgagctaaatggagaagactgaaacaggagaatcctcaaagcaacaaaaaggatgcgttggacagtttggacacttcctgtgagcagggtcaagacttgcccagtgaacagaataaaggtgcctctttggatcgttcgcagtgttcaccctccccagcctctcaggaagaccccgactcggagatctcagaggattccgaccaggaggtggacatcgagggggataaaggctactttaatgctggatgacagtcatcggccatgtttagagaccggactttagaataatgttttgctacagaccaactggaaaattcggggggagagagagagagagaaagagagagagagagagagagagagagagagataacaattcttgtagagttttgaaaatgtttggtgcactggctaattaacaaacatgcattgcgttgagaccttaactttggtttaacatacggtatctataccagtttttaagttgttttgataaagtgactaaatgtgacctcattttttaaaaagtgaatttatttctatttatgagaggtaatttgaacttttgtctaaagcttaaattatgactttaaaggttttaagttttaggaggctgatcttgactgactttcctaagtctgtagctattccctcgactttagaggtgcacttaggtgggggcaacatttggggaatccatggaacactttgaaagggtatacccaacttttacatggcatcttacattgctgccttaactccaaagccatttcagagcacttggctcccggtgtctgttcttacaagcaagatagttgataagattctaaaaatcttgttttgctcagccagtgcctctgaccctggtgttaagggatgacgcagtccctacaggcagggaaactgactcatttgagactgatctcacacgaactagaaatagcaggacaattcaatgtaagtagattgtagatagtgtgttttatagaaactgtgtctataacatgtatatagcattacttcttgtaaaaactctgccaaaatgatgtttgtttgtttggttggttttttgtaatttaatgaattaactttcatatacattcatcatcagatgaacgaccctgtttgaattctcattctttggttaaaatacctaaacatgacacctaaagagc

SEQ ID NO: 19 - DNA, 1670 bp, Homo sapiens
ataaattgtgcaagggtgctatagacgcacaaacgaccgcgagccacaaatcaagcacacatatcaaaaaacaaatgagctcttattttgtaaactcattttgcggtcgctatccaaatggcccggactaccagttgcataattatggagatcatagttccgtgagcgagcaattcagggactcggcgagcatgcactccggcaggtacggctacggctacaatggcatggatctcagcgtcggccgctcgggctccggccactttggctccggagagcgcgcccgcagctacgctgccagcgccagcgcggcgcccgccgagcccaggtacagccagccggccacgtccacgcactctcctcagcccgatccgctgccctgctccgccgtggccccctcgcccggcagcgacagccaccacggcgggaaaaactccctaagcaactccagcggcgcctcggccgacgccggcagcacccacatcagcagcagagagggggttggcacggcgtccggagccgaggaggacgcccctgccagcagcgagcaggcgagtgcgcagagcgagccgagcccggcgccgcccgcccaaccccagatctacccctggatgcgcaagctgcacataagtcatgacaacataggcggcccggaaggcaaaagggcccggacggcctacacgcgctaccagaccctggagctggagaaggagttccacttcaaccgttacctgacccgcagaaggaggattgaaatagcacatgctctttgcctctccgagagacaaattaaaatctggttccaaaaccggagaatgaagtggaaaaaagataataagctgaaaagcatgagcatggccgcggcaggaggggccttccgtccctgagtatctgagcgtttaaagtactgagcagtattagcggatcccgcgtagtgtcagtactaaggtgactttctgaaactcccttgtgttccttctgtgaagaagccctgttctcgttgccctaattcatcttttaatcatgagcctgtttattgccattatagcgcctgtataagtagatctgctttctgttcatctctttgtcctgaatggctttgtcttgaaaaaaaatagatgttttaacttatttatatgaagcaagctgtgttacttgaagtaactataacaaaaaaagaaaagagaaaaaaaaacacacaaaaagtcccccttcaatctcgtttagtgccaatgttgtgtgttgcactcaagttgtttaactgtgcatgtgcgtggaagtgttcctgtctcaatagctccaagctgttaaagatatttttattcaaactacctatattccttgtgtaattaatgctgttgtagaggtgacttgatgagacacaacttgttcgacgtgtagtgactagtgactctgtgatgaaaactgtgactccaagcggtgtgtccctgcgtgcctttataggaccctttgcacgaactctggaagtggctcttataagcgcagcttcagtgatgtatgtttttgtgaacaaagttacaaatattgtccaagtctggctgttttaagcaaactgtgatcagcttttttttttttttttttttttttgtatttgtttttaaggaaaaaatactgactggaacaaaaaataaactttctattgtaagttc

SEQ ID NO: 20 - DNA, 1877 bp, Mus musculus
ctccacccaactcccccattagtgcacgagtttacctctagaggtcatcaggcaggatttacgactggacaacaaaagcacgtgattcgaagtcgtaccccatatttgggtgcctacgtaggagggaaccgagtacatgtcccagtcatttccataattcatcataaattgtgcaagggtgctatagacgcacaaacgaccgcgagccacaaatcaagcacacatatcaaaaaacaaatgagctcttattttgtaaactcattttgcggtcgctatccaaatggcccggactaccagttgcataattatggagatcatagttccgtgagcgaacaattcagggactcggcgagcatgcactccggcaggtacggctacggctacaatggcatggatctcagcgtcggccgttcgggttccggccactttggctccggcgagcgcgcccgcagctacgcggctggggccagtgcggcgcccgccgagcccaggtacagccagccggccacgtccacgcactcgccaccgcccgacccgctgccctgctcagcggtggccccctcgcccggcagcgacagccaccacggcgggaaaaactccctgggcaactccagcggcgcctcggccaacgccggcagcacccacatcagcagcagagagggggttggcacggcgtccgcagccgaggaggacgcccctgccagcagcgagcaggcgggcgcccagagcgagccgagcccggcgccgcccgctcagccccagatctacccctggatgcgcaagctgcacattagtcacgacaatataggtggcccagaaggcaaaagggcccggacggcctacactcgctaccagaccctggagctggagaaagaattccacttcaaccgctacctgacccgccgaagaaggatcgaaatagctcatgccctttgcctctccgagagacaaattaaaatctggttccaaaacaggaggatgaagtggaaaaaagataataagctgaaaagcatgagtatggccgcggcagggggggctttccgcccctgagcatctgagcggccaaagtactgagcagtagtagccgggcagctctctgtagtgtcagtactaaggtgactttctgaaactccccttgtgttccttctgtgaagaagccctgttctcgttgccctaattcatcttttaatcatgagcctgtttattgccattatagcgcctgtataagtagatctgcttctgttcatctctttgtcctgaatggctttgtcttgaaaaaaaaatagatgttttaacttatttatatgaagcaagctgtgttacttgaagtaactaaaacaaaaaaaaaaaaaaaagaaaagagaaaaaaaaactacacacacaaaaagcccccccacctcgtttagtgccaatgttgtgtgttgcacttgagttctttaatgtgcatgtacgtggaagtgttcctgtctcaatagctccaagctgttaaagatatttttattcaaactacctatattccttgtgtaattaatgctgttgtagaggtgacttgataagacacaaattaacttgttcaacgtgtagtggctagtggctctgtgacgaaaactgtgactccaagcggtgtgtccctgcgtgcctttgtaggaccctttgcacgaactctggaagtggctcttataagcgcagcttcagtgatgtatgtttttgtgaaaaagttacaaatattgtccaagtctggctgtttaagcaaactgtgatcagcttttttttttttttgtatttgtttttaaggaaaaaaaacactgactggaaacaaaacaaaataaactttctattgtaagttctcttggtctgatttatgccaaatagcaagc

SEQ ID NO: 21 - DNA, 950 bp, Homo sapiens
gcgttgcaggtcactgtagcgggacttcttttggttttctttctctttggggcacctctggactcactccccagcatgaaggcgctgagcccggtgcgcggctgctacgaggcggtgtgctgcctgtcggaacgcagtctggccatcgcccggggccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcgacctgcaggtagtcctggccgagccagcccctggaccccctgatggcccccaccttcccatccagacagccgagctcactccggaacttgtcatctccaacgacaaaaggagcttttgccactgactcggccgtgtcctgacacctccagaacgcaggtgctggcgcccgttctgcctgggaccccgggaacctctcctgccggaagccggacggcagggatgggccccaacttcgccctgcccacttgacttcaccaaatcccttcctggagactaaacctggtgctcaggagcgaaggactgtgaacttgtggcctgaagagccagagctagctctggccaccagctgggcgacgtcaccctgctcccaccccacccccaagttctaaggtctcttcagagcgtggaggtgtggaaggagtggctgctctccaaactatgccaaggcggcggcagagctggtcttctggtctccttggagaaaggttctgttgccctgatttatgaactctataatagagtatataggttttgtaccttttttacaggaaggtgactttctgtaacaatgcgatgtatattaaactttttataaaagttaacattttgcataataaacgatttttaaacacttgtgta

SEQ ID NO: 22 - DNA, 964 bp, Mus musculus
gcactgtttgctgctttaggtgtctcttttcctccctctctatctctactctccaacatgaaggcgctgagcccggtgcgcggctgctacgaggcggtgtgctgcctgtcggaacgtagcctggccattgcgcgaggccgcggtaagagcccgtcgaccgaggagcctcttagcctcttggacgacatgaaccactgctactcgcgcctgcgggaactggtgccgggagtcccgcgaggcactcagcttagccaggtggaaatcctgcagcgtgtcatagactacatcctcgaccttcaggtggtcctggcagagccggcgcctggacccccggacggtccgcatctcccgatccagacagctgagctcactccggaacttgtgatctccaaggacaagaggagcttttgccactgacccggtcgtcctggcacctcccgaacgcaggtgctggcgcccgttccgcttgggaccctgggactctgggaccctctctccagccggaagcctgagggcatggatgagcttcgatcttaacccagccctcttcacttaccctgaactcaacgcctcgaggctggacctggagcccgagagaaggactgaacttgggtggcctgaagagctagcacacgctggtcagcagctgggcaacgtcactctgtccccaccctgactcaagtctaaaagactggcttttccgagaatggggtgtcgagagggtgtggggggatgcgagtggctgccctgcgcactctgccaaggcagcataagagctgttcttctggtttccttggagaaaagctctgctgccctgattatgaactctataatagagtatatagcttttgtaccttttttacaggaaggtgactttctgtaatcatgtgatgtatattaaactttttataaaagttaacattttgcataataaaccatttttgaacactttgaaaaaaaaaaa

SEQ ID NO: 23 - DNA, 6255 bp, Homo sapiens
actctaacaagtgactgcgcggcccgcgcccggggcggtgactgcggcaagccccctgggtccccgcgcggcgcatcccagcctgggcgggacgctcggccgcggcgaggcgggcaagcctggcagggcagagggagccccggctccgaggttgctcttcgcacccgaggatcagtcttggccccaaagcgcgacgcacaaatccacataacctgaggaccatggatgctgatgagggtcaagacatgtcccaagtttcagggaaggaaagcccccctgtaagcgatactccagatgagggcgatgagcccatgccgatccccgaggacctctccaccacctcgggaggacagcaaagctccaagagtgacagagtcgtggccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgttggtaaacctcacaaatgtggatattgtggccgaagctataaacagcgaagctctttagaggaacataaagagcgctgccacaactacttggaaagcatgggccttccgggcacactgtacccagtcattaaagaagaaactaatcacagtgaaatggcagaagacctgtgcaagataggatcagagagatctctcgtgctggacagactagcaagtaacgtcgccaaacgtaagagctctatgcctcagaaatttcttggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaaagccctcccgcgcccccaccccagaccccgagccaccccaggaaaagcacaaggactgccgccttctcgctcccgccagcagcatagactggactggaccagacaatgttgtgtttggatttgtaactgttttttgttttttgtttgagttggttgattggggtttgatttgcttttgaaaagatttttatttttagaggcagggctgcattgggagcatccagaactgctaccttcctagatgtttccccagaccgctggctgagattccctcacctgtcgcttcctagaatccccttctccaaacgattagtctaaattttcagagagaaatagataaaacacgccacagcctgggaaggagcgtgctctaccctgtgctaagcacggggttcgcgcaccaggtgtctttttccagtccccagaagcagagagcacagcccctgctgtgtgggtctgcaggtgagcagacaggacaggtgtgccgccacccaagtgccaagacacagcagggccaacaacctgtgcccaggccagcttcgagctacatgcatctagggcggagaggctgcacttgtgagagaaaatactatttcaagtcatattctgcgtaggaaaatgaattggttggggaaagtcgtgtctgtcagactgccctgggtggagggagacgccgggctagagcctttgggatcgtcctggattcactggctttgcggaggctgctcagatggcctgagcctcccgaggcttgctgccccgtaggaggagactgtcttcccgtgggcatatctggggagccctgttccccgctttttcactcccatacctttaatggcccccaaaatctgtcactacaatttaaacaccagtcccgaaatttggatcttctttctttttgaatctctcaaacggcaacattcctcagaaaccaaagctttatttcaaatctcttccttccctggctggttccatctagtaccagaggcctcttttcctgaagaaatccaatcctagccctcattttaattatgtacatctgtttgtagccacaagcctgaatttctcagtgttggtaagtttctttacctaccctcactatatattattctcgttttaaaacccataaaggagtgatttagaacagtcattaattttcaactcaatgaaatatgtgaagcccagcatctctgttgctaacacacagagctcacctgtttgaaaccaagctttcaaacatgttgaagctctttactgtaaaggcaagccagcatgtgtgtccacacatacataggatggctggctctgcacctgtaggatattggaatgcacagggcaattgagggactgagccagaccttcggagagtaatgccaccagatcccctaggaaagaggaggcaaatggcactgcaggtgagaaccccgcccatccgtgctatgacatggaggcactgaagcccgaggaaggtgtgtggagattctaatcccaacaagcaagggtctccttcaagattaatgctatcaatcattaaggtcattactctcaaccacctaggcaatgaagaatataccatttcaaatatttacagtacttgtcttcaccaacactgtcccaaggtgaaatgaagcaacagagaggaaattgtacataagtacctcagcatttaatccaaacaggggttcttagtctcagcactatgacattttgggctgactacttatttgttaggcgggagctctcctgtgcattgtaggataattagcagtatccctggtggctacccaatagacgccagtagcaccccgaattgacaacccaaactctccagacatcaccaactgtcccctgcgaggagaaatcactcctgggggagaaccactgacccaaatgaattctaaaccaatcaaatgtctgggaagccctccaagaaaaaaaatagaaaagcacttgaagaatattcccaatattcccggtcagcagtatcaaggctgacttgtgttcatgtggagtcattataaattctataaatcaattattccccttcggtcttaaaaatatatttcctcataaacatttgagttttgttgaaaagatggagtttacaaagataccattcttgagtcatggatttctctgctcacagaagggtgtggcatttggaaacgggaataaacaaaattgctgcaccaatgcactgagtgaaggaagagagacagaggatcaagggctttagacagcactccttcaatatgcaatcacagagaaagatgcgccttatccaagttaatatctctaaggtgagagccttcttagagtcagtttgttgcaaatttcacctactctgttcttttccatccatccccctgagtcagttggttgaagggagttattttttcaagtggaattcaaacaaagctcaaaccagaactgtaaatagtgattgcaggaattcttttctaaactgctttgccctttcctctcactgccttttatagccaatataaatgtctctttgcacaccttttgttgtggttttatattgtaacaccatttttctttgaaactattgtatttaaagtaaggtttcatattatgtcagcaagtaattaacttatgtttaaaaggtggccatatcatgtaccaaaagttgctgaagtttctcttctagctggtaaagtaggagtttgcatgacttcacactttttttgcgtagtttcttctgttgtatgatggcgtgagtgtgtgtcttgggtaccgctgtgtactactgtgtgcctagattccatgcactctcgttgtgtttgaagtaaatattggagaccggagggtaacaggttggcctgttgattacagctagtaatcgctgtgtcttgttccgccccctccctgacaccccagcttcccaggatgtggaaagcctggatctcagctccttgccccatatcccttctgtaatttgtacctaaagagtgtgattatcctaattcaagagtcactaaaactcatcacattatcattgcatatcagcaaagggtaaagtcctagcaccaattgcttcacataccagcatgttccatttccaatttagaattagccacataataaaatcttagaatcttccttgagaaagagctgcctgagatgtagttttgttatatggttccccaccgaccatttttgtgcttttttcttgttttgttttgttttgactgcactgtgagttttgtagtgtcctcttcttgccaaaacaaacgcgagatgaactggacttatgtagacaaatcgtgatgccagtgtatccttcctttcttcagttccagcaataatgaatggtcaacttttttaaaatctagatctctctcattcatttcaatgtatttttactttaagatgaaccaaaattattagacttatttaagatgtacaggcatcagaaaaaagaagcacataatgcttttggtgcgatggcactcactgtgaacatgtgtaaccacatattaatatgcaatattgtttccaatactttctaatacagttttttataatgttgtgtgtggtgattgttcaggtcgaatctgttgtatccagtacagctttaggtcttcagctgcccttctggcgagtacatgcacaggattgtaaatgagaaatgcagtcatatttccagtctgcctctatgatgatgttaaattattgctgtttagctgtgaacaagggatgtaccactggaggaatagagtatccttttgtacacattttgaaatgcttcttctgtagtgatagaacaaataaatgcaacgaatactctgtctgccctatcccgtgaagtccacactggcgtaagagaaggcccagcagagcaggaatctgcctagactttctcccaatgagatcccaatatgagagggagaagagatgggcctcaggacagctgcaataccacttgggaacacatgtggtgtcttgatgtggccagcgcagcagttcagcacaacgtacctcccatctacaacagtgctggacgtgggaattctaagtcccagtcttgagggtgggtggagatggagggcaacaagagatacatttccagttctccactgcagcatgcttcagtcattctgtgagtggccgggcccagggccctcacaatttcactaccttgtcttttacatagtcataagaattatcctcaacatagccttttgacgctgtaaatcttgagtattcatttacccttttctgatctcctggaaacagctgcctgcctgcattgcacttctcttcccgaggagtggggtaaatttaaaagtcaagttatagtttggatgttagtatagaattttgaaattgggaattaaaaatcaggactggggactgggagaccaaaaatttctgatcccatttctgatggatgtgtcacaccttttctgtcaaaataaaatgtcttggaggttatgactccttggtgaaa

SEQ ID NO: 24 - DNA, 5451 bp, Mus musculus
gtcagggtcccgaagccgcgtgccgtgcgcgcaggccgggtgggctgtgggacaagccgagcgggaggcgagtcgcaagcgccaacccaaagtttgcacggtgcggggcgaggggcgcgcgctccgggctgccgcaggtggcggcgcggtgagcccgggccaggtgccccggcagcggggcggcgctgtcgtgcgggacagccgggctgccaggggctcggagccgggtcggagcccgcggggggcggggagtgtggcgagaaatggggaacaatgcgagtgagcaacttgaggaagtcattgtgaaagaaagctgggaattgctccgcagccaacttagcagggcactctaacaagtgcctgcgcggccgcgcccgggccggggacaggggcagcccggcgcagtacagcccatcccgggacgctcggccgcggctgccggagacccggtaggtcccgcggggtgcaggagcccccagatccccggctgctcttcgcgccccaggatcattcttggcccccaaagcgcggcgcacaaatccacataacctgaagacaatggatgtcgatgagggtcaagacatgtcccaagtttcaggaaaggagagccccccagtcagtgacactccagatgaaggggatgagcccatgcctgtccctgaggacctgtccactacctctggagcacagcagaactccaagagtgatcgaggcatggccagtaatgttaaagtagagactcagagtgatgaagagaatgggcgtgcctgtgaaatgaatggggaagaatgtgcagaggatttacgaatgcttgatgcctcgggagagaaaatgaatggctcccacagggaccaaggcagctcggctttgtcaggagttggaggcattcgacttcctaacggaaaactaaagtgtgatatctgtgggatcgtttgcatcgggcccaatgtgctcatggttcacaaaagaagtcatactggtgaacggcctttccagtgcaaccagtgtggggcctcctttacccagaaaggcaacctcctgcggcacatcaagctgcactcgggtgagaagcccttcaaatgccatctttgcaactatgcctgccgccggagggacgccctcaccggccacctgaggacgcactccgttggtaagcctcacaaatgtggatattgtggccggagctataaacagcgaagctctttagaggagcataaagagcgatgccacaactacttggaaagcatgggccttccgggcatgtacccagtcattaaggaagaaactaaccacaacgagatggcagaagacctgtgcaagataggagcagagaggtcccttgtcctggacaggctggcaagcaatgtcgccaaacgtaagagctctatgcctcagaaatttcttggagacaagtgcctgtcagacatgccctatgacagtgccaactatgagaaggaggatatgatgacatcccacgtgatggaccaggccatcaacaatgccatcaactacctgggggctgagtccctgcgcccattggtgcagacaccccccggtagctccgaggtggtgccagtcatcagctccatgtaccagctgcacaagcccccctcagatggccccccacggtccaaccattcagcacaggacgccgtggataacttgctgctgctgtccaaggccaagtctgtgtcatcggagcgagaggcctccccgagcaacagctgccaagactccacagatacagagagcaacgcggaggaacagcgcagcggccttatctacctaaccaaccacatcaacccgcatgcacgcaatgggctggctctcaaggaggagcagcgcgcctacgaggtgctgagggcggcctcagagaactcgcaggatgccttccgtgtggtcagcacgagtggcgagcagctgaaggtgtacaagtgcgaacactgccgcgtgctcttcctggatcacgtcatgtataccattcacatgggctgccatggctttcgggatccctttgagtgtaacatgtgtggttatcacagccaggacaggtacgagttctcatcccatatcacgcggggggagcatcgttaccacctgagctaaacccagccaggccccactgaagcacaaagatagctggttatgcctccttcccggcagctggacccacagcggacaatgttgggagtggatttgcaggcagcatttgttcttttatgttggttgtttggcgtttgatttgcgttggaagataagtttttaatgttagtgacaggattgcattgcatcaggaacattcacaacatccatccttctagccagttttgttcactggtagctgaggtttcccggatatgtggcttcctaacactctccccacccaccccaccccccaaaacagagcctgaatcttcatgaagtgaataaaacaattatccaagaaggagtaaggtggatcttgccctaagcagagtttatgccacaaagattctccaaatcccccaagacagcacagccactggggttgagccatctcagggagctctgcaggtgagccagaggaccagatataaggcagctggggaggagcagggacatcagcctgtgcagagaccaaggccaaaggttgaactttgaaagactattaagtcatatattgtatggcaatatggtgtctggacaagttgtgcaatgtgctgaagggaagggattggagagccttgaagactcttcttcatttgcctgatcaacccgacctccagagggtttgttgcccagtaagacgagctcagtgctcttgtgatcatttttctcttatcgtttccatgccgttgatggccctgaagctcatcactgcattttagaacccaatcctgaaattgggaccttttttttaaacttctgatactgtaaaacttcttggaagccaaagctttcttccaagccccatcctcagttatcctggttcctgttcttccccgagctgatagtaccaggacctgttattccacaaaagcacaggcatccgtcacttcaattcaatccctgttcagattatagatatggactttgctatcttgataaatgtcttctctatgttattttgtctgaaaaacctataaaaccattattaagaatgaccatttttagatggaagaaatgagcccagcatctcagtggctaaaacacaaaatatccatgcttttaaacaaaattgttaaatattccgaagctctctagtataaacaccaagtagcatgtgttttcacataaagaagacaggggccatgcaacctttatcaagtggaggtattagaatgttgtaatgtttggagacacagtgtgaccagtacaggttcccagagaggaatgcccaccatatcacagaaaggtagaggtgggatctggtatagccagaccaagacagggatgtcacgctgaagccaagtcagttagctgaagattctcaacaggaaggcctctcttaagagtcagtaatagggttgttaccatccaccacctcaacaaaacaaaaagcttataattgtaaatgtttacagcactgtcttcgcagaaactttctgaggtgattccaaagaactagaggggagatggtctataacagctcttgaagtaaacgaggttcttagtctcagctctcctgacatatagggcttgatcattactggtagggattgttctgtgaattgcttactactacccctggtctctccccagtagatgccaggaacattctagctgatacctaactgtcttcccaggtgttcgagggagcaaaccactgatctaaactctaaacgctgaagtacgcaggttttctaaaaatgacaagcccttgaaacctttcccagtaggcagcctcgagctggacttgtgtctttggaatgctgatgaattctatagatcagcattgcaaatacacttcaaatacgtctgagttcaagtgcagggactgagttcaccaaggtgtgaaatgtgctcaaaaagttcaaaagtgtgtgtttctttgtttctaaaacattgtggcatctttttcatttgtttctaaaactttttttttagaaacaaatgaagcacttggaaagtgaaagtaaaattacaaatataaggatttacactgaagagagaaaaattttaggaactatagctgtgaaaagattttgttcaaaaggcaggctagccttacccaaattcatatatggcaggtgtcaacctcccaagcttacagttagcaggcagcttttgctcactcatccttagccatgagagccattaagtgtggtccaagaaagatggctccaaaccctacccccgacccaccagtggtattcagagattaaagcagaattgtaaatagtggcttcaggagctcttttttagaatgctttgccccttcctctcactgccttttttagccaatataaatgtcaatttgcacaccttttgttgtggttttatattgtaacagcatttttttgaaactattgtatttaagataaggtttcatattatgtccacaagtaattaaattatgtttgaaggtggctatatgctgtatcagaagttgatgatgtttttctttagctggtaaaggagggttttgcatgacctcactgtttgttctgtggtttgttctgttgtatgatgtgtgtcttgagttttgctgtgtgatgaagtgcgctgagattccagtgccctcaagttgtgttttaagtagctatcagaggcaagagggttcctaagagcaggttgacctgttggcgacagatggcaatcaccatttctcattccttcttctccctgttaccccagcttcctgtcccaggtcccttctgtgattcttaccttagtgtgcatgtgtgtctgtcctggtgagagtcaggagcatcgatatgttatcattgcattatcaccaagggcacgcacagcctagcacctgttgcttcagataccgtcacactctgtttccaatttagatacaaccacataataaaatgttagagtcttcaatgggaagcagaggtgcttgttataaagatgggggcttatgcttgtgtcacattttgtgttcttttcttcttttgtttggttttaacttaattgtgacccttgtaacatcatcttgccaaaaaaaaaaaaaaagttgaactggatttatgtagacatgtcaagacgtactatctatttctttgtcagttatagcaataagagtggataaactctaaaatccagatctcccacaatgaacatccgtgttctttctatgatttttctttctttatggtgagccacaattaaacttgagatgtacagccacccaaacccaggaagctcatgtgcatctggtgctatggcactcactgtgaataagtgtgaccagatattaatatgcaatattgtttccaatcctttctaatacattttttc

SEQ ID NO: 25 - DNA, 9515 bp, Homo sapiens
atttcttggttttgtttatcagccgatctgtttgctggatttgggcttggaatgacccacctgtaaagtgcttttccttcctcctccccttgaactctgcagggggcttggcttggagggggcaagggagggaaagagagaagggggaaacacaaaaaacttctttctttctccctccgtttatcttcagcccgacattgtcacctcctctttgaggggttagaagaagctgagatctcccgacagagctggaaatgcattgcactttgactatggaaacagaggctattgatggctatataacgtgtgacaatgagctttcacccgaaagggagcactccaatatggcaattgacctcacctcaagcacacccaatggacagcatgcctcaccaagtcacatgacaagcacaaattcagtaaagctagaaatgcagagtgatgaagagtgtgacaggaaacccctgagccgtgaagatgagatcaggggccatgatgagggtagcagcctagaagaacccctaattgagagcagcgaggtggctgacaacaggaaagtccaggagcttcaaggcgagggaggaatccggcttccgaatggtaaactgaaatgtgacgtctgtggcatggtttgcattgggcccaatgtgcttatggtacataaaaggagtcacactggtgaacgccccttccactgtaaccagtgtggagcttcttttactcagaagggcaaccttctgagacacataaagttacactctggagagaagccgttcaaatgtcctttctgtagctacgcctgtagaagaagggacgccctcacaggacacctcaggacccattctgtgggtaaacctcacaagtgcaactactgtggacgaagctacaagcagcgcagttcactggaggagcacaaggaacgctgccacaactatctccagaatgtcagcatggaggctgctgggcaggtcatgagtcaccatgtacctcctatggaagattgtaaggaacaagagcctattatggacaacaatatttctctggtgccttttgagagacctgctgtcatagagaagctcacggggaatatgggaaaacgtaaaagctccactccacaaaagtttgtgggggaaaagctcatgcgattcagctacccagatattcactttgatatgaacttaacatatgagaaggaggctgagctgatgcagtctcatatgatggaccaagccatcaacaatgcaatcacctaccttggagctgaggcccttcaccctctgatgcagcacccgccaagcacaatcgctgaagtggccccagttataagctcagcttattctcaggtctatcatccaaataggatagaaagacccattagcagggaaactgctgatagtcatgaaaacaacatggatggccccatctctctcatcagaccaaagagtcgaccccaggaaagagaggcctctcccagcaatagctgcctggattccactgactcagaaagcagccatgatgaccaccagtcctaccaaggacaccctgccttaaatcccaagaggaaacaaagcccagcttacatgaaggaggatgtcaaagctttggatactaccaaggctcctaagggctctctgaaggacatctacaaggtcttcaatggagaaggagaacagattagggccttcaagtgtgagcactgccgagtccttttcctagaccatgtcatgtacaccattcacatgggttgccatggctaccgggacccactggaatgcaacatctgtggctacagaagccaggaccgttatgagttttcatcacacattgttcgaggggagcacacattccactaggccttttcattccaaaggggacccctatgaagtaaagaactgcacatgaagaaatactgcacttacaatcccacctttcctcaaatgttgacataccttttattttttttaatattattactgttgataattcttattttgtggaggcagtgtcatttgctctgcctaattacgataaggaagaaacagaagagagaaggggcgggaatattgtttctttatcacctggcttgtttattttgtgggaatttaagagcagtccatttctaccaaggcatatcatgctttgaaaaatcacttgattcataaagattcacctaagagattctgatttgccactgatattcagaattatgatggaagacaggaaagttcagagttttctgggtaggactttggtggtttaaaaatggtataagtaactttattcttgaaagaagaatgtgtttcaaactgtaaaccaattttttgttcttcagagatcatggaacacaaacacattgttattttcagtgataactcctaagaggagctgagttgttgtgggttctatgtttacttcccctatggaatttataattcagtatgttttacactgtaccatatagcaaaacttttaaactacaggtagttaagggccacctacaatacatctgaggtcctgtgatcttatttttctaaacgtaagcactgtttttccatagttttgatgactggcattttatagacaccctggcagccttacttttaacacctttaaggaatagtatttttatgtagttttcagaataacatatggtctaagagtggataaaaggcagtcaataatttctgggagggacttctactttcataaatttgtttgagaggttttcttttaaagttgtaatgtgatggcagcatagtatatgtatttgtttctaaaagtatgcttacgattgtcactttatcagcatttaatcagtgttaaccagtcagcagaaaaatataattatgctaacagtagggggagaaaacccacttagaaatcccttttctggtatttctcttttcactagtttttttcaagatgtgacctcccggtgttctgtccatagttcattcatcctttactcttcgagtagaaggtcttaaaagtcttcctgtcggctgtttctttcaaaatctcctcagagcaattgctaatttggcctgaatctggtaacttgaaccctgtaaggttacagaactagggctatttattttagcatttcttcagtagtatttactactcttgttgcaaagaaaagggaatgggacttctttgtaacctgtaccttggacaacagataaaagaaacaaaaaaataagaaagtttacttttacccttcttggagtctagaatgtgacagaacccccaaaggaaagtcctgcacatttttctgtttccaaaacatttaattgtgtaagtccttgtcagaaatgaatctcaatcccttagtatagaattccccttacatggtataggttgccatatttcatgtgcagattttaatttcatttatgtgggcgctctgttttttctttgcagtccagccacattagaggggaggaaccgagtgatattgattcaagtcattttagggggacatacttggaaggcagaacttgctgcttctgtttggggaggacagacctgactgtgactggattatctgataaccatttgtgaatactgaaattctgttaggcagtaactgataactgctctaaaggatcattaaataggatgctgaaattatgtatcttaatacagtgtggtatgagaattaccaagtcaagagaattgtggacataagcaagtttggccccaatactgctcttaactcattttccagcttactatttgctatttaaatggtaggcaccagctaagcacttctaagcactaacacagctagaactaggcaaaaatggttagaactcagctctcttctactagtccctgtcataattatttttgggaaaatgtccaaactgccccctttaaatctaagggaatgcaccaaaacagagatatatagaatgtcaaccatttcatttttttttttctgcatgccttggtacatagtgaacatacaacctatttaaagataaagcatgtttttgagactcgctcacccccccccacccaaccactcccaaataataattgggatgccattttttttccttttggatgaggtaaataattttaaggttcacaattttgtcttttactgcaatttaaggaaacatttggatgtcagtcaatatgttcataattttggctgtgtgcgaatttctgctggcattatctatgaattttcttcctacttatttttttttcagtatatgaacaatcatgtatctacctgccccaggatgaaactaaatttaggtggaccctaaaccttatgaagacagtgctgaggcactttccttttctgatttcatctttttgggaatctgttttattgaaggtagttagtagttgagagtgcatttgctacaagcatatacttgtatcttcctagcttcatgaggaacagaaagaggtggatatggctcagggtgtggcagggacaattgaggacaaagtcaattcaaatttgtgggtcagaaagaatttttgtggacgtagtgtttttggagaaactctggatggttatatgtgcatgccttttcttcaaaaggaaatacgcaaggttgtagcatctaaaaataaacataagagtcagacaccaaataaatcaagttttacataacagttgtatgcccagtttgtttaggtgagatttcacattacagaaagtatttgaggagcatgaaaatgggttatcttctgtattttccagtttggcaaaagttcagaatttcatcacattgctttgccctaattttgcccagaattttatcttagcctctctctgacagtgatgaatcatgctcaaaagccattctaattggacctttttaagacagggaaagggatcagtaggcggattggaagaaatttcaagtcattgaaatattccattgagatttcctaaagggacaaaattgggaaaataagaaactacgacttagatttggctacgtagtagaaagtatctcccctacatacatacaggcaattgtatgtatgaatcatagggtatatgtgtgtgtatactacacacacattcttttaaagagaattcatggaaaaaaaagcagttggagtgatcagatgtattgcaaaaacatacagagaatttaaatgacagttaataccaagaaattagttgggtttactttatcaggtcgtaataggaatcactaaagaagttactagtgtgtctttaggaccagtggcaactcttaaactaaaactttgggtccttattatctacttacagaacaaagtgaaacaaacaatgattaagctgattggatatacattcaaagatatttaatgtaaagttttttggaatacgaagaaaattcagaaaataaatattatcaacagttacttattggcaaatagagaaagacaagaatagtttagtgagcccggtattttgtttttatagtttttatctcagttgtacaactcacaaaaccatgaagtctttggtattttataaatgtttaacaaaatttacatcagattaaggcatttagatgaaaattattatgttctcactatcttccaaattttatttcatcctatctccaaaatgatttcttagggtacaaaaagagcagacggggctgtaaaaatacaagcaaaaaactgtgtgcccctagtttcaggcagaacttaaactgtcagaggtactagctacatgatttgttttttaactttggattgttcacgtccaaaaatggataaattacatttgtgtttatcatcagttgcattttatgtattattttaataaatactatctgaatgaagactattctaaaccagaaaattccccaaatccaaaagaaaaaaaaagtgggaagaggtgaaattgaagtttgtgtatatgaaagttatcttagacatatttttaattctccagtttctgcaaaataattaaaatatacagtaactggtctcctaaatcctgaatttaatgtattaaatacttatgttctttatattggtgcctttttaaaatgcattgagagtgttggttagctgttgcagctgtacaacacttttaatatgcatttttaaaaatcacttaaaattgagtactatataattcatctctgcatttttagtgcaaatctttagagcaatttctaatagagaaattttcagctcagctgttaaaaggaaaaggaaactttgaaactagactttactacctttttagtttcatagtatttctgaatatgattacaagattatgcaggtaaaatatagagtgaaactttacctgtgaattgaattataatttgtgtttttgttttgtttttaaggaagaataagttctgtatcaaacaagaatttattagataattttttggtcaataaaatacagtattcatttggattttcatctccagactagtattgttctagtcttggaatctgtattttctaatctgttagaaaatagagattgaaaattgatggaataatgtgaaaaagcaggtaattaattctccttgaacaaagcaaaactgaacagtcatatcacattgctattctccaaagcataatctcaaatggtttcatatcatggttgtgtattacttgcaatgggtgtgttaggatatgacagctttttaaaaaaatgagctgctggttatacaaagcaaatggcatatgaccaagaagctgtgatatgctagtgtttctttttatcatagtgtattactaggccaaataatgacaccttgaatatttttacatttattgcagaaaccttaaactttggaatttccataaggtttttatgtaatattctatttctagctttttagttttatcttgctgtactgtaagtttgaggatatttttcacctgcactcttaggaataagttcataattctgtttatggggctttcctcccataacactgcatttgtatattttctgtataaaatatgtgttgtgtattaacctttatcccatacagagagtggtacatgaatgactagttttctaagatgtcctttttattgtgaataaaatataaaagttaaaggccctctgctaagtcacataaagtacagcatataagttcatataggtacaaataaatgagtttgcagtgaattgggccttcaaattacctcaagtgacagatagtaagaaaagcttcttgagcaggtggaggtcactgaatcccctactatgcacttaccaagattttacttactttaatttactggaaattgattttttaaaaaatgactacactgtaacaagggaagggatctgggtttttttgttgttttattcttgttttttttaagtagttcaaattctgaaactgtgatttaaaaattttttacagtcaagcattctgattttgaacataactcccttccctttctgtgtaacaaaggtctctctgttatctcttaaattttgttacatctccctcagcctctttctttgtccgtctcccttctgtcattgtctatggatgtttacctctctgttctcctaaaagtttgaagattaggtcaactcttatttctagttcattggtaatttaatcttaatttttttttcgtgatttttgttggttgtataatctgctgacgtatttttatactcaagtgtagttttctattaaaaagaaaagtggttggattaaaaatagtaagctatgtaaccctcatgttactttcactttcaaatattgggtacctaaaacattacttcagagattatgtaatcctattatagtatgtttgctttcctttattgttggattttacattctgatttggctttcctccaaaaaatgtatatcatgaaagactagacagttatttgcaagtgtttagaaaggtgttaaaaatgtaaagcaaagagtcttaactttctcctaattgggagaaaaatgctttaacattactataataatattccaggtttggagggggtctccaggccccatatttgctgttaatagttggaccttttagaccatgtgttatttgcaatcccagaatgattgcttctgctattagttaaaaagatactattcttttctttctgtacaagtgcaatactccccttgaagtcttaaaaactatggtgatttttttttcttttctgacctattcttcctttagctaatgacaaaaagaaactcataaaagtcatagtatgttaaaggacacaacaagcaaagagaaaaacactccacaatcaaaagattacagaatgtggaaaccactagtctgatctcatggtatctttatttaagctaaatttccatggaaattagtaatcttttgcttgaaaaatgtgtcctaaagttgaactttttacagattgaatcttcttagaccctcgcccaatgctctaaattaagaacctaatacttaatatttttattttacttctccccttttagaaataaacttttaaataaaagcaaagcacttagctgagttttaaacacttacatatcacctattggagaaattttttttaaaaatatttggagcagtcctgttttcatacaaatttaagtaagaggtatttttcttatacatatttatatgtagtgtgctaattttctttttttatacctgtgtccctgtagtaaaactgctgtaatataaatacatgttttgttaaaagataacatttctttggcatttcttttaaaggcagttactgcatttctgcatttgtacagtatgtgtcttggccattttagatattctttctttaacaataccaaaggtaattagactattttaaagactaattgcttgacagtttctagggtattttgtgttttagaagcaaaaaaagaaaaaaaaataggtcaaaccagtaaacctcattttttttcaaactaataatttggggaaataaaaactattgtttaaaaaagaaatatatatatatatatataaatatatatgtaaagttaaaattccataccttgtatgtcaggtttgctaagtgtaatgtagtttttttaaggctcaaataccatacctcagaaaatgaggtttactatggaaatactgaaacagtctttgcagctgtgtgacaagtcactctactacatactgatttggagacctccgctaaatagttttatcactgcagactaaaatgtgggacttgtatcttctttgtttttaatgcacacacatacatgttctgtgcatgtatgtggttactgtgtatatgtgtatgagtgttgtatatgcatgtgtgagtgtgtgtctgtatgtgtgtacaactaaagaagctgcagaaactttgtaatactttgtgaaaaggattatattataaaggtttgtactgtctgagtgcacagctactggaataaatttagggaatctcaggaacaagcatataatttgtccaagatttatttcttctcagaagtgtaagtgcagtttttaattctgtatattatttaatattttaccaataaaataaacttctgacataaaaa

SEQ ID NO: 26 - DNA, 9457 bp, Mus musculus
tgtttgctggattcggtcttggaatgacccacctgtaaagtgctttccctcctcctcggcttcaactctgcagggggctttgcctggagggggcagggagggaaggggggggggacacaaaaaacttctttctttctttctttctttctccgtttctcttcagcccgacattgtcacctcctccttgaggggttagaagaagctgggagctcccgacagagctggaaatggtgatgactgttttttaatcagaggacaatttcttttcactgcactttgactatggaaacagacgcaattgatggctatataacatgtgacaatgagctttcacccgaaggggaacacgccaatatggccattgacctcacctcaagcacacccaatggacagcacgcctcgccaagtcacatgacaagcacaaattctgtaaagctggaaatgcagagtgatgaagagtgtgacaggcagcccctgagccgtgaggatgagatcaggggccacgatgaggggagcagcctagaagaacccctaattgagagcagcgaggtggccgacaacaggaaagtccaggaccttcaaggcgagggaggaatccggcttccgaatggtaaactgaaatgtgacgtctgtggcatggtttgcattgggcccaatgtgcttatggtacataaaaggagtcacactggtgagcggcccttccactgtaaccagtgcggagcttcttttacccagaagggcaaccttctgagacacataaagttacactctggagagaagcccttcaaatgtcctttctgtagctatgcttgtagaagaagggacgctctcacaggacacctcaggacccattctgtgggtaaacctcacaagtgtaactactgtggccgaagctacaagcagcgcagctcactggaggaacacaaggaacgctgtcacaactatctccagaatgtcagcatggaggctgccgggcaggtcatgagtcaccatgtaccgcctatggaagattgtaaggaacaagagcctatcatggacaacaatatttctctggtgccttttgagagacctgctgtcatagagaagctcacggcaaatatgggaaagcgcaaaagctccactcctcagaagtttgtgggggaaaagcttatgcgattcagctacccagatattcattttgatatgaacttaacatatgagaaggaggctgagctgatgcagtctcatatgatggaccaagccatcaacaatgcaatcacctaccttggagctgaggcccttcaccctctgatgcagcatgcaccaagcacaatcgctgaggtggccccagttataagctcagcttattctcaggtctatcatccaaacaggatagaaagacccattagcagggaaacatctgatagtcacgaaaacaacatggatggccccatctctctcatcagaccaaagagtcgaccccaggaaagagaggcctcgcccagcaatagctgcctcgattctactgactcagaaagtagccatgatgaccgccagtcctaccaaggaaaccctgccttaaatcccaagaggaaacaaagcccagcttacatgaaggaggatgtcaaggctttggatgctaccaaggcccccaagggctctctgaaggacatctataaggttttcaatggagaaggagaacagataagggccttcaagtgtgagcactgccgagtcctttttctagaccatgtcatgtacaccattcacatgggttgccatggctaccgggacccactggaatgcaacatctgtggctacagaagccaggaccgctacgaattttcatcacacattgttcgaggggagcacacattccactaggccttttcattccaaaggggacccctatgaagaactgcacatgaagaaatactgcacttacagtcccaccttccctcggatggcgacatgctgtcttctggatgctgtcactgtctataattcttattttgtggacaaaatgtcatttgctctgcctaactacaatgaggaagaaacaaaagaaaagggatgggatgttcaatgataacttggcttgtttatttcgtgagcatttaaagcagttcattgcagccatgcatccttgttaaggcctatcataatttaggagatcattcagttcatagaggttcatccaagagattctgatctgccattcatattcaggattgtgatagaaggcaggaaagttgagagttttctgggtaggatgcttggcaatttaaaatggtctaagtcattttactctcaaagaagtttcaaaatgtaaaccgattttattttctgttcgttagagatcatggaacacaaaaacaatgttattttccataactactaggatgagttgaattgttgtgggttctgtgtttacctcccctacggaatttataattgagtatgttttacactgtatcatatagcaaaaattttaaactacaggtagtcaagggccgctacgatacatctgaggtcctttgatcttatttttctaaacttggcgcactgtttttccatagttttgatgactggcattttatagacaccctggcagccttacttttaacacctgtaacaaatagtattttttatgtagttttcagaataacatatggtctcaagagtgggtaagaggcagtcagtaatttccaggaagaattctgcttttcacaatttgagatttttttttaagctgtaatatgatggcagcttagtatacatgtttgcttctaaaggtgtgtttacgatcgtcactttatcagcattcaatcagtgttaacaagtcagcagaaaagtgtaattaggcgaacagtagggggaattcccactctaagaacccttttctggtatttctcttcaagctgtgaccactcagtgttctgtccacagtccattcctctttgactcttggaatagaaggtcttaaaaagccttgctagcagttgcttcttttaaaatcttggagcaactgttaattggacctgaaagtggtaacttgaaccctggaaggtcttttatttactagggctatttattccactatttcttcagtaataagagttactacctttgctggagacaaaagggaacttctttgttacctgaataacatagtgcaggtcaaacaaacaagcaaaaagttttctttcagccttcccggagttgagaatgtgaccatctttaggagaaggtcccgcactttaaaaataaaaacaaaaaatccaactctagagttccttatcagaaatgaatctcaaccccttagtcaagaattttctgtcgtctcttaatttgcacgtgagaaatacttccatatctcatgctcaggttttaatctgatctttgtgagttctcaattgtttttttttttttttttctttttcttttctgaacttgacttgcatttgtgggtaggattggagcacactgttatcaaggcattcgggggaatcaacttggcagacataacttgctgcttgtgtttgaggaaaacaaacctgaccataactggattatttgacagtcatttgtaaatgctgagaggtctgctgtgtggtaacacatcgctgctccaacagtcccttgagtgacatgcccaaaccatgtcactgcactttgccttgggaagtaccacgactggagaagtacccacggatgagcagtgtgctccgaactcgctttgcaggtttctgtctgatcatttaaggaacttcttagctcttccatagccagagcgaggtagttctgacttgcctcttttactaatccttatcttagttctttctggagaaatatccaactggttccttaacatgtaagagaatgcaccaaaccagagctatatcaaatgccaattgttggttttgtttttttttttttggcgggggggggggggggggttgtatcccttggtacattttaaaaacatagcttcctcggggatgaaacatctcatcatgccttccctatccccacttaccctacacactctggaatcacagtgaaaaggatatttatttctgaatgaggtaaataagtttaaggctcctgacttctttgttttaactgcaacttgaggaagcatctggatgtctgtcagtgtgtccctttcggctctttgtgaacacctgcaggcatctgctatctcttccttctctctgctagcacctgctctattaacaatcatgtacctacctgccctgggatgacactgaatttaggtgtactggaagagttgtgtccttcccctacgaaggtggtaccgatgtgctctgcctttctgatcccattttattggaaaacctgatgtactcaaaggcgtgaactataagggtagattggctgtacatatgcacttagccttttctttctttttcagctgcatggcacaagatacagaaaaaaaagtagatagctctgctgtgacagggacaaatggggacaagctgaattcaagatttgtgggttacgataaactttgtggacatagtgctgtctgagggaccctcaatgactgtatttgcctctttttcaccaaagagaataggagacatggtagcacctaaaaataggtatgaaggccagataccaaataaatcaaatttacataaccgttacaagaccagtttatttaggtgacattttacagttgagagagaatttgaggggcacgaaaatgggttatcctctgtgttttccagtttgtcaggaatttatcagatggctctgccctaattttgcccagatttttttttttttatcaccagttccttctggagaaacatgctgcgtctctatagtagtgattgattgcttgttataaaggctttttagaacagtgagagccgccagcccaatgagcggtagagggaagtgtgaaacactgtcattcaaacattccgttaataattctctggataaaatggggatgtgagggccaaccattaagatttagaatgaagttggctgcctggctggcatacatacaggtcagtgtttgaatgcagtacagtttgcatgcacatgcatattgcacattcttttaatgaaaattcacagaaaagaagcaattagcattgccagttgtattacaaaagcttagagttagaagtacagtacagttactacgccagcagttgggtctaattcagcaggttgtattaagaaagccccatgttgtcagtgtgtctttaaggctaatggcaatttaaatcacaaatctccattgtctgcatccagaaaaacaaacaaagcaaggcaaaaaacaatgaaacaatgaaaagatattgagttgactggagaatatttgaagaaatatagattttttttgtgagaggaagaagttgcaggaagtaatttagcaaccttggcttgtgtttgtgcttctctgacccagttgcacacctcacaagtttggtctttccaaaccttcatcttctctcctaaatgacaagttagggtagaaagattcatggaagaggctggagaggacaaggagagctgtctgcccctagattcaggactgcgccttacactgtccaagtcacctgccacatactgtcttagccttgaaattgtagattggtggtgatcgtcagttgtacttcatgttattttaataactactgtctgtataattattctacagcaaaagctcccccaaatccaaaaggggaaaaaagtgagggatgaattgaaactttgtatatataaaagttattttataaatgttttagtcttccagtttctgcaaaataattaaaatatacagtaactggtctcttaaaccctgaacttaatgtattaaatacttataaaattttatattggtgccttttaaaaatgcattgagagtgttggttagctctttagctctaccacacttttactgtgtattttttaagaaaaaaaaaaatcacataaaatctaagtactctctaattcacctttgtgttcttagaagaagaagaaaaaaactgctaaaagaatttccagacttttagctgagctggcaaaagtgacaaaaaaaaaaaaaaaaaaaacaaactttgaaaccagattttaatctttttagcttgatgatatttctggacattattctatggtagaggcaaaatagaaggtagactcacatatcagcaatgaattggattatgctatatgttttgatttcttcttgaggaagaatatgttttgcatcacacaggggtttctttgtataatttggagtcagtaattacagtattgctttgctttgttttaaatacccgactgatgttactctagtcttgggtcttctgtctcttctgtcttctaacctgtggttgttttgtgaaatggcgtgggaatgcagatcattgtctcctcttgaacaagacaaagctcagcacactagtcccgtgactggtctccaaaatgtcatctcagatgtgtagtctggggtcgtctgtcacttctaaggggtgtcttcaactatgacagctttttaaaaacgagctgctggctgggtaaagcagacatcatatgaccaagaagctgtgatacgctagtgtttccttctgtcatcgtgttgtactcttaggccaaataatgacaccttgactgtttttacatttactacagaaacctaaacttttggaattttcaaaggtttttatgtaatgttctatttcacagcggtttccttttctcttgctgtactgagtctgtgagtattttttttttcagatgcactcttggaaataagttctgaattctgtttatggcgtttctctcgcttgacactccgtgtatagatgttctgtataagtttgttatgaatccacctttctcatgtatggacagtgagaagtacatgaacaactagtttcctaagatgacttttatattatgaattaatatgaaatttacaggtcatgtgcaccttaagtgcagaatcaaaacttcctataggtccacataagcagttctcagtgaaccgggccttcagtttacctcaagtgacatgtagtaaggaaagcccgggaacatggaggtcactctctgcagcacagtgcacttagtagcatgttgctgaatctcagttacagaaaatggatatttaaaggaacacactggaaggatgcaagggattgagggactgctgatattttcgttgttgctgttgctttgtgcttttcaaaaagttgttcaaattatggaactgtgatctaaaaacggtttgacagtggagcaccagagaggatgtccaccactcacttgcccttcggtacgactaagctctgagtttgcgtcttcaactctaccttcctctcccctcctcatccctactctgtctgtggccctcctctcggtgtcatttcctcttggtggctctcttgacactcctactagttaggaggtcaactctgaacttctgactcattaaatcttaatttttgtctcagtggttttagttagctgtgtagtctgctggcgtatttttatacccacgtgtagttttatatttttaaagcgtggttgagttgacatggtgcactccgtagtccttattttgcttcactctcaagtaggaggcacctaacgtgactccagaaattctgtaatcagagtatccgtctggctcctttgtgcttggattttgatttgtttggctttcttcttaaatacaaatcatacaagagtagactttccccccaccatagaggataaaaatgtaaagcaaagagtcttaactttctcccaaccggggggcggggcgagtgctgtcccacttctgcagtgatgttccaggctcagaggggtctccagaccccaggcttgcctgtcatcgggtctcttcgaccctgttatttgcagctttgggatggtttcttttgctatggagtttaacagctcttcttttttccataaaagtgcaatacccctcttgaagtcttaaaaactatggtaattttcttcttttctttctctgacatttgtctcattagccaatgatgaaaagaaatctttggaaattctaacatgtcataggacataggcaaatatggaaaccactctgctatgaaaagttgaaaaaaaaaacgtattaacaaacgactcctctgactttctagaaacttcgtttagactaaattttctttgaaatcatgaatgaaagtttatcctgaggctgaaatataatttttctcccactcttgtctaatgctcttaagttaaataactaatatttaatgttttcatttctctcattttggaaaataaatatcaaagcacttaggtcaggtttaaatcaatattacctgttggaaaaaaatttttttacatatttaaagcagacctgtttcaatgcagatggagaagtatcttttgtattattcatatttatatgtagtgtgttcttttctttttttaatacctgtgtttctgtagtaagactgctgtaatgtaaatacacgttttattaaaaagtaacatttctttggcatttcttttgatggcacatactgtatttttacagtatatgtcttggctacttaacttttttttctttaataataccaaaggtaattagactattttaaggactaattgcttgacagtttctagactactttgatttttttagaagaaaaaagaaaaaaggataaaaaaaagtcaaaccagtgaacctcattttttcaaactaataatttggggaaataaaaactattgtttaaaaagaaatatatataaatatatatataaatatctgtaaaattaaaatcccagaccttgtatgtcaggtttgctcagtgtaatgtagggtttttttttgttttgtttttgtttttgtttttaaaggctcaaatacctcagaaaatggggtttactatggaaatactgcgacagtctctgcagctgtgtgagctgtcattctgctgcatactgattgggagacctccactaaacagttttatcactgcagactaaaatgtgggacttgtattttctttgtttttaatgcacacacgtgcatgctctgtgcgtgtatgtgggtaccgtgtatatgtgtgtgagtgttgtatatgcatgtgtgaatgtgtgtgtgtgtgcgtgcttgtgtgtgtgtgtgcgtgcttgtgtgtgtgtgtgtgtgtgtgtgtgtgtgtacaactgaagaagctgcaaaaactttgtaatactttgtgaaagggttatattataaaggtttgtactgtctgagtgcacagctactggaataaacttagccaatctcaggaacaagcatataatttgtccaagatttatttcttctcagaagtgtaagtgcagtttttaattctgtatattatttaatattttaccaataaaataaacttctgacagaaattatttggtacaaaaaaaaaaaaaaaaaaaaaaaaaaaaaaaaaaaaaa

SEQ ID NO: 27 - DNA, 9788 bp, Homo sapiens
aaccccgcgcaccggcaggtcgccggctcctgcccttttcagtctgcgccgacgcggctgccggatcccggggactccccgcgccgggaatctcccgccagctgcgcgctgagtccaggcgacggcaggagcacgtggagaggccgagtagccacagcggcagctccagcccggcccggcagcgacatggaagatatacaaacaaatgcggaactgaaaagcactcaggagcagtctgtgcccgcagaaagtgcagcggttttgaatgactacagtttaaccaaatctcatgaaatggaaaatgtggacagtggagaaggcccagccaatgaagatgaagacataggagatgattcaatgaaagtgaaagatgaatacagtgaaagagatgagaatgttttaaagtcagaacccatgggaaatgcagaagagcctgaaatcccttacagctattcaagagaatataatgaatatgaaaacattaagttggagagacatgttgtctcattcgatagtagcaggccaaccagtggaaagatgaactgcgatgtgtgtggattatcctgcatcagcttcaatgtcttaatggttcataagcgaagccatactggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctcacggggcatcttaggacacattctgtggagaaaccctacaaatgtgagttttgtggaaggagttacaagcagagaagttcccttgaggagcacaaggagcgctgccgtacatttcttcagagcactgacccaggggacactgcaagtgcggaggcaagacacatcaaagcagagatgggaagtgaaagagctctcgtactggacagattagcaagcaatgtggcaaaacgaaaaagctcaatgcctcagaaattcattggtgagaagcgccactgctttgatgtcaactataattcaagttacatgtatgagaaagagagtgagctcatacagacccgcatgatggaccaagccatcaataacgccatcagctatcttggcgccgaagccctgcgccccttggtccagacaccgcctgctcccacctcggagatggttccagttatcagcagcatgtatcccatagccctcacccgggctgagatgtcaaacggtgcccctcaagagctggaaaagaaaagcatccaccttccagagaagagcgtgccttctgagagaggcctctctcccaacaatagtggccacgactccacggacactgacagcaaccatgaagaacgccagaatcacatctatcagcaaaatcacatggtcctgtctcgggcccgcaatgggatgccacttctgaaggaggttccccgctcttacgaactcctcaagcccccgcccatctgcccaagagactccgtcaaagtgatcaacaaggaaggggaggtgatggatgtgtatcggtgtgaccactgccgcgtcctcttcctggactatgtgatgttcacgattcacatgggctgccacggcttccgtgaccctttcgagtgtaacatgtgtggatatcgaagccatgatcggtatgagttctcgtctcacatagccagaggagaacacagagccctgctgaagtgaatatctggtctcagggattgctcctatgtattcagcatcgtttctaaaaaccaatgacctcgcctaacagattgctctcaaaacatactcagttccaaacttcttttcataccatttttagctgtgttcacaggggtagccagggaaacactgtcttccttcagaaattattcgcaggtctagcatattattacttttgtgaaacctttgttttcccatcagggacttgaattttatggaatttaaaagccaaaaaggtatttggtcattatcttctacagcagtggaatgagtggtcccggagatgtgctatatgaaacattctttctgagatatatcaaccacacgtggaaaagcctttcagtcatacatgcaaatccacaaagaggaagagctgaccagctgaccttgctgggaagcctcacccttctgcccttcacaggctgaagggttaagatctaatctccctaatctaaatgacagtctaagagtaagtaaaagaacagccataaaataagtatctgttacgagtaactgaagaccccattctccaagcatcagatccatttcctatcacaacatttttaaaaaatgtcatctgatggcacttctgcttctgtcctttaccttcccatctccagtgaaaagctgagctgctttgggctaaaccagttgtctatagaagaaaatctatgccagaagaactcatggttttaaatatagaccatcatcgaaactccagaaatttatccactgtggatgatgacatcgctttcctttggtcaaggttggcagagcaagggtataaagggggaaattgtttggcagcaccaacagaaaacaaacaaacaaaaaacagctacctaaaacttcttgaaagagttcatggagaattggtgatacagacccaaagcaaatttgccaatgatattttccacaaaaaaagtccaaaaagtatggctcagcctccccctccccacaggagaggaattggagatagatggcatgtgtgtttagatcggagttgagctccggaatggggtgaggagggacacctctattgagaggttctccttgatcaggcaggcttcggccctttttttcccatttaaatggaactgctgtattccatgaaaattcctgaaagtctgatcacggttctgcagatgtataagtcatccttgtcactcataatatgtacatactatcaggaggagtgctgttatcatggtaaaattagcactggaataggaggtcacaaaatgctggctaattagctatgtgactttgagaaatcgtttaactttttttttttttttttttttgagacaggatctcactctgttgcccaggctggagtgcagtggtgcaatcatggctcagtgcagcctcgacctccccaggctcaggtgatcctcccacctcagcctcttgagtactgggacaacaagtgcacaccaccatgtctggctacattttgttctttttgtagagataggggtctcactatgttgcccatgctggtcttgaactcctgggctcaagcaatcagcccgcctcagcctcctaaagtgctgggattacaggtgtgagccaccacacccagccttatttaactcttaaaactcagtttccggccaggctcggtggctcacacctgtaatcccaacactttgggaagccgaggcaggcgcatcatttgaggtcaggagttcgagaccagcctgacccacatggtgaaaccctgtctctactaaaaatacaaaaattagctgggcagtagtggcacatgcctgtaatcccagctactccggaggctgaggcagaaaaatcgcttaagcctgggaggttgaggttgcggtgagtggagatcacactactgcactccagtctgggcgacagagtgagaccctgtctcaaacaaaacaaaacaaaaacaaacaaacaaaaacaaaaaaaactcagtttcctcatccataaaataggaattagatttcaatgttctcttaggtcccttctagctttaattcatatgtgattatgcagtaaccacaaggtattttttaaacctcctaatgtatggatattaagcagaagagtatttatatgaatacatgtttcacattcctttggtatgaaaatggtgtgttaagtttttcctttaaccactgagttgtgaatgtgaagaaggtggtggagaggaacaaaaaacagaaaggtattttgatcttgccacaaagcatacacacaaattggcacatgcagctgtttgccaaagccttctttttttttttactttttaagaaattatgttagggaaaataaattctgcttccagggacaacttcatggagcctatttacaaattaagagtcagcttaatttgtaacatttctaccagagccaagaatcccaaattcctggtagattagtgttttatttctaaggggcttatgcattcggctccaactcaactcgtctatgtgctgccagtaattaaaatgttccacctcagactgcacaaatggcttatccttctttgtggcatggcgtctgtctcaggaaaaaaggttttatgaaattccatggcaacagtcccaacatgtttgagacttcagctaaaggaatggatgtattttggtgtgtagtcttcagtatatcactgtatttccgtaatactagactccaagctatgccagattgcttattccctttgtgaaagaggagttgctcattacgttcttgaaatatcgcacatcctgttggttcttcaagggacaagagaaagagaatttggaagcagggattagtagaagagaaaacgagggaaaggaagcctttccaccagattagtgttcaagtctttgcagaggagaccaactttttttgttttcttttgttttgagacagtctctcgctctgttgcccaggctggagtgcagtggcgcgatctcggctcacggcaacctccgcctcccgggttcaagcaattctcctgcctcagcctcccaagtagctgggattacaggtgctcaccaccaagcccggctaatttttgtatttttagtagagacaaggtttcaccatgttggccaggccagtctcaaactcctgacctcaggtgatctgcccgccttggcctcccacagtgctgggattacaggcatgagctaccgcacccagcctgagaccaccttttgcatctcaagattgtgaaaccaaggcccattccaccagcctggggactctttttatagatatgatcctcctttttcctgtgactaatgaatttgctgcatgatttctattcttctgaggttagttttctgagtaaggtgaccactcacaaaggcactttctttgtggcattctgagcctagattggggcccatcaattccagaaaaaatttatgtgtggaaactctgcatccttaagtcttgaagttgaaccagatatgcagtggttaccatcacacagataaacgctgccttctgtacataccccttatgctgtactaattaacaaaccccttgccagggctggggaggtgagggtgaaggagaatcttagcagaagggcagagtcaggacttgcatctgccactgctgggcactgaagccctggagcagcttcagatagtacctgtactttctcatgcagactccctctgaacaagagccttgtaggcccctctccttcatttcccaccagcctcttatcaggcgggctttccaccatacacccaggaggccacggtctgaggaacaaccaaacccatgcaaagggccgggcgcgatagctcacgcctgtaatgccagcactttgggaggctggggcaggcagatcacctgaggttgggagttcgagacctgcctgaccaacatggagaaacccccatctctactaaaaatacaaaattagccgggcgtgatggcacatgcctgtaatcccagctactcaggaggctgaggcaggagaatcgcttgaacccgggaggcggaggttgcggtgagccgagatggcaccactgcactccagcctcggcaacaagagcgaaactctgtctaaaacaaaaacaaacaaacaaacaaaaaaacccaggcaaagtttccttgcagccaaggtgacagaactgggctgagggtggaaaagaaacagaaccagtgctccaggtgttttttaattttttaatttatttttattttttttgtatatgtatatatatgtatgtatattttagaggaccagggtctcactatgttgcctaggccagactcaaactcctgtgctcaagcaatcctgcctcagcctcccaagtagctgggattacaggcatgcacaaacaatgcccagctctccaaatgttttctgtcactacctgaagtgttgcatcggtacttcctacggaaagaaaactaaatagaagtgtctctcccgtgagcccccaccactaccaccagaaaaaaaaaagagagaaaatgaactcatcagtctttagtttcctcaagttattctcccaaaaagacattcgccttggcacagataagccagctaatcttatgctttatgacccactgtgagctgttcctgacacagcttctgactttgtcagtgacaaaatttctcaccttttaaatgcagtgcttaacattttgttaggcccatactcaaaatcggccagatataaaatgacctcagattttgatctcctaggctcaaacaatcctcctacctcagcctcccaagtagctgggactataggcacaccaccatgcacagctaattttttttgtatttttctgcagagatggcgtttcgccatactgcccaggctagtctcaaaatcctgggctcaagcaatctgcccacctcagcctcccaaagtgctggaactacaggcaagagccactgcgcccagccacaacctcagatttctttggcaaacagaaatgtttaaaaacacaaaattttgctcaggtgaaacactgtgttactatcaaatctcacatccacataaagtttttcttttcggctttgtttcgtgaggaacagacagaacaaagtttttccaggtagcatctgtatcactattattctcctatttcctgtaccacccccacctccccaagccctactgaatgtgaggtttagaatgttttaaggagggtcaggtgcggtggctcacgcctgtaatcccagcactttgggaggccaaggcgggcggatcacctgagtttgggagttcgagaccagcctgaccaacatggagaaaccctgtctctactaaaaatacaaaattagccaggcgtggtggcacatgcctgtaatcccagctacttaggaggctgaggcaggagaatcgcttgaacccaggaggaggaggttgtggtgagccgagatcgtgccattgcactccagcctgggtgacagagtgagactccatctcgaaaaaaaaaatacaaaaattagctgggtgtggtggtgcacacctgtaatcccagctactcgggaggctgacgcaggagaattgcttgaacctgggaggtggaggttgcagtgagccgagatcgcgccattgcaatccagcctggacaacagagtgagactccatctcaaaaaaaaaaaaaaaaagaatgttttaaggaaaaaaatagtactgttacatataatcccaggtgataagaccacaatggaaatgtttaagtcctcactttaaagagtaccccactgagaagaggtatgttggactctagcagagatttggaaactctgggacactcaagatgtgaaagagcctggctatctgaggactcaaagagtcagcatcgggacttgtgagctcaagaagagaaaagggagtggtgaaactttgtcctaaaagttagcaccaggaacagaagaaaaaaacccgatatatagtgatacctcatcttttagagaatgggaagctatttttgtgttcacacagaaagtatagttcaaaaaacctctatatccagagttcagacaaggagaatgatttgagatataagtgccgatgaaggaggtcaattttgatctgaaaccagcagctggacctgggccacctcaggaaaaggactctgttctccaaggcagcacgactgaatggttctgagaataagccagggttcaggactcctgaccctttaggaccatggactcagaagagcctgaaggacaattgtgggctttaaacttctgagagcttgtaaagtaacacaagactgtgcctctcccttgccccagctgtagatagtctttgccccaccattgttatgaagatacacagggttttgcagtttgaataaattggatacaagtttcctctttttttttttctttttgagacaaagtctcgctctgtttccccaggctgagtgcagtggcacaatcaaggcttacttgccgcctcaacctcctgggctcaagcaacgagccatcctcccgtcttagcctcccaactagctgagactacaggcgtgggtcaccacacccagctaatttttgtactttttgtagagacagggtctcaccatgttgcccaggctggtcctgaactcctgggctcaagtaatctgcccacctcagcctcccaaagtgttggggttacaggcgtgaggcaccgcggctggcctgagtttcttcttaatactgtatcacaattgtgggctgtcttatgtgttgatatcgattgagctatttgaaataggaatgttaatgggtgtattaaatttttgtaaggatataacaatatctaccttccaaggatgttgtgaggttttccatgattttgtatatgagctaatgttacctttgaggggtggtgtgcattatgttggatgattgtaaattttcagtggaaaatgtaccgtgtcctaaatttaaagacatgaaaaatatcccaagatcatactagatcataatagcaattcctttacaaatgaattatggaggtaactgatctctaacagtttccttcatgttgttttaatgcacaagggcagaggatctgctgacccttggaaccagcgtgagctaaccacgtgctatagacacttcatggtgtcgcacccagggaagtcaaagcgctttgctccctcactgtctgtgagtcctcagccattagtaccccaccccccgctgctccaaaacttgagttatttcaaatgtttctcactgttcatctctccactgaccccactccagaaagcctggagagagtcccaagatgccacccaccttccccaatccctcgccacagatctgtgtctatctcacactctgtaagtgccgctttgcttcttcctctcttgaaaagactgagaacacacattttaacatgttaggaaaatggggcagcctaaaaaatgactgatcccaccgccagtgactcatgtatactccaggctagcagacaaggccctttttggtgggcctgcttctgtgggttcacagaaaccaaattactgtgggttgcaaagaattagcaggtcatttacaaagcagacatcccttcacccagactgtggttttgcatgctcaggttctcagtctatgagctttggtgcaggatcattttggctactggaaaaaccatagcttattttaaatttctggttgccaaagccaccacacgtgtggtctgtggatgaccattgtctgcagaatgacgaggaaggaacagaatgtggtttggggctcagggtggccttcccactgggagggaaggcgggagggagcccttgccctgggttttgacacagcctgtgctcacagcctctcctctcatctgcatttctcagaaatgccctccctgcccagtggtgactttccctcgtcactcctatggagttctacctggagcccagccatgtgtggaactgtgaagtttactcctctgtaaagatggtttaaagaaagtcagcttctgaaatgtaacaatgctaacccttgctggaaccctgtaagaaatagccctgctgatagttttctaggtttatcatgtttgatttttacactgaaaaataaaaaaatcctggtatgtttgaaa

SEQ ID NO: 28 - DNA, 4098 bp, Mus musculus
ttccacgcgatcagggttcctcaggcttgacattcaaaagtgggtgcggaacccgcggcactcggagcgtgctttaaagcggccgccagccagcgccgctctaacctcgcgccccggctgccggcggctcccgccctgcatctgcgccgacgcgaccgagcgatcccggggcctccctgcgcccggaatctcccgccagccgcgcgggtccccacggcagcagcacgtggagcggccgcggagcctgagcgacagctgcagcccgcgcggcccgcggcgacatggaagatatacaaccgactgtggagctgaaaagcacggaggagcagcctctgcccacagagagcccagacgctctgaatgactacagcttgcccaaacctcatgagatagaaaacgtggacagtagagaagccccagccaatgaagacgaagatgcaggagaagattcgatgaaagtgaaagatgaatacagcgacagagatgagaacattatgaagccggagcccatgggagatgcagaagagagtgaaatgccttacagctatgcaagagaatacagcgactatgaaagcattaagctggagagacacgtgccctatgacaacagcagaccaaccggtgggaagatgaactgcgacgtgtgcgggttatcctgcattagcttcaacgtcttgatggttcataagcgaagccataccggcgaacgcccgttccagtgtaatcagtgcggggcatcttttactcagaaaggtaacctcctccgtcatattaaactgcacacgggggaaaaaccttttaagtgtcacctctgcaactacgcatgccaaaggagagatgcgctcacgggacaccttaggacacattctgtggagaagccgtacaagtgtgagttctgcggaagaagctacaagcagagaagctccctggaggagcacaaggaacgctgccgagcttttcttcagaaccctgacctgggggacgctgcaagtgtggaggcaagacacatcaaagccgagatgggaagtgagagagctctcgtcctggacagattagcaagcaatgtggctaagcgaaaaagctcgatgcctcagaaattcatcggtgagaagcggcactgcttcgatgccaactacaatcccggctacatgtacgagaaggagaacgagatgatgcagacccggatgatggaccaagccatcaataacgccatcagctatctaggggctgaagcccttcgccccttagtccagactccgcctgctcccacctctgagatggtcccagtcatcagcagtgtgtaccccatagcacttactcgggccgatatgcccaatggggccccgcaggagatggaaaagaaacggatcctcctgccagagaagatcttgccttctgaacgaggtctgtcccccaataacagtgcccaggactccacagacaccgacagcaaccacgaggatcgccaacatctctaccagcaaagccacgtggtcctcccccaggcccgcaatgggatgcctcttctgaaggaggtccctcgctcttttgaactcctcaagccccctcccatctgcctgagggactccatcaaagtgatcaacaaagaaggggaggtgatggatgtgtttcgatgtgaccactgccacgtcctcttcctagattatgtgatgttcaccatccacatggggtgccatggtttccgtgatccctttgagtgtaacatgtgtggctatcgaagccacgatcgctatgagttctcctctcacatcgccagaggagagcacagagccatgttgaagtgagcatctgtcctcaatgcgagggtcaacattgttttttaaagctgatggtagccttatccagtagactgaactcaaacccacagtcccacccagttctgttagctctcaatcatgtccacatgaacagtcagggaaataactgtcttcattcagaaactgtttgcagagctatcatgttactctgtgaaacctcccttcccatcagttgagtttgatgggatttaaaagccaaagaagtatctggttcattatcttttgtagcaatagacagccatctactggagctcccggccaattggaacagtcctccgtgggccgcaggagtcattcactggaatacagcagccatatgtggaaaagccctttgatcctatgcccagatccacacagcggaagagccgcccagctgcccttgctcttgggcaccaacacaaggcagaagccttagcgtgtagtccccctcccttcacgttctaacagcaaggaagaggtggccgtacagtaaggacctgctgatagtaatggaagaccctattcttggagcatcaacccacttcctatcaaacaaaactgaagccacctgagcaaggacccttctcctttacctacggtcacaagggtgaacctcagatgatttggcctgagccagaccctgatcctcctgcctccgtctggaggaaaattctactccagaagaactgatgcgtttaaatattgggtatcattgaaaccgcagagtcatgcaccgaggggtggtatttgtctcctttgattgggattggtagagcaagtggtggggacatagtttaagggtacagtgcctgccaggtacaaccaccacaccagggggcaaagagggaagaaagaaaatggagggaagagaggttggctgagtagagcgtgtaaggttgactggttggcaacccttgtcaccttggtggagtactggctgaaaccccaaagaacatttgaaaagtgatttttggcaaaggagccaaaatgtgctgcttagttccccaccacctcccaccccacaggcgtgagatcagggacagaaccacagatgttctctcaggacggtgtgacatcacactctcctgggttagaaatgcatggtttttattatattactctattttcaaaacagggtctcctggtgccccggcaagcctcaaactcattgctggctgaagctgcccagcactctgagcctcctgcctcagcctacagagtgctgggttgagagggttatcagcaccgtggtgttgggcttctgctgtcctaaagaggagctgccacatcccactgagagtctgggaaggctgatcttgttctgtagatagaccaggtagtcacctttgtcacacaggcaccaagacatcaagattcttggaaaaatgctccataaacaagcactggaatcgaggccaccaaccgtgcttgccaagctcatatttgcaactaattggttaactctaaaagacaaatggcctcaaccataaaataagagtagcatccaaaccctcttgatggtcacttctggctttgatacatgattattcaatagccacaggttttttgcttttgtttttgtttttttaacttcttgatgtgtgggcattaagcagacaagtctgcgtgtgagaatgtgtttcatatgtctttgatgtgaaaatatagttgtgttaagtttttccttcagctactgagtcatgaccatgaaggaggcgatgaggggggactaaggacaggaagggttttgatcatgcagtcgtgtatacacgagctggcatctgcagcagccgtgtacacacgagctggcatctacagcttcttgccggagcccttttcttcctttccttttaaaagaacttgtgttagggaaaaataagttctgcttacagggacggcttcatggagcctatttacaaattaagagcttagcttttatgaacatttctgccagagccaggaatcccaaattcccggtatgttagcattttccttctaaggggcttatggattcggctcttactcattcatgtatacgatgccagtaattaaaacattcctcctcaaacacacaaatagcttagcctgccttgtagcatggtgtctgtctcagggtaaggggggtgggttatgagattccgtggcaacaattctgtggcaacaatcccgtgtcagcttaagggctaggtgtcttcagcacatctctgcatctcagcagaatcggatgtgagctgcaccacattgttcatgcccactgtgaaagaggacctgcctagtctag

SEQ ID NO: 29 - DNA, 4553 bp, Homo sapiens
agcaggctgccggctcggacggcggcgacgcggcaggacagggcggataagaaggagcggccggagactggagctgggttcgtggcgggccggctgatccggacggagactcaacctctgtatggcttttcaattttagttgaaattctctctccatggccttttggagtcccttgctgtatctccgaagtaaatttcaattactctacatttcatcaatgttggagtggcaagaaaaaggaggaacagatttggggtgaaaagttttgttttctaggtctaacaaactgtaaatgttaaaaaagcaaagatgggtgaaaaaaaaccagagcctttggacttcgtgaaagattttcaggaatacctgactcagcagacccatcacgtgaacatgatttctggatcagttagtggggacaaagaagcagaggctcttcagggagctggaacagatggtgatcaaaatggacttgatcacccatctgttgaagtttccttggatgaaaactcaggaatgttagtagacgggtttgaaaggacctttgatgggaagcttaagtgtcggtactgcaactatgccagcaaaggaacagcccggcttattgaacacatcagaatccacacaggtgaaaaacctcatcgatgtcatctttgtccatttgcatctgcttatgagcgtcatctggaagcccatatgcgttctcatactggagaaaaaccatacaaatgtgaattatgttccttccgctgcagtgatcgaagtaacttgtcccatcatcgaaggcgcaagcataaaatggtaccaattaaaggtactaggtcttccttaagcagcaagaaaatgtggggggttttacagaagaaaacaagcaatctgggctatagcagaagagcactaatcaacttaagtccaccttccatggtggttcagaaaccagactaccttaacgattttacccacgaaatcccaaatatccagactgactcctatgaaagtatggcaaaaaccacaccaactggtggccttccaagggacccccaagaactcatggttgataaccctttgaatcagctctcgactctagcagggcagttgtccagtctgccacccgaaaaccaaaaccctgcatcccctgatgtagttccctgccctgatgaaaagcctttcatgattcagcagccctctacccaagcagtagtttctgccgtatcagcaagtattcctcagagctcctctcccacaagcccagaacctcggccatcccatagtcaaaggaactatagtccagtggcaggtccaagcagtgagccaagtgcccacacgagcactcccagcataggaaacagccagccaagcaccccagccccagccctgccggtccaggaccctcagcttctgcaccactgccagcactgtgatatgtactttgcagacaacatcctttacactattcatatgggatgtcatgggtatgaaaatccttttcagtgtaatatatgtggatgcaaatgtaaaaacaagtatgattttgcctgtcattttgcaagagggcaacataaccaacattgattgaaaatagtcatattttacttagttttgctgtttttgtggtttggttttttttgttttttgttttggtcatccctaataaagtgtctgctaattcaaggcttatacattatatttatagaatataaatttgtcagtggaataaaatttcccctttttttcataaaaatctggtcagggtcatttatatattagaacagttagacacattggtgtctcttttttcctttcctttcgacattggagaattggagtgcagtcataatcttacaagatgttcatttgaatttctcacatttatggtccataaaaacttcaaggcttatccatacttttgatgtttcaatatgcattgaactggatgttatttctgccatatttcaaaatggtagaataaattacagaatttattactactcatttcagtgtttagtacagaaattgccttaaaaattgctattaattgaaatatcatttagttcacattccttaagttgaatcggtagtttcatttcaactgataatagtaaagctatttcagtttagtaaaaattttcttttttccacaagaggaaatgtaaaacagttaagacttgaaattgaaaattcttttaatatttaaaataacttgtttattcaatgtctaaacacatgtcagttttccactggatttttattttcacaggtaaatacactagagtgctagatgcctttttcccctgtcagtttgactttcatttaaatccttcattctgatatcattactgttaggtgaggtggataactataatgagaatctcttattcttcctattcctttgatgaccaaagagataaagtgaagtcatccttattattaaaatgcagcctccaaacaaatttctcaagattccttttccttccttctatccattttctgcccataatttccaagaaaaggtgggtatggaaacacatgagagaatgtgatagtgaagtcatatttacaaaactgagaacatgtccaaaatggattcttgtctcctcttcagaattagccatttaaaatattttctgtgacttcaaattgtaattcttatttgcagttttaccagtcttcatgtacagtgacgaaactgtgtagaaactaaatcatcgctttagtaagaaaggataaaaaggtgtggaagtgaatataaaatgcatctaaacatgacatttaatttgtttataaaaataagacttactaaatatagagtaattcattttgaataggaggctattgtttttatattgtgtaataactcacgtactctgaagagagcttggtcaaacaataaaatacattgttactaacttggtttcttttctgtgtactttgcaaaaattctatttttaattttgttcatatgttgaatgtgcccctaattggcatcttaaagagaatagtaagcatctattaaccaaaaaagaactctaatagtaaaggaaagggaaatattggtggtatgtacccacaaaacccccaagtgccaagttaatggaatctctgctttccctttcagatgctagaaagccactgtaatgagttcttgcagtttagcatccagtctaagctactgcattgtttaaagagcagcatcaaggacactttctccaaactggaactctcttctttgtcaaatcttgtactttaaaattctacaattctgttacattgttgtttaaatcacagactgctcagatccattttactgcagtagtttccaagtgtgtaacttggctttagtatttatcagttgccagaaagaaacaggttgtcatttggaagtttttgtggttattttttcccatttttattcttcagataaaagcagtaccccaaaatagaaaatgaaaattttcatgaaacaaagagaactcccttgttaaaaccagcttattaactctgtattctgtcaaatgcatttttttctaacaactgaccatggatgttgtgaaggtgcattttaatttaaacatggaaaagatttttttcataattacatactagaatgtaaaattataattttgccatgacttaaagagcacagttgatatcccaaaggttttgatgctaagaagctacagttattctaaatgcactaaaatgtttgaggcaaatctaccttagaggcttttttggtatggtattttttaaaatatttagattttatttaaatttcctgtgagttattctgtatttgaaaagatgttcgtgtcttcccctctgtattgaatgtttcactcattttatttttaatcaaatattttatagaaatgagttgttgggaagagtttaacatgcactatttatagtactttgccgttaacaggcaatgttctgaaactaaatttatttttgttcagtgaacataagtttagatttttaaagttggtagataatttatctccactaatatttttttaagaaactgtgaagagattaactgggaataattttatttcagattttactaatgtagtatgtagctacaacttcttgaacttcaagttaaggctagacatttactttgaaaaaattccactgggtgtttccagggctatttcattttagaaataagtgtttgccattcttctgcaaaaactggacaaggggaatactacaaaaaatactcagagataaaatcctcatttcaagttctacaaaatatttatcaaatgaatgttaatttttttttaattccctgctaaagacgttttcattagtcttagagggtatatgctttctagaacttgtttttgttaacatgtgctttgatgtaaagaacatattttgtatgcaaaacataacttgcattatggttgtacaatacactatattgtttagggattccggaaagcagtttaatgcagaaataactatatctagtatgcagttcatattgtgaatgaagctttgcttttgtaataaataaataagactttctaatgacaaaaaaaaa

SEQ ID NO: 30 - DNA, 4440 bp, Mus musculus
cagctgcgacgctgacgaacgctgaggcggtggcggtggcggcgacggcagtgaaaagggctgtggggattcctcggtgcggttgtcaggagatataacagactttcaacttaaagaaagaaagaaaaaaaaaagcaccaagatgggtgaaaagaaaccagagcctttggactttgtaaaagatttccaggaatacctgacccagcagacgcatcatgtgaacatgatttcgggatcagttagtggggacaaagaagcagagactcttcagggagctggaacagatggcgatcagaatggccttgatcacccgtctgttgaagtttccctggatgaaaactcaggaatgttagtagacgggtttgagaggaccttcgatgggaagctcaagtgtcggtactgcaactatgccagcaaaggaacggccaggctcattgagcacatcaggatccacacaggcgagaagcctcacaggtgtcacttatgtccatttgcatctgcttacgagcgccacctggaagcccatatgcgctctcacactggggaaaaaccatacaaatgtgaattgtgctccttccgctgcagtgaccgaagtaacctgtcccaccatcgaaggcgcaaacataaaatggtaccaattaaaggtaccaggtcttccttaagcagcaagaaaatgtggggggttttacaaaagaaaacaagcaatctggggtatagcagaagagcactaatcaacttaagtccaccttccatggtggtccagaaaccagactaccttaatgattttacacacgaaatcccaaatattcagactgactcctatgaagctatggctaaaactacaccaactggtggcctgccaagggacccccaagaactcatggttgacaaccctttgaatcagctctctactttagcaggacagttgtccagcttgccaccagaaaaccaaaaccctgcctctcctgatgtagatgcctgccctgatgaaaagcctttcatgattcagcaaccctctgcccaagcagttgtctctgctgtgtcagcaagtattcctcagagctcctcccccacaagtccggaaccgcggccatcgcatagtcagaggaactacagtccagtggcagggcccagcagtgaaccaagtgcccacaccagtactcccagcataggaaacagccagccaagcactccagctccaaccctgccggtgcaggatcctcagcttctacaccactgccagcactgtgacgtgtactttgcagacaacgtcctttacactgtccacatggggtgccatgggtatgacagcccctttcagtgtaacgtatgtgggtgcaaatgtaaagacaagtacgattttgcctgtcattttgcaagagggcaacacaaccagcactgaacacaatcaacattgttctcacttggtttggccttttgggagtttgtagtttggtttttagtttgatcatccctaataaggtatatgctaatttaaaatttatacattctagtctataaaatagaagtttgatagtgaaaaagggttttgttaacttttcataaaagtctggtcagggttatttatgcattagatagttagatgcattgctgtctttttcatttacacctatgagagttaaagtacagttgtaatcagtttcctacacttgtccataaggattcccagggttatctgtactcctgatgcttcaggatatacattacagctaatgttctttctgccatatgtcaaaatggcacacttctcataaaatcttttataacatttcagtgtttatagacaaactgcctttaagtttgctcctaattaaaatactattttcattcacataacttaagttgaattagtttcattacaaccaaaaccagtagttatttcagtgtaataaatatttctcttcaaggagaagcataaaacagttctttcaaatttaaattataaaatgaaaatcttggaagttgaaatttggggctaaattgaaaattgtgtttggtatttgagatatctgttcatttaatgtgtaaacatgttagtttttagcttttcatttatatatgtatttatatattttgagtattattttaaagccacaggtaaatatactagagtgctagatgccttttttcaatctgatctttcatgtaaattctgcattcacctgttactggtgtcaggtaagattgacagccacactgattattttattcttcccagtctttgatgaccaaagaggaagtgagcgcatcatcccactgagatgcttccagagcctccgaggctcctttcctgccctctgtgcataattgcccgtaatgtccacgaagggttggtctagaaacccagagacgtaataagtgaggaaacagctacaaacccagagcatgcccagacagagcctccttgcctctccagagcagagcaggccatggaaagtcctccctgtgagtctttatggacagagacggtgggatcgtcaccacagtgggaaaggataaatagagttattatgaagcacatttaaatgtgtcatttaacttacttagaattagagtggttcattttatataggaagccattatttttgtattgtgtaataagtagcttactctgaagagagcctggcaaaacaataaaacattgtcacttactattggtttctgtgtaccttgcaaatttttcatttttatttggttaatagcttgaaaatattctcaattggcttcttaaagaaataccctgtcaagcatttattaaccaaaaaatagcctcttacagtaaataagagggaactatgagagtgtctacccgtgaagccccaagtgccctggtaatggacctgctctccctccctgtgctggaaagccattccgagggtgcctgtgcgtacacagccagtcttggctcctcccaactgcactgttaaagcgcagccccgaagacactttctagtgattttggtctgttgcttaaatcatttactagattagttttagggcaacactttccaaatacataacttgactgttgtgcttatcagctgccagaaagaaacaggttattgttgggaaaccttgctgtttttaaagtttgattcaattaaaagcaatacttccaaatagacagtgaaaacttcccaaaagaggaagaagcccagtgtactgccgcacatctgtagtcccagctgcttgaagcagaggcgggaagagtgctagagcccaggaggtccctgccaggccgggcaacactgcaagacacagtctcaaaggccaagcagccgaaagggctctcctcacagccagctttatcacctctacgttctgggccaaacagtgacatgggcattttggttttcctaacaactaacgatgaatgttctgaaggtacatttcattttaaatacggaaaaaacttttcataattacagacaagaaggaaaaacccccgattttccaataatataaagagcacagttggtcttccaaaggtgtccatgtgtgaagctacaagtacttcgaaatgcactaaaattttgaagcaaatctaccttaggaacttttttgaaattgtacttttaaaggtttaggcttttgcttaaatattctgagttaaattctgtactaggaaagatgttggattctttccttaaatactaaataagttcctcatcttatttttaatcaaatgttttatagaaatgagttgagaaaagttgaccatgcactatttatagtactttgccataacaggcaatgttctaaaactaaatttatttttgtttagtgaacatgagtttagatttttaaagttggtaatttatctccactaatatttttttaaagaaactgtgaagagattaacagaataattttatttcagattttactaatgtagtgtgtagctacaacttgacattcaagtgaagactagacttctgttttgaaatttttccatcaggtagtttcagtggtttcattttagaaatgtgtttgcagatgtttgacaaagggaatattactaaaaaagaaaagcaaaaaaaaaaaaaacttatcaaatgaagaattgttagaaaaatttaaattccctgctaaagacatttttgttagtcttagggggtaggtatttgctttctagaacttgtttttgttgatagtatttgctttctagaacttgtttttgttgatagctggatgtatggaacctgttttctatgcgaggtgcaagtcttgcactacagttgtaagatatattatattgtttagggatcaagaaagcaatctatgctgaagtaactatagtatgtagttcctattgtgaataatgctttgcttttgtaatgtattaataaaaagtaacactttctaatgaaaaaaaaaaaaaaaaaaaaaaaaaaaaaaaaaaaaaaaaaaaaaaaaaaa

SEQ ID NO: 31 - DNA, 2008 bp, Homo sapiens
gagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagaggtaagagcccggcccaccctctccagatgccagtccccgagcgccctgcagccggccctgactctccgcggccgggcacccgcagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 32 - DNA, 1876 bp, Mus musculus
tgagataaaacccgtcccggcggctccggtaccagggtccagcctgtgacagcccatccaggctcggacgctggattaacacctgtgacatcacgtgtgcgctcccgctacatctgtagtcacatctacagccaccttggcagaggaatctacactgagttctgaacctaggtgaccacaagttctcaaacctcatctgtgaccctcaacaccctaatacctgggtcacacttgtgaaactgaaattcctacctgttaccaacacttgtaacgtttaaaacacatttgtgaaacttggtcacactatctgtggctacaacctaaaaccatagaggcacccaaggggccttatttgcaatagctgactgctccagtgactacaaggcatcacagagtagtagcatctactttgattctcccagactgcctgtgtagacggagcaatggctgaagtgaggggggtccagcgagtgctgtttggagactggctattgggggaggtcagcagcggccagtacgaggggctgcagtggctgaacgaggctcgcacagtcttccgcgtaccctggaagcatttcggtcgtagggatctggatgaagaagatgcacagatcttcaaggcctgggctgtggcccgagggaggtggccacctagtggagttaacctgccacccccagaggctgaggctgctgagcgaagagagcgaagaggctggaagaccaacttccgctgtgcactccacagcacagggcgttttatcttgcgccaagacaattcaggggatccagttgatccgcataaggtgtacgaacttagccgggagcttggatctactgtgggcccagccacggaaaatagggaagaagtgagcctcagcaatgctctgcccacacagggtgtgtccccaggatcatttctggcaagagaaaatgctgggctccaaaccccaagccctctgctttctagtgatgccggggacctcttgcttcaggttctgcagtacagccacatactggaatccgagtctggggcagaccccgtcccaccacaggctcctggccaggagcaagaccgtgtttacgaggaaccctatgcagcatggcaggtggaagctgtccccagtcccaggcctcaacagccagctctcaccgagcgcagccttgggttcctggatgtgaccatcatgtacaagggccgcacagtgctacaggcagtggtggggcaccccagatgcgtgttcctgtacagccccatggccccagcagtaagaacttcagagccccagccggtgatctttcccagtcctgctgagctcccagatcagaagcagctgcactacacagagacgcttctccagcatgtgtctcccggccttcagctggagcttcgaggaccgtcactgtgggccctgcgtatgggcaagtgcaaggtgtactgggaggtaggcagccctatgggcactaccggcccctccaccccaccccagctgctggagcgcaaccgccacacccccatcttcgacttcagcactttcttccgagaactggaggagtttcgggctcggaggcggcaagggtcaccacactacaccatctacctgggttttgggcaagacttgtcagcagggaggcccaaggagaagaccctgatcctggtgaagctggagccatgggtatgcaaggcatacctggagggcgtgcagcgtgagggtgtgtcctccctggacagcagcagtctcggcttgtgcttgtctagcaccaacagtctctacgaagacatcgaacacttcctcatggacctgggtcagtggccttgactcagaatcccaactcccaataaatagttcaaaatcagtggaaaaaaaaaaaaaaaaaa

SEQ ID NO: 33 - DNA, 7281 bp, Homo sapiens
gcagtcacagacacttgagcacacgcgtacacccagacatcttcgggctgctattggattgactttgaaggttctgtgtgggtcgccgtggctgcatgtttgaatcaggtggagaagcacttcaacgctggacgaagtaaagattattgttgttattttttttttctctctctctctctcttaagaaaggaaaatatcccaaggactaatctgatcgggtcttccttcatcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatgatcagattattacttactagtttttttttttttcttgcagtgtgtgtgtgtgctataccttaatggggaaggggggtcgatatgcattatatgtgccgtgtgtggaaaaaaaaaaagtcaggtactctgttttgtaaaagtacttttaaattgcctcagtgatacagtataaagataaacagaaatgctgagataagcttagcacttgagttgtacaacagaacacttgtacaaaatagattttaaggctaacttcttttcactgttgtgctcctttgcaaaatgtatgttacaatagatagtgtcatgttgcaggttcaacgttatttacatgtaaatagacaaaaggaaacatttgccaaaagcggcagatctttactgaaagagagagcagctgttatgcaacatatagaaaaatgtatagatgcttggacagacccggtaatgggtggccattggtaaatgttaggaacacaccaggtcacctgacatcccaagaatgctcacaaacctgcaggcatatcattggcgtatggcactcattaaaaaggatcagagaccattaaaagaggaccatacctattaaaaaaaaatgtggagttggagggctaacatatttaattaaataaataaataaatctgggtctgcatctcttattaaataaaaatataaaaatatgtacattacattttgcttattttcatataaaaggtaagacagagtttgcaaagcatttgtggctttttgtagtttacttaagccaaaatgtgtttttttccccttgatagcttcgctaatattttaaacagtcctgtaaaaaaccaaaaaggactttttgtatagaaagcactaccctaagccatgaagaactccatgctttgctaaccaagataactgttttctctttgtagaagttttgtttttgaaatgtgtatttctaattatataaaatattaagaatcttttaaaaaaatctgtgaaattaacatgcttgtgtatagctttctaatatatataatattatggtaatagcagaagttttgttatcttaatagcgggaggggggtatatttgtgcagttgcacatttgagtaactattttctttctgttttcttttactctgcttacattttataagtttaaggtcagctgtcaaaaggataacctgtggggttagaacatatcacattgcaacaccctaaattgtttttaatacattagcaatctattgggtcaactgacatccattgtatatactagtttctttcatgctatttttattttgttttttgcatttttatcaaatgcagggcccctttctgatctcaccatttcaccatgcatcttggaattcagtaagtgcatatcctaacttgcccatattctaaatcatctggttggttttcagcctagaatttgatacgctttttagaaatatgcccagaatagaaaagctatgttggggcacatgtcctgcaaatatggccctagaaacaagtgatatggaatttacttggtgaataagttataaattcccacagaagaaaaatgtgaaagactgggtgctagacaagaaggaagcaggtaaagggatagttgctttgtcatccgtttttaattattttaactgacccttgacaatcttgtcagcaatataggactgttgaacaatcccggtgtgtcaggacccccaaatgtcacttctgcataaagcatgtatgtcatctattttttcttcaataaagagatttaatagccatttcaagaaatcccataaagaacctctctatgtccctttttttaatttaaaaaaaatgactcttgtctaatattcgtctataagggattaattttcagaccctttaataagtgagtgccataagaaagtcaatatatattgtttaaaagatatttcagtctaggaaagattttccttctcttggaatgtgaagatctgtcgattcatctccaatcatatgcattgacatacacagcaaagaagatataggcagtaatatcaacactgctatatcatgtgtaggacatttcttatccattttttctcttttacttgcatagttgctatgtgtttctcattgtaaaaggctgccgctgggtggcagaagccaagagaccttattaactaggctatatttttcttaacttgatctgaaatccacaattagaccacaatgcacctttggttgtatccataaaggatgctagcctgccttgtactaatgttttatatattaaaaaaaaaaaatctatcaaccatttcatatatatcccactactcaaggtatccatggaacatgaaagaataacatttatgcagaggaaaaacaaaaacatccctgaaaatatacacactcatacacacacacgcacaggggaataaaataagaaaatcattttcctcaccatagacttgatcccatccttacaacccatccttctaacttgatgtgtataaaatatgcaaacatttcacaaatgttctttgtcatttcaaaatactttagtatatcaatatcagtagataccagtgggtgggaaagggtcattacatgaaaatatgaagaaatagccatattagttttttaacctgcaatttgcctcagcaacaaagaaaaagtgaatttttaatgctgaagataaagtaagctaaagtaccagcagaagccttggctatttatagcagttctgacaatagttttataagaacatgaagagaacagaatcacttgaaaatggatgccagtcatctcttgttcccactactgaattcttataaagtggtggcaagatagggaagggataatctgagaatttttaaaagatgatttaatgagaagaagcacaattttgattttgatgagtcactttctgtaaacaatcttggtctatctttacccttataccttatctgtaatttaccatttattgtatttgcaaagctagtatggtttttaatcacagtaaatcctttgtattccagactttagggcagagccctgagggagtattattttacataacccgtcctagagtaacattttaggcaacattcttcattgcaagtaaaagatccataagtggcattttacacggctgcgagtattgttatatctaatcctattttaaaagatttttggtaatatgaagcttgaatactggtaacagtgatgcaatatacgcaagctgcacaacctgtatattgtatgcattgctgcgtggaggctgtttatttcaacctttttaaaaattgtgttttttagtaaaatggcttattttttcccaaaggtggaatttagcattttgtaatgatgaatataaaaatacctgtcatccccagatcatttaaaagttaactaaagtgagaatgaaaaaacaaaattccaagacactttttaaaagaatgtctgccctcacacacttttatggatttgtttttcttacatacccatcttttaacttagagatagcattttttgccctctttattttgttgtttgtttctccagagagtaaacgctttgtagttctttctttaaaaaacattttttttaaagaagaagaagccacttgaaccctcaataaaggctgttgcctaagcatggcatacttcatctgttctcatttgtgccatctgccgtgatgtcgtcacttttatggcgttaatttcctgccactacagatcttttgaagattgctggaatactggtgtctgttagaatgcttcagactacagatgtaattaaaggcttttcttaatatgttttaaccaaagatgtggagcaatccaagccacatatcttctacatcaaatttttccattttggttattttcataatctggtattgcattttgccttccctgttcatacctcaaattgattcatacctcagtttaattcagagaggtcagttaagtgacggattctgttgtggtttgaatgcagtaccagtgttctcttcgagcaaagtagacctgggtcactgtaggcataggacttggattgcttcagatggtttgctgtatcatttttcttctttttcttttcctggggacttgtttccattaaatgagagtaattaaaatcgcttgtaaatgagggcatacaagcatttgcaacaaatattcaaatagaggctcacagcggcataagctggactttgtcgccactagatgacaagatgttataactaagttaaaccacatctgtgtatctcaagggacttaattcagctgtctgtagtgaataaaagtgggaaattttcaaaagtttctcctgctggaaataaggtataatttgtattttgcagacaattcagtaaagttactggctttcttagtgatgcagtgtccgtggtgcatttttttaaattaatgttttgctgtttaaatttattcaattttattgtgttttttaaaaagcagcttatgccacaagcacaaatgagctttctcagttttcaactcaaaatattatatgaaaagatggatatgtgtgtctgtgtgggaggggtgtgaattggatgacggttcttagggtattacagaattcagccagtggtggaagtctaaggttcttattgaatggttcaacttctttatcaaagaagttccatgaagatctttgataacctgtttaaatgtacagaatttttcaaaagccactgtgaaaagcagtaccataagttattttttaactaaaatctgtagaaagtgtttccaaaagagttccttttttcttcttcgaagtaatttctaaaagttgaaaacataggcttataaatctgaataaattagtgaattcatacattttagagactatcaaatatatccattattaacattcaataataatctctaaaggggaatgtccttaaaaagattgatagaaagattaatttaaaaactgaaaaggaaaacaagacaccaccagttagccagctggagaacttggacttttgcattagtagtgatatctgctccaaaaagaatgcagttcgaggaaatgttgcatgtggcttcaaaatagcagtgctttctgctttgttctcttgtattcattttcagcaaacatgtattaagcaattattatatgtaactcatgtggtgattccaggatgaatgaagttgtagccctttctcttaggaaacttataaactaataggagataaaaggcattctcagggaagtctacaaagtgctatgagggagccactttggccatcctggggcagagcagaccagaggctcagagcatggacacttcctcatccacccagcagaagcctttcacttgattttgaaggtgggcaggattgagaaaggcccagaaggggatgagttcaaagctgtttgtgggcatgtgacccctgcagtcacatatggccctagcttgtgtttcgtttagtgttctgctatcattatcctgataatatcctgataataaaattatccttaataatttta

SEQ ID NO: 34 - DNA, 6451 bp, Mus musculus
gagcagttctgtgttcttttgccagcactgacaaaggtctggttgtcaatgatacctttacagctaaatttactccagagtgacatgaacaggtgcaccctggcctgccagacacttgtgcagagggatcacgcatctcaccgcttgacgatcaagggggcaaagcttcggtgttcatagaaaaggagaggaggcgagcgcagcccaaactggggggtttctcttcaaagccagctggtctggctttattctacaggaatttttttacctgtcagagtttggacaacaaagccctcagcaggtgctgacgggaacaacttcctggagaagcagaaaggcactggtgccaacaaaagcattgaaaactgtgaagtaacctctggagagatgaagtgaagcgtggaaggccaatggctgtggcagtgaagaagtgcagagggaacgaatgcagggatttgggaactgagctgtgcgcgtgctgaagaaggaaacgtgtttgaaggaaacaggaaagagaaagaaaaggaaggaaaaaatacataatttcagggacgagagagagaagaaacacggggactatggggagaaaaaagattcagattacgaggataatggatgagcgtaacagacaggtgacttttacgaagaggaaatttggattgatgaagaaggcttatgagctgagcgtgctgtgcgactgtgagattgcactgatcatcttcaacagcaccaacaagctgttccagtacgccagcactgacatggataaggtgttgctcaagtacaccgagtacaacgagccgcacgagagccggacaaactcagacattgtggagacattgagaaagaagggcctcaatggctgtgacagcccagatcccgatgcagacgattcagtaggtcacagccctgagtctgaggacaagtacaggaaaattaacgaagatattgatctaatgatcagcaggcaaagattgtgtgctgttccacctcccagctttgagatgccagttaccatcccagtgtccagccataacagtttggtgtacagcaatcctgtcagcacactgggaaaccccaatcttctgccactggcccacccgtctctgcagaggaatagtatgtctcctggtgtaacacatagacctccaagtgcaggtaacacaggcggtctgatgggcggagatctgacatccggtgcaggcaccagcgcagggaatggatacggcaacccccggaactcaccaggcctgctggtctcacctggtaacctgaacaagaatatacaagccaaatctcctccccctatgaatctaggaatgaataatcgtaagccagatctccgcgttcttatcccacctggcagcaagaacacgatgccatcagtgtctgaggatgtggatctgctgttgaatcaaaggataaataactcccagtcggctcagtcattggctaccccggtggtttccgtagcaactcctactttaccaggacaaggaatgggaggatatccatcagccatttcaacaacatatggtactgagtactctctgagtagcgcagatctgtcatctctgtctggcttcaacactgccagtgcgctccacctcggctctgtaactggctggcagcagcagcacctacataacatgccgccatctgccctcagtcagttgggagcttgcactagcactcatttatctcagagttcaaatctctccctgccttctactcaaagcctcagcatcaagtcagaacctgtttctcctcctagagaccgtaccaccaccccttcgagatacccacaacacaccacgcgccacgaggcggggaggtctcctgttgacagcttgagcagctgtagcagttcctacgatgggagcgaccgagaggatcaccggaacgaattccactcccccattggactcaccagaccttcgccggacgaaagggaaagtccttcagtcaagcgcatgcgactctctgaaggatgggcaacatgatcacattattacttaatagtttttttttttcttgcagtgtgtgtgtgtgctataccttaatggggaagggggggtcgatatgcattatatgtgccgtgtgtggaaaaaaaaaaaaaagtcaggtactctgttttgtaaaagtacttttaaattgcctcagtgatacagtataaagataaacagaaatgctgagatacgcttagcacttgagttgtacaacagaacacttgtacaaaatagattttaaggctaacttcttttcactgttgtgaccctttgcaaaatgtatgttacaatagatagtgtcatgttgcaggttcaacgttatttacatgtaaatagacaaaaggaaacatttgccaaaagcggcagatctttactgaaagagagagcagctgttatgcaacatatagaaaaatgtatagaggtttggacagacccggcaagtggtgactaccggtaattgtaggaacacgcctgtcacctaacatccaagcacgctcacaaacctgcaggcatatcattggcgtatggcactcattcaaaaggatcagaaaccattcagagaggaccatacctaccttaaaagaaaagagaagaaaggaaaggaaaggaaagggaaaaaaaagtgtggcgtttgagagctaacatatttaattaaataaataaatctgggtctgcatctcttattaaataaaaatataaaaatatgtacattacattttgcttattttcatataaaaggtaagaaagagtttgcaaagcatttgtggctttttgtagtttacttaagccaaaatgtgtttttccctcaatagcttcgctaatattttaaacagtcctgtaaaaacccaccaaggactttttgtatagaaagcactaccctaagccatgaggatctccatgctttgctaaccaagataactgttttctctttgtagaagttttgtttttgaaatgtgtatttctaattatataaaatattaagaatcttttaaaaatctgtgaaattaacatgcttgtgtatagctttctaatatatataatattatggtaatggcagaagttttgttttcttaatagcgggaggggggtatatttgtgcagttgcacatttgagtaactattttctttctgatttcttttactctgcatacattttataagttcaaggtcagctgtcaaaaggataagctgtggggttagaacatattacattgcaacatcctaaattgtttttaaaacgtcagcaaactattgggtcaactgacatccattgtatatactaattggtttctttcaaactatttttttttttggtttggttttgaattgttggtttcttttcattttatcaaaagcagggcccctttcagatctctcttcatttcaccatacatcttggaattctgtaagtttgtatcctaacttgcccatattctaaatttacatagtttattttcagccttgagttggatgttttttagaaatatgcccagagtaagaagctgtgttggagcctaagtcctgcaagctgggtcctgggggcgaccgacctgctttacttgatggataagttacagagttctggaggagaacaaggtgaaagcctgggtgctggagaggaaggaaacagttaaagggtagttacttggtatccaccttcatgattttaactgacccataacaatgttgtcagcagtataggactgttgaacagcctggtgtgtcaggacccccaaatgtcacttctgcctaaagcatgtatgtcacctatttttttcttcaataaagaaatttaacagccatttcaagaaatcccactttaaaaaaaaaaaaaaacaaacctttctctatgtcccccccccttttttttaattttcaagataatttaactcttgtctaatattcttgtgtaagggattaattttcagacccctttaaaagtgagtgccataaagagtcaatatatagtgttgaagtggtatttcaggctaggaattattcccttctcttggaatgtgaagatctgtcgattcatcctgtcattaatcctgacatacagaacaaagaatgcagtaacattagccctgccaggtcacgtgtaggacgtgcttatccagttttttccccctttacctgcatagttgctacatacatgtttctcactgtaaaaggctgccgctgggtggcagaagccaaaagaccttattaacgaggctatatttttcttaacttgatctgcaatccagaattagaccacaatgcacctttggtggtatccatataggatgctagcctgccttgtagtaatgttttatatgttaaaaacaaaataaagacatcaaccatttcacatatattctactactaaaggtatcaatggaacatgaagacgagtatttaggcagaagcaaaacaggaaaccatccttacaaacatgcttacctgcacatctgtttgcatggtcatgtgtacattcaaacatgcacataaagtgaacaagaaagtcagccattatttcacgccagacttgattccatcctgagaattcatgtttataattaaatgtgtgttaaatatgcaagcatatcacaaatgttccttgtcctttcaaagtgcatttgttcatcaagatcaatagaatcttaccagtgggtaggaaaggatcatataaaatatgaagaaatagccatattaaattttttgctgtcatttgcctccgcaacaaagcgagtgtaagtgtctaatgctgaagctagagtgaactcatgtgccagcagaggcctcctctctttatagcaattctgacaataggtttgtttgtttgtttgtttgtttttacaaactcaagaaaaacagactcacttgaaaaaatggatgccagtcacctggttcccactactgtcgtagcagatagcgaagggacaatctgagacttgtagagatgatttaatgagaagcacaatttttattgtgaccagtcacgttctatcaccaggcactgtcttgtctttgcccttgccttttctgtaactcaccatttattgtatttacaaatctagtaagggttttgatcacaccaagttctttgtattgagaagtctagggcagagctctgggggattgttattttacattacctggcctagagtagcatattagacaacagtcatcattgcaagtaggaggaccgtaaatggcattttacatgactgcaagtattgttataccgaatcctattttaaaagagttttagtaacggtgagctgagaaattctggtcatggtgagcagcaggcgcaagccatgcagcctgtggactgtgcgcattgatatgtggaggctgttgatttcaaccttttttaaacttgtgttttttagtaaaattgcttatttttttcccaaaggtggaatttaatgttttgtagtgatgaatatgaaaatgcctgtcatctttagatcattgaaaggttaactaacatgagaatttcagagcaaattccaatactcttttaaaaagcgtgtctgccctctcttacttctatgtatttgtttatcgacacactcaccctttctgagatagcattttgctcccttgatccccccctttttttgtcccttcgaagagtaaatgctttgtatttctttctttaaaaaatttactttaaagaagaagccacttgaaccttcaataaaggctgttgcctaaacatggcgtacttcatctgttcccgcctctgccatctgctacggtgtcgtcagttgtatggcattaatttcctgccactagagatattttgaagattgcttgaatgctggtgtctgttagaatgcttctgactacagatgtaattaaaggcttttcttactatgttttaaccaaagatgtggagcaatccaagccgcatatctactgcattaaatttgtccatttggttattcataatcgggtattgcattttgccttccctgtccatacctcaaattgattgatacctcagtttaattcagagaggttcgctaagtgttggattctagttgtggtttgaatgcagcagtgtactcttgaagtaagcaggcaagggtcactgcaggcatgaagcttggattgcttcagatggtttgccgtatcgtttttcttccttttccttttctggggacttgtttccattcaatgacaattaaaatagcttgtaaatgagggcatacaagcatttgcaacaagtattcaaatagaggctcacagcggcataagctggactttgtcgccactagatgacaagatggtatacctaagctaaccacacgtgtgtatctcaagggacttaaattcagctgtctgtagtgaattcaagtgggaaattttcaaaagtttctcctgctggaaataaggtataatttgtattttgcagacaattcagtaaagttactggctttcttagtgat

SEQ ID NO: 35 - DNA, 3684 bp, Homo sapiens
acactttaatatcaacctgtttcctcctcctccttctcctcctcctccgtgacctcctcctcctctttctcctgagaaacttcgccccagcggtgcggagcgccgctgcgcagccggggagggacgcaggcaggcggcgggcagcgggaggcggcagcccggtgcggtccccgcggctctcggcggagccccgcgcccgccgcgccatggcccgaagaccccggcacagcatatatagcagtgacgaggatgatgaggactttgagatgtgtgaccatgactatgatgggctgcttcccaagtctggaaagcgtcacttggggaaaacaaggtggacccgggaagaggatgaaaaactgaagaagctggtggaacagaatggaacagatgactggaaagttattgccaattatctcccgaatcgaacagatgtgcagtgccagcaccgatggcagaaagtactaaaccctgagctcatcaagggtccttggaccaaagaagaagatcagagagtgatagagcttgtacagaaatacggtccgaaacgttggtctgttattgccaagcacttaaaggggagaattggaaaacaatgtagggagaggtggcataaccacttgaatccagaagttaagaaaacctcctggacagaagaggaagacagaattatttaccaggcacacaagagactggggaacagatgggcagaaatcgcaaagctactgcctggacgaactgataatgctatcaagaaccactggaattctacaatgcgtcggaaggtcgaacaggaaggttatctgcaggagtcttcaaaagccagccagccagcagtggccacaagcttccagaagaacagtcatttgatgggttttgctcaggctccgcctacagctcaactccctgccactggccagcccactgttaacaacgactattcctattaccacatttctgaagcacaaaatgtctccagtcatgttccataccctgtagcgttacatgtaaatatagtcaatgtccctcagccagctgccgcagccattcagagacactataatgatgaagaccctgagaaggaaaagcgaataaaggaattagaattgctcctaatgtcaaccgagaatgagctaaaaggacagcaggtgctaccaacacagaaccacacatgcagctaccccgggtggcacagcaccaccattgccgaccacaccagacctcatggagacagtgcacctgtttcctgtttgggagaacaccactccactccatctctgccagcggatcctggctccctacctgaagaaagcgcctcgccagcaaggtgcatgatcgtccaccagggcaccattctggataatgttaagaacctcttagaatttgcagaaacactccaatttatagattctgattcttcatcatggtgtgatctcagcagttttgaattctttgaagaagcagatttttcacctagccaacatcacacaggcaaagccctacagcttcagcaaagagagggcaatgggactaaacctgcaggagaacctagcccaagggtgaacaaacgtatgttgagtgagagttcacttgacccacccaaggtcttacctcctgcaaggcacagcacaattccactggtcatccttcgaaaaaaacggggccaggccagccccttagccactggagactgtagctccttcatatttgctgacgtcagcagttcaactcccaagcgttcccctgtcaaaagcctacccttctctccctcgcagttcttaaacacttccagtaaccatgaaaactcagacttggaaatgccttctttaacttccacccccctcattggtcacaaattgactgttacaacaccatttcatagagaccagactgtgaaaactcaaaaggaaaatactgtttttagaaccccagctatcaaaaggtcaatcttagaaagctctccaagaactcctacaccattcaaacatgcacttgcagctcaagaaattaaatacggtcccctgaagatgctacctcagacaccctctcatctagtagaagatctgcaggatgtgatcaaacaggaatctgatgaatctggaattgttgctgagtttcaagaaaatggaccacccttactgaagaaaatcaaacaagaggtggaatctccaactgataaatcaggaaacttcttctgctcacaccactgggaaggggacagtctgaatacccaactgttcacgcagacctcgcctgtggcagatgcaccgaatattcttacaagctccgttttaatggcaccagcatcagaagatgaagacaatgttctcaaagcatttacagtacctaaaaacaggtccctggcgagccccttgcagccttgtagcagtacctgggaacctgcatcctgtggaaagatggaggagcagatgacatcttccagtcaagctcgtaaatacgtgaatgcattctcagcccggacgctggtcatgtgagacatttccagaaaagcattatggttttcagaacacttcaagttgacttgggatatatcattcctcaacatgaaacttttcatgaatgggagaagaacctatttttgttgtggtacaacagttgagagcagcaccaagtgcatttagttgaatgaagtcttcttggatttcacccaactaaaaggatttttaaaaataaataacagtcttacctaaattattaggtaatgaattgtagccagttgttaatatcttaatgcagatttttttaaaaaaaacataaaatgatttatctgtattttaaaggatccaacagatcagtattttttcctgtgatgggttttttgaaatttgacacattaaaaggtactccagtatttcacttttctcgatcactaaacatatgcatatatttttaaaaatcagtaaaagcattactctaagtgtagacttaataccatgtgacatttaatccagattgtaaatgctcatttatggttaatgacattgaaggtacatttattgtaccaaaccattttatgagttttctgttagcttgctttaaaaattattactgtaagaaatagttttataaaaaattatatttttattcagtaatttaattttgtaaatgccaaatgaaaaacgttttttgctgctatggtcttagcctgtagacatgctgctagtatcagaggggcagtagagcttggacagaaagaaaagaaacttggtgttaggtaattgactatgcactagtatttcagactttttaattttatatatatatacattttttttccttctgcaatacatttgaaaacttgtttgggagactctgcattttttattgtggtttttttgttattgttggtttatacaagcatgcgttgcacttcttttttgggagatgtgtgttgttgatgttctatgttttgttttgagtgtagcctgactgttttataatttgggagttctgcatttgatccgcatcccctgtggtttctaagtgtatggtctcagaactgttgcatggatcctgtgtttgcaactggggagacagaaactgtggttgatagccagtcactgccttaagaacatttgatgcaagatggccagcactgaacttttgagatatgacggtgtacttactgccttgtagcaaaataaagatgtgcccttattttaccta

SEQ ID NO: 36 - DNA, 3777 bp, Mus musculus
gggagtgtccaaacctctttgtttgatggcatctgtttacagagttacactttaatatcaacctgtttcctcctcctccttctcctccttctcctcctcctcctcggtgacctccttctcctcccctttctccggagaaacttcgccccggcggtgcggagcgccgctgcgcagccgggggaggacgcaggcaaggcggagggcagcgggaggcggcaaccggtgcggtccccggggctcttggcggagccccggcccgcctcgccatggcccggagaccccgacacagcatctacagtagcgatgaagatgatgaagacattgagatgtgtgaccatgactacgatgggctgctgcccaaatctggaaagcgtcacttggggaaaactaggtggacaagggaagaggatgagaagctgaagaagctggtggaacagaacggaacagacgactggaaagtcattgccaattatctgcccaaccggacagatgtgcagtgccaacaccggtggcagaaagtgctgaaccctgaactcatcaaaggtccctggaccaaagaagaagatcagagagtcatagagcttgtccagaaatatggtccgaagcgttggtctgttattgccaagcacttaaaagggagaattggaaagcagtgtcgggagaggtggcacaaccatttgaatccagaagttaagaaaacctcctggacagaagaggaggacagaatcatttaccaggcacacaagcgtctggggaacagatgggcagagatcgcaaagctgctgcccggacggactgataatgctatcaagaaccactggaattccaccatgcgtcgcaaggtggaacaggaaggctacctgcaggagccttccaaagccagccagacgccagtggccacgagcttccagaagaacaatcatttgatggggtttgggcatgcctcacctccatctcagctctctccaagtggccagtcctccgtcaacagcgaatatccctattaccacatcgccgaagcacaaaacatctccagtcacgttccctatcctgtcgcattgcatgttaatatagtcaacgtccctcagccggctgcggcagccatccagagacactataacgacgaagaccctgagaaggaaaagcgaataaaggagctggagttgctcctgatgtcaacagagaacgagctgaagggacagcaggcattaccaacacagaaccacacttgcagctaccccgggtggcacagcacctccattgtggaccagaccagacctcatggggatagtgcacctgtttcctgtttgggagaacaccatgccaccccatctctgcctgcagatcccggctccctacctgaagaaagtgcctcaccagcaaggtgcatgatcgtccaccagggcaccattctggacaatgttaagaacctcttagaatttgcagaaacactccagtttatagattctgattcttcgtggtgtgatctcagcagttttgaattctctgaagaagcggcagctttttcacctagccagcagcccacaggcaaagccttccagcttcagcaaagagagggccatgggactagatctgcaggagagcctagcctgagggtgaccaggcgagtgctgagcgaggcatccctcggcccagactcaccccaagcgaggcacagcaaggttccgctggtcgtcctacgaaaaaggcggggccaggccagccccctagccgctggagagcctagcccctccctctttgctgacgtcatcagctcaactctcaagcgttcccctgtcaaaagcctacccttctctccctcgcagttcttgaacacttccagcaaccatgaaagctcgggcttagatgcacctaccttaccctccactcctctcattggtcacaaactgacaccatgtcgagaccagactgtgaaaacccagaaggaaaattccatctttagaactccagctatcaaaaggtcaatcctcgaaagctctcctcgaactcccacaccattcaaacatgcccttgcagctcaagaaattaaatacggtcccctgaagatgctacctcagaccccctcccatgcagtggaggacctacaagatgtgattaagcaggaatcggatgaatctggaattgtggctgagtttcaagagagtggaccaccgttactgaaaaaaatcaagcaggaggtggagtcgccaactgagaaatcgggaaacttcttctgctcaaaccactgggcagagaacagcctgagcacccagctgttctcgcaggcgtctcctgtggcagatgccccaaatattcttacaagctctgttttaatgacacctgtatcagaagatgaagacaatgtcctcaaagcctttaccgtacctaagaacaggcccctggtgggtcccttgcagccatgcagtggtgcctgggagccagcatcctgtgggaagacagaggaccagatgacggcctccggtccggctcggaaatacgtgaacgcgttctcagctcgaactctggtcatgtgagacatttccagaaaagcattatggttttcagaacacttaaaagttgactttcgacacatggctcctcagcgtggagcgctccatggctgagagaagagcctgattttgttgtggtacaacagttgagagcagcaccaagtgcatttttagttgcttgagatctcacttgatttcacacaactaaaaaggatttttttttttaaaaataataataatgaataacagtcttacctaaattattaggtaatgaattgtgaccatttgttaatatcataatcagattttttaaaaaaaataaaatgatttatttgtattttagaggatacaacagatcagtatttttgactgtggtgaatttaaaaaaaaaatttacacaaagaaatatcccagtattccatgtatctcagtcactaaacatacacagagagatttttaaaaaccaggagaagcattattttgaatgttagctaaatcccaagtaatacttaatgcaaccctctaggagctcatttgtggctaataatcttggaaatatctttattatactaaaccatttcatgaggagaattttgttgtcagcttgcttgaaaagttattactgtatgaaatagttttattgaaaaaattatatttttattcagtaatttaattttgtaaatgccaaatggagaaatgtgttcgctgctatggttttagcctgtagtcatgctgctagctagtgtcagggggcaatagagcttagatggaaaaaagagaaagagactcggtgttagataacggactatgcactagtattccagacttttttatttttatatatatgtaccttttccttttgtaattggaaaacttatttgggagaattttgcatttgttgtacatttttgttttttaggattttttttttttgttgttattgtcgatttataaaagcattgcacttctttttctttttttgggagatttgtgttgtttatgtcatatgttttgttttgagttcagcctgaatgttcatccgtttgggcgtttttctgacttggaagaacattctctgtaggtttctaagtgtacagagccggaactgcctcgtggttcctgggcttcagggaagacaaatatggaagtcaacagccagtttctgccttgagagcatttgcaagaatgctggccttgaattctgaaatgacagtgtatctactgccttgtagcaaaataaagctatcctcttattttacatacttcc

SEQ ID NO: 37 - DNA, 5540 bp, Homo sapiens
attcgcctcacaaacaaccacagaaccacaagtgcggtgcaaactttctccaggaggacagcaagaagtctctggtttttaaatggttaatctccgcaggtcactaccagccaccgagaccaacagagtcatttaaggctgcaagcagtatttacaacagagggtacaagttctatctgaaaaaaaaaggagggactatggcatcaaacagcctcttcagcacagtgacaccatgtcagcaaaacttcttttgggatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcggcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtggtagccctcggagaggtaccagatgggactgtggttactgtcatggcgggtaacgatgaaaattattctgctgagctccggaatgcctctgctgttatgaaaaaccaagtagcaaggttcaacgatctgagatttgtgggccggagtggacgaggcaagagtttcaccttgaccataaccgtcttcacaaatcctccccaagtagctacctatcacagagcaattaaagttacagtagatggacctcgggaacccagaaggcacagacagaagcttgatgactctaaacctagtttgttctctgaccgcctcagtgatttagggcgcattcctcatcccagtatgagagtaggtgtcccgcctcagaacccacggccctccctgaactctgcaccaagtccttttaatccacaaggacagagtcagattacagaccccaggcaggcacagtcttccccgccgtggtcctatgaccagtcttacccctcctacctgagccagatgacgtccccgtccatccactctaccaccccgctgtcttccacacggggcactgggcttcctgccatcaccgatgtgcctaggcgcatttcagatgatgacactgccacctctgacttctgcctctggccttccactctcagtaagaagagccaggcaggtgcttcagaactgggccctttttcagaccccaggcagttcccaagcatttcatccctcactgagagccgcttctccaacccacgaatgcactatccagccacctttacttacaccccgccagtcacctcaggcatgtccctcggtatgtccgccaccactcactaccacacctacctgccaccaccctaccccggctcttcccaaagccagagtggacccttccagaccagcagcactccatatctctactatggcacttcgtcaggatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatattgaaattcctcagcagtggcccagtggtatctgggggccacatcccacacgtatcaatatatacatatatagagagagtgcatatatatgtatatcgattagctatctacaaagtgcctattttttagaagatttttcattcactcactcagtcatgatcttgcagccataagagggtagatattgagaagcagaaggctcaagagagacaattgcaatcgagcttcagattgtttactatttaagatgtacttttacaaaggaacaaagaagggaaaaggtatttttgtttttgttgtttggtctgttatcatcaataacctgttcatatgccaattcagagaggtggactccaggttcaggagggagaagagcaaagccgcttcctctctgtgctttgaaacttcacaccctcacggtggcagctgtgtatggaccagtgccctccgcagacagctcacaaaaccagttgaggtgcactaaagggacatgaggtagaatggatgcttccatcacagtaccatcattcagaataactcttccaatttctgctttcagacatgctgcaggtcctcatctgaactgttgggttcgtttttttttttttttttcctgctccaagaaagtgacttcaaaaataactgatcaggatagattattttattttactttttaacactccttctccccttttcccactgaaccaaaaagaaatcccatccctaaaacctgccttctccttttatgcaaaactgaaaatggcaatacattattatagccataatggtatagatagtgattgcgtttggctatgtgttgttttctttttttttaaattatgaatatgtgtaaaatctgaggtaacttgctaacgtgaatggtcatataactttaaagatatatttataattatttaatgacatttggacccttgaaacatttcttagtgtattgatatgttgacttcggtctctaaaagtgctctttattaaataacaaatttcttcagtggtctagagccatatctgaaatattgctaagcaatttcagttcatccaggcacaatgtgattttaaaaaatacttccatctccaaatattttagatatagattgtttttgtgatgtatgaaggaaatgttatgtttagttctttcagatctttgaatgcctctaacacagctttgccttctaaagcggtaattagggatttaaaaaacaacctttagccctttatcagcatgaaatgctggagtgatgtggttttctaatttctttggggtaattatgactcttgtcatattaaaaagacaagcacaagtaaatcattgaactacagaaaaatgttctgtggtttcatagttaagcaaaactctaaatcgccaggcttcatagcaaagacatagtcagctaaaagccgcacatgtggatagagggttcaattatgagacacctagtacaggagagcaaaattgcaccagagattcttaaccaaccagccttaccaaacaacacaacaggggaaccccaatctgccttacccaaggccccactggcagctttccacagaatttgcatttagaggagcagaatgacatcactgtcctttgggagtaggtcctctgaaaaggcagcaggttccagcaggtagctgagctgagaggacatatggcccacggggacctacagacagcctttgacatttgtatttcttacaatggagggccaaggagggcaaggggctgtggagtttggtgtctactagtgtgtatgaatttgagctagagtccttctgtggcatgcactttgaccactcctggcagtcacatggcagatttccaagtgcaaatccttaatccaaacaaggatcatctaatgacaccaccaggccaatccctgctctcctccccgaaaagtcagggtcccttcattggaatcctccacccacccaagcagaatttagcagagatttgccttcaaaccctaacggcccccttgttctctggtccttctcaaacccacctttgtaggccacccagcattgcaggacagcgtgtggggcagctggacctgtgcttcctgcctgggagtctcccttggaattcatcctgactccttctaataaaaatggatgggaaagcaaaacactttgccttctaaaggccgtataccaagtatgcttagataaataagccacttttctattacttaagtaagaaggaagtagtaattgatactatttattgtttgtgtgtggtagcttgaagcacaccactgtccatttatttgtaagtgtaaaatatgtgtgtttgtttcagcagcacttaaaaaagccagtgtctggttacacatttcaattttaattaattgacataaaaatgctaccgccagtgccagctgcatcctatttaattaaaaaggtactatatttgtacattattttttaatgttaaaagggcttttttaagtttacagtacacataccgagtgactttagggatgcttttgtgttgaaatgttactatagtggctgcaggcagcaacccagaaacactttagaagctttttttccttgggaaaaattcaagcacttcttccctccaccctcactccaaccaccccaatgggggtaattcacatttcttagaacaaattctgcccttttttggtctagggattaaaattttgtttttctttctttctttttttttttttttcactgaacccttaatttgcactgggtcatgtgtttgatttgtgatttcaagaccaaagcaaagtcttactactactgtggaaccatgtactagttcctgggaattaaaatagcgtggttctctttgtagcacaaacattgctggaatttgcagtcttttcaatgcagccacatttttatccatttcagttgtctcacaaattttaacccatatcagagttccagaacaggtaccacagctttggttttagattagtggaataacattcagcccagaactgagaaactcaacagattaactatcgtttgctctttagacggtctcactgcctctcacttgccagagccctttcaaaatgagcagagaagtccacaccattagggaccatctgtgataaattcagaagggaggagatgtgtgtacagctttaaggattccctcaattccgaggaaagggactggcccagaatccaggttaatacatggaaacacgaagcattagcaaaagtaataattatacctatggtatttgaaagaacaataataaaagacacttcttccaaaccttgaatttgttgtttttagaaaacgaatgcatttaaaaatattttctatgtgagaattttttagatgtgtgtttacttcatgtttacaaataactgtttgctttttaatgcagtactttgaaatatatcagccaaaaccataacttacaataatttcttaggtattctgaataaaattccatttcttttggatatgctttaccattcttaggtttctgtggaacaaaaatatttgtagcattttgtgtaaatacaagctttcatttttattttttccaattgctattgcccaagaattgctttccatgcacatattgtaaaaattccgctttgtgccacaggtcatgattgtggatgagtttactcttaacttcaaagggactatttgtattgtatgttgcaactgtaaattgaattatttggcatttttctcatgattgtaatattaatttgaagtttgaatttaattttcaataaaatggcttttttggttttgtta

SEQ ID NO: 38 - DNA, 5740 bp, Mus musculus
atgcttcattcgcctcacaaacaaccacagaaccacaagtgcggtgcaaactttctccaggaagactgcaagaaggctctggcgtttaaatggttaatctctgcaggtcactaccagccaccgagaccaaccgagtcagtgagtgctctaaccacagtccatgcaggaatatttaaggctgcaagcagtatttacaacagagggcacaagttctatctggaaaaaaaaggagggactatggcgtcaaacagcctcttcagcgcagtgacaccgtgtcagcaaagcttcttttgggatccgagcaccagccggcgcttcagccccccctccagcagcctgcagcccggcaagatgagcgacgtgagcccggtggtggctgcgcagcagcagcaacagcagcagcagcagcagcaacagcagcagcaacaacagcaacagcaacaacagcagcagcagcagcagcagcaggaggcggccgcagcagcagcggcggcagcggcggcggcagcagcggcggcggccgcagtgccccgattgaggccgccgcacgacaaccgcaccatggtggagatcatcgcggaccacccggccgaactggtccgcaccgacagtcccaacttcctgtgctccgtgctgccctcgcactggcggtgcaacaagaccctgcccgtggccttcaaggttgtagccctcggagaggtaccagatgggactgtggttaccgtcatggccgggaatgatgagaactactccgccgagctccgaaatgcctccgctgttatgaaaaaccaagtagccaggttcaacgatctgagatttgtgggccggagcggacgaggcaagagtttcaccttgaccataacagtcttcacaaatcctccccaagtggccacttaccacagagctattaaagtgacagtggacggtccccgggaaccaagaaggcacagacagaagcttgatgactctaaacctagtttgttctctgatcgcctcagtgatttagggcgcattcctcatcccagtatgagagtaggtgtcccgcctcagaacccacggccctccctgaactctgcaccaagtccttttaatccacaaggacagagtcagattacagatcccaggcaggcacagtcttccccaccgtggtcctatgaccagtcttacccctcctatctgagccagatgacatccccatccatccactccaccacgccgctgtcttccacacggggcaccgggctacctgccatcactgacgtgcccaggcgtatttcagatgatgacactgccacctctgacttctgcctctggccttcctctctcagtaagaagagccaggcaggtgcttcagaactgggccctttttcagaccccaggcagttcccaagcatttcatccctcactgagagccgcttctccaacccacgaatgcactacccagccacctttacctacaccccgccagtcacgtcaggcatgtccctcggcatgtccgccaccactcactaccacacgtacctgccaccaccctaccccggctcttcccaaagccagagtggacccttccagaccagcagcactccatatctctactatggtacttcgtcagcatcctatcagttcccaatggtacccgggggagaccggtctccttccaggatggtcccaccatgcaccaccacctcgaatggcagcacgctattaaatccaaatttgcctaaccagaatgatggtgttgacgctgacggaagccacagcagttccccaactgttttgaattctagcggcagaatggatgagtctgtttggcggccatattgaaattcgtcaaccatggcccagtggcatgggggccacatcccgcatgtgttaatatatacatatataaagagagtgcctatatatgtatattgattagctaactagaagatttctcattcaatccctagtcatgatcttgcaaccctaagagggtgggggcagtcataactgggtttcatattgtttactatttaagatgtcccctttaccaaggaacaaaccgtcaaaggtgttgtctggtctgttttcataagtgacctgttcccacgccggttcagagaggtggactctgggtctgggaggaaggagagacacttcctctctgtgctttgaaaccacagcctctgctgtgtggcagccggtacactctgcagacccgcttacagagtcagatgtggtgcactcagaaagggacaagaggcagagtggctgcttctgtccgctgccgtccactctgccgtccacctgttccaaagttttccttcagacttgctgcaggtactcatttgaacttttgagttcactttttttttttcctattctaagaaagtgacttcaaaaatactgatcaggacagataattttattttaccttttatattttctcacttcccccatttaaccaaaaagaaatcccgttccccctcccccgttccttctgcttctccctttatgcaaactgaaaatggcaatgccttattattatagccataatggtatagtgtttgagttggctgtgtgttatgtgtttttttctttttttttcttttttaaattatgaatatgtgtaaaatctgaagtaacttgctaacgtgaatggtcatataactttaaagatatatttataattatttaatgacatttggacatttggaacatttcttagtgtaatgatatgttgacttcggtctctaaaagtgtgcttcttcttcaataccaagtttcttcagtgggctagagccatatcggaaatattgctaagcaatctcaattccttcaggcataatgtgatttttttttttttttgaagataactcccatctccaaatagtttagatgtagtttgttttcacgatgtatgaaggagatgctctgtttctttctttcaggcatttgattgcctctgacacagctttgccttttaaagcaataattagggattaaaataacaaaaacaaaacaaaagccacctatagccctttaacacttaacgtggcccctttactagcatgaaatgctggagacatgtggtttcctaatttctccattttgggggtggtgggaggggggagggtggccattatgactcttatcatattaaaaagccaagcacaagtgattggttgaactgcagaaaagtgttctgtggtctctgagttgagcaaaactctaaattgcaggcttcgtggttgagggcctagtcagctgaaagccacgcgtgtggtaaaggctcaggcatggcttggagaacctaggaacacattaggagcctgcacctaccagcctcaccatacagccattcaggggaacccaaaaagtgccttacccaaggagggcccccagcagctttccaggaagtcgaatgaagtcgctgtcctcggggaactggtcagctgaagtagcaaccggtagctgatgtcagtagacaacagaacctgtggggacctccaggaaacctttgacattggaggctttcattaggcagggccaacaagagcagggaaggccatgtacccattggtatctgccattgtgtgtgagtttgagatccagccctccttggaggatgtactgtgatcattcctggtaccttagggccaatccctaagtgtggcttcctaatccaggccagggatcattcagtgataccaccaggccaatcccagcattcctcctgcacaaagtgtttgtgtgtggggggatagattggggtggggccaccttcatttgaatcctgagtcattctaagagttctgcaagcttttgccttcagcaccctataccccctcgctctctgttccttctcaggttgacctttgtcccaatgcgggacagtccagaggcagatggggacctatgtgtgcctccaacctgcgttttcctcagaattcatcctgactccctctgacacagattgaggggggggggaagaaacccaacccgcaccaaagcaaaacactttgccttctaaaggctgtgcaccaagtagacgcagatggtcagcccacctttgtgtttccttaagatggaaattgtaactgatgctatttattgtttgtgtgtggtagcttgaagcacaccacggtccatgtgtttgtctgatacctatttcagcagcatttacaaaagccagtgtctggttacacttttcagttttcattaatcaacatgaaaatgttaccattggtgccagctgcaccctatttaatttttttaagggcactatatttgtacatttcgtttttaatgttaaagggcttcttaaagtttacagtacagttatcaagggaatagaggggatgcattagtgcctaaatgttattctagtggctgcaggcagcaacccagaagcagttttgaaaacaggttgtttccctctgtcctcccttatttgggaaaattcaagtgctttcttcacctttcaggcacctcacggtgactcccgttacttagagcagtctgtcgtcgtcttcttcttcttcctcttcctcttcctcttcctcttcctcttcctcttcctcttcctcttcctcttcctcttcttcttcttcttcttcttcttcttcttcttcttcttcctcctagggtttaaaatcccccttcctcttcccttatctcttaaactcttcatttgcactgggtcacacgtatgatttgtggttttaagaccaaagcaatgtcttattactcttctggagccgtttatgtgtactaaccaacccttccctccacttccctgggtttagatgcacacggttctcaaaggagcacaaacatggccagattcacagtgggacccacacagccatgttaaaaaaaaaaacaaaaaaaaacctcttcacttgtctgagaattttaacctgggccccaattgctaacgggcaccatggcttgggtttcaggttagggaacgttgcccagtgagtaacagaaagacttaactgatttaattagtttgccctcatccttcactccaagaccctaagaaaccgatcagagaaacatctccacaccattagaggttgagaagggaggagccatgtgggggttccctccgctcggaggaaaggcactgactgacctagttagagtggtagcagaagcacccatggtatctgagagagcaataaacaataaaagatgatcctcccaagcttagaatttgttgttcttaaagaagacgatgcatctaaaacaaagaatttttttttctatgtgggaactttcttcgttgtctgtttacttcatgtttacaaataattgtttgctttttaatgcagtactttaaaaaatatatcagccaaaaccataacttacagtaatttttttaggtattctgaataaaattccatgtcttttgatatgtctactgttcttaggtttctgtggaacaacaacaacaacaacaacaaaattgtagcattttgtgtaaatacagctttcgttcttattttttatttttctgattgctattgcccaagatttgctttctatgcacgtattgtacaaattgtgctttgtgccacaggtcatgatcgtggatgagtttactctgaacttcaaagggactatttgtattgtatgttgcaactgtaaattgaattatttggcatttccccctctcatgattgtaatattaatttgaagtttgaatttaattttcaataaaaaggctttttttttccttttggttttg

SEQ ID NO: 39 - DNA, 4978 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 40 - DNA, 4520 bp, Mus musculus
aattatgcatggaggcgtgtcttggccagtggcggctgggtggggattggctggaggggctgtaattcagcggtttccggagctgcagtgtagacagggagggggaacctggggttccgacgtcgcggcggagggaacgagccctaaccggatcgctgaggtacaaccccgctcggtgtcgcctgaccgcgtcggctaggagaggccaggcggccctcgggagcccagcagctcgcgcctggagtcagcgcaggccggccagtcgggcctcagccccggagacagtcgagacccctgactgcagcaggatggctcagtggaaccagctgcagcagctggacacacgctacctggagcagctgcaccagctgtacagcgacagcttccccatggagctgcggcagttcctggcaccttggattgagagtcaagactgggcatatgcagccagcaaagagtcacatgccacgttggtgtttcataatctcttgggtgaaattgaccagcaatatagccgattcctgcaagagtccaatgtcctctatcagcacaaccttcgaagaatcaagcagtttctgcagagcaggtatcttgagaagccaatggaaattgcccggatcgtggcccgatgcctgtgggaagagtctcgcctcctccagacggcagccacggcagcccagcaagggggccaggccaaccacccaacagccgccgtagtgacagagaagcagcagatgttggagcagcatcttcaggatgtccggaagcgagtgcaggatctagaacagaaaatgaaggtggtggagaacctccaggacgactttgatttcaactacaaaaccctcaagagccaaggagacatgcaggatctgaatggaaacaaccagtctgtgaccagacagaagatgcagcagctggaacagatgctcacagccctggaccagatgcggagaagcattgtgagtgagctggcggggctcttgtcagcaatggagtacgtgcagaagacactgactgatgaagagctggctgactggaagaggcggcagcagatcgcgtgcatcggaggccctcccaacatctgcctggaccgtctggaaaactggataacttcattagcagaatctcaacttcagacccgccaacaaattaagaaactggaggagctgcagcagaaagtgtcctacaagggcgaccctatcgtgcagcaccggcccatgctggaggagaggatcgtggagctgttcagaaacttaatgaagagtgccttcgtggtggagcggcagccctgcatgcccatgcacccggaccggcccttagtcatcaagactggtgtccagtttaccacgaaagtcaggttgctggtcaaatttcctgagttgaattatcagcttaaaattaaagtgtgcattgataaagactctggggatgttgctgccctcagagggtctcggaaatttaacattctgggcacgaacacaaaagtgatgaacatggaggagtctaacaacggcagcctgtctgcagagttcaagcacctgacccttagggagcagagatgtgggaatggaggccgtgccaattgtgatgcctccttgatcgtgactgaggagctgcacctgatcaccttcgagactgaggtgtaccaccaaggcctcaagattgacctagagacccactccttgccagttgtggtgatctccaacatctgtcagatgccaaatgcttgggcatcaatcctgtggtataacatgctgaccaataaccccaagaacgtgaacttcttcactaagccgccaattggaacctgggaccaagtggccgaggtgctcagctggcagttctcgtccaccaccaagcgggggctgagcatcgagcagctgacaacgctggctgagaagctcctagggcctggtgtgaactactcagggtgtcagatcacatgggctaaattctgcaaagaaaacatggctggcaagggcttctccttctgggtctggctagacaatatcatcgaccttgtgaaaaagtatatcttggccctttggaatgaagggtacatcatgggtttcatcagcaaggagcgggagcgggccatcctaagcacaaagcccccgggcaccttcctactgcgcttcagcgagagcagcaaagaaggaggggtcactttcacttgggtggaaaaggacatcagtggcaagacccagatccagtctgtagagccatacaccaagcagcagctgaacaacatgtcatttgctgaaatcatcatgggctataagatcatggatgcgaccaacatcctggtgtctccacttgtctacctctaccccgacattcccaaggaggaggcatttggaaagtactgtaggcccgagagccaggagcaccccgaagccgacccaggtagtgctgccccgtacctgaagaccaagttcatctgtgtgacaccaacgacctgcagcaataccattgacctgccgatgtccccccgcactttagattcattgatgcagtttggaaataacggtgaaggtgctgagccctcagcaggagggcagtttgagtcgctcacgtttgacatggatctgacctcggagtgtgctacctcccccatgtgaggagctgaaaccagaagctgcagagacgtgacttgagacacctgccccgtgctccacccctaagcagccgaaccccatatcgtctgaaactcctaactttgtggttccagatttttttttttaatttcctacttctgctatctttgggcaatctgggcactttttaaaatagagaaatgagtgagtgtgggtgataaactgttatgtaaagaggagagcacctctgagtctggggatggggctgagagcagaagggagcaaggggaacacctcctgtcctgcccgcctgccctcctttttcagcagctcggggttggttgttagacaagtgcctcctggtgcccatggcatcctgttgccccactctgtgagctgataccccaggctgggaactcctggctctgcactttcaaccttgctaatatccacatagaagctaggactaagcccagaggttcctctttaaattaaaaaaaaaaaaaataagaattaaagggcaaaacacactgacacagcatagcctttccatatcaaggaatactcagttaacagcctctccagcgctgtcttcaggctgatcatctatataaaccctggaatggttgcagatcaaatctgtaaaagagatccgagagctgtggcttggcctctggttcaaacacaaaggctagagagaacctagatatccctgggttttgtttacccagtatgcttgtcggttggaggtgtgaggtaggccaagggcactggaaagcctttgtcatcaccctactccctccccaacccagactccagaccctgtttcagggtcagcctgccctgtgggtgccttactgggcctagggtcaacctgccttcctttcccacttgaccttgctggtagtatgtccccttcccatgtccaaaggccctctgtcctgcttctattgggaatccctgcctcaggaccttgtgtcgagagggattgccttacaggtttgaacctgcctcagactacaggccctcagcaaagctcagggagtatggtccttattctatgcgcttggttcccagggatatctgtaaccacagggcaaaagctgacatatactccaggtctgccctcatatgagtggtgtattcttggcctcccctgagactggcaactgtctgctccccattgggtctcccaggtgaggtggaacacagttcctgcacctactgtggcctccatgtcgcttgcttgcttcgctcactcagcttactggaacactgagtgttcaaggcaagcctttcctgacagaggcatggctagattcagtgactcaaagccacctcattcagctgatcagtgtctgtggaattgtttccttccagttaaccagtgtctgaattaagggcagtgaggacattgtctccaagacgaactgcttgccttgaccaccccagccttctgcttcgagacagttactgctctcccaccccatcaatgttctttagttatacaataagctgaacttataaactgaaagggtatttaggaaggcaaggcttgggcatttttatggctttcaatcctggggacccaggaacaaggtgagggcttctctggggctggtgttgtacctcaggggctctgggaagtctgtgtgcctgggttaaccacccatagtgagcccctggaactgcccactttccctctccttggccccacttggccccagcctcacccagcctgcagactgcttagcctttcagtgcagtggcttgtgttctggccactgcactcagattccaatgtaaactttctagtgtaaaaatttatattattgtgggttgttttttgttgttgtttgtttttgtatattgctgtaactactttaacttccagaaataaagattatataggaactgtctggc

SEQ ID NO: 41 - DNA, 8232 bp, Homo sapiens
gatgtctacagtatcacctttcttgtagattggcacttatgcggccaaaggaacaacaccatgttttctaaaaggctggagaaaataccacaggtgcctctcctctttcccttcatcttcatcattttgacaaattactcaaagatggagggtgcagttgaaagccagccatcattttttaaaacttctcaggacattgtaacatgcacttgggttgagaactgctactcgagcttctccaggaggcccttggagcaaatgttttgtaaacaccaatctaagaacattatctcttggacgggcatggtggctcacacctgtaatcccagcactttgggaggccaaggtttgtgtgattttgctaaaatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtacagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaaaagggtccaagttgccacattgcttcattaaaacaagagaccacttccttaacagctgtattatcttaaacccacataaacacttctccttaacccccatttttgtaatataagacaagtctgagtagttatgaatcgcagacgcaagaggtttcagcattcccaattatcaaaaaacagaaaaacaaaaaaaagaaagaaaaaagtgcaacttgagggacgactttctttaacatatcattcagaatgtgcaaagcagtatgtacaggctgagacacagcccagagactgaacggcaatctttccacactgtggaacaatgcatttgtgcctaaacttcttttggaaaaaaaaaatataattaatttgtaagtctgaaaaaaaaatatttaatttaaaaaaaattgtaaacttgcaataatgaaaaagtgtacttctgaagaaaactacatgaacgtttttgttggtattcaagtcagctagtgtttataattactggatattgaattaggggaagctcggctgccctagtaacaaaaccagcaaacgtcctgatgacaacgaagtgatgacattagccattccttagggtaggaggaacagatggatcttatagacctatgacaaatatatatataaatatatatataaatatatattaaaaatttagtgactatggtaagcttttgttcatttgtttcagacttttttctcctgtaaaaaaatagtactgattaacttttttaaaagaaagattttactgtaaatatggatttttttttttttggtcttatttctgtccctttccctggtttgttatcgtaacctgtagtgccaactctgcttccagaggggtagtgcaggatgaaatgctgaccctgatgttgcttctcattcataaataagtagaaagttgtttctccagtcttttgggaacacaggacttaaaagtcacatcatgtgtagatattacaagcagcattaccaagacatggcaaaaagagtttgtctgaattgtaatgttgcgtttgtgaacctattctgggattttcagaggtacaaggttagaatgctacaatgttaccactgtgccttccaatgtttatatcatcggaaacataacataatcaaagtggctgtgatttaacaaaatgattaaagtgttacctacctgtgtagccgaagtagtgtgcagtgaggcgtttctgaatacatggtcagatttttggaaaaaaacaaaaacaaaaaaaacaagtaaagttcaaaaaccgtcaaatgagaaaattgcaagtagtgtgacagagctgattgattttgttgctttcttgattttttttttcaaaatgggtttactaaaatgtagatgacttaactgcctcctccttcgtctgaaaaatgccaatattcaatcatcatgcagcattataacaagccttataagtcctaaagcattaagttgcacttttttgaggaggggtagtgcagtatttctctggccagtatgaatgaagtttatacttaccatatttgatagaaacatagatcaagctatggcacagcgactcatcagatagctagctttgacgtctgggcacaattgaaccaacttccatcgtgaatctttataatgattgactttggtgtatagtgcagtaaacaaatagtgctcctagttaagtatttgtcagcatccttttgtctctaacttgtttctatttttacagccacacaattcttggcatgtattaagaaaaaaaaaaatccctgttcaagtagtttttccacctatcagcactgagtaaatgccataaatccattgaaatggtctaaatgttccatctgttctcctgttttgccagttatatagtaatgaaatacatttgtaaattttatgcaacaaatggcaaacgtatcattattttgaaattgtgtatgtaaaagttatatttttacatgtagactcttgttattatgtgttttaatacattgtatcagtttttgtttttttttaaaaactgtggtttaaaaagaagtctcatttaaatgaaatagctacaagaatcagaattttatgttcatttctgaaaatgtaagaacaaataagatagttaccacgtggtcatcttttacaaacccataaacattttgattagctgtgtgtgtgttgaaaaactgtaaatatgttcagtagcgataaaactaaaataactttgatttgttgataagttcctaaaatgtggaggtggattaaaaccttaggagaatagcagaaatcaaacttcatgaaaagttattttggggctttcctgtgaaatgtatgaacaaagaggctcagagaaggacatggaagacaataatgtatactctctcctcctccctgaataatgaaaaccatgtgtatttgttccctccgtatgttaaagatttccttttagtggtacattctgcactcattttgtatagtctaccaaggcgggtatccctaggaacaatattatataggaagcaggtatactctgatcacattcaggataagtgtacagaagaaaatacggtgtttactctttagggaactggaaacactccctgcattgatgtacattttaagaatggcacttttgatacatgttatcataaaggtgcttaatagagctgaattaaagtttttcaaatctgtaaacaaagcaaaaaagtaaattgtagtcatttgattattttttaaattggtgctttatattttgttctcactcagagtaaaagctgcaatttattgttcaccagctttgatgtattcattactcagtaatgcaatacctctattgttgaattccctttggaaataagtgaaaattctaacggccactgaaagctgctcgctaggttttgcttggtggagaaacataatctgcacctatccatattaattgggttgtatccccattaaaaaagaaaaaaagggaatgtggcctttttagtgtgttttttattgttgttgttttgtaattatcaaacccaggtaagatattggtatcctgcactggattttcaaatgaagttcagcagaagacagttaagattaaagtactatacaaaaatttcaaaagggtccatactacgctatctgtatgacgacacttaggctggggatctctttcagaaactcggactttaaaagcaacttggagcagttgatccacctccacattcaagtaatttatgaatatgcagaatagggatctgttcatctagaaatttttaccatttgtcttctgtgtagctgcaaggaacactaatgtttatacaactgtcagtccacccagtggtgcaactggttctgattcagtcttccgattcctttttatttttcactttttcctatttctgaatttttttttttatttgtgatcttgattttgatgaggggttggggagtggggagggagtcgaaccaagacttggagttaagaggattttcatcttttgcatccaacaggcagaatatgatctgtgtccaaaagtgaacttgagtcaggaatgaatcaatttcagcataaacaagcacaaaaatttagtctgctggctgactggaagcaaaaaagtcaagatggaatatgatgaattccaacacaatggggcaccaaggcctttaggcctctctttttattttgctttggttttgtttgtttttctttagagacatgctctttctcatgggacttgaagtggactcatctttgtgcagtgctggttttgccatactcatttcaagtattatagacatatgtaatggtgaaaatatatgaactgtggcctttttcattcttgttacttgtgatgcaattaagtgaagataagaaaaaaaaaaaaaaagcagagatttaccatgtatcagtgcctggctttttgttataaagctttgtttgtctagtgctcttttgctataaaatagactgtagtacaccctagtaggaaaaaaaaaaaactaaatttaaaaataaaaaatatatttggcttatttttcgcaggagcaatccttttataccatgaatattacaaaaaaattgtcagattctgaatatttcttctttgtagatttttggaatcattatgagtaaaagtttgttactttattttactatttaaaagatgttattttaccatgtgttaccaagatgaaactgtatgggtagcttttttgtttgttttttgttttgtttttgtttttgtttttgtttttagttgtaggtcgcagcggggaaattttttgcgactgtacacatagctgcagcattaaaaacttaaaaaaattgttaaaaaaaaaaaaagggaaaacatttcaaaaaaaaaaaaaaagataaacagttacaccttgttttcaatgtgtggctgagtgcctcgattttttcatgtttttggtgtatttctgatttgtagaagtgtccaaacaggttgtgtgctggagttccttcaagacaaaaacaaacccagcttggtcaaggccattacctgtttcccatctgtagttattcgatgaagtcatgtacatgaccgttctgtagcaataaatgtgccatttttataaactgtttctgacacttgtttcatttcattttgcattgtccatatagctatgattctcttctgtaagtaaaacgcatctatatttcattttccaagtgttggaggtattgacagcttaacaaacaaaacatacaaaaaaaatcacaaaaacaaattgaaaagcaaagcacatgattgatcaaggaagagatgcccttaatgaaaatggaacgggatgcatgcaaaacaaaaagaaaactgtctagaggattaactaattgaaggaatataattaatgtgtgtgtaacactgaagctatgcatttgaagagctctgaactgcaccagtgttttcggttgtgctgcaggttgctaagtcaagtcagccttaaccttttgcaccagttggtcggctgtttggcagaacattctcagatcttttcagtcaaaaatctaagatgatttattttgtatcactttgttaaaagctgaatattgttaactacagttaatattaacactgtatttatactttctcaaactacatccgccccaccacttctggttgcctctgttgactattaatccagatgtaaacaaccagatgtttttttctaacttgtacaaactgacgtgtgtcaactatcatggaaggaaaaaaatgtacagattaaaattattcagtgttatgtactgtaagttaatatttttgtagaatggacatcaatctactttgcaaaatttggaggctatttcaacattgcactgtagaaatgtaaagtaatgtatgcaatgtaaaggaaagcccgcggtagctgagcgcttcataacagaatgttctaatcaagtacgtggtatttggggatgtctccaatattgctcttgtattctttctaattgggtttagtgactagttgaaggaaaatgttataacgccatttggttcacatgtgaagtgccctccatagccaaatgttgggatttttttttttttcgtttttggttggactgtttgcagatatttaaattttatgaaatttccaaagattttggttgataacccccttttaccttctaaatgatttgagatgttcttatgttcttactgtgtgttttaaatatatataaaagagccacaagcatttagtcttttagtattatatttgatcatataactactactgaaaaggggtgggggggaatgtgcctgtgcacccctacagtatcttgtgtagtgtctattaacattgttctcttttagaggaccacattaaaaggtacattaatctta

SEQ ID NO: 42 - DNA, 7462 bp, Mus musculus
ggagcagccgcggccgcagcgccttctctttataagccgcagtgcccggatgtgaatggattacaatgtatctttcagggaaacctattattatcaatgtgactcctcgggggagtcaatgatggtgttggggaggaggatgatgatgagacgcctctaaacttggaacaagtttaggactttgaaagagaagagaaaaaaaaatacaaccaacaagaccgaagaacaattataactatccagtgttgattatttttataaacaatacgaaaaagttgtcggatttttttttttaatgattactttttggggggagggaattttgttacagtttgatgatggaaaatgcaaaaaccgagccaggtgcataatcttgtaatctgtggctaaccctggaacaggactgacttctatttaaaatactcttttgggggaacactcatgtgagacactaagttcttgcagaagatttttgtctctctttttaaagtctctttccttggaatattgtgagcatatttgtggccattgaaggtttgtgtgattttgctaaaatgcatcaccaacagcgaatggctgccttagggacggacaaagagctgagtgatttactggatttcagtgcgatgttttcgcctcctgtaagcagtgggaaaaatggaccaacttctttggcgagtggacatttcactggctcaaatgtagaagacagaagtagctcagggtcctggggaactggaggccatccaagcccgtccaggaactatggagatgggactccctatgaccacatgactagcagggatcttgggtcacatgacaatctctctccaccttttgtcaattccagaatacaaagtaaaacagaaaggggctcatactcatcttatgggagagaaaacgttcagggttgccaccagcagagtctcctcggaggggacatggatatgggcaatccaggaaccctttcgcccaccaaacctggctcccagtactatcagtattcaagcaataatgcccgccggaggcctcttcacagtagtgccatggaggtacagacaaagaaagtccgaaaagttcctccgggtttgccgtcttcagtctacgctccttcagccagcactgccgactacaacagggactcgccaggctatccttcctccaagccagcagccagcactttccctagctccttcttcatgcaagatggccatcacagcagcgacccttggagctcctccagcgggatgaatcagcccggctacggagggatgctgggcaattcttctcatatcccacagtccagcagctactgtagcctgcatccacatgaacgtttgagctatccatcccactcctcggcagacatcaactccagtcttcctccgatgtccacgttccatcgtagtggcacaaaccattacagcacctcttcctgcacaccccctgccaacggaacagacagtataatggcaaacagaggaactggggcagcaggcagctcgcagactggagacgctctagggaaagccctagcttcgatctattctcctgaccacacgaacaacagcttttcctccaatccttcaactcctgtgggctcccctccttcactctcagcaggcacagctgtttggtctagaaatggaggacaggcctcgtcatctcccaattatgaaggacccttgcactcactgcaaagccgaatcgaagaccgtttggaaagactggacgatgcgattcatgttctccggaaccacgcagtgggcccgtccacagctgtgcctggtggccatggggacatgcatgggatcatgggaccctcccacaacggagcgatgggtagcctgggctcagggtacggaactagtcttctctcagccaacagacactcgctcatggttggggcccaccgtgaagatggcgtggctctgagaggcagccattctctcctgccaaaccaggttccggtcccacaacttccggtccagtctgcaacttcccctgacttgaacccaccccaagacccttacagagggatgccaccaggcctccagggccagagcgtgtcttctggtagctctgagatcaaatccgacgacgagggcgatgagaacctgcaagacacaaaatcttctgaggacaagaaattagatgacgacaagaaggatatcaaatcaattactaggtcaagatctagcaataacgatgatgaggacctgaccccagagcagaaggctgagcgcgagaaggaacggaggatggccaataatgcccgtgagcgcctgagggtccgagatatcaacgaggctttcaaggagcttggccgtatggtgcagctccacctgaagagcgacaagccccagaccaagctcctgattctccaccaggccgtggctgtcatcctcagcctggagcagcaagttcgagaaaggaatctgaacccgaaagctgcctgtctgaaaagaagggaggaagagaaggtgtcctcagagcctcccccactctccttggctggcccacaccctgggatgggagacgcagcgaatcacatgggacagatgtgaaaaggtccaagttgctaccttgcttcattaaacaagagaccacttccttaacagctgtattaccctaaacccacataaacactgctccttaaccccgtttttttttgtaatataagacaagtctgagtagttatgaatcgcagacgcaagaggtttcagcattcccaattatcaaaaaacagaaaaacaaacaaaaaaatgaatgaaagaaagaaagaaagaaaaaaatgcaacttgagggacgacttctttaacatatcactctgaatgtgcgaagcggtatgtacaggctgagacacagcccagagactgaatggcaatcctcccacactgtggagcaatgcatttgtgcctaaacttcttttggaaaaaaaaaatataattaatttgtaagtctgaaaaaaatatttaatttaaaaaaaaattgtaaacttgcaataatgaaaaagtgtacttctgaagaaaacgacatgaacgtttttgttggtattcacgtcagctagtgtttctaattaccggatattgaataggggaagcccggctgccctcgtaacaaaaccagcaaacgtcctgatggcaacgaagtgatgacattagccattccttagggtaggagggacagatggatgttatagacctatgacaaatatatatataaatatatatataaatatatattaaaaatttagtgactatggtaagcttttgttgatttgtttcagacttttttctcctgtaaaaaaatagtactgattaacttttttaaaagaaagattttactgtaaatatggatttttttttttgtctgatttttgtcccttcccccggtttgttatcgtaacctgtagtgccaactctgcttccggaggggcagtgcaggacgaaatgctgaccctgaagttgcttctcattcacaaatagtaaaaagttgtttctccagtcttttgggaacacaggacttaaaagtcacatcatgtgtaggaattacatgcagcattgcccgggcgaggcaaaaagcgtttgtctggcttgtggcgctgcccttgttaccctcccctgggattttcagaggtacacggttagaatgctacaatgttaccactgtgccttccaatgtttatatcatcggaaacataacataatcaaagtggctgtgatttaacaaaaaaaacgattcaagtgttacctacctgtgtagccgaagtagtgtgcagtgaccgagacgtttctgaatacatggtcagattttttttggaaaaaatacaaaaattaaaaaaaaaaaaaaaaaagaaaaaaagaaaaaaaaactaaaaaagaaagtcaagttcaaaaaccgtcaaatgagaaaattgcaaggagtgtgacagagctgattgatttttgttgctttcttgatttttttttcaaaatgggtttactaaaaagtagatgacttacctgcctcctccttcgtctgaaaaaaaaaaaaaatgccaaccaatcatgcagcattataacaagccttataagccctaaagcattaagttgcacttttgtgaggaggggtaacgcagtattctctctggccagtatgagtgaagtttatacttaacatttgatagaaacatagatcaaactacggcacagcaactcatcagatagctagcgttgactctgggcacaattgaaccaattcccatcgtaagtctttccaacaatggactttggtgtgtagtgcagtgaacacataggactcctacgtaagcatttgtcagcatccttttgtctctaactcggttctgttttgacagccacacaatcttggcatgtattaaggaaaaaaaaaatccctgttgaagtagtttttccacctatcagcactgagtaaatgccataactccgcggaaatggtctaaataccccatttgttgtcctgaactaccagttacatagtgacgaaacacatctgtaaattttatgcaaccaatggcaaacctatcattattttgaaactgtgtatgtaaaagttatatttttacatgtagactcttgttattatgtgttttaatacattgtatcagttttttgtttttttttaaactgtgtggttttaaaaagtcatttaaatgaaatagtgagctacaagaatctgaaatttatgttcatttctgaaaatgtaagaacaaataagatagttaccacgtggtcaccttttacaaacccatgaacattttgattagctgtgtgcatgtgtgtgcgtgtgtgctcttgcgcgtgtgtgtgtgtgtgtgtgtgtgtgtatgtgtgtgtgttgaaaattgtaaatatgttcagtagcgataaaactaaaatgctttgatttgttgagaagttcctaaaatgtggaggtggattgaaaatgtaggggaatagcaggaatcaaatctcataaaaagttcttcggggactttgctgtgatgcggtggcacaaaggtgctccaagaagggcaaagaagaaagacagtaacttcccctcctccctgaacgatggaaaacatgtgtacgtggtctctcaccatgttaaagattttctttccgtgatacattctgcactcattttgtatagtctgccaaggcgggtatccctaggaacaatattattatataggaaacaggtatcgtccgatcacattcaggagaagcgtatagaaaagaatatggtgtttactctttagggaactggaaatcctccccgcattgatggatatcttcagagtggcacttttgatacatgttatcataaaggtgcttactaaagcagaatcaaagtttttcaactctgtaaacaaagcaaaaaattaaatttttaaattaaatcatttgagattttttttttcaattggtgcttttatattttgttctcactcggacagagaaaaagctgcaatttcatgttctcaccagctttgatgtattcattacttggtaatgtaatatggctattgtcaaattccttttggaaataagcaaaagactccccaaaggccagcagcagctgctggctactttctgcttggtgcggaaacctgatctgaccctctttgtagtaatcaggtgtatctccagttttaaaaagaaggagaaaggaaatgtggccgttttaacgtgttggttttgttttgttttgttttgttttgttttccttgttgttattttgtaattatcaaacccaggtaagatattggtattcctgcactggatttttgaaagaaacttagcagaagtcaggattaaaatactacacaaacatttcataagtgttcatcctatactagacatacacgaaggcggtgaggcttaggtgcaggatcggtttaagagactttgaaagcaacttggaacagttgatccacctccacattaaagtaaattatgaatatgcagaattagggatctgtccatcttggagttgttactctttttgtcttctgtatggcagtgatgaacactaatgctttgacagctttcagcccacccagtggtccaactgattccaattcagtcttccgattcctggttttgttttgtctttccactttttttttcctatcttgattttattgttattgttattgttattattattattattattattattaatattcatgatgtttgttttgatgagggattgggaagtgggagggagtcgaactgagactaggggctgagaggatttttttttttccatctttgcatccaacaggcagaatatggtctgtgtccaaaacggaacttaagtcaggaaggaaaccattcagcataaacaagcacaacatgtagtctgccggctgactggaagcataaataaataaataaataacacatacggaggtgaagatggaatgtgctggattccaagacaatggggcaccaaggcctgagggcctcctcttgactttgctttggttatgtttgttttctttagagatgtcttttctcatgggacttgaagtgactcatctctgtgcagtactggtttcgccatatgctcatttcaagtattatagacatatgtaatggtgaaatatatgaactgtggcctttttcattcttgttacttgtgatgcaattaagtgaagataagaaaaaaaaaaagcagagatttaccatgtatcagtgcctggctttttgttataaagcttcgtctgtctagtgctctttttgctataaaaaatagactgtagtacaccctagtaggaaaaaaaaactaaatttaaaaataaaaaaatatatttggcttatttttcgcaggagtaatccttttataccatggatattacaaaaaaaaatgtcagattctgagtatttcttctttgtagatttttggaatcattatgagtaaaagtttgttactttattttgctatttaaaagatgttattttaccatgtgttactgaaaggaaactgtatggtagagcttctgtttgtttttgttttcaggttttttttgtttgtttgtttttagttgtaggtcgcagtatgaactttttttttgtttctcgttatgttttgttttgtttttgcgactgtacacatagctgcagcattaaaaacttttaaaaaattgtttaaaaaaaaagaaaaaaagggaaaacgttttaaaaaaaaagataaacagttacaccttgttttcaatgtgtggctgagtgcctcaattttttcatgtttttggtgtatttctgatttgtagaagtgtccaaacaggttgtgtgccggacttccttcaagaggcccacagcccagcttggtctagacctgttcccatctgtagttactcgatgaagtcatgtacatgaccgttctgtagcaataaatgtgccatttttataaactgtctctgacacttttttcatttcacctctcgtgcacatag

SEQ ID NO: 43 - DNA, 6104 bp, Homo sapiens
gagccgagcgcggtggtgaggccgcctcagcgaaaaaaatgtccgcctgaagagacccacaagttctattcggggggaccgacagcccgccccgggaggaaggggcggccaggcccgaaagccgcctccccctcccagacccgagagctcgtgcggggcaaagtgaaccgagccgctgggcggtgcaaggggaagcccaagcccgttctcccggccaaagtgaactttaatcggggtggttggatgcggagacggggcggcagaagtggccgaagatgaatccccagcaacaacgcatggccgctatagggaccgacaaggagctgagcgacctactggacttcagtgcgatgttttccccacctgttaatagtgggaaaactagaccaactacactgggaagcagtcaattcagtggatcaggtattgatgaaagaggaggtacaacatcttggggaacaagtggtcaaccaagtccttcctatgattcatctagaggttttacagacagccctcattacagtgatcacttgaatgacagtcgattaggagcccatgaaggcttgtccccaacacctttcatgaactcaaatctgatgggaaaaacatcagagagaggctcattttccctgtacagcagagatactggattaccaggctgtcaatctagtctcctgagacaagatctggggcttgggagcccagcacagctatcttcttcaggaaaacctgggacagcatactattcattctctgctacaagttccaggaggagaccactccatgactctgcagcgcttgatcccttgcaagcaaaaaaagtcagaaaggtgcctcctggtttgccttcttctgtatatgcaccatccccaaattcagatgatttcaaccgtgaatctcctagttatccatctcctaagccaccaaccagtatgttcgctagcactttctttatgcaagatgggacccacaattcttctgacctttggagttcatcaaatgggatgagccagcctggttttggtggaattctggggacctccacttcccacatgtctcaatccagtagttatggcaaccttcattcacatgaccgcttgagttatcctccacactcagtttcaccaacagacataaacacgagtcttccaccaatgtccagctttcatcgcggcagtaccagcagttcaccttacgttgctgcctcacacactcctcccatcaatggatcagacagcattctaggaaccagagggaatgctgctggaagctcacagacaggtgatgcacttggaaaggctttggcatctatttattctcctgaccataccagcagtagttttccgtcaaatccatcaacaccagttggatcaccttcacctctcacaggtaccagtcagtggccaagacctggagggcaagcaccttcatccccaagctatgaaaactcactccactccctgaaaaatcgagttgagcagcaacttcacgagcatttgcaagatgcaatgtccttcttaaaggatgtctgtgagcagtctcgaatggaggatcgtttagacagactggatgatgcaatccatgtgctgcggaaccatgctgtgggaccttccaccagtttgcctgctggtcacagtgatatacatagtttattgggaccatcccataatgcaccaattggaagcctcaattcaaactatggaggatcaagccttgttgcaagcagtcgatcagcttcaatggttggaactcatcgggaagactctgtcagtctcaatggcaatcattcagtcctgtctagtacagtcactacttcaagcacagacctgaaccataaaacacaagaaaattatagaggtggcttgcaaagtcagtctggaactgttgttacaacagaaatcaagactgaaaacaaagaaaaggatgaaaaccttcatgaacctccttcatcagatgacatgaagtcagatgatgaatcctcccaaaaagatatcaaggtttcatctagaggcagaacaagcagtactaatgaagatgaggatttgaaccctgaacagaagatagaaagggagaaggagaggcggatggctaacaatgccagagaacgcttacgcgtgcgggatattaatgaagcattcaaagagcttggccgaatgtgtcagcttcacttgaagagtgaaaaaccccaaacaaaactccttattcttcatcaagccgtggcagtcatccttagtctagaacagcaagtcagagagaggaaccttaaccccaaagcagcctgccttaagagaagggaagaagaaaaagtttctgccgtatcggcagagccgccaaccacactgccaggaacccatcctgggcttagtgaaactaccaaccctatgggtcatatgtaaacatcagccagttccagagttatcagtaggctagatagaaggtgacctctcctcataaggacttggacaactcagattatctgaagacacaaacctgacaggagggagaagaaaaaacaaaacacttgaaccaagaaactcaaatgtaatcctacgatcaaagcaactggtcaacacttccatcagaagtgaagataggaagctcatcagatagaacatcagcccatgagatgtttgcaacaaatcttttgttgcaagcagtgtgtcgcttctgcacaatcagagactgtctcgatctctccactcaccgtggaagttgccttgtgcctaaactgaattgacaaatgcattgtaactacaaattttatttattgttatgaaactgtaaggtctacatataaagggaaaaagttaatgtggaaagctgatctacactcagctgatgccagcatacattaaagcggttcacgtgcagagaacaaagcagtgacaaccattggcccttagcattcccggcatacctattagtgtcttaaaaaggaagggaaaagtcttttgttgccctctcctatcctcttgccatatgaatagcgttttccatgaaataggaaaatattacttggtatagcatttctcttgctctcattttttgatttatttttattttctctttgtgggtgttatatttgatctctaaatctgaacagtttatggtcacagtccagcctcctccgtgcagccctgtgtgctttgcacatttaccttacagtggtaagcagagaccatctgtgaccatagcctagctagcattttaaaaggggaaattttgttctctaggttttcccccaaataaacattgctttatttctaataataaccaagacttttcaagcttctagatctcataggaaagcttgtaatagcaaaattgtaaattacaagggaagaatctactttttagaaatcgctttgttttccaagcagtaagtactacatacagtacttgtaaagtgttagctgtaagtaagcacaaaatacatttaaaatacaaagacgattttttcaggctgtgattatggtgaacataacaaaacccagtagtcaccaaggcaggtagtgtgataaatgaacacaccactctgaggctaattacctaatggaatacaagagcaatggtcacccgtatttccttatcctagcctttatttctctgtcatttggatggctggtcaatggggaagaattgagtgggtgatttaatcaactgcaaaccatctgcccctgtcccaaaatgatgagccagattagcattaaaccagtacttgtcagtccatcttaatactgttcattaaggcactctctgtctctaatccttaggagttgttttaaaagacataatcactttgaacttccatgaaacctgtcttccaccacaacaaccctgggagagaaaaacatgctaaaggaggtatcttggcttaataattccttatagccaatatcaacagtggcaatcagcacacagaggaaaggacccaaatcactatgtagcttaaagatttctgttaatttgaaagaacaaaaacaagacagaacttctggtactctaatcaggatgattcctaacaagtcagtcatttgtgaacttagtggactttttggttactttaatttgcatatattctccagttacatcggactctatctgtggccttgttcttcatttcagtgttaatcagctaaacagaagttgttgcttatgatgtgtgagtgaacatatgccactgcctggcctttttttcttcagagcttgttgtctttttcgctatattagactttgcagtatgcccagaagctttccttcataaaatagaaagaaaaaaacatttggcttatttttcactgtagctagtcttttatacaataatcttgtaagaaaatttcttgaattctaaatattactctttctagatttttgaaatcaaaaagttttcagtaaaaagtttcttactttattttattatattaggtagtaaaaaatgtagggttatttaccataacctgttcattaatatcagaaatttacaatagcattttaagaccatagtaggattctagcataccgtgtagtacctatggagtattgtaagagctaattgttggagatgaattgcttctcatcttgttctccagtttccattgttggtttattgcagatttgtatcctgtgtcaaattcaaggtattattgataaaccttttcaaccagcagcaagaagttcaaatttttttctgtcactgtaacagaaaacacaatatgtatataacatttatgtagcaataaatgtgccatcttttttttaacacagtaaaatagtgagttttttacatttctctttctcaaataataatgtattttgttttattttctccatctcattcgtcccagaaacactcacactgcttttcctaactgcattaccgacattatctgggaaacccttcaggacagaatcaggcttgtggagctaagttggcaatctggtctagagcttctctagcttgtgcttttctcctcttgccctcactactgacggtggccttttaaccttttcctaaagattgaccaaacagcaactagtagttatagaaaatctactcatttgtagatacagagaaaaatgaagaagatggaaaaagactcaaaagaggctttttaagttattcttcaaagcacttttcacatttccccataccctttctcacaaaaaaagtgtcataattaagtaatggtattgtttactgtttaaaagttaaaagatcaaaaatttgcttttatcccagtttttaaccacaaaaaaaagcgtagggattatccatgaggacttcatgccaagcaagaacctcaaacaaactagacaaactttttttttgacagtgaatgactttttgtaggacctgtgcgtgcgaaacccatggcaattgtcacatcctcttggtatgctggcagattgcttctcttggtgaattatgaaatccactgttcacattgggtgcctaacagaacattttgcttcttgtgggatttagtgaaaactattaaacttgttaagttgattttatacaaaacgataaataaaaagctctaagaagaaaatgtataatcttagagctgaaataaaatatagagaccatctagtaaatgacctcattaatatatctgtgaaaactgagactcagattgtatgtctctaagaacacataattagtaacagatcaagacacttaaaactttccctacaaaacctccctgccttgactttctctttctctttgcagatttctaggccgcttctgctcagtgtcttcattttcttccatatttgttttatttcatttttcttttctatagttcatgttttctttttccttggaaactcaaaatttaaacacacgtcgtgtgtgtgtgtgtgtgtgtgtctgtgtgtgtgtgacttaaagaatcttaagctttggcattaaatagtcctcgattcaaatctaagctcaacatctgattaacttcattttcctatctgaaaaatggagataacattagaattgtgtaagtattgaatgaaacaatgtatggaaagctcttatggttcttgtcacctaagaagtacttaataaatgataaatatttttaaataaataatattataaccaa

SEQ ID NO: 44 - DNA, 4707 bp, Mus musculus
ggaaaaaaaaaaaaatgtccgcctgaggagacccacaagttctatcggggggacagccagcccgcccggggagccacgggcggctaagcccgagagccgcccgagtcgcgccgactcaagcactcgcgggggcccgaggcaacgcagccgccggcggcgcgtggggagggggaaagcctgagcccgcgctcccggccaaagtgaactttaatcgggaaggttggatgcggagcccgggcggcaggacctgctagaagtggctgaagatgaatccccagcagcagcgcatggccgcgatagggaccgacaaggagctgagcgacctgctggacttcagtgcgatgttttctccacctgttaatagtgggaaaacgagaccaacaacactgggaagtagtcagttcagcgggtcaggtatggatgagcgtggaggaacaacatcttggggaacaagtggtcaaccaagcccctcctatgattcatctagaggttttacagacagccctcattacagtgatcacttgaatgacagtcgattaggaacccacgaaggcttgtccccaacacctttcatgaactcaaatctgatagggaaaacatcagagagaggctcattttccctgtacagcagagactctggactctcaggctgtcagtctagtctcctgagacaagatctaggacttgggagcccggcacagctgtcttcttcaggaaagcctggaacaccatactactcattctctgccacaagttcgagaagaagaccgctccatgattctgtagccctagatcctttacaagcgaagaaagtaagaaaggtgcctcctggcctaccttcttctgtatatgcaccatccccaaattctgacgatttcaaccgtgaatctcctagttacccatctcccaagccaccaaccagtatgttcgctagcactttctttatgcaagatgggacccacagttcttctgacctttggagttcatcgaatgggatgagccagcctggttttggtggaattctggggacctccacatcccacatgtctcagtccagtagttatggcagccttcattcacatgaccgcttgagttatcctccacactcagtgtcaccaacagacataaacacaagtcttccgccaatgtccagcttccaccgtggtagtaccagcagctcaccatatgttgccgcctcacatactcctcccatcaatggatcagatagcatcctaggaaccagagggaatgctgctggaagctcacagacgggtgatgcacttgggaaggccttggcatctatttattcccctgaccacacaagcagtagttttccatcaaacccatcaacaccagtgggatctccttcacctctcacaggtaccagtcagtggcccagagctggagggcaagctccttcatctccaagctatgaaaactcacttcactccctgaaaaatcgagttgagcagcaacttcacgagcatttgcaagatgcaatgtccttcttaaaggatgtctgtgagcagtctcgaatggaagaccgcttagacaggctggatgatgctatccatgtgctacgaaaccatgcagttggaccttctaccagtctgcctactagccacagtgacatacacagtttgctgggaccatcccataatgcatcaattggaaacctcaattcaaactatggaggatccagccttgttacaaatagtcgatcagcttcgatggtcggaacacatcgggaagattcagtcagtctcaatggcaatcattcggtcctgtctagtactgttgctgcctcaaacacagaactgaaccataaaacaccagaaaatttcagaggtggtgtacaaaatcagtctggaagtgttgttccaacagaaatcaagactgaaaacaaagaaaaagatgaaaaccttcatgaacctccttcatcagatgacatgaaatcagatgatgagtcctcccagaaagacatcaaggtctcatctaggggcagaacaagcagtaccaatgaagacgaggatctgaatccagaacagaaaatcgaaagggagaaggaaaggcggatggctaacaatgccagagagcgcctgcgcgtgcgggatattaacgaggcgttcaaggagcttggccgaatgtgtcagcttcatttgaagagtgaaaaacctcagacaaaacttctcattcttcatcaggccgtggcagtcatccttagtctagaacagcaagtgagagagaggaacctcaaccccaaagcagcctgccttaagagaagagaagaagaaaaagtctctgctgcgtcagcagagccgcccaacacgttgccaggagcccatcctgggcttagtgagtctaccaaccctatgggtcatctgtaaacatcagccagttccagagtcatcagtaggctaaatagaaggtgacctctcctcataagatttggacaactcagattatctgaagacacaaacctggcaggagggagaagaaaaagcaaaacacttgaaaccagaaactcatatgtaatcctgtgatcaaagcaactggtcagcacttcatcagacgtgagcataggaagctcagcagagaccgtcggccgtgaggtgtttgcagcatatcactctgctgtaatcagtgtgtcgcttctgcacaatcagagactgtctcatctctccactcaacgtggaagttgccttgtgcctaaactgaattgacaaatgcattgtaactacaaattttatttattgttatggaactgtgaggtctacatataaagggaaaagttcatgtgggaagctgatgtacactcagctgatgccagcattgttaaagctgttcacagagcagtggcaaccattggcccttagcattcccggcatacctgttagtgtcttaaaaaggaagggagtcctttgttgccctctccgaccttcgccatatgaatagtgatttccatgaaataggaaaaatattacttcgtatagcatttctctcttgtttttttcactcatttttatttcctctttgtgggtgttatatttgatctgagtctgcatagtttatggtcacagtccagaaccctccttgcagtcctgtatgctttgttcatgtccttgaagtgataagcagacaccatctgtgaccatagcctagctaatattttgaaaggggaagttttgtcccctggatttgcccccaaataaacattgctttatttctaataatcactaagacttttcaggcttctaggtttcatagtaaagctataatagcaagaagtgtaacttacaagggagagtttactttttaggaattgctttgttttccgagcagtaagtactacacaatatagtacttgtaaagtgttagctgataagtaagcacagaatgcattcagtacaatacaaagatgacttttcctggtgagtctccgggacaggcagtgtgatgaatgcactcaaccgctctgaggctaattacctatggaatccaagagcaatggtcacggttccttaccctagctttacttctgtcctttgagttggctggtccgtggggggtggggcaggagggtgacttaatcacctgcaaaccacctgcccccaccccaagaagagccagattagcaccgagctgtacctgtcagtctgtcttagcattatgcattaaggcaccctctgtctctaatcccttacagttgtttttaagacacagtaatcactttaaacttccatgaaatctgtcttccaccacagcaccctgggagagaaaaacatgctaagcgtgatggtcttggctaagtaactccttaaagccaatagcagtggcagtctgcacagaagaaaaatcccaagtcgttctgtaacttagagacaccggagaattttgaaagaacaaaaaccatgaagacagcacttcagatcctccatcaggactctggtgaacacgtcagtctttggcgaacttagtggacttaatttgtatatgttctccagttagatcagactctatctgtggccttgttcttcatttcagtgttaatcagctaaaacagcagttgttgctatgatgtgtgagtgaacataagccactgcctggccttttttcttcagagcttgtcgtctttttcgctatattagactttgcagtatgcccagaagctttccttcataaaatagaaagaaaaaaacatttggcttatttttcactgtagctagtcttttatacaataatcttgtaagaaaatttcttgaattctaaatattactctttctagatttttgaaatcaaaagttttcagtaaaaagtttcttactttattttattatattaggtagttaaagaaaatgtagggttatttaccataacctgttcattaatatcagaaatttacaatagcattttaagagcatagtaggttctagcataccatgtagttcctatggagtattgtgagagcgaattgttggagatgagctgcttttcatcttgttctccagtttccattgttggtttattgcagatttgtattctgtgtcaaattcaaggtattattgataaaccttttcaaccagcagcaagaagttcaaatttcttttttctgtcgctgtaacagaaaacacagtatgtatataacatttatgtagcaataaatgtgccatccttttttttaacatagtaaactagtgagttttttac

SEQ ID NO: 45 - DNA, 4116 bp, Homo sapiens
gtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatagagcatgaatttttttcatcttctctggcgacagttttccttctcatctgtgattccctcctgctactctgttccttcacatcctgtgtttctagggaaatgaaagaaaggccagcaaattcgctgcaacctgttgatagcaagtgaatttttctctaactcagaaacatcagttactctgaagggcatcatgcatcttactgaaggtaaaattgaaaggcattctctgaagagtgggtttcacaagtgaaaaacatccagatacacccaaagtatcaggacgagaatgagggtcctttgggaaaggagaagttaagcaacatctagcaaatgttatgcataaagtcagtgcccaactgttataggttgttggataaatcagtggttatttagggaactgcttgacgtaggaacggtaaatttctgtgggagaattcttacatgttttctttgctttaagtgtaactggcagttttccattggtttacctgtgaaatagttcaaagccaagtttatatacaattatatcagtcctctttcaaaggtagccatcatggatctggtagggggaaaatgtgtattttattacatctttcacattggctatttaaagacaaagacaaattctgtttcttgagaagagaatattagctttactgtttgttatggcttaatgacactagctaatatcaatagaaggatgtacatttccaaattcacaagttgtgtttgatatccaaagctgaatacattctgctttcatcttggtcacatacaattatttttacagttctcccaagggagttaggctattcacaaccactcattcaaaagttgaaattaaccatagatgtagataaactcagaaatttaattcatgtttcttaaatgggctactttgtcctttttgttattagggtggtatttagtctattagccacaaaattgggaaaggagtagaaaaagcagtaactgacaacttgaataatacaccagagataatatgagaatcagatcatttcaaaactcatttcctatgtaactgcattgagaactgcatatgtttcgctgatatatgtgtttttcacatttgcgaatggttccattctctctcctgtactttttccagacacttttttgagtggatgatgtttcgtgaagtatactgtatttttacctttttccttccttatcactgacacaaaaagtagattaagagatgggtttgacaaggttcttcccttttacatactgctgtctatgtggctgtatcttgtttttccactactgctaccacaactatattatcatgcaaatgctgtattcttctttggtggagataaagatttcttgagttttgttttaaaattaaagctaaagtatctgtattgcattaaatataatatgcacacagtgctttccgtggcactgcatacaatctgaggcctcctctctcagtttttatatagatggcgagaacctaagtttcagttgattttacaattgaaatgactaaaaaacaaagaagacaacattaaaacaatattgtttctaa

SEQ ID NO: 46 - DNA, 5150 bp, Mus musculus
aggtgcggatccgggtgcgggtgcgggtggaagtggggcgccctctcctcggacctggggtctgctgcccacaggtgacctgtcatcccgcagagagaacgccaggagccggatcgcttgcccaactcttggcgctgcttggctctcttatcctgccgttctcacttctaggtcgtttcagctctgctccataccctgagccggcgccacgccgccgcgcatgcaactggcatataacttgctgtgtgtggtgattgcttgtgttgaatcccgaacctgcacccggagacagcccaaggatgtcacagtggttcgagcttcagcagctggactccaagttcctggagcaggtccaccagctgtacgatgacagtttccccatggaaatcagacagtacctggcccagtggctggaaaagcaagactgggagcacgctgcctatgatgtctcgtttgcgaccatccgcttccatgacctcctctcacagctggacgaccagtacagccgcttttctctggagaataatttcttgttgcagcacaacatacggaaaagcaagcgtaatctccaggataacttccaagaagatcccgtacagatgtccatgatcatctacaactgtctgaaggaagaaaggaagattttggaaaatgcccaaagatttaatcaggcccaggagggaaatattcagaacactgtgatgttagataaacagaaggagctggacagtaaagtcagaaatgtgaaggatcaagtcatgtgcatagagcaggaaatcaagaccctagaagaattacaagatgaatatgactttaaatgcaaaacctctcagaacagagaaggtgaagccaatggtgtggcgaagagcgaccaaaaacaggaacagctgctgctccacaagatgtttttaatgcttgacaataagagaaaggagataattcacaaaatcagagagttgctgaattccatcgagctcactcagaacactctgattaatgacgagctcgtggagtggaagcgaaggcagcagagcgcctgcatcgggggaccgcccaacgcctgcctggatcagctgcaaagctggttcaccattgttgcagagaccctgcagcagatccgtcagcagcttaaaaagctggaggagttggaacagaaattcacctatgagcccgaccctattacaaaaaacaagcaggtgttgtcagatcgaaccttcctcctcttccagcagctcattcagagctccttcgtggtagaacgacagccgtgcatgcccactcacccgcagaggcccctggtcttgaagactggggtacagttcactgtcaagctgagactgttggtgaaattgcaagagctgaactataacttgaaagtgaaagtctcatttgacaaagatgtgaacgagaaaaacacagttaaaggatttcggaagttcaacatcttgggtacgcacacaaaagtgatgaacatggaagaatccaccaacggaagtctggcagctgagttccgacacctgcaactgaaggaacagaaaaacgctgggaacagaactaatgaggggcctctcattgtcaccgaagaacttcactctcttagctttgaaacccagttgtgccagccaggcttggtgattgacctggaggtctttgttccctttcagaccacctctcttcctgtcgtggtgatctccaacgtcagccagctccccagtggctgggcgtctatcctgtggtacaacatgctggtgacagagcccaggaatctctccttcttcctgaaccccccgtgcgcgtggtggtcccagctctcagaggtgttgagttggcagttttcatcagtcaccaagagaggtctgaacgcagaccagctgagcatgctgggagagaagctgctgggccctaatgctggccctgatggtcttattccatggacaaggttttgtaaggaaaatattaatgataaaaatttctccttctggccttggattgacaccatcctagagctcattaagaagcacctgctgtgcctctggaatgatgggtgcattatgggcttcatcagcaaggagcgagaacgcgctctgctcaaggaccagcagccagggacgttcctgcttagattcagtgagagctcccgggaaggggccatcacattcacatgggtggaacggtcccagaacggaggtgaacctgacttccatgccgtggagccctacacgaaaaaagaactttcagctgttactttcccagatattattcgcaactacaaagtcatggctgccgagaacataccagagaatcccctgaagtatctgtaccccaatattgacaaagaccacgcctttgggaagtattattccagaccaaaggaagcaccagaaccgatggagcttgacgaccctaagcgaactggatacatcaagactgagttgatttctgtgtctgaagtccacccttctagacttcagaccacagacaacctgcttcccatgtctccagaggagtttgatgagatgtcccggatagtgggccccgaatttgacagtatgatgagcacagtataaacacgaatttctctctggcgacatttttttcccatctgtgattccttcctgctactgttccttcatatgcagtatttctagggaaatgcaagaaagaaagagcatcacatttgctgagcactgctggtagaaagtggatatttctctaattagaaacctgttactctgaaggacttcatgcatcttactgaaggtgaaatggaaagtcacttaacacaaaatggattttgtaaacaaagaccaagagatccacccaagcaccaggactagagtgcgagtatttggggcaaggtgaggagaacggtcactttagtaatggtctgtaatcagtgcccaagtgctgcacatcactggaaagagacatacttatgggggaggggccttcttgatggaggaatgtttctgtcccgggagacattggcacttcccctctcctggatggccggaagtcttccactgttttacatatggcacagttcaaagtcaactttagatccaatgctctatcaaactatagtgggcatccttcatgtgagtgggaagaaaacaaccgtgctccttactgcagcttctgccaaggcatggttgctctcctcagggactagctttgttggtggcaatggctacacaaaactaaacaccaacagaagtaagaccattttcatgagtactccatcaagttaaagggtttttgttgtctttttggtcatggattgaataaaattgtctttgcacatccattaagggggccagctttcttaaagcaatttttctttttttttaactaaaattagatataggtgaactcatgttttttagtgggctgaacttatcggttttagctggttgtcttaattagccataaacttggagaaagcagtgacttcttgaatccttagccaaatatgagtatcagataattttattatttttttttcgagacagggtttctctgtgtagccctggctattctggaactcactctgtagatcaggctggcctggaactcagaaatccgcctgtctctgcctcccgagtgctgggattaaaggtgtgcaccaccaatgcctggtgagataactttaaagaactccctataaatgcatgagaaccactgttactgatgaatgtggttttttgacaactacattcacaaatggcctgtcttgtgttttgtcaccgttttgagggatgatgttttgtggcacgtgtgtgatcacagcctgatggttctggtcgtgggttggttcttctgggccagctttcacagactgctgcgcagctgcacctacagtgctgccccataatactgtttcactttggtgaagatcagcccaccttacaccccgagtgcaggtgtgaaccacggtaagtgtgcacagtccttagggaaaacagggacgcagaggcctgcctcctctcttttccatgccaaaatgaaatgaccaagaaacaaaacatttaaaaagttgtttctaaatgctgagacctaaccattgcttatatactgttgtctgttgaaacagtttgttacaatttcattctgttgaactaggtgagactttaagaaatgttgaaattatgttaatttcctattattatttaatataaagatatttaaaatgtctagtgttatgagttggtttaatatatatctcatgtatgtatcagtcctattttaagcgctttttaaaaaagacttgtttaggtatatattttatgtatatgggtgttttatctgaatgtatggacaccagaagagagcactgggtcccatgagactacagtcaaatgggatcacagtcagatgcaattttgagctgccatgtgggtgctgggatttgaactcagggcctctggaagaacagccagtgcttttaactgctgaaccatctcttgggacgcctatgggtcctattttaaaggaagaccgatatttcccaagtgtcacccttgcttactctactgcagacacaggaactatggcagtttaaggtaggtcagcgggcaggcccagcggtgaggcctctctgaggtagaggcctggggatcaggcgtttagtcattcaaggccggcagggctccatgggatcacatccaagtcagggcaagacatccacttacactttcaggtagctgcctgttagacgtcaccaagcatgtcctagcttaagtatgacgaggatggcattttctaagtacagagcatatatgtagcacttgggataagctgatctagggacttcctgaggcggcagagcttaaagtggactccattgactgcttggagactactgtggaatgaaggcatgtctcagtaaacagaacaaacctgtggcatctgtctgcctcagggaggcatcagacaaagctggaaaggatttagaaaattcttttacctactctgggagctgggaggctgtccttcatcccgagggcattatttatctgaaggcatcttttgtttaagattcattcatttgttcctcaaatatgttcagtgcctgccttgcaccaggcactattctagatgcgttgaaggcactaataaaagaaatgcctcacagtaaaaaaaaaaaaaaa

SEQ ID NO: 47 - DNA, 5961 bp, Homo sapiens
aaagtgaatggcgaaggactgaagggatccccccttcgggtccccggccgccctgttcaccctcgttcatcctcctttccgaagctcgctctcgaaggcaggagcgaccggcgcctttggctgaggaggaggagaaggaggaatcgcgccaggcggagcgtcaggtcccgttttcctctccggcgtctccaatacaaagattacggtgcagaaggaaattgcactcgtctcctccgcgcccccggtacccaacacaatgcaccagccgcctgagtccaccgccgcggccgccgccgctgcagacattagcgctaggaagatggcgcacccggcaatgttccctcgaaggggcagcggtagtggcagcgcctctgctctcaatgcagcaggtaccggcgtcggtagtaatgccacatcttccgaggattttccgcctccgtcgctgcttcagccgccgccccctgcagcatcttctacgtcgggaccacagcctccgcctccacaaagcctgaacctcctttcgcaggctcagctgcaggcacagcctcttgcgccaggcggaactcaaatgaaaaagaaaagtggcttccagataactagcgttactcctgctcagatctccgctagtatcagctctaacaacagtatagcagaggacactgagagctatgatgatctggatgaatctcacacggaagatctctcttcttcggagatccttgatgtgtcactttccagggctactgacttaggggagcccgaacgcagctcctcagaagagaccctaaataacttccaggaagccgagacacctggggcagtctctcccaaccagccccaccttcctcagcctcatttgcctcaccttccacaacagaatgttgtgatcaatgggaatgctcatccacaccacctccatcaccaccatcagattcatcatgggcaccacctccaacatggtcaccaccatccatctcatgttgctgtggccagtgcatccattactggtgggccaccctcaagcccagtatctagaaaactctctacaactggaagctctgacagtatcacaccagttgcaccaacttctgctgtatcatccagtggttcacctgcatctgtaatgactaatatgcgtgctccaagtactacaggtggaataggtataaattctgttactggcactagtacagtaaataatgttaacattactgctgtgggtagttttaatcctaatgtgacaagcagcatgcttggtaatgttaatataagtacaagcaatattcctagtgctgctggtgtgagtgttgggcctggagttaccagtggtgttaatgtgaatatcttgagtggcatgggcaatggtactatttcttcctctgctgctgttagcagtgttcctaatgcagctgcagggatgactgggggatcggtttcaagtcagcagcaacaaccaacagttaacacttcgaggttcagagttgtgaagttagattctagttctgagccctttaaaaaaggtagatggacttgcactgagttctatgaaaaagaaaatgctgtacctgctacagaaggtgtgctgataaataaagtggtggagactgtaaagcaaaatccgatagaagtgacttctgaaagggagagcactagtgggagttcagtgagcagtagtgtcagcacactgagtcactatacagagagtgtgggaagtggagagatgggagcccctactgtggtggtgcagcagcagcagcagcaacaacaacaacaacagcaacaaccagctctccaaggtgtgaccctccaacagatggattttggtagcactggtccacagagtattccagcagttagtataccacagagtatttctcagtcacagatctcacaagtacaattacagtctcaagaactgagctatcagcaaaagcaaggtcttcagccagtacctctgcaagccactatgagtgctgcaactggtatccagccatcgcctgtaaatgtggttggtgtaacttcagctttaggtcagcagccttccatttccagtttggctcaaccccagctaccatattctcaggcggctcctccagtgcaaactccccttccaggggcaccaccaccccaacagttacagtatggacaacagcaaccaatggtttctacacagatggccccaggccatgtcaaatcagtgactcaaaatcctgcttcagagtatgtacaacagcagccaattcttcaaacagcaatgtcctccggacagcccagttctgcaggagtaggagcaggaacaacagtgattcctgtggctcagccacagggtatccagctgccagtgcagcccacagcagtcccagcacaacctgcaggggcatctgtccagcctgttggccaggctccggcagcagtgtctgctgtacctactggcagtcagattgcaaatattggtcagcaagcaaacatacctactgcagtgcagcagccctctacccaggttccaccttcagttattcagcagggtgctcctccatcttcgcaagtggttccacctgctcaaactgggattattcatcagggagttcaaactagtgctccaagccttcctcaacaattggttattgcatcccaaagttccttgttaactgtgcctccccagccacaaggagtagaaccagtagctcaaggaattgtttcacagcagttgcctgcagttagttctttgccctctgctagtagtatttctgttacaagtcaggttagttcaactggtccttctggaatgccttctgccccaacaaacttggttccaccacaaaatatagcacaaacccctgctacccaaaatggtaatttggttcaaagtgttagtcaacctcccttgatagcaactaatacaaatttgcctttggcacaacagataccactaagttctacccagttctccgcacaatcattagctcaggcaattggaagccaaattgaagatgccaggcgtgcagcggagccctccttagttggcttacctcagactatcagtggtgacagtgggggaatgtcagcagtttcagatgggagtagcagcagcctagcagcctctgcttctcttttcccgttgaaggtgctaccgctgacgacacccctggtggatggcgaggatgagagctcctctggtgcaagtgtggtagctattgacaacaaaatcgagcaagctatggatctagtgaaaagccatttgatgtatgcggtcagagaagaagtggaggtcctcaaagagcaaatcaaagaactaatagagaaaaattcccagctggagcaggagaacaatctgctgaagacactggccagtcctgagcagcttgcccagtttcaggcccagctgcagactggctccccccctgccaccacccagccacagggcaccacacagccccccgcccagccagcatcgcagggctcaggaccaaccgcatagctgcctatgcccccgcagaactggctgctgcgtgtgaactgaacagacggagaagatgtgctagggagaatctgcctccacagtcacccatttcattgctcgctgcgaaagagacgtgagactgacatatgccattatctcttttccagtattaaacactcatatgcttatggcttggagaaatttcttagttgggtgaattaaaggttaatccgagaattagcatggatataccgggacctcatgcagcttggcagatatctgagaaatggtttaattcatgctcaggagctgtgtgcctttccatcccttccggctccctacccctcacttccaagggttctctctcctgcttgcgcttagtgtcctacatggggttgtgaagcgatggagctcctcactggactcgcctctctcctctcctccccccaggaggaacttgaaaggagggtaaaaagactaaaatgagggggaacagagttcactgtacaaatttgacaactgtcaccaaaattcataaaaaacaatagtactgtgcctctttcttctcaaacaatggatgacacaaaactatgagagtgacaaaatggtgacaggtagctgggacctaggctatcttaccatgaaggttgttttgcttattgtatatttgtgtatgtagtgtaactattttgtacaatagaggactgtaactactatttaggttgtacagattgaaatttagttgtttcattggctgtctgaggaggtgtggacttttatatatagatctacataaaaactgctacatgacaaaaaccacacctaaagaaattttaagaatttggcacagttactcactttgtgtaatctgaaatctagctgctgaatacgctgaagtaaatccttgttcactgaagtctttcaattgagctggttgaatactttgaaaaatgctcagttctaactaatgaaatggatttcccagtaggggtttctgcatatcacctgtatagtagttatatgcatatgtttctgtgcatgttctctacacaattgtaaggtgtcactgtatttaactgttgcacttgtcaactttcaataaagcatataaatgttgataaacaagtgtttttcatatgaccctgttaacataatggcagtcatttccacaactgtttccaggtaaagttaacaatttgactagtaaaatcctgaagggcagtggaatgattggaaaagtaggggcgtgatttgactgcatgaagtaaaaggaaattgacacattgagttgttcagcaggtaagagaagagacagtggaggagtccagctcaattctaactactgtgtgacttggacaggttgcttcactcctggacggatgtaggggtttgaaagagatgatctcttacattcccctttggctttaaaattctaggagtccttgaaaatcttaattttttactgaactgagaggaagaaggagccttattctgtgtataattggaataagcagatctaggatcaaaagacggaagttggagaagctgatttccactttaagaaccctgtcaccctgggttgggatgtttctcctagtgtaggatgagtttctagctcctgggataattcgttttgtctaagcaaaagatgatttgaggtgggaccagatgagcaaggatgtccttgcagttgattctgcatgactataaaacaggccaaaattaaaaccacgaaggaatcctgaggcagattggcccctggcacctgaggagtgagcctgagggatcccactctgcagtaggagtaacgtgagcgagcaagtgaatggggtgggtttaccggctctgtaatctatcaacctagggcctcagcagtggccccctgcccagtaggaattggaccaatcccaagttctaaggcttgtcttaagtccttgcagaacaaggacaaactttctgaatcattcatttctctcatctagagctggtgggagattgagaggtgaatgggacatccaagatccctaaaaagaattgttcgatagcgtgcatgtgttataaagtggtgacacgggcatcctgttgaaatgatggatggctcactgccataggctgatagcagttgtcataaagatattttgggggaatttgaaaaggacgtaaagaaaaatctttcatattggcttgttggttatataacttcaaatttaataaaggaatacttacgtagtaattacatttccttgaaaaaactatagtgaatagaaatccctagccatttcattttttatgtttttaatgaagatctttaaaataccataggtggtaatcgtggaaaatttgaaaaatctcatgtcagtgtattaagatggtggagaagtttttttctccattatttaatggaactttgggtctttttattaaaaatgtgagactcatgaaattttggcagctgaatatttgtgaaataagaatgaattattaaaagatgtctttccata

SEQ ID NO: 48 - DNA, 4601 bp, Mus musculus
ctggagggggaagtgaatggcgaaggactgaaggggtcccccttcggttccccagccacctggttcacccggttcatcctcccttcccgaagttagccctcgaaggcacgagcagccggcgccttcggcagaggaggaggaggaggagggatcgcgtcgggcggagcttcaggtcctgttcgtctctccggcttctccatacaaagattacggtgcagaaggaaattgcacttgcctcctccgccccccggtacccagcacaatgcaccagccgccggagtccaccgccgcggcggccgctgctgcagacattagtgctaggaagatggcgcacccggcaatgttccctcgaaggggcagcggtggtggcagcgcctctgctctcaatgcagcaggtacgggcgtcagtggtgctgctccatcttctgaagattttcctcctccttcgctgctccagccgccgcctcctgctgcatcttccacgcagggaccccagcctccgcctccacaaagcctgaacctcctctcgcaggctcagctgcagggacagcctcttgcgccaggcggaactcagatgaaaaagaaaagtggcttccagataactagcgttactccggctcagatctctgccagcatcagctccaacaacagcatcgcggaggacaccgagagctacgacgatctggatgagtcgcacacggaagatctctcctcttccgagatcctcgacgtctccctttccagggccactgacttaggtgagcctgaacgaagctcctcagaggagactctgaataacttccaggaagctgagacacctggggcagtctctcctaaccagccccaccttcctcagcctcatttgcctcaccttccacaacagaacgttgtgatcaatgggaatgcccatccccaccacctccatcatcaccatcaccctcatcatgggcaccaccttcaccatgggcaccatcattcatcacatgctgctgtggccggtccatccattcccggagggccaccctcgagcccagtgtccaggaaactctctacaactggaagttctgatggtggtgtgccagtcgcacctcctcctgcagtaccatcgagtggcttaccagcatcagtgatgacgaacatccgtactccaagtactacaggaagcctaggtataaattcggttactggcacgagcgctacgaataatgttaacattgctgctgtgggtagtttcagtcccagtgtgacgaacagcgtgcatggtaatgctaatataaatacaagcaatatccctaatgctgctagcataagtggtgggcctggagttaccagtgttgttaattcgagtatcttgagtggcatgggcaatggtaccgtttcttcctctcctgttgctaacagtgtccttaatgcagctgcaggtatcactgtgggagtggtttccagtcagcagcagcagcagcagcagcagcaaccaacagttaacacatcgaggttcagggttgtgaagttagactctacttctgaaccctttaaaaaaggtcgatggacttgcacagaattctatgagaaggaaaacgctgtgccagctacagaaggcgtggccgtcaataaagtggtggagacggtgaagcaaacccccacggaagcatcgtcctcggagagggagagcactagtgggagttctgtgagcagtagtgtgagcacactgagtcactacacggagagtgtgggaagtggagagatgatgggagccccggctgtggtggcgccgcagcagccgccgctaccaccagcgcctccaggtcttcaaggtgtggctctccaacagctagagttcagtagccctgctccacagagtattgcggcggttagtatgccacagagtatttctcagtcacagatgtcacaagtacagttacagcctcaagagttgagctttcagcagaagcagactcttcagcctgtccctctgcaagccaccatgagtgccgcaactggtatccagccttcccctgtcagcgtggtcggcatcacggcggctgtaggtcagcagccttctgtttccagcctggctcaaccgcagctgccatattctcagacagctcctcccgtgcaaactcctcttccaggggcaccaccccagcagttacaatatgggcagcagcaaccaatggttcctgcgcagatagccccaggccatggccagccagtgactcaaaatccaacttcagagtatgtgcagcagcagcagcagccaatatttcaagcagcattgtcctctgggcagtccagttccacgggcacgggggcaggaatatcagtgattcctgtggctcaggcacaggggatccagctgccagggcagcccacagcagtacaaacacaacctgcaggggcggctgggcagcccattggccaggctcaaacagcagtgtccactgtaccaactggcggtcagattgcaagtatcggtcaacaggcaaacatacccactgcagtacagcagccctctacccaagttacaccttcagttattcagcaaggtgctcctccatcttcacaagtagtcctacctgctccaaccgggatcattcatcagggagttcaaacccgtgcttcaagccttccacaacaattggtcattgcaccccagagtaccttggtaactgtgcctccccagccacagggagtagaaaccgtggcccaaggggttgtttcccagcagttgcccacaggcagtcctctgccctctgctagcactatttctgttacaaatcaggttagttcagccgctccttctggaatgccttctgtcccaacaaacttagttccaccacagaatatagcacaacccccagccacccaaaatggcagtttggttcaaagtgttagtcaatctcccttgatagccactaacataaatttgcctttggcacaacagataccactaagttctactcagttctctacacaatcattagctcaggccattggaagccaaatggaagatgccaggcgcccagcggagccctccttaggcggcttacctcagactatgagtggtgacagtgggggaatgtcagcagtttcagatgggagtagcagcagcctagcagcccctgcttctctcttcccgttgaaggtgctaccgctgacaacacccctggtggatggcgaggacgagagctctggtgcaagtgtggtagctatcgacaacaaaatagagcaagctatggatctggtgaaaagccatttgatgtatgcggtgagggaggaagtggaagttctgaaggagcagatcaaagaactaatagagaaaaactcccagctggagcaggagaacaatctgctgaagacgctggccagtccggagcagctcgcccagtttcaggcccagctgcagactggctcccctccggccaccacgcagccacaggggaccacacagccccctgcacagccagcatcccagggctcaggatcaaccgcatagcctcctaggccccaacagaactggctgctgctgctgctgtctgaactgaacagaccgaagagatgtgctagagagaagccgcctccacagtcacccatttcattgctgtctacgaaagagacgtgagactcacacgctgttctcgctttctccccagtattaagcactcataagcttttggcttgaagaaatgtactagttgagtgaattaaaggttaatcagagagtgagcagggatgtgccctgtgcaacgtggcagatgtctgaggaatggtttaattgaccccgaggagctctgtgccttttcaaccctccccagccgcccaccctgcttctgagagctcgggcggctcgccttcgtggggctcgcctgcgtggggttcggaaagtgggctgctcctggattctgcgctctcttctccttcccttcaaagaactcggagaggccagaaacaagactgcaatggggggcggggggagggatgatgcagtccttatacaaaaccgacaactgtcaccaaagcttataaaacacgatagtactgtccctcttttctgaaccatcagaagacacaaaactgttagtgacacaacggtgacaggtagctgggacctaggctatcttattatgaaggttgttttgcttgttgtatatttgtgtatgtagtgtaacgaatttgtaccatagaggactgtccgtaactactgtttagcttctacacattgaaatgtagatgtttcattggctgtctgaaaaggtgtggcttgtccttcctagagagatctacttaaaaactgctttgtgacaaaaaccacacctgaagaaattttaagaatttggcccagttagtcactctgtgtaatcccggaatctagctgctgaagtcttgcgaagtaaactccccgtgaccgatgtcagttaagctggtgatacctggagaagtggtcagttgctaaggaagtggatttcccagtaggggtttctgcacctcacctgtatagtcgttctgcgcatgtcccccacacagtccccactgtatttacctgttctacttgtcacctttcaataaagcatatcaaatgttgatacaaaaaaaaaaaaaaa

SEQ ID NO: 49 - protein, 436 aa, Homo sapiens
MAGVPESLNLMCDRNGGRRLRQWLIEQIDSSMYPGLIWENEEKSMFRIPWKHAGKQDYNQEVDASIFKAWAVFKGKFKEGDKAEPATWKTRLRCALNKSPDFEEVTDRSQLDISEPYKVYRIVPEEEQKCKLGVATAGCVNEVTEMECGRSEIDELIKEPSVDDYMGMIKRSPSPPEACRSQLLPDWWAQQPSTGVPLVTGYTTYDAHHSAFSQMVISFYYGGKLVGQATTTCPEGCRLSLSQPGLPGTKLYGPEGLELVRFPPADAIPSERQRQVTRKLFGHLERGVLLHSSRQGVFVKRLCQGRVFCSGNAVVCKGRPNKLERDEVVQVFDTSQFFRELQQFYNSQGRLPDGRVVLCFGEEFPDMAPLRSKLILVQIEQLYVRQLAEEAGKSCGAGSVMQAPEEPPPDQVFRMFPDICASHQRSFFRENQQITV

SEQ ID NO: 50 - protein, 424 aa, Mus musculus
MCDRNGGRRLRQWLIEQIDSSMYPGLIWENDEKTMFRIPWKHAGKQDYNQEVDASIFKAWAVFKGKFKEGDKAEPATWKTRLRCALNKSPDFEEVTDRSQLDISEPYKVYRIVPEEEQKCKLGVAPAGCMSEVPEMECGRSEIEELIKEPSVDEYMGMTKRSPSPPEACRSQILPDWWVQQPSAGLPLVTGYAAYDTHHSAFSQMVISFYYGGKLVGQATTTCLEGCRLSLSQPGLPKLYGPDGLEPVCFPTADTIPSERQRQVTRKLFGHLERGVLLHSNRKGVFVKRLCQGRVFCSGNAVVCKGRPNKLERDEVVQVFDTNQFIRELQQFYATQSRLPDSRVVLCFGEEFPDTVPLRSKLILVQVEQLYARQLVEEAGKSCGAGSLMPALEEPQPDQAFRMFPDICTSHQRPFFRENQQITV

SEQ ID NO: 51 - protein, 262 aa, Homo sapiens
MLALEAAQLDGPHFSCLYPDGVFYDLDSCKHSSYPDSEGAPDSLWDWTVAPPVPATPYEAFDPAAAAFSHPQAAQLCYEPPTYSPAGNLELAPSLEAPGPGLPAYPTENFASQTLVPPAYAPYPSPVLSEEEDLPLDSPALEVSDSESDEALVAGPEGKGSEAGTRKKLRLYQFLLGLLTRGDMRECVWWVEPGAGVFQFSSKHKELLARRWGQQKGNRKRMTYQKLARALRNYAKTGEIRKVKRKLTYQFDSALLPAVRRA

SEQ ID NO: 52 - protein, 267 aa, Mus musculus
MLALEAAQLDGPHLSCLYPEGVFYDLDSCKPFSYPDSDGGLDSTWGWTEAPPAPAIAPYEAFDPATAAFSHSQTVQLCYSHGPNPSTYSPMGTLDPAPSLEAPGPGLQVYPPEDFTSQTLGSLAYAPYPSPVLSEEEDIMLDSPALEVSDSESDEALLAGSEGRGSEAGARKKLRLYQFLLGLLLRGDMRECVWWVEPGAGVFQFSSKHKELLARRWGQQKGNRKRMTYQKLARALRNYAKTGEIRKVKRKLTYQFDSALLPASRHV

SEQ ID NO: 53 - protein, 648 aa, Homo sapiens
MAAPVKGNRKQSTEGDALDPPASPKPAGKQNGIQNPISLEDSPEAGGEREEEQEREEEQAFLVSLYKFMKERHTPIERVPHLGFKQINLWKIYKAVEKLGAYELQSMALGERIGWPLLEPQSSCQTAVLRVPARAAGAARTPPPGGARRPRPHEVPLQVTGRRLWKNVYDELGGSPGSTSAATCTRRHYERLVLPYVRHLKGEDDKPLPTSKPRKQYKMAKENRGDDGATERPKKAKEERRMDQMMPGKTKADAADPAPLPSQEPPRNSTEQQGLASGSSVSFVGASGCPEAYKRLLSSFYCKGTHGIMSPLAKKKLLAQVSKVEALQCQEEGCRHGAEPQASPAVHLPESPQSPKGLTENSRHRLTPQEGLQAPGGSLREEAQAGPCPAAPIFKGCFYTHPTEVLKPVSQHPRDFFSRLKDGVLLGPPGKEGLSVKEPQLVWGGDANRPSAFHKGGSRKGILYPKPKACWVSPMAKVPAESPTLPPTFPSSPGLGSKRSLEEEGAAHSGKRLRAVSPFLKEADAKKCGAKPAGSGLVSCLLGPALGPVPPEAYRGTMLHCPLNFTGTPGPLKGQAALPFSPLVIPAFPAHFLATAGPSPMAAGLMHFPPTSFDSALRHRLCPASSAWHAPPVTTYAAPHFFHLNTKL

SEQ ID NO: 54 - protein, 590 aa, Mus musculus
MAAPPAKGNTEQSEEGDLPQLPVSPKPDDEQSRSQSPTQLQDSPEAGGEQEEEQAFLVSLYKFMKERHTPIERVPHLGFKQINLWKIYKAVEKLGAYELVTGRRLWKNVYDELGGSPGSTSAATCTRRHYERLVLPYVRHLKGEDDKPLPPTKPRKQYKMAKELRGDDGTTEKLKKAKDSEERRVEQTTPGKTKSDATGQTQLPCQGSSRDSTEQLGPVSGPSPPLTGASSCPEAYKRLLSSFYCKGAHGIMSPLAKKKLLAQVSKAEALQCQEEGCRHGARSPNKDIQDSPQNLRGPAENSEHQLTPREGLQAPGGSTRMEAQVGPCPTAPMFSGCFHAYPTEVLKPVSQHPRDFFSGLKDRVLLGPPGKEEGPTTKESHLVWGGDANHPSAFHKGSTRKRSFYPKPKACWVSPMAKVPTERPGAPSPHPSSPGLGSKRGLEEEGFAHGGKKLRAVSPFLKEVDSKETGGKPAAPGLAVSCLLGPTPGPTPPEAYRGTMLRCPLNFTGSADPLKGQASLPFSPLVIPAFPAHLLATTGSSPMAASLMHFPPTPYDAVLRNRLGPASSAWHMPPVTTYAAPHFFHLNTKL

SEQ ID NO: 55 - protein, 835 aa, Homo sapiens
MSRRKQGKPQHLSKREFSPEPLEAILTDDEPDHGPLGAPEGDHDLLTCGQCQMNFPLGDILIFIEHKRKQCNGSLCLEKAVDKPPSPSPIEMKKASNPVEVGIQVTPEDDDCLSTSSRGICPKQEHIADKLLHWRGLSSPRSAHGALIPTPGMSAEYAPQGICKDEPSSYTCTTCKQPFTSAWFLLQHAQNTHGLRIYLESEHGSPLTPRVGIPSGLGAECPSQPPLHGIHIADNNPFNLLRIPGSVSREASGLAEGRFPPTPPLFSPPPRHHLDPHRIERLGAEEMALATHHPSAFDRVLRLNPMAMEPPAMDFSRRLRELAGNTSSPPLSPGRPSPMQRLLQPFQPGSKPPFLATPPLPPLQSAPPPSQPPVKSKSCEFCGKTFKFQSNLVVHRRSHTGEKPYKCNLCDHACTQASKLKRHMKTHMHKSSPMTVKSDDGLSTASSPEPGTSDLVGSASSALKSVVAKFKSENDPNLIPENGDEEEEEDDEEEEEEEEEEEEELTESERVDYGFGLSLEAARHHENSSRGAVVGVGDESRALPDVMQGMVLSSMQHFSEAFHQVLGEKHKRGHLAEAEGHRDTCDEDSVAGESDRIDDGTVNGRGCSPGESASGGLSKKLLLGSPSSLSPFSKRIKLEKEFDLPPAAMPNTENVYSQWLAGYAASRQLKDPFLSFGDSRQSPFASSSEHSSENGSLRFSTPPGELDGGISGRSGTGSGGSTPHISGPGPGRPSSKEGRRSDTCEYCGKVFKNCSNLTVHRRSHTGERPYKCELCNYACAQSSKLTRHMKTHGQVGKDVYKCEICKMPFSVYSTLEKHMKKWHSDRVLNNDIKTE

SEQ ID NO: 56 - protein, 773 aa, Mus musculus
MSRRKQGKPQHLSKREFSPEPLEAILTDDEPDHGPLGAPEGDHDLLTCGQCQMNFPLGDILIFIEHKRKQCNGSLCLEKGVDKPPSPSPIEMKKASNPVEVGIQVTPEDDDCLSTSSRGICPKQEHIADKLLHWRGLSSPRSAHGALIPTPGMSAEYAPQGICKDEPSSYTCTTCKQPFTSAWFLLQHAQNTHGLRIYLESEHGSPLTPRVGIPSGLGAECPSQPPLHGIHIADNNPFNLLRIPGSVSREASGLAEGRFPPTPPLFSPPPRHHLDPHRIERLGAEEMALATHHPSAFDRVLRLNPMAMEPPAMDFSRRLRELAGNTSSPPLSPGRPSPMQRLLQPFQPGSKPPFLATPPLPPLQSAPPPSQPPVKSKSCEFCGKTFKFQSNLVVHRRSHTGEKPYKCNLCDHACTQASKLKRHMKTHMHKSSPMTVKSDDGLSTASSPEPGTSDLVGSASSALKSVVAKFKSENDPNLIPENGDEEEEEDDEEEEEEEEEEEEELTESERVDYGFGLSLEAARHHENSSRGAVVGVGDEGRALPDVMQGMVLSSMQHFSEAFHQVLGEKHKRSHLAEAEGHRDTCDEDSVAGESDRIDDGTVNGRGCSPGESASGGLSKKLLLGSPSSLSPFSKRIKLEKEFDLPPAAMPNTENVYSQWLAGYAASRQLKDPFLTFGDSRQSPFASSSEHSSENGSLRFSTPPGELDGGISGRSGTGSGGSTPHISGPGPGRPSSKEGRRSDTCPSHTPVRRSTPRAQDVWQFSDGSSRTLKF

SEQ ID NO: 57 - protein, 653 aa, Homo sapiens
MPASRLRDRAASSASGSTCGSMSQTHPVLESGLLASAGCSAPRGPRKGGPAPVDRKAKASAMPDSPAEVKTQPRSTPPSMPPPPPAASQGATRPPSFTPHTHREDGPATLPHGRFHGCLKWSMVCLLMNGSSHSPTAINGAPCTPNGFSNGPATSSTASLSTQHLPPACGARQLSKLKRFLTTLQQFGSDISPEIGERVRTLVLGLVNSTLTIEEFHSKLQEATNFPLRPFVIPFLKANLPLLQRELLHCARLAKQTPAQYLAQHEQLLLDASASSPIDSSELLLEVNENGKRRTPDRTKENGSDRDPLHPEHLSKRPCTLNPAQRYSPSNGPPQPTPPPHYRLEDIAMAHHFRDAYRHPDPRELRERHRPLVVPGSRQEEVIDHKLTEREWAEEWKHLNNLLNCIMDMVEKTRRSLTVLRRCQEADREELNHWARRYSDAEDTKKGPAPAAARPRSSSAGPEGPQLDVPREFLPRTLTGYVPEDIWRKAEEAVNEVKRQAMSELQKAVSDAERKAHELITTERAKMERALAEAKRQASEDALTVINQQEDSSESCWNCGRKASETCSGCNAARYCGSFCQHRDWEKHHHVCGQSLQGPTAVVADPVPGPPEAAHSLGPSLPVGAASPSEAGSAGPSRPGSPSPPGPLDTVPR

SEQ ID NO: 58 - protein, 620 aa, Mus musculus
MSQASTTTLESGALLSGPRGLQNGSPAHRKEKAAAMPDSPAEVKTQPRSTPPSMPPPPPTSSQGATRPPSFTPHTHGEDGPATSLPHGRFHGCLKWSMVCLLMNGSSHSPTAIHGAPSTPNGFSNGPATSSTASLSTQHLPPACGARQLSKLKRFLTTLQQFGSDISPEIGERVRTLVLGLVNSTLTIEEFHAKLQEATNFPLRPFVIPFLKANLPLLQRELLHCARLAKQTPAQYLAQHEQLLLDASATSPVDSSELLLEVNENGKRRTPDRTKENGSDRDPLHPDHLSKRSCTLSPAQRCSPSNGLPHPTPPPPPHYRLEDMAMAHHFRDSYRHPDPRELRERHRPLAIPGSRQEEVIDHRLTEREWAEEWKHLNSLLNCIMDMVEKTRRSLTVLRRCQEADREELNHWIRCYSDSEEGKKGPTPISARSLNSCSGPEGSQLDVHRDFTPRTLSGYMPEEIWRKAEEAVNEVKRQAMSELQKAVSDAERKAHELITTERAKMERALAEAKRQASEDALTVINQQEDSSESCWNCGRKASETCSGCNAARYCGSFCQHKDWEKHHHVCGQSLQGPAAAVADPLPGQPDATASPSEAGSAGPSRPCSPGPPGPLDAAVPR

SEQ ID NO: 59 - protein, 520 aa, Homo sapiens
MEVLESGEQGVLQWDRKLSELSEPGDGEALMYHTHFSELLDEFSQNVLGQLLNDPFLSEKSVSMEVEPSPTSPAPLIQAEHSYSLCEEPRAQSPFTHITTSDSFNDDEVESEKWYLSTDFPSTSIKTEPVTDEPPPGLVPSVTLTITAISTPLEKEEPPLEMNTGVDSSCQTIIPKIKLEPHEVDQFLNFSPKEAPVDHLHLPPTPPSSHGSDSEGSLSPNPRLHPFSLPQTHSPSRAAPRAPSALSSSPLLTAPHKLQGSGPLVLTEEEKRTLIAEGYPIPTKLPLSKSEEKALKKIRRKIKNKISAQESRRKKKEYMDSLEKKVESCSTENLELRKKVEVLENTNRTLLQQLQKLQTLVMGKVSRTCKLAGTQTGTCLMVVVLCFAVAFGSFFQGYGPYPSATKMALPSQHSLQEPYTASVVRSRNLLIYEEHSPPEESSSPGSAGELGGWDRGSSLLRVSGLESRPDVDLPHFIISNETSLEKSVLLELQQHLVSAKLEGNETLKVVELDRRVNTTF

SEQ ID NO: 60 - protein, 521 aa, Mus musculus
MEVLESGEQSVLQWDRKLSELSEPGETEALMYHTHFSELLDEFSQNVLGQLLSDPFLSEKSESMEVEPSPTSPAPLIQAEHSYSLSEEPRTQSPFTHAATSDSFNDEEVESEKWYLSTEFPSATIKTEPITEEQPPGLVPSVTLTITAISTPFEKEESPLDMNAGGDSSCQTLIPKIKLEPHEVDQFLNFSPKEASVDQLHLPPTPPSSHSSDSEGSLSPNPRLHPFSLSQAHSPARAMPRGPSALSTSPLLTAPHKLQGSGPLVLTEEEKRTLVAEGYPIPTKLPLTKSEEKALKKIRRKIKNKISAQESRRKKKEYMDSLEKKVESCSTENLELRKKVEVLENTNRTLLQQLQKLQTLVMGKVSRTCKLAGTQTGTCLMVVVLCFAVAFGSFFQGYGPYPSATKMALPSQHPLSEPYTASVVRSRNLLIYEEHAPLEESSSPASAGELGGWDRGSSLLRASSGLEALPEVDLPHFLISNETSLEKSVLLELQQHLVSSKLEGNETLKVVELERRVNATF

SEQ ID NO: 61 - protein, 485 aa, Homo sapiens
MSYFVDSAGSSPVPYSAPRPAVVRQGPSNTYEDPRMNCGFQSNYHQQRPCYPFWDEMATQEVPTGLEHCVSDMECADVPLLTPSSKEMMSQALKATFSGFTKEQQRLGIPKDPRQWTETHVRDWVMWAVNEFSLKGVDFQKFCMNGAALCALGKDCFLELAPDFVGDILWEHLEILQKEDVKPYQVNGVNPAYPESRYTSDYFISYGIEHAQCVPPSEFSEPSFITESYQTLHPISSEELLSLKYENDYPSVILRDPLQTDTLQNDYFAIKQEVVTPDNMCMGRTSRGKLGGQDSFESIESYDSCDRLTQSWSSQSSFNSLQRVPSYDSFDSEDYPAALPNHKPKGTFKDYVRDRADLNKDKPVIPAAALAGYTGSGPIQLWQFLLELLTDKSCQSFISWTGDGWEFKLSDPDEVARRWGKRKNKPKMNYEKLSRGLRYYYDKNIIHKTAGKRYVYRFVCDLQSLLGYTPEELHAMLDVKPDADE

SEQ ID NO: 62 - protein, 440 aa, Mus musculus
MKAAVDLKPTLTIIKTEKVDLELFPSPDMECADVPLLTPSSKEMMSQALKATFSGFTKEQQRLGIPKDPRQWTETHVRDWVMWAVNEFSLKGVDFQKFCMSGAALCALGKECFLELAPDFVGDILWEHLEILQKEDVKPYQVNGANPTYPESCYTSDYFISYGIEHAQCVPPSEFSEPSFITESYQTLHPISSEELLSLKYENDYPSVILQDPLQTDTLQTDYFAIKQEVLTPDNMCLGRASRGKLGGQDSFESVESYDSCDRLTQSWSSQSSFNSLQRVPSYDSFDYEDYPAALPNHKPKGTFKDYVRDRADLNKDKPVIPAAALAGYTGSGPIQLWQFLLELLTDKSCQSFISWTGDGWEFKLSDPDEVARRWGKRKNKPKMNYEKLSRGLRYYYDKNIIHKTAGKRYVYRFVCDLQSLLGYTPEELHAMLDVKPDAD

SEQ ID NO: 63 - protein, 1123 aa, Homo sapiens
MNSPNESADGMSGREPSLEILPRTSLHSIPVTVEVKPVLPRAMPSSMGGGGGGSPSPVELRGALVGSVDPTLREQQLQQELLALKQQQQLQKQLLFAEFQKQHDHLTRQHEVQLQKHLKQQQEMLAAKQQQEMLAAKRQQELEQQRQREQQRQEELEKQRLEQQLLILRNKEKSKESAIASTEVKLRLQEFLLSKSKEPTPGGLNHSLPQHPKCWGAHHASLDQSSPPQSGPPGTPPSYKLPLPGPYDSRDDFPLRKTASEPNLKVRSRLKQKVAERRSSPLLRRKDGTVISTFKKRAVEITGAGPGASSVCNSAPGSGPSSPNSSHSTIAENGFTGSVPNIPTEMLPQHRALPLDSSPNQFSLYTSPSLPNISLGLQATVTVTNSHLTASPKLSTQQEAERQALQSLRQGGTLTGKFMSTSSIPGCLLGVALEGDGSPHGHASLLQHVLLLEQARQQSTLIAVPLHGQSPLVTGERVATSMRTVGKLPRHRPLSRTQSSPLPQSPQALQQLVMQQQHQQFLEKQKQQQLQLGKILTKTGELPRQPTTHPEETEEELTEQQEVLLGEGALTMPREGSTESESTQEDLEEEDEEDDGEEEEDCIQVKDEEGESGAEEGPDLEEPGAGYKKLFSDAQPLQPLQVYQAPLSLATVPHQALGRTQSSPAAPGGMKSPPDQPVKHLFTTGVVYDTFMLKHQCMCGNTHVHPEHAGRIQSIWSRLQETGLLSKCERIRGRKATLDEIQTVHSEYHTLLYGTSPLNRQKLDSKKLLGPISQKMYAVLPCGGIGVDSDTVWNEMHSSSAVRMAVGCLLELAFKVAAGELKNGFAIIRPPGHHAEESTAMGFCFFNSVAITAKLLQQKLNVGKVLIVDWDIHHGNGTQQAFYNDPSVLYISLHRYDNGNFFPGSGAPEEVGGGPGVGYNVNVAWTGGVDPPIGDVEYLTAFRTVVMPIAHEFSPDVVLVSAGFDAVEGHLSPLGGYSVTARCFGHLTRQLMTLAGGRVVLALEGGHDLTAICDASEACVSALLSVELQPLDEAVLQQKPNINAVATLEKVIEIQSKHWSCVQKFAAGLGRSLREAQAGETEEAETVSAMALLSVGAEQAQAAAAREHSPRPAEEPMEQEPAL

SEQ ID NO: 64 - protein, 1121 aa, Mus musculus
MLLVPKAQGLVEMLQTIYETESCFSADGMSGREPSLEILPRTPLHSIPVAVEVKPVLPGAMPSSMGGGGGGSPSPVELRGALAGPMDPALREQQLQQELLVLKQQQQLQKQLLFAEFQKQHDHLTRQHEVQLQKHLKQQQEMLAAKRQQELEQQRQREQQRQEELEKQRLEQQLLILRNKEKSKESAIASTEVKLRLQEFLLSKSKEPTPGGLNHSLPQHPKCWGAHHASLDQSSPPQSGPPGTPPSYKLPLLGPYDSRDDFPLRKTASEPNLKVRSRLKQKVAERRSSPLLRRKDGTVISTFKKRAVEITGTGPGVSSVCNSAPGSGPSSPNSSHSTIAENGFTGSVPNIPTEMIPQHRALPLDSSPNQFSLYTSPSLPNISLGLQATVTVTNSHLTASPKLSTQQEAERQALQSLRQGGTLTGKFMSTSSIPGCLLGVALEGDTSPHGHASLLQHVLLLEQARQQSTLIAVPLHGQSPLVTGERVATSMRTVGKLPRHRPLSRTQSSPLPQSPQALQQLVMQQQHQQFLEKQKQQQMQLGKILTKTGELSRQPTTHPEETEEELTEQQEALLGEGALTIPREGSTESESTQEDLEEEEEEEEEEEEDCIQVKDEDGESGPDEGPDLEESSAGYKKLFADAQQLQPLQVYQAPLSLATVPHQALGRTQSSPAAPGSMKSPTDQPTVVKHLFTTGVVYDTFMLKHQCMCGNTHVHPEHAGRIQSIWSRLQETGLLGKCERIRGRKATLDEIQTVHSEYHTLLYGTSPLNRQKLDSKKLLGPISQKMYAMLPCGGIGVDSDTVWNEMHSSSAVRMAVGCLVELAFKVAAGELKNGFAIIRPPGHHAEESTAMGFCFFNSVAITAKLLQQKLSVGKVLIVDWDIHHGNGTQQAFYNDPSVLYISLHRYDNGNFFPGSGAPEEVGGGPGVGYNVNVAWTGGVDPPIGDVEYLTAFRTVVMPIAQEFSPDVVLVSAGFDAVEGHLSPLGGYSVTARCFGHLTRQLMTLAGGRVVLALEGGHDLTAICDASEACVSALLSVELQPLDEAVLQQKPSVNAVATLEKVIEIQSKHWSCVQRFAAGLGCSLREAQTGEKEEAETVSAMALLSVGAEQAQAVATQEHSPR

SEQ ID NO: 65 - protein, 270 aa, Homo sapiens
MQYPHPGPAAGAVGVPLYAPTPLLQPAHPTPFYIEDILGRGPAAPTPAPTLPSPNSSFTSLVSPYRTPVYEPTPIHPAFSHHSAAALAAAYGPGGFGGPLYPFPRTVNDYTHALLRHDPLGKPLLWSPFLQRPLHKRKGGQVRFSNDQTIELEKKFETQKYLSPPERKRLAKMLQLSERQVKTWFQNRRAKWRRLKQENPQSNKKEELESLDSSCDQRQDLPSEQNKGASLDSSQCSPSPASQEDLESEISEDSDQEVDIEGDKSYFNAG

SEQ ID NO: 66 - protein, 271 aa, Mus musculus
MQFPHPGPAAAPAVGVPLYAPTPLLQPAHPTPFYIDDILGRGPAAPTPTPTLPSPNSSFTSLVSSYRTPVYEPTPVHPAFSHHPAAALAAAYGPSGFGGPLYPFPRTVNDYTHALLRHDPLGKPLLWSPFLQRPLHKRKGGQVRFSNDQTVELEKKFETQKYLSPPERKRLAKMLQLSERQVKTWFQNRRAKWRRLKQENPQSNKKDALDSLDTSCEQGQDLPSEQNKGASLDRSQCSPSPASQEDPDSEISEDSDQEVDIEGDKGYFNAG

SEQ ID NO: 67 - protein, 270 aa, Homo sapiens
MSSYFVNSFCGRYPNGPDYQLHNYGDHSSVSEQFRDSASMHSGRYGYGYNGMDLSVGRSGSGHFGSGERARSYAASASAAPAEPRYSQPATSTHSPQPDPLPCSAVAPSPGSDSHHGGKNSLSNSSGASADAGSTHISSREGVGTASGAEEDAPASSEQASAQSEPSPAPPAQPQIYPWMRKLHISHDNIGGPEGKRARTAYTRYQTLELEKEFHFNRYLTRRRRIEIAHALCLSERQIKIWFQNRRMKWKKDNKLKSMSMAAAGGAFRP

SEQ ID NO: 68 - protein, 270 aa, Mus musculus
MSSYFVNSFCGRYPNGPDYQLHNYGDHSSVSEQFRDSASMHSGRYGYGYNGMDLSVGRSGSGHFGSGERARSYAAGASAAPAEPRYSQPATSTHSPPPDPLPCSAVAPSPGSDSHHGGKNSLGNSSGASANAGSTHISSREGVGTASAAEEDAPASSEQAGAQSEPSPAPPAQPQIYPWMRKLHISHDNIGGPEGKRARTAYTRYQTLELEKEFHFNRYLTRRRRIEIAHALCLSERQIKIWFQNRRMKWKKDNKLKSMSMAAAGGAFRP

SEQ ID NO: 69 - protein, 119 aa, Homo sapiens
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELTPELVISNDKRSFCH

SEQ ID NO: 70 - protein, 119 aa, Mus musculus
MKALSPVRGCYEAVCCLSERSLAIARGRGKSPSTEEPLSLLDDMNHCYSRLRELVPGVPRGTQLSQVEILQRVIDYILDLQVVLAEPAPGPPDGPHLPIQTAELTPELVISKDKRSFCH

SEQ ID NO: 71 - protein, 519 aa, Homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 72 - protein, 515 aa, Mus musculus
MDVDEGQDMSQVSGKESPPVSDTPDEGDEPMPVPEDLSTTSGAQQNSKSDRGMASNVKVETQSDEENGRACEMNGEECAEDLRMLDASGEKMNGSHRDQGSSALSGVGGIRLPNGKLKCDICGIVCIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGMYPVIKEETNHNEMAEDLCKIGAERSLVLDRLASNVAKRKSSMPQKFLGDKCLSDMPYDSANYEKEDMMTSHVMDQAINNAINYLGAESLRPLVQTPPGSSEVVPVISSMYQLHKPPSDGPPRSNHSAQDAVDNLLLLSKAKSVSSEREASPSNSCQDSTDTESNAEEQRSGLIYLTNHINPHARNGLALKEEQRAYEVLRAASENSQDAFRVVSTSGEQLKVYKCEHCRVLFLDHVMYTIHMGCHGFRDPFECNMCGYHSQDRYEFSSHITRGEHRYHLS

SEQ ID NO: 73 - protein, 526 aa, Homo sapiens [sequence omitted]

SEQ ID NO: 74 - protein, 526 aa, Mus musculus
METDAIDGYITCDNELSPEGEHANMAIDLTSSTPNGQHASPSHMTSTNSVKLEMQSDEECDRQPLSREDEIRGHDEGSSLEEPLIESSEVADNRKVQDLQGEGGIRLPNGKLKCDVCGMVCIGPNVLMVHKRSHTGERPFHCNQCGASFTQKGNLLRHIKLHSGEKPFKCPFCSYACRRRDALTGHLRTHSVGKPHKCNYCGRSYKQRSSLEEHKERCHNYLQNVSMEAAGQVMSHHVPPMEDCKEQEPIMDNNISLVPFERPAVIEKLTANMGKRKSSTPQKFVGEKLMRFSYPDIHFDMNLTYEKEAELMQSHMMDQAINNAITYLGAEALHPLMQHAPSTIAEVAPVISSAYSQVYHPNRIERPISRETSDSHENNMDGPISLIRPKSRPQEREASPSNSCLDSTDSESSHDDRQSYQGNPALNPKRKQSPAYMKEDVKALDATKAPKGSLKDIYKVFNGEGEQIRAFKCEHCRVLFLDHVMYTIHMGCHGYRDPLECNICGYRSQDRYEFSSHIVRGEHTFH

SEQ ID NO: 75 - protein, 509 aa, Homo sapiens
MEDIQTNAELKSTQEQSVPAESAAVLNDYSLTKSHEMENVDSGEGPANEDEDIGDDSMKVKDEYSERDENVLKSEPMGNAEEPEIPYSYSREYNEYENIKLERHVVSFDSSRPTSGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRTFLQSTDPGDTASAEARHIKAEMGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLK

SEQ ID NO: 76 - protein, 507 aa, Mus musculus
MEDIQPTVELKSTEEQPLPTESPDALNDYSLPKPHEIENVDSREAPANEDEDAGEDSMKVKDEYSDRDENIMKPEPMGDAEESEMPYSYAREYSDYESIKLERHVPYDNSRPTGGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRAFLQNPDLGDAASVEARHIKAEMGSERALVLDRLASNVAKRKSSMPQKFIGEKRHCFDANYNPGYMYEKENEMMQTRMMDQAINNAISYLGAEALRPLVQTPPAPTSEMVPVISSVYPIALTRADMPNGAPQEMEKKRILLPEKILPSERGLSPNNSAQDSTDTDSNHEDRQHLYQQSHVVLPQARNGMPLLKEVPRSFELLKPPPICLRDSIKVINKEGEVMDVFRCDHCHVLFLDYVMFTIHMGCHGFRDPFECNMCGYRSHDRYEFSSHIARGEHRAMLK

SEQ ID NO: 77 - protein, 419 aa, Homo sapiens
MGEKKPEPLDFVKDFQEYLTQQTHHVNMISGSVSGDKEAEALQGAGTDGDQNGLDHPSVEVSLDENSGMLVDGFERTFDGKLKCRYCNYASKGTARLIEHIRIHTGEKPHRCHLCPFASAYERHLEAHMRSHTGEKPYKCELCSFRCSDRSNLSHHRRRKHKMVPIKGTRSSLSSKKMWGVLQKKTSNLGYSRRALINLSPPSMVVQKPDYLNDFTHEIPNIQTDSYESMAKTTPTGGLPRDPQELMVDNPLNQLSTLAGQLSSLPPENQNPASPDVVPCPDEKPFMIQQPSTQAVVSAVSASIPQSSSPTSPEPRPSHSQRNYSPVAGPSSEPSAHTSTPSIGNSQPSTPAPALPVQDPQLLHHCQHCDMYFADNILYTIHMGCHGYENPFQCNICGCKCKNKYDFACHFARGQHNQH

SEQ ID NO: 78 - protein, 419 aa, Mus musculus
MGEKKPEPLDFVKDFQEYLTQQTHHVNMISGSVSGDKEAETLQGAGTDGDQNGLDHPSVEVSLDENSGMLVDGFERTFDGKLKCRYCNYASKGTARLIEHIRIHTGEKPHRCHLCPFASAYERHLEAHMRSHTGEKPYKCELCSFRCSDRSNLSHHRRRKHKMVPIKGTRSSLSSKKMWGVLQKKTSNLGYSRRALINLSPPSMVVQKPDYLNDFTHEIPNIQTDSYEAMAKTTPTGGLPRDPQELMVDNPLNQLSTLAGQLSSLPPENQNPASPDVDACPDEKPFMIQQPSAQAVVSAVSASIPQSSSPTSPEPRPSHSQRNYSPVAGPSSEPSAHTSTPSIGNSQPSTPAPTLPVQDPQLLHHCQHCDVYFADNVLYTVHMGCHGYDSPFQCNVCGCKCKDKYDFACHFARGQHNQH

SEQ ID NO: 79 - protein, 516 aa, Homo sapiens
MPVPERPAAGPDSPRPGTRRAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 80 - protein, 457 aa, Mus musculus
MAEVRGVQRVLFGDWLLGEVSSGQYEGLQWLNEARTVFRVPWKHFGRRDLDEEDAQIFKAWAVARGRWPPSGVNLPPPEAEAAERRERRGWKTNFRCALHSTGRFILRQDNSGDPVDPHKVYELSRELGSTVGPATENREEVSLSNALPTQGVSPGSFLARENAGLQTPSPLLSSDAGDLLLQVLQYSHILESESGADPVPPQAPGQEQDRVYEEPYAAWQVEAVPSPRPQQPALTERSLGFLDVTIMYKGRTVLQAVVGHPRCVFLYSPMAPAVRTSEPQPVIFPSPAELPDQKQLHYTETLLQHVSPGLQLELRGPSLWALRMGKCKVYWEVGSPMGTTGPSTPPQLLERNRHTPIFDFSTFFRELEEFRARRRQGSPHYTIYLGFGQDLSAGRPKEKTLILVKLEPWVCKAYLEGVQREGVSSLDSSSLGLCLSSTNSLYEDIEHFLMDLGQWP

SEQ ID NO: 81 - protein, 473 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVSEDVDLLLNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 82 - protein, 466 aa, Mus musculus
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPSFEMPVTIPVSSHNSLVYSNPVSTLGNPNLLPLAHPSLQRNSMSPGVTHRPPSAGNTGGLMGGDLTSGAGTSAGNGYGNPRNSPGLLVSPGNLNKNIQAKSPPPMNLGMNNRKPDLRVLIPPGSKNTMPSVNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLSIKSEPVSPPRDRTTTPSRYPQHTTRHEAGRSPVDSLSSCSSSYDGSDREDHRNEFHSPIGLTRPSPDERESPSVKRMRLSEGWAT

SEQ ID NO: 83 - protein, 761 aa, Homo sapiens
MARRPRHSIYSSDEDDEDFEMCDHDYDGLLPKSGKRHLGKTRWTREEDEKLKKLVEQNGTDDWKVIANYLPNRTDVQCQHRWQKVLNPELIKGPWTKEEDQRVIELVQKYGPKRWSVIAKHLKGRIGKQCRERWHNHLNPEVKKTSWTEEEDRIIYQAHKRLGNRWAEIAKLLPGRTDNAIKNHWNSTMRRKVEQEGYLQESSKASQPAVATSFQKNSHLMGFAQAPPTAQLPATGQPTVNNDYSYYHISEAQNVSSHVPYPVALHVNIVNVPQPAAAAIQRHYNDEDPEKEKRIKELELLLMSTENELKGQQVLPTQNHTCSYPGWHSTTIADHTRPHGDSAPVSCLGEHHSTPSLPADPGSLPEESASPARCMIVHQGTILDNVKNLLEFAETLQFIDSDSSSWCDLSSFEFFEEADFSPSQHHTGKALQLQQREGNGTKPAGEPSPRVNKRMLSESSLDPPKVLPPARHSTIPLVILRKKRGQASPLATGDCSSFIFADVSSSTPKRSPVKSLPFSPSQFLNTSSNHENSDLEMPSLTSTPLIGHKLTVTTPFHRDQTVKTQKENTVFRTPAIKRSILESSPRTPTPFKHALAAQEIKYGPLKMLPQTPSHLVEDLQDVIKQESDESGIVAEFQENGPPLLKKIKQEVESPTDKSGNFFCSHHWEGDSLNTQLFTQTSPVADAPNILTSSVLMAPASEDEDNVLKAFTVPKNRSLASPLQPCSSTWEPASCGKMEEQMTSSSQARKYVNAFSARTLVM

SEQ ID NO: 84 - protein, 755 aa, Mus musculus
MARRPRHSIYSSDEDDEDIEMCDHDYDGLLPKSGKRHLGKTRWTREEDEKLKKLVEQNGTDDWKVIANYLPNRTDVQCQHRWQKVLNPELIKGPWTKEEDQRVIELVQKYGPKRWSVIAKHLKGRIGKQCRERWHNHLNPEVKKTSWTEEEDRIIYQAHKRLGNRWAEIAKLLPGRTDNAIKNHWNSTMRRKVEQEGYLQEPSKASQTPVATSFQKNNHLMGFGHASPPSQLSPSGQSSVNSEYPYYHIAEAQNISSHVPYPVALHVNIVNVPQPAAAAIQRHYNDEDPEKEKRIKELELLLMSTENELKGQQALPTQNHTCSYPGWHSTSIVDQTRPHGDSAPVSCLGEHHATPSLPADPGSLPEESASPARCMIVHQGTILDNVKNLLEFAETLQFIDSDSSWCDLSSFEFSEEAAAFSPSQQPTGKAFQLQQREGHGTRSAGEPSLRVTRRVLSEASLGPDSPQARHSKVPLVVLRKRRGQASPLAAGEPSPSLFADVISSTLKRSPVKSLPFSPSQFLNTSSNHESSGLDAPTLPSTPLIGHKLTPCRDQTVKTQKENSIFRTPAIKRSILESSPRTPTPFKHALAAQEIKYGPLKMLPQTPSHAVEDLQDVIKQESDESGIVAEFQESGPPLLKKIKQEVESPTEKSGNFFCSNHWAENSLSTQLFSQASPVADAPNILTSSVLMTPVSEDEDNVLKAFTVPKNRPLVGPLQPCSGAWEPASCGKTEDQMTASGPARKYVNAFSARTLVM

SEQ ID NO: 85 - protein, 521 aa, Homo sapiens
MASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 86 - protein, 528 aa, Mus musculus
MASNSLFSAVTPCQQSFFWDPSTSRRFSPPSSSLQPGKMSDVSPVVAAQQQQQQQQQQQQQQQQQQQQQQQQQQQQQEAAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSSLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSASYQFPMVPGGDRSPSRMVPPCTTTSNGSTLLNPNLPNQNDGVDADGSHSSSPTVLNSSGRMDESVWRPY

SEQ ID NO: 87 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 88 - protein, 770 aa, Mus musculus
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMDLTSECATSPM

SEQ ID NO: 89 - protein, 773 aa, Homo sapiens
MFSKRLEKIPQVPLLFPFIFIILTNYSKMEGAVESQPSFFKTSQDIVTCTWVENCYSSFSRRPLEQMFCKHQSKNIISWTGMVAHTCNPSTLGGQGLCDFAKMHHQQRMAALGTDKELSDLLDFSAMFSPPVSSGKNGPTSLASGHFTGSNVEDRSSSGSWGNGGHPSPSRNYGDGTPYDHMTSRDLGSHDNLSPPFVNSRIQSKTERGSYSSYGRESNLQGCHQQSLLGGDMDMGNPGTLSPTKPGSQYYQYSSNNPRRRPLHSSAMEVQTKKVRKVPPGLPSSVYAPSASTADYNRDSPGYPSSKPATSTFPSSFFMQDGHHSSDPWSSSSGMNQPGYAGMLGNSSHIPQSSSYCSLHPHERLSYPSHSSADINSSLPPMSTFHRSGTNHYSTSSCTPPANGTDSIMANRGSGAAGSSQTGDALGKALASIYSPDHTNNSFSSNPSTPVGSPPSLSAGTAVWSRNGGQASSSPNYEGPLHSLQSRIEDRLERLDDAIHVLRNHAVGPSTAMPGGHGDMHGIIGPSHNGAMGGLGSGYGTGLLSANRHSLMVGTHREDGVALRGSHSLLPNQVPVPQLPVQSATSPDLNPPQDPYRGMPPGLQGQSVSSGSSEIKSDDEGDENLQDTKSSEDKKLDDDKKDIKSITRSRSSNNDDEDLTPEQKAEREKERRMANNARERLRVRDINEAFKELGRMVQLHLKSDKPQTKLLILHQAVAVILSLEQQVRERNLNPKAACLKRREEEKVSSEPPPLSLAGPHPGMGDASNHMGQM

SEQ ID NO: 90 - protein, 670 aa, Mus musculus
MHHQQRMAALGTDKELSDLLDFSAMFSPPVSSGKNGPTSLASGHFTGSNVEDRSSSGSWGTGGHPSPSRNYGDGTPYDHMTSRDLGSHDNLSPPFVNSRIQSKTERGSYSSYGRENVQGCHQQSLLGGDMDMGNPGTLSPTKPGSQYYQYSSNNARRRPLHSSAMEVQTKKVRKVPPGLPSSVYAPSASTADYNRDSPGYPSSKPAASTFPSSFFMQDGHHSSDPWSSSSGMNQPGYGGMLGNSSHIPQSSSYCSLHPHERLSYPSHSSADINSSLPPMSTFHRSGTNHYSTSSCTPPANGTDSIMANRGTGAAGSSQTGDALGKALASIYSPDHTNNSFSSNPSTPVGSPPSLSAGTAVWSRNGGQASSSPNYEGPLHSLQSRIEDRLERLDDAIHVLRNHAVGPSTAVPGGHGDMHGIMGPSHNGAMGSLGSGYGTSLLSANRHSLMVGAHREDGVALRGSHSLLPNQVPVPQLPVQSATSPDLNPPQDPYRGMPPGLQGQSVSSGSSEIKSDDEGDENLQDTKSSEDKKLDDDKKDIKSITRSRSSNNDDEDLTPEQKAEREKERRMANNARERLRVRDINEAFKELGRMVQLHLKSDKPQTKLLILHQAVAVILSLEQQVRERNLNPKAACLKRREEEKVSSEPPPLSLAGPHPGMGDAANHMGQM

SEQ ID NO: 91 - protein, 706 aa, Homo sapiens
MNPQQQRMAAIGTDKELSDLLDFSAMFSPPVNSGKTRPTTLGSSQFSGSGIDERGGTTSWGTSGQPSPSYDSSRGFTDSPHYSDHLNDSRLGAHEGLSPTPFMNSNLMGKTSERGSFSLYSRDTGLPGCQSSLLRQDLGLGSPAQLSSSGKPGTAYYSFSATSSRRRPLHDSAALDPLQAKKVRKVPPGLPSSVYAPSPNSDDFNRESPSYPSPKPPTSMFASTFFMQDGTHNSSDLWSSSNGMSQPGFGGILGTSTSHMSQSSSYGNLHSHDRLSYPPHSVSPTDINTSLPPMSSFHRGSTSSSPYVAASHTPPINGSDSILGTRGNAAGSSQTGDALGKALASIYSPDHTSSSFPSNPSTPVGSPSPLTGTSQWPRPGGQAPSSPSYENSLHSLKNRVEQQLHEHLQDAMSFLKDVCEQSRMEDRLDRLDDAIHVLRNHAVGPSTSLPAGHSDIHSLLGPSHNAPIGSLNSNYGGSSLVASSRSASMVGTHREDSVSLNGNHSVLSSTVTTSSTDLNHKTQENYRGGLQSQSGTVVTTEIKTENKEKDENLHEPPSSDDMKSDDESSQKDIKVSSRGRTSSTNEDEDLNPEQKIEREKERRMANNARERLRVRDINEAFKELGRMCQLHLKSEKPQTKLLILHQAVAVILSLEQQVRERNLNPKAACLKRREEEKVSAVSAEPPTTLPGTHPGLSETTNPMGHM

SEQ ID NO: 92 - protein, 706 aa, Mus musculus
MNPQQQRMAAIGTDKELSDLLDFSAMFSPPVNSGKTRPTTLGSSQFSGSGMDERGGTTSWGTSGQPSPSYDSSRGFTDSPHYSDHLNDSRLGTHEGLSPTPFMNSNLIGKTSERGSFSLYSRDSGLSGCQSSLLRQDLGLGSPAQLSSSGKPGTPYYSFSATSSRRRPLHDSVALDPLQAKKVRKVPPGLPSSVYAPSPNSDDFNRESPSYPSPKPPTSMFASTFFMQDGTHSSSDLWSSSNGMSQPGFGGILGTSTSHMSQSSSYGSLHSHDRLSYPPHSVSPTDINTSLPPMSSFHRGSTSSSPYVAASHTPPINGSDSILGTRGNAAGSSQTGDALGKALASIYSPDHTSSSFPSNPSTPVGSPSPLTGTSQWPRAGGQAPSSPSYENSLHSLKNRVEQQLHEHLQDAMSFLKDVCEQSRMEDRLDRLDDAIHVLRNHAVGPSTSLPTSHSDIHSLLGPSHNASIGNLNSNYGGSSLVTNSRSASMVGTHREDSVSLNGNHSVLSSTVAASNTELNHKTPENFRGGVQNQSGSVVPTEIKTENKEKDENLHEPPSSDDMKSDDESSQKDIKVSSRGRTSSTNEDEDLNPEQKIEREKERRMANNARERLRVRDINEAFKELGRMCQLHLKSEKPQTKLLILHQAVAVILSLEQQVRERNLNPKAACLKRREEEKVSAASAEPPNTLPGAHPGLSESTNPMGHL

SEQ ID NO: 93 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 94 - protein, 755 aa, Mus musculus
MSQWFELQQLDSKFLEQVHQLYDDSFPMEIRQYLAQWLEKQDWEHAAYDVSFATIRFHDLLSQLDDQYSRFSLENNFLLQHNIRKSKRNLQDNFQEDPVQMSMIIYNCLKEERKILENAQRFNQAQEGNIQNTVMLDKQKELDSKVRNVKDQVMCIEQEIKTLEELQDEYDFKCKTSQNREGEANGVAKSDQKQEQLLLHKMFLMLDNKRKEIIHKIRELLNSIELTQNTLINDELVEWKRRQQSACIGGPPNACLDQLQSWFTIVAETLQQIRQQLKKLEELEQKFTYEPDPITKNKQVLSDRTFLLFQQLIQSSFVVERQPCMPTHPQRPLVLKTGVQFTVKLRLLVKLQELNYNLKVKVSFDKDVNEKNTVKGFRKFNILGTHTKVMNMEESTNGSLAAEFRHLQLKEQKNAGNRTNEGPLIVTEELHSLSFETQLCQPGLVIDLEVFVPFQTTSLPVVVISNVSQLPSGWASILWYNMLVTEPRNLSFFLNPPCAWWSQLSEVLSWQFSSVTKRGLNADQLSMLGEKLLGPNAGPDGLIPWTRFCKENINDKNFSFWPWIDTILELIKKHLLCLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDDPKRTGYIKTELISVSEVHPSRLQTTDNLLPMSPEEFDEMSRIVGPEFDSMMSTV

SEQ ID NO: 95 - protein, 1073 aa, Homo sapiens
MHQPPESTAAAAAAADISARKMAHPAMFPRRGSGSGSASALNAAGTGVGSNATSSEDFPPPSLLQPPPPAASSTSGPQPPPPQSLNLLSQAQLQAQPLAPGGTQMKKKSGFQITSVTPAQISASISSNNSIAEDTESYDDLDESHTEDLSSSEILDVSLSRATDLGEPERSSSEETLNNFQEAETPGAVSPNQPHLPQPHLPHLPQQNVVINGNAHPHHLHHHHQIHHGHHLQHGHHHPSHVAVASASITGGPPSSPVSRKLSTTGSSDSITPVAPTSAVSSSGSPASVMTNMRAPSTTGGIGINSVTGTSTVNNVNITAVGSFNPNVTSSMLGNVNISTSNIPSAAGVSVGPGVTSGVNVNILSGMGNGTISSSAAVSSVPNAAAGMTGGSVSSQQQQPTVNTSRFRVVKLDSSSEPFKKGRWTCTEFYEKENAVPATEGVLINKVVETVKQNPIEVTSERESTSGSSVSSSVSTLSHYTESVGSGEMGAPTVVVQQQQQQQQQQQQQPALQGVTLQQMDFGSTGPQSIPAVSIPQSISQSQISQVQLQSQELSYQQKQGLQPVPLQATMSAATGIQPSPVNVVGVTSALGQQPSISSLAQPQLPYSQAAPPVQTPLPGAPPPQQLQYGQQQPMVSTQMAPGHVKSVTQNPASEYVQQQPILQTAMSSGQPSSAGVGAGTTVIPVAQPQGIQLPVQPTAVPAQPAGASVQPVGQAPAAVSAVPTGSQIANIGQQANIPTAVQQPSTQVPPSVIQQGAPPSSQVVPPAQTGIIHQGVQTSAPSLPQQLVIASQSSLLTVPPQPQGVEPVAQGIVSQQLPAVSSLPSASSISVTSQVSSTGPSGMPSAPTNLVPPQNIAQTPATQNGNLVQSVSQPPLIATNTNLPLAQQIPLSSTQFSAQSLAQAIGSQIEDARRAAEPSLVGLPQTISGDSGGMSAVSDGSSSSLAASASLFPLKVLPLTTPLVDGEDESSSGASVVAIDNKIEQAMDLVKSHLMYAVREEVEVLKEQIKELIEKNSQLEQENNLLKTLASPEQLAQFQAQLQTGSPPATTQPQGTTQPPAQPASQGSGPTA

SEQ ID NO: 96 - protein, 1077 aa, Mus musculus
MHQPPESTAAAAAAADISARKMAHPAMFPRRGSGGGSASALNAAGTGVSGAAPSSEDFPPPSLLQPPPPAASSTQGPQPPPPQSLNLLSQAQLQGQPLAPGGTQMKKKSGFQITSVTPAQISASISSNNSIAEDTESYDDLDESHTEDLSSSEILDVSLSRATDLGEPERSSSEETLNNFQEAETPGAVSPNQPHLPQPHLPHLPQQNVVINGNAHPHHLHHHHHPHHGHHLHHGHHHSSHAAVAGPSIPGGPPSSPVSRKLSTTGSSDGGVPVAPPPAVPSSGLPASVMTNIRTPSTTGSLGINSVTGTSATNNVNIAAVGSFSPSVTNSVHGNANINTSNIPNAASISGGPGVTSVVNSSILSGMGNGTVSSSPVANSVLNAAAGITVGVVSSQQQQQQQQQPTVNTSRFRVVKLDSTSEPFKKGRWTCTEFYEKENAVPATEGVAVNKVVETVKQTPTEASSSERESTSGSSVSSSVSTLSHYTESVGSGEMMGAPAVVAPQQPPLPPAPPGLQGVALQQLEFSSPAPQSIAAVSMPQSISQSQMSQVQLQPQELSFQQKQTLQPVPLQATMSAATGIQPSPVSVVGITAAVGQQPSVSSLAQPQLPYSQTAPPVQTPLPGAPPQQLQYGQQQPMVPAQIAPGHGQPVTQNPTSEYVQQQQQPIFQAALSSGQSSSTGTGAGISVIPVAQAQGIQLPGQPTAVQTQPAGAAGQPIGQAQTAVSTVPTGGQIASIGQQANIPTAVQQPSTQVTPSVIQQGAPPSSQVVLPAPTGIIHQGVQTRASSLPQQLVIAPQSTLVTVPPQPQGVETVAQGVVSQQLPTGSPLPSASTISVTNQVSSAAPSGMPSVPTNLVPPQNIAQPPATQNGSLVQSVSQSPLIATNINLPLAQQIPLSSTQFSTQSLAQAIGSQMEDARRPAEPSLGGLPQTMSGDSGGMSAVSDGSSSSLAAPASLFPLKVLPLTTPLVDGEDESSGASVVAIDNKIEQAMDLVKSHLMYAVREEVEVLKEQIKELIEKNSQLEQENNLLKTLASPEQLAQFQAQLQTGSPPATTQPQGTTQPPAQPASQGSGSTA